(19) United States
(12) Patent Application Publication
(10) Pub. No.: US 2024/0382560 A1
(43) Pub. Date: Nov. 21, 2024

(54) METHODS AND COMPOSITIONS FOR TREATING CANCER AND ENHANCING IMMUNE CHECKPOINT INHIBITOR EFFICACY

(71) Applicants: Adeyemi Adesokan, Epalinges, Lausanne (CH); Romain Wyss, Epalinges, Lausanne (CH); Yong Miao, Epalinges, Lausanne (CH); Gnubiotics Sciences SA, Epalinges, Lausanne (CH)

(72) Inventors: Adeyemi Adesokan, Epalinges (CH); Romain Wyss, Epalinges (CH); Yong Miao, Epalinges (CH)

(73) Assignee: Gnubiotics Sciences SA, Epalinges, Lausanne (CH)

(21) Appl. No.: 18/691,864

(22) PCT Filed: Sep. 14, 2022
(86) PCT No.: PCT/IB2022/000515

§ 371 Date: Mar. 13, 2024

Related U.S. Application Data
(60) Provisional application No. 63/244,227, filed on Sep. 14, 2021.
(60) Provisional application No. 63/273,105, filed on Oct. 28, 2021.
(60) Provisional application No. 63/296,431, filed on Jan. 4, 2022.
(60) Provisional application No. 63/308,011, filed on Feb. 8, 2022.

(51) Int. Cl. A61K 38/17; A61K 39/395; A61P 35/00

(52) U.S. Cl. CPC A61K 38/1709; A61K 39/3955; A61P 35/00

(57) ABSTRACT

Provided herein are methods and compositions for treating a tumor or increasing the anti-cancer effectiveness of an immune checkpoint inhibitor using glycopeptide containing compositions.

### CROSS-REFERENCE TO RELATED PATENT APPLICATIONS

This application claims the benefit under 35 U.S.C. § 119(e) of U.S. Provisional Application Ser. No. 63/244,227, filed Sep. 14, 2021; U.S. Provisional Application Ser. No. 63/273,105, filed Oct. 28, 2021; U.S. Provisional Application Ser. No. 63/296,431, filed Jan. 4, 2022; and U.S. Provisional Application Ser. No. 63/308,011, filed Feb. 8, 2022, all of which are incorporated herein by reference in their entireties.

### BACKGROUND

Annually, cancer causes about 8.8 million deaths worldwide, accounting for about 15.7% of all deaths. Thus, new cancer treatments are needed. Recently, a number of immune checkpoint inhibitors have been developed that have been successful in increasing the lifespan of some cancer patients and, in some cases, causing permanent remission. However, a number of cancers are resistant or can become resistant to immune checkpoint inhibitors. Thus, therapies that enhance the efficacy of immune checkpoint inhibitors are needed.

### SUMMARY OF THE DISCLOSURE

Some aspects of the present disclosure are directed to a method of treating a tumor in a subject in need thereof comprising, or consisting essentially of, or consisting of administering to the subject a composition comprising, or consisting essentially of, or consisting of glycopeptides obtained from gastrointestinal mucins, wherein the composition comprises, or consists essentially of, or consists of less than about 25% (w/w) free glycans and wherein the composition comprises glycopeptides of multiple different oligosaccharide structures. In one aspect, the subject has been diagnosed with cancer, in one aspect, a cancer that is resistant or can become resistant to an immune checkpoint inhibitor. In a further aspect, the subject diagnosed with the cancer is treated. Subjects not diagnosed with the cancer do not receive the composition.

In some embodiments, the composition is administered in an amount effective to one or more of: increase T cells expansion, preferably tumor specific CD4 and CD8 T cell expansion and IFN-gamma-producing CD4 and CD8 T cell expansion; increase T cells trafficking and infiltration to the tumor, preferably CD4 and CD8 T cell, and IFN-gamma-producing CD4 and CD8 T cell, preferably CD8 T cell expansion; instruct T cells to target antigens on the tumor cells using glycans on the glycopeptides as molecular mimicry of glycans on cancer cells (glycosylation of tumor proteins generates neo-antigens that can serve as targets for tumor-specific T cells and these neo-antigens may share epitopes with GNU glycans through molecular mimicry); increase the level or activity of TNFα-producing CD8+ T cells, preferably tumor infiltrating TNFα-producing CD8+ T cells; increase the level or activity of TNFα-producing CD4+ T cells, preferably tumor infiltrating TNFα-producing CD4+ T cells; increase the level or activity of IL-17-producing CD4+ T cells (Th17), preferably tumor infiltrating IL-17-producing CD4+ T cells; increase the level of perforin, increase the level or excretion activity of perforin secreting CD8+ T cells, preferably tumor infiltrating CD8+ T cells; increase and stimulate antigen-presenting cells (e.g., dendritic cells (DCs)) to prime and activate T cells; promote a Th1-like immune tone favoring an anti-tumor response by modulating the microbiome composition and function and modulating the phenotype of immune cells (glycans and glycopeptides and specific bacteria modulated by glycans and glycopeptides can mediate an adjuvant effect on dendritic cells and other immune cells to trigger a type 1 interferon or an IL-12 fingerprint, which is associated with protective type 1 T helper cell (TH1 cell) and CD8+ T cell antitumor immune responses), induce pro-inflammatory cytokines or other molecules (e.g., IFN-gamma and granzyme B); bind and stimulate Toll-like Receptors (TLR) and initiate T-cell immunity by inducing maturation of DCs and by acting as co-stimulatory receptors on T cells; reduce expression of immune checkpoint molecules on T-cells or cancer cells; reduce expression of PD1 receptor on T cells and/or PD1-Ligand on cancer cells or reduce or eliminate interactions between PD1 receptor and PD1-Ligand; reduce expression of CTLA4 on CD4+ T cells and/or CD8+ T cells, preferably tumor infiltrating CD4+ T cells and/or CD8+ T cells; increase the level or activity of granzyme B producing CD8+ T cells; reduce immunosuppressor cells (e.g., Treg cells), myeloid-derived suppressor cells, and/or anti-inflammatory M2 macrophages, preferably tumor infiltrating immunosuppressor cells (e.g., Treg cells, FoxP3+ CD4 T cells), myeloid-derived suppressor cells, and/or anti-inflammatory M2 macrophages; reduce a level or activity of macrophages, preferably tumor infiltrating macrophages; increase a level or activity of pro-inflammatory M1 macrophages, preferably tumor infiltrating pro-inflammatory M1 macrophages, or a ratio of M1 macrophages to M2 macrophages; reduce immune-suppressive IL-10 and/or TGF-beta production in a tumor micro-environment (TME) and/or in systemic circulation; reduce the level or activity of RANTES in a tumor micro-environment (TME) and/or in systemic circulation; reduce or eliminate interactions between tumor-associated glycan and inhibitory immune receptors (e.g. lectin); reduce or eliminate galectin shedding by the tumor in a tumor micro-environment (TME) or reduce or eliminate interactions of tumor-shed galectin and glycans present on immune cells; bind to Dendritic Cell-Specific Intercellular adhesion molecule-3-Grabbing Non-integrin (DC-SIGN) and increase internalization and presentation of antigen by APC cells; produce microbiota-derived metabolites and/or microbiota-derived structural components or fragments thereof that activate anti-tumor immune cells or that directly affect tumor cells and the way they respond to T cells; and/or increase expression of chemokines, inflammasome-related genes, antigen presentation-related genes, and/or enhance the anti-tumor activity of a CAR-T therapy or Immune Checkpoint Inhibitor (ICI) therapy.

In some embodiments, the tumor to be treated is refractory to immune checkpoint inhibitor therapy. In some embodiments, the microenvironment of the tumor (TMI) is characterized by one or more of decreased T-cell, B-cell, and/or antigen-presenting cell function, decreased IL-2 production, generation of exhausted T cells, increased circulating soluble IL-2 receptor, and/or the presence of one or more immunosuppressive cell populations. In some embodiments, the total glycoprotein content of the composition is 12% or less (w/w). In some embodiments, the composition comprises, or consists essentially of, or consists of at least one sialylated glycopeptide-bound oligosaccharide. In some embodiments, administration of the composition comprising glycopeptides increases the sensitivity of the tumor to checkpoint-inhibitor therapy, decreases tumor weight, and/or decrease tumor volume. In some embodiments, the method further comprises administering an immune checkpoint inhibitor before, after, or simultaneously with the composition comprising glycopeptides. In some aspects, these clinical endpoints are measurements of treatment of the subject. Methods to determine such endpoints are known in the art and in some aspects, described herein.

In some embodiments, the composition is administered in an effective amount to reduce or prevent a rise in the level of Monocyte Chemoattractant Protein-1 (MCP-1) caused by the immune checkpoint inhibitor in a tumor micro-environment (TME) and/or in systemic circulation. In some aspects, these clinical endpoints are measurements of treatment of the subject. Methods to determine such endpoints are known in the art and in some aspects, described herein.

In some embodiments, the total protein content of the composition that is administered in the above methods is 12% or less (w/w) (e.g., 12%, 11%, 10%, 9%, 8%, 7%, 6%, 5%, 4%, 3%, 2%, 1%, or lower). In some embodiments, the composition that is administered in the above methods comprises, or consists essentially of, or consists of at least one sialylated glycopeptide-bound oligosaccharide.

In some embodiments, the composition is obtained from porcine gastric mucins, wherein the composition is obtained without subjecting the mucins or a partially purified fraction thereof to conditions or reagents that cause complete release of oligosaccharides from glycopeptides; and wherein the total oligosaccharide content of the composition is greater than about 10% (w/w); the ratio of glycopeptides:free glycans is greater than about 4:1 (w/w) (e.g., 5:1, 6:1, 7:1, 8:1, 9:1, 10:1 or higher); and the total protein content of the composition is about 12% or less (w/w) (e.g., 12%, 11%, or lower).

In some embodiments, the composition comprises at least one glycopeptide-bound oligosaccharide having a general formulae selected from Hex1HexNAc1, HexNAc2, Hex1HexNAc1Sul1, HexNAc1deHex1Sul1, HexNAc2Sul1, NeuAc1HexNAc1, Hex2HexNAc1, Hex1HexNAc1deHex1, Hex1HexNAc2, HexNAc2deHex1, Hex1HexNAc1deHex1Sul1, Hex1HexNAcSul1, Hex2HexNAc1deHex1, NeuAc1Hex1HexNAc1, Hex1HexNAc1deHex2, Hex2HexNAc2, Hex1HexNAc2deHex1, Hex2HexNAc1deHex1Sul1, Hex1HexNAc1deHex2Sul1, Hex1HexNAc3, Hex1HexNAc2deHex1Sul1, NeuAc1Hex1HexNAc1deHex1, Hex2HexNAc2Sul1, NeuAc1Hex1HexNAc2, Hex1HexNAc3Sul1, Hex2HexNAc2deHex1, Hex1HexNAc2deHex2, Hex1HexNAc3deHex1, Hex2HexNAc3, Hex2HexNAc2deHex1Sul1, Hex1HexNAc4, Hex1HexNAc3deHex1Sul1, Hex3HexNAc2deHex1, NeuAc1Hex2HexNAc2, Hex2HexNAc2deHex2, Hex2HexNAc3deHex1, Hex3HexNAc2deHex1Sul1, Hex2HexNAc2deHex2Sul1, Hex2HexNAc4, Hex2HexNAc3deHex1Sul1, Hex3HexNAc3deHex1, Hex2HexNAc4deHex1, Hex3HexNAc3deHex1Sul1, and Hex4HexNAc3deHex1Sul1. In some embodiments, the composition comprises at least one or both of NeuAc1HexNAc1 and NeuAc1Hex1HexNAc1.

In some embodiments, the composition comprises glycopeptide-bound oligosaccharides having at least 10, at least 20, at least, 30, at least 40, at least 44, or all of the general formulae listed above. In some embodiments, each formula is present in the composition makes up between 0.1% and 10% (e.g., 0.1%, 0.2%, 0.3%, 0.4%, 0.5%, 0.6%, 0.7%, 0.8%, 0.9% 1.5%, 2%, 2.5%, 3%, 3.5%, 4%, 4.5%, 5%, 5.5%, 6%, 6.5%, 7%, 7.5%, 8%, 8.5%, 9%, 9.5%, 10%,) of all glycopeptide-bound oligosaccharides in the composition.

In some embodiments, the composition is identified herein as GBX and comprises at least one glycopeptide-bound oligosaccharide having a general formulae selected from Hex1HexNAc1, Hex1HexNAc1deHex1, Hex1HexNAc1deHex1Sul1, Hex1HexNAc1deHex2, Hex1HexNAc1deHex2Sul1, Hex1HexNAc1Sul1, Hex1HexNAc2, Hex1HexNAc2deHex1, Hex1HexNAc2deHex1Sul1, Hex1HexNAc2deHex2, Hex1HexNAc2Sul1, Hex1HexNAc3, Hex1HexNAc3deHex1, Hex1HexNAc3deHex1Sul1, Hex1HexNAc3Sul1, Hex1HexNAc4, Hex1HexNAcSul1, Hex2HexNAc1, Hex2HexNAc1deHex1, Hex2HexNAc1deHex1Sul1, Hex2HexNAc2, Hex2HexNAc2deHex1, Hex2HexNAc2deHex1Sul1, Hex2HexNAc2deHex2, Hex2HexNAc2deHex2Sul1, Hex2HexNAc2Sul1, Hex2HexNAc3, Hex2HexNAc3deHex1, Hex2HexNAc3deHex1Sul1, Hex2HexNAc4, Hex2HexNAc4deHex1, Hex3HexNAc2deHex1, Hex3HexNAc2deHex1Sul1, Hex3HexNAc3deHex1, Hex3HexNAc3deHex1Sul1, Hex4HexNAc3deHex1Sul1, HexNAc1deHex1Sul1, HexNAc2, HexNAc2deHex1, HexNAc2Sul1, NeuAc1Hex1HexNAc1, NeuAc1Hex1HexNAc1deHex1, NeuAc1Hex1HexNAc2, NeuAc1Hex2HexNAc2, and NeuAc1HexNAc1.

In some embodiments, the composition comprises glycopeptide-bound oligosaccharides having at least 10, at least 20, at least, 30, at least 40, at least 45, or all of the general formulae listed above. In some embodiments, the composition comprises at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%,) of all glycopeptide-bound oligosaccharides in the composition.

In some embodiments, the composition is identified herein as GCX and comprises at least one glycopeptide-bound oligosaccharide having a general formulae selected from Hex1HexNAc1, HexNAc2, Hex1HexNAc1Sul, Hex1NAc2Sul1, NeuAc1HexNAc1, NeuGc1HexNAc1, Hex1HexNAc1deHex1, Hex2HexNAc1, Hex1HexNAc2, HexNAc3, Hex1HexNAc2Sul1, NeuAc1Hex1HexNAc1, NeuGc1Hex1HexNAc1, Hex2HexNAc1deHex1, Hex1NAc3Sul1, NeuAc1HexNAc2, NeuGc1HexNAc2, Hex1HexNAc2deHex1, Hex2HexNAc2, Hex1HexNAc3, Hex1HexNAc2deHex1Sul1, NeuAc1Hex1HexNAc1deHex1, Hex2HexNAcSul1, NeuGc1Hex1HexNAc1deHex1, Hex1HexNAc3Sul1, NeuAc1Hex1HexNAc2, NeuGc1Hex1HexNAc2, Hex2HexNAc2deHex1, Hex3HexNAc2, Hex1HexNAc3deHex1, Hex2HexNAc3, NeuAc1Hex1HexNAc2Sul1, NeuAc2Hex1HexNAc1, NeuGc1Hex1HexNAc2Sul1, Hex2HexNAc2deHex1Sul1, NeuAc1NeuGc1Hex1HexNAc1, NeuGc2Hex1HexNAc1, Hex1HexNAc3deHex1Sul1, NeuAc1Hex1HexNAc2deHex1, Hex2HexNAc3Sul1, NeuGc1Hex1HexNAc2deHex1, NeuAc1Hex2HexNAc2, Hex2HexNAc2deHex2, NeuGc1Hex2HexNAc2, Hex2HexNAc3deHex1, NeuAc1Hex2HexNAc2Sul1, Hex2HexNAc2deHex2Sul1, NeuGc1Hex2HexNAc2Sul1, Hex1HexNAc4deHex1, NeuAc1Hex1HexNAc3Sul1, Hex1HexNAc3deHex2Sul1, NeuAc2Hex1HexNAc2, NeuGc1Hex1HexNAc3Sul1, Hex2HexNAc3deHex1Sul1, NeuAc1Hex2HexNAc2deHex1, NeuGc2Hex1HexNAc2, NeuGc1Hex2HexNAc2deHex1, NeuGc1Hex3HexNAc2, Hex1HexNAc4deHex1 Sul1, NeuAc1Hex1HexNAc3deHex1, Hex2HexNAc3deHex2 NeuGc1Hex2HexNAc3, NeuAc1Hex2HexNAc2deHex1Sul1, Hex2HexNAc3deHex2Sul1, NeuAc1Hex2HexNAc3deHex1Sul1, NeuGc1Hex2HexNAc3deHex1Sul1, Hex2HexNAc4deHex2Sul1, Hex2HexNAc5deHex2Sul1, NeuAc1Hex5HexNAc4deHex1, and NeuAc1Hex4HexNAc4deHex2Sul1.

In some embodiments, the composition comprises glycopeptide-bound oligosaccharides having at least 10, at least 20, at least, 30, at least 40, at least 45, at least 50, at least 55, at least 60, at least 65, (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65) or all of the general formulae listed above. In some embodiments, the composition comprises at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%,) of all glycopeptide-bound oligosaccharides in the composition.

In some embodiments, the composition comprises glycopeptide-bound oligosaccharides having the following formulae: Hex1HexNAc1deHex1, Hex1HexNAc3deHex1Sul1, Hex1NAc3Sul1, NeuAc1Hex1HexNAc1, Hex1HexNAc2deHex1, NeuAc1Hex1HexNAc1deHex1, Hex1HexNAc3deHex1Sul1, NeuAc1Hex1HexNAc1, Hex1HexNAc2deHex1Sul1, Hex1HexNAc2Sul1, Hex1HexNAc1, and Hex1HexNAc3Sul1. In some embodiments, each of the fourmulae represents between about 3% and 10% (e.g., about 3%, 4%, 5%, 6%, 7%, 8%, 9%, or 10%) of all glycopeptide-bound oligosaccharides in the composition.

In some embodiments, the composition comprises at least one glycopeptide-bound oligosaccharide having a structure selected from Galβ1-3GalNAcol, GlcNAcβ1-4Galol, GlcNAcα1-4Galol, HexNAc-GlcAol, GlcNAcβ1-6GalNAcol, Galβ1-4(6S)GlcNAcol, 6SGlcNAc-Fucol, Galβ1-4(6S)GlcNAcol, (S)Galβ1-GlcNAcol, (S)Galβ1-GlcNAcol, 6SGlcNAcβ1-6GalNAcol, 6SGlcNAcβ1-3GalNAcol, NeuAc-HexNAcol, NeuAcα2-6GalNAcol, Galβ1-4GlcNAcβ1-3Gal minus H₂O, Fucα1-2(GalNAcα1-3)Galol, Gal-GlcNAc-Fucol, Gal-GlcNAc-Fucol, Fucα1-2Galβ1-4GlcNAcol, Fucα1-2Galβ1-3GlcNAcol, Fucα1-2Galβ1-3GalNAcol, Galβ1-4GlcNAcβ1-3Gal, GlcNAcα1-4Galβ1-4GlcNAc minus H₂O, GlcNAc-GlcNAc-Fucol, Galβ1-3(GlcNAcβ1-6)GalNAcol, GlcNAcα1-4Galβ1-3GalNAcol, GlcNAcα1-4Galβ1-4GlcNAcol, Fucα1-2Galβ1-4(6S)GlcNAcol, Galβ1-4(Fucα1-3)(6S)GlcNAcol, 6SGalβ1-3(Fucα1-4)GlcNAcol, SGalβ1-3(GlcNAcβ1-6)GalNAcol, Galβ1-3(6SGlcNAcβ1-6)GalNAcol, Galβ1-4(6S)GlcNAcβ1-6GalNAcol, Fucα1-2Galβ-4GlcNAcβ1-3Gal minus H₂O, Galβ1-3(NeuAcα2-6)GalNAcol, NeuAcα2-3Galβ1-3GalNAcol, Fucα1-2Galβ1-4(Fucα1-3)GlcNAcol, Fucα1-2Galβ-4GlcNAcβ1-3Gal, GlcNAcα1-4Galβ1-4GlcNAcβ1-3Gal minus H₂O, Fucα1-2(GalNAcα1-3)Galβ1-4GlcNAcol, Fucα1-2(GalNAcα1-3)Galβ1-3GalNAcol, GlcNAcα1-4Gal(Fuc)GlcNAcol, GlcNAcα1-4Gal(Fuc)GlcNAcol, Fucα1-2Galβ1-3(GlcNAcβ1-6)GalNAcol, GlcNAcα1-4Galβ1-4GlcNAcβ1-3Gal, Galβ1-3(Galβ1-4GlcNAcβ1-6)GalNAcol, GlcNAcα1-4Galβ1-4GlcNAcβ1-3Galol, Galβ1-3(Galβ1-4GlcNAcβ1-6)GalNAcol, Galβ1-4GlcNAcβ1-3Galβ1-3GalNAcol, Fucα1-2Galβ1-4(6S)GlcNAcβ1-4Gal minus H₂O, Fucα1-2(S)Galβ1-4GlcNAcβ1-4Gal minus H₂O, Fucα1-2Gal(Fuc)(6S)GlcNAcol, Fucα1-2Galβ1-3(6S)GlcNAcβ1-4Galol, GlcNAcα1-4Galβ1-3(GlcNAcβ1-6)GalNAcol, GlcNAcα1-3Galβ1-4GlcNAcβ1-6GalNAcol, Fucα1-2(GalNAcα1-3)Galβ1-4(6S)GlcNAcol, Fucα1-2Galβ1-4(6S)GlcNAcβ1-3GalNAcol, Fucα1-2Galβ1-3(6S-GlcNAcβ1-6)GalNAcol, Fucα1-2Galβ1-3(NeuAcα2-6)GalNAcol, Galβ1-3[Galβ1-4(6S)GlcNAcβ1-6]GalNAcol, GlcNAcα1-4Galβ1-3(NeuAcα2-6)GalNAcol, GlcNAcα1-4Galβ1-3[(6S)GlcNAcβ1-6]GalNAcol, GalNAcα1-3(Fucα1-2)Galβ1-4GlcNAcβ1-3Gal minus H₂O, GlcNAcα1-4Galβ1-3(NeuAcα2-6)GalNAcol, Fucα1-2(GalNAcα1-3)Gal-(Fuc)GlcNAcol, Galβ1-3(Fucα1-4)GlcNAcβ1-3Galβ1-3GalNAcol, Galβ1-4(Fucα1-3)GlcNAcβ1-3Galβ1-3GalNAcol, Fucα1-2Galβ1-4GlcNAcβ1-3Galβ1-4GlcNAcol, Galβ1-3(Fucα1-2Galβ1-4GlcNAcβ1-6)GalNAcol, Fucα1-2Galβ1-3(Galβ1-4GlcNAcβ1-6)GalNAcol, Fucα1-2(GalNAcα1-3)Galβ1-4GlcNAcβ1-6GalNAcol, Fucα1-2Galβ1-3(GalNAcβ1-4GlcNAcβ1-6)GalNAcol, Galβ1-4GlcNAcβ1-3(GlcNAcβ1-6)Galβ1-3GalNAcol, GlcNAcα1-4Galβ1-3(Galβ1-4GlcNAcβ1-6)GalNAcol, GalNAcα1-3(Fucα1-2)Galβ1-4(6S)GlcNAcβ1-3Gal minus H₂O, Fucα1-2Galβ1-4(6S)GlcNAcβ1-3Galβ1-3GalNAcol, Fucα1-2Galβ1-3[Galβ1-4(6S)GlcNAcβ1-6]GalNAcol, GlcNAcα1-4Galβ1-3(GalNAcβ1-4GlcNAcβ1-6)GalNAcol, GlcNAcβ1-3[Fucα1-2Galβ1-4(6S)GlcNAcβ1-6]GalNAcol, GalNAcα1-3(Fucα1-2)Galβ1-3(6SGlcNAcβ1-6)GalNAcol, Fucα1-2Galβ1-4GlcNAcβ1-3(Galβ1-4GlcNAcβ1-6)Gal, Fucα1-2Galβ1-4GlcNAcβ1-3(Galβ1-4GlcNAcβ1-6)Gal, Galβ1-4GlcNAcβ1-3Galβ1-3(NeuAcα2-6)GalNAcol, Fucα1-2Galβ1-3(Fucα1-2Galβ1-4GlcNAcβ1-6)GalNAcol, Galβ1-3[Fucα1-2(GalNAcα1-3)Galβ1-4GlcNAcβ1-6]GalNAcol, GlcNAcα1-4Galβ1-3(Fucα1-2Galβ1-4GlcNAcβ1-6)GalNAcol, Fucα1-2Galβ1-3(GlcNAcα1-4Galβ1-4GlcNAcβ1-6)GalNAcol, Fucα1-2Galβ1-4(6S)GlcNAcβ1-3(Galβ1-4GlcNAcβ1-6)Gal minus H₂O, Fucα1-2Galβ1-3[Fucα1-2Galβ1-4(6S)GlcNAcβ1-6]GalNAcol, GlcNAcα1-4Galβ1-3(GlcNAcα1-4Galβ1-4GlcNAcβ1-6)GalNAcol, Gal-GlcNAcβ1-3(GlcNAcα1-4Galβ1-4GlcNAcβ1-6)GalNAcol, GalNAcα1-3(Fucα1-2)Galβ1-4(6S)GlcNAcβ1-3Galβ1-3GalNAcol, Fucα1-2Galβ1-4GlcNAcβ1-3Galβ1-4GlcNAcβ1-3Galβ1-3GalNAcol, GlcNAcα1-4Galβ1-3[GalNAcα1-3(Fucα1-2)Galβ1-4GlcNAcβ1-6]GalNAcolFucα1-2Galβ1-4(6S)GlcNAcβ1-3Galβ1-4GlcNAcβ1-3Galβ1-4GlcNAcol, and Fucα1-2Galβ1-4(6S)GlcNAcβ1-3Galβ1-4GlcNAcβ1-3(Galβ1-4GlcNAcβ1-6)Gal minus H₂O. In some embodiments, the composition comprises Galβ1-3GalNAcol (T antigen) and NeuAcα2-3Galβ1-3GalNAcol (Sialyl T antigen). In some embodiments, the composition comprises Galβ1-3(Fucα1-4)GlcNAcβ1-3Galβ1-3GalNAcol (Lewis A) and Galβ1-4(Fucα1-3)GlcNAcβ1-3Galβ1-3GalNAcol (Lewis X).

In some embodiments, the composition comprises at least 10 different glycopeptide-bound oligosaccharides having a structure selected from Galβ1-3GalNAc, GlcNAcα1-4Gal, GlcNAcβ1-4Gal, Fucα1-2(GalNAcα1-3)Gal, Fucα1-2Galβ1-3GalNAc, Fucα1-2Galβ1-3GlcNAc, Fucα1-2Galβ1-4GlcNAc, Gal-GlcNAc-Fuc, 6SGalβ1-3(Fucα1-4)GlcNAc, Fucα1-2Galβ1-4(6S)GlcNAc, Galβ1-4(Fucα1-3)(6S)GlcNAc, Fucα1-2Galβ1-4(Fucα1-3)GlcNAc, Fucα1-2Gal(Fuc)(6S)GlcNAc, (S)Galβ1-GlcNAc, Galβ1-4(6S)GlcNAc, Galβ1-3(GlcNAcβ1-6)GalNAc, GlcNAcα1-4Galβ1-3GalNAc, GlcNAcα1-4Galβ1-4GlcNAc, Fucα1-2(GalNAcα1-3)Galβ1-3GalNAc, Fucα1-2(GalNAcα1-3)Galβ1-4GlcNAc, Fucα1-2Galβ1-3(GlcNAcβ1-6)GalNAc, GlcNAcα1-4Gal(Fuc)GlcNAc, Fucα1-2(GalNAcα1-3)Galβ1-4(6S)GlcNAc, Fucα1-2Galβ1-3(6S-GlcNAc(31-6)GalNAc, Fucα1-2Galβ1-4(6S)GlcNAcβ1-3GalNAc, Fucα1-2(GalNAcα1-3)Gal-(Fuc)GlcNAc, Galβ1-3(6SGlcNAcβ1-6)GalNAc, GlcNAcα1-3Galβ1-4GlcNAcβ1-6GalNAc, GlcNAcα1-4Galβ1-3(GlcNAcβ1-6)GalNAc, Fucα1-2(GalNAcα1-3)Galβ1-4GlcNAc(31-6GalNAc, Fucα1-2Galβ1-3(GalNAcβ1-4GlcNAcβ1-6)GalNAc, GalNAcα1-3(Fucα1-2)Galβ1-3(6SGlcNAcβ1-6)GalNAc, GlcNAcβ1-3[Fucα1-2Galβ1-4(6S)GlcNAcβ1-6]GalNAc, GlcNAcα1-4Galβ1-3[(6S)GlcNAcβ1-6]GalNAc, GlcNAcα1-4Galβ1-3(GalNAcβ1-4GlcNAcβ1-6)GalNAc, Galβ1-4(6S)GlcNAcβ1-6GalNAc, SGalβ1-3(GlcNAcβ1-6)GalNAc, Galβ1-4GlcNAcβ1-3Gal, Fucα1-2Galβ-4GlcNAcβ1-3Gal, Fucα1-2(S)Galβ1-4GlcNAcβ1-4Gal, Fucα1-2Galβ1-3(6S)GlcNAcβ1-4Gal, Fucα1-2Galβ1-4(6S)GlcNAcβ1-4Gal, Galβ1-3(Galβ1-4GlcNAcβ1-6)GalNAc, Galβ1-4GlcNAcβ1-3Galβ1-3GalNAc, GlcNAcα1-4Galβ1-4GlcNAcβ1-3Gal, Fucα1-2Galβ1-3(Galβ1-4GlcNAcβ1-6)GalNAc, Fucα1-2Galβ1-4GlcNAcβ1-3Galβ1-4GlcNAc, GalNAcα1-3(Fucα1-2)Galβ1-4GlcNAcβ1-3Gal, Galβ1-3(Fucα1-2Galβ1-4GlcNAcβ1-6)GalNAc, Galβ1-3(Fucα1-4)GlcNAcβ1-3Galβ1-3GalNAc, Galβ1-4(Fucα1-3)GlcNAcβ1-3Galβ1-3GalNAc, Fucα1-2Galβ1-3[Galβ1-4(6S)GlcNAcβ1-6]GalNAc, Fucα1-2Galβ1-4(6S)GlcNAcβ1-3Galβ1-3GalNAc, GalNAcα1-3(Fucα1-2)Galβ1-4(6S)GlcNAcβ1-3Gal, Fucα1-2Galβ1-3(Fucα1-2Galβ1-4GlcNAcβ1-6)GalNAc, Fucα1-2Galβ1-3[Fucα1-2Galβ1-4(6S)GlcNAcβ1-6]GalNAc, Galβ1-3[Galβ1-4(6S)GlcNAcβ1-6]GalNAc, Galβ1-4GlcNAcβ1-3(GlcNAcβ1-6)Galβ1-3GalNAc, GlcNAcα1-4Galβ1-3(Galβ1-4GlcNAcβ1-6)GalNAc, Fucα1-2Galβ1-3(GlcNAcα1-4Galβ1-4GlcNAcβ1-6)GalNAc, Galβ1-3[Fucα1-2(GalNAcα1-3)Galβ1-4GlcNAcβ1-6]GalNAc, GlcNAcα1-4Galβ1-3(Fucα1-2Galβ1-4GlcNAcβ1-6)GalNAc, GalNAcα1-3(Fucα1-2)Galβ1-4(6S)GlcNAcβ1-3Galβ1-3GalNAc, Gal-GlcNAcβ1-3(GlcNAcα1-4Galβ1-4GlcNAcβ1-6)GalNAc, GlcNAcα1-4Galβ1-3(GlcNAcα1-4Galβ1-4GlcNAcβ1-6)GalNAc, GlcNAcα1-4Galβ1-3[GalNAcα1-3(Fucα1-2)Galβ1-4GlcNAcβ1-6]GalNAc, Fucα1-2Galβ1-4GlcNAcβ1-3(Galβ1-4GlcNAcβ1-6)Gal, Fucα1-2Galβ1-4(6S)GlcNAcβ1-3(Galβ1-4GlcNAcβ1-6)Gal, Fucα1-2Galβ1-4GlcNAcβ1-3Galβ1-4GlcNAcβ1-3Galβ1-3GalNAc, Fucα1-2Galβ1-4(6S)GlcNAcβ1-3Galβ1-4GlcNAcβ1-3Galβ1-4GlcNAc, Fucα1-2Galβ1-4(6S)GlcNAcβ1-3Galβ1-4GlcNAcβ1-3(Galβ1-4GlcNAcβ1-6)Gal, 6SGlcNAc-Fuc, HexNAc-GlcA, GlcNAcβ1-6GalNAc, GlcNAc-GlcNAc-Fuc, 6SGlcNAcβ1-3GalNAc, 6SGlcNAcβ1-6GalNAc, Galβ1-3(NeuAcα2-6)GalNAc, NeuAcα2-3Galβ1-3GalNAc, Fucα1-2Galβ1-3(NeuAcα2-6)GalNAc, GlcNAcα1-4Galβ1-3(NeuAcα2-6)GalNAc, Galβ1-4GlcNAcβ1-3Galβ1-3(NeuAcα2-6)GalNAc, NeuAc-HexNAc, and NeuAcα2-6GalNAc.

In some embodiments, the composition comprises at least 10 glycopeptide-bound oligosaccharides having a structure selected from Galβ1-3GalNAc, GalNAcα1-3GalNAc, GlcNAcβ1-6GalNAc, 3SGalβ1-3GalNAc, 6SGlcNAcβ1-3GalNAc, 6SGlcNAcβ1-6GalNAc, NeuAcα2-6GalNAc, NeuGcα2-6GalNAc, Fucα1-2(GalNAcα1-3)Gal, Fucα1-2Galβ1-3GalNAc, Fucα1-2Galβ1-4GlcNAc, Galβ1-4GlcNAcβ1-3Gal, Galβ1-3(GlcNAcβ1-6)GalNAc, Galβ1-4GlcNAcβ1-3GalNAc, GlcNAcβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Fucα1-2Galβ-4GlcNAcβ1-3Gal, 6SGlcNAcβ1-3(GlcNAcβ1-6)GalNAc, GlcNAcβ1-3(6S-GlcNAcβ1-6)GalNAc, GalNAcα1-3(NeuAcα2-6)GalNAc, GlcNAcβ1-3(NeuAcα2-6)GalNAc, GalNAcα1-3(NeuGcα2-6)GalNAc, GlcNAcβ1-3(NeuGcα2-6)GalNAc, Fucα1-2(GalNAcα1-3)Galβ1-3GalNAc, Fucα1-2Galβ1-3(GlcNAcβ1-6)GalNAc, Fucα1-2Galβ1-3GlcNAcβ1-3GalNAc, Galβ1-3(Galβ1-4GlcNAcβ1-6)GalNAc, Galβ1-3GlcNAcβ1-3Galβ1-3GalNAc, GlcNAcβ1-3(Galβ1-4GlcNAcβ1-6)GalNAc, Fucα1-2Galβ1-3(6S-GlcNAcβ1-6)GalNAc, Fucα1-2Galβ1-3(NeuAcα2-6)GalNAc, Galβ1-3[Galβ1-4(6S)GlcNAcβ1-6]GalNAc, Fucα1-2Galβ1-3(NeuGcα2-6)GalNAc, Galβ1-4GlcNAcβ1-3(6SGlcNAcβ1-6)GalNAc, GlcNAcα1-4Galβ1-3[(6S)GlcNAcβ1-6]GalNAc, GlcNAcβ1-3[Galβ1-4(6S)GlcNAcβ1-6]GalNAc, GlcNAcα1-4Galβ1-3(NeuAcα2-6)GalNAc, NeuAcα2-3(GalNAcβ1-4)Galβ1-3GalNAc, GalNAcβ1-4(NeuGcα2-3)Galβ1-3GalNAc, NeuGcα2-3Galβ1-3(GlcNAcβ1-6)GalNAc, Fucα1-2Galβ1-3(Galβ1-4GlcNAcβ1-6)GalNAc, Galβ1-3(Fucα1-2Galβ1-4GlcNAcβ1-6)GalNAc, Galα1-3Galβ1-4GlcNAcβ1-3Galβ1-3GalNAc, Galβ1-3(Galα1-3Galβ1-4GlcNAcβ1-6)GalNAc, Fucα1-2(GalNAcα1-3)Galβ1-4GlcNAcβ1-3GalNAc, GlcNAcβ1-3(Fucα1-2Galβ1-4GlcNAcβ1-6)GalNAc, GlcNAcα1-4Galβ1-3(Galβ1-4GlcNAcβ1-6)GalNAc, NeuAcα2-3Galβ1-3[(6S)GlcNAcβ1-6]GalNAc, NeuAcα2-3Galβ1-3(NeuAcα2-6)GalNAc, NeuGcα2-6Galβ1-3(6SGlcNAcβ1-6)GalNAc, Fucα1-2Galβ1-3[Gal 01-4(6S)GlcNAcβ1-6]GalNAc, Galβ1-3[Fucα1-2Galβ1-4(6S)GlcNAcβ1-6]GalNAc, NeuAcα2-3Galβ1-3(NeuGcα2-6)GalNAc, NeuGcα2-3Galβ1-3(NeuGcα2-6)GalNAc, Fucα1-2Galβ1-4(6S)GlcNAcβ1-3(GlcNAcβ1-6)GalNAc, Fucα1-2Galβ1-4GlcNAcβ-3(6SGlcNAcβ1-6)GalNAc, GalNAcα1-3(Fucα1-2)Galβ1-3(6SGlcNAcβ1-6)GalNAc, GlcNAcβ1-3[Fucα1-2Galβ1-4(6S)GlcNAcβ1-6]GalNAc, Fucα1-2Galβ1-4GlcNAcβ1-3(NeuAcα2-6)GalNAc, Galβ1-4GlcNAcβ1-3[Galβ1-4(6S)GlcNAcβ1-6]GalNAc, Fucα1-2Galβ1-4GlcNAcβ1-3(NeuGcα2-6)GalNAc, NeuAcα2-3Galβ1-3(Galβ1-4GlcNAcβ1-6)GalNAc, Fucα1-2Galβ1-3(Fucα1-2Galβ1-4GlcNAcβ1-6)GalNAc, NeuGcα2-3Galβ1-3(Galβ1-4GlcNAcβ1-6)GalNAc, Galβ1-3[Fucα1-2(GalNAcα1-3)Galβ1-4GlcNAcβ1-6]GalNAc, NeuAcα2-3Galβ1-3[Galβ1-4(6S)GlcNAcβ1-6]GalNAc, Fucα1-2Galβ1-3[Fucα1-2Galβ1-4(6S)GlcNAcβ1-6]GalNAc, NeuGcα2-3Galβ1-3[Galβ1-4(6S)GlcNAcβ1-6]GalNAc, GlcNAcβ1-3[GalNAcα1-3(Fucα1-2)Galβ1-3GlcNAcβ1-6]GalNAc, NeuAcα2-3(GalNAcβ1-4)Galβ1-3(6SGlcNAcβ1-6)GalNAc, 6SGlcNAcβ1-3[Fucα1-2Galβ1-(Fucα1-)GlcNAcβ1-6]GalNAc, NeuAcα2-3(GalNAcβ1-4)Galβ1-3(NeuAcα2-6)GalNAc, GlcNAcβ1-3[NeuGcα2-3Galβ1-4(6S)GlcNAcβ1-6]GalNAc, Fucα1-2Galβ1-4GlcNAcβ1-3[Galβ1-4(6S)GlcNAcβ1-6]GalNAc, Galβ1-3[GalNAcα1-3(Fucα1-2)Galβ1-4(6S)GlcNAcβ1-6]GalNAc, Galβ1-3GlcNAcβ1-3[Fucα1-2Galβ1-4(6S)GlcNAcβ1-6]GalNAc, Galβ1-4GlcNAcβ1-3[Fucα1-2Galβ1-3(6S)GlcNAcβ1-6]GalNAc, NeuAcα2-3Galβ1-3(Fucα1-2Galβ1-4GlcNAcβ1-6)GalNAc, NeuGcα2-3(GalNAcβ1-4)Galβ1-3(NeuGcα2-6)GalNAc, NeuGcα2-3Galβ1-3 (Fucα1-2Galβ1-4GlcNAcβ1-6)GalNAc, Galα1-3Galβ1-3(NeuGcα2-3Galβ1-4GlcNAcβ1-6)GalNAc, GalNAcα1-3(Fucα1-2)Galβ1-3GlcNAcβ1-3(6SGlcNAcβ1-6)GalNAc, GlcNAcβ1-3[GalNAcα1-3(Fucα1-2)Galβ1-(6S)GlcNAcβ1-6]GalNAc, GlcNAcα1-4(Fucα1-2)Galβ1-4GlcNAcβ1-3(NeuAcα2-6)GalNAc, Fucα1-2Galβ1-4GlcNAcβ1-3(Fucα1-2Galβ1-4GlcNAcβ1-6)GalNAc, Galβ1-(Fuc)GlcNAcβ1-3(Fucα1-2Galβ1-4GlcNAcβ1-6)GalNAc, NeuGcα2-3Galβ1-3(GlcNAcα1-4Galβ1-4GlcNAcβ1-6)GalNAc, NeuAcα2-3Galβ1-3[Fucα1-2Galβ1-4(6S)GlcNAcβ1-6]GalNAc, Fucα1-2Galβ1-3[GalNAcα1-3(Fucα1-2)Galβ1-3(6S)GlcNAcβ1-6]GalNAc, Fucα1-2Galβ1-3[GalNAcα1-3(Fucα1-2)Galβ1-4(6S)GlcNAcβ1-6]GalNAc, Fucα1-2Galβ1-4GlcNAcβ1-3[Fucα1-2Galβ1-4(6S)GlcNAcβ1-6]GalNAc, NeuAcα2-3Galβ1-3[GalNAcα1-3(Fucα1-2)Gal-(6S)GlcNAcβ1-6]GalNAc, GlcNAcα1-4Galβ1-3[NeuGcα2-6Gal-(Fuc)(6S)GlcNAcβ1-6]GalNAc, GlcNAcα1-4(Fucα1-2)GlcNAcβ1-3[Fucα1-2)Galβ1-4(6S)GlcNAcβ1-6]GalNAc, GalNAcα1-3(Fucα1-2)Galβ1-4GlcNAcβ1-3[GalNAcα1-3(Fucα1-2)Galβ1-4(6S)GlcNAcβ1-6]GalNAc, and NeuAcα2-Galβ1-4GlcNAcβ1-2Manα1-3(Galβ1-4GlcNAcβ1-2Manα1-6)Manβ1-4GlcNAcβ1-4(Fucα1-6)GlcNAc.

In some embodiments, the composition comprises glycopeptide-bound oligosaccharides having at least 10, at least 20, at least 50, at least 75, or all of the structures provided above (e.g., the composition comprises at least 10, 20, 50, 75, or 99 glycopeptides, wherein each of the glycopeptides comprises a different glycopeptide-bound oligosaccharide from the above list).

In some embodiments, the composition has a salt content of less than about 2%. In some embodiments, the composition has a pH of less than 7.5 (e.g., pH 7.4, 7.3, 7.2, 7.1, 7, 6.9, 6.8, 6.7, 6.6, 6.5, 6.4, 6.3, 6.2, 6.1, or 6). In some embodiments, the composition has a free glycan content of less than 0.1% by weight.

In some embodiments, the composition comprising a mixture of glycopeptides is obtained from porcine intestinal mucins or a partially purified fraction thereof, wherein: the composition is obtained without subjecting the mucins or the partially purified fraction thereof to conditions or reagents that release oligosaccharides from glycopeptides; the composition has a total glycan content of greater than about 10%; the composition comprises glycopeptide-bound oligosaccharides having each of the following general formulae: Hex1HexNAc1, HexNAc2, NeuAc1HexNAc1, NeuGc1HexNAc1, Hex1HexNAc1Fuc1, Hex1HexNAc2, Hex1HexNAc2Sul1, NeuAc1Hex1HexNAc1, NeuGc1Hex1HexNAc1, NeuAc1HexNAc2, NeuGc1HexNAc2, Hex1HexNAc2Fuc1, Hex1HexNAc2Fuc1Sul1, NeuAc1Hex1HexNAc1Fuc1, Hex1HexNAc3Sul1, Hex2HexNAc2Fuc1, Hex1HexNAc3Fuc1Sul1, Hex2HexNAc2Fuc2Sul1, and the composition does not substantially contain insoluble particles having a diameter greater than 7 μm or compounds having a molecular weight of greater than about 3 kDa. In some embodiments, the composition comprises glycopeptide-bound oligosaccharides having each of the following general formulae: Hex2HexNAc3deHex1Sul2, Hex2HexNAc3deHex1Sul2, NeuGc1Hex2HexNAc3, NeuAc1Hex2HexNAc2deHex1Sul1, Hex2HexNAc4Sul2, Hex2HexNAc4Sul2, NeuAc1Hex2HexNAc3Sul1, Hex2HexNAc3deHex2Sul1, Hex2HexNAc3deHex2Sul1, Hex2HexNAc3deHex2Sul1, Hex2HexNAc3deHex2sul1, Hex2HexNAc3deHex1Sul3, NeuGc1Hex2HexNAc3Sul1, NeuGc1Hex2HexNAc3Sul1, Hex2HexNAc3deHex2Sul2, Hex2HexNAc3deHex2Sul2, Hex2HexNAc3deHex2Sul2, Hex2HexNAc3deHex2Sul2, NeuGc1Hex2HexNAc3deHex1, NeuGc1Hex2HexNAc3Sul2, Hex3HexNAc3deHex1Sul2, NeuGc1Hex2HexNAc2deHex2Sul1, Hex2HexNAc4deHex1Sul2, NeuAc1Hex2HexNAc3deHex1Sul1, Hex2HexNAc3deHex3 Sul1, NeuGc1Hex2HexNAc3deHex1Sul1, NeuGc1Hex2HexNAc3deHex1Sul1, Hex3HexNAc3deHex2Sul1, NeuGc1Hex2HexNAc2deHex2Sul2, NeuGc1Hex3HexNAc2deHex1Sul2, Hex2HexNAc4deHex2Sul1, Hex2HexNAc4deHex2Sul1, Hex2HexNAc4deHex2Sul1, NeuGc1Hex2HexNAc4Sul1, Hex3Hex4deHex1Sul1, Hex2HexNAc3deHex3Sul2, Hex2HexNAc3deHex3Sul2, NeuGc1Hex2HexNAc3deHex1Sul2, NeuGc1Hex2HexNAc3deHex1Sul2, NeuGc1Hex2HexNAc3deHex1Sul2, NeuGc1Hex3HexNAc2deHex2Sul1, NeuAc1Hex2HexNAc4deHex1, Hex2HexNAc4deHex2Sul2, Hex2HexNAc4deHex2Sul2, NeuGc1Hex2HexNAc4deHex1, Hex2HexNAc3deHex4Sul1, Hex3HexNAc4deHex1Sul2, NeuGc1Hex2HexNAc3deHex2Sul1, NeuGc1Hex2HexNAc3deHex2Sul1, NeuGc1Hex3HexNAc3deHex1Sul1, NeuGc1Hex2HexNAc2deHex3 Sul2, Hex2HexNAc4deHex3 Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc3deHex2Sul2, Hex3HexNAc3deHex3 Sul2, NeuGc1Hex3HexNAc3deHex1Sul2, Hex2HexNAc5deHex2Sul1, Hex2HexNAc4deHex3Sul2, NeuGc1Hex2HexNAc4deHex2Sul1, NeuGc1Hex2HexNAc4deHex2Sul1, NeuGc1Hex3HexNAc4deHex1Sul1, Hex2HexNAc5deHex3 Sul1, NeuGc1Hex2HexNAc4deHex2Sul2, NeuGc1Hex2HexNAc4deHex2Sul2, NeuGc1Hex2HexNAc4deHex2Sul2, NeuGc1Hex3HexNAc4deHex1Sul2, Hex3HexNAc4deHex4Sul2, NeuAc1Hex5HexNAc4deHex1, NeuAc1Hex4HexNAc4deHex2Sul1, NeuGc1Hex3HexNAc4deHex4Sul1, NeuGc1Hex4HexNAc4deHex3Sul1, and NeuGc1Hex3HexNAc4deHex4Sul2. In some embodiments, the composition comprises at least one, two or all three of NeuAc1HexNAc1, NeuAc2Hex1HexNAc1, and NeuAc1Hex1HexNAc1.

In some embodiments, the sialic acid content of the composition is greater than 25% and less than 50% (e.g., 25, 30, 35, 40, 45, or 49%).

In some embodiments, the composition comprises glycopeptide-bound oligosaccharides having at least 7, 14, 21 or all 28 of the following structures: Galp 1-3GalNAc, GlcNAcβ1-6GalNAc, NeuAcα2-6GalNAc, NeuGcα2-6GalNAc, Fucα1-2Galβ1-3GalNAc, Gal+GlcNAcβ1-6GalNAc, Galβ1-3(GlcNAcβ1-6)GalNAc, Galβ1-3GlcNAcβ1-6GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galp 1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, Fucα1-2(GalNAcα1-3)Galβ1-3GalNAc, Fucα1-2Galβ1-4GlcNAcβ1-6GalNAc, Fucα1-2Galβ1-3(GlcNAcβ1-6)GalNAc, Fucα1-2Galβ1-3(6S-GlcNAcβ1-6)GalNAc, Fucα1-2Galβ1-3(NeuAcβ2-6)GalNAc, GlcNAcβ1-3[Galβ1-4(6S)GlcNAcβ1-6]GalNAc, Galβ1-4GlcNAcβ1-3[(6S)GlcNAcβ1-6]GalNAc, Galβ1-3(Fucα1-2Galβ1-4GlcNAcβ1-6)GalNAc, Fucα1-2Galβ1-4(6S)GlcNAcβ1-6[GlcNAcβ1-3]GalNAc, GlcNAcβ1-3[Fucα1-2Galβ1-3(6S-)GlcNAcβ1-6]GalNAc, Fucα1-2Galβ1-3[Fucα1-2Galβ1-4(6S)GlcNAcβ1-6]GalNAc, and NeuAcα2-3Galβ1-3(NeuAcα2-6)GalNAc.

In some embodiments, the composition comprises glycopeptide-bound oligosaccharides having at least 10, 20, 30, 40, 50, 60, 70, 80, 90, 100, 110, 120, 130, 140, 150, 160, or all of the following structures: Galβ1-3GalNAcol, GlcNAcβ1-3GalNAcol, GalNAcα1-3GalNAcol, SGalβ1-GlcNAc, 3SGalβ1-3GalNAcol, 6SGlcNAcβ1-6GalNAcol, 6SGlcNAcβ1-3GalNAcol, NeuAcα2-6GalNAcol, NeuGcα2-6GalNAcol, Fucα1-2(GalNAcα1-3)Galol, Fucα1-2Galβ1-4GlcNAcol, Fucα1-2Galβ1-3GalNAcol, Galβ1-4GlcNAcβ1-3Galol, Galβ1-3(GlcNAcβ1-6)GalNAcol, Galβ1-4GlcNAcβ1-3GalNAcol, 6SGal(Fuc)GlcNAc, 3SGal-GlcNAcβ1-3Galol, GlcNAcβ1-3(GlcNAcβ1-6)GalNAcol, Galβ1-3(6SGlcNAcβ1-6)GalNAcol, 3SGalβ1-3GlcNAcβ1-3GalNAcol, Galβ1-3(NeuAcα2-6)GalNAcol, NeuAcα2-3Galβ1-3GalNAcol, Galβ1-3(NeuGcα2-6)GalNAcol, NeuGcα2-3Galβ1-3GalNAcol, Fucα1-2Galβ-4GlcNAcβ1-3Galol, 6SGlcNAcβ1-3(GlcNAcβ1-6)GalNAcol, GlcNAcβ1-3(6S-GlcNAcβ1-6)GalNAcol, GlcNAcβ1-3(NeuAcα2-6)GalNAcol, GalNAcα1-3(NeuAcα2-6)GalNAcol, GlcNAcβ1-3(NeuGcα2-6)GalNAcol, GalNAcα1-3(NeuGcα2-6)GalNAcol, Galβ1-3(Fucα1-4)GlcNAcβ1-3GalNAcol, Fucα1-2(GalNAcα1-3)Galβ1-3GalNAcol, Fucα1-2Galβ1-3GlcNAcβ1-3GalNAcol, Fucα1-2Galβ1-3(GlcNAcβ1-6)GalNAcol, Galβ1-3(Galβ1-4GlcNAcβ1-6)GalNAcol, Galβ1-3GlcNAcβ1-3Galβ1-3GalNAcol, Fucα1-2Gal(Fuc)(6S)GlcNAc, 6SGalβ1-4(Fucα1-3)GlcNAcβ1-3Galol, GlcNAcβ1-3(Galβ1-4GlcNAcβ1-6)GalNAcol, 6SGalβ1-4(Fucα1-3)GlcNAcβ1-3GalNAcol, Fucα1-2Galβ1-3(6S-GlcNAcβ1-6)GalNAcol, Fucα1-2Galβ1-3(NeuAcα2-6)GalNAcol, Galβ1-3[Galβ1-4(6S)GlcNAcβ1-6]GalNAcol, 3SGalβ1-3GlcNAcβ1-3Galβ1-3GalNAcol, Fucα1-2Galβ1-3(NeuGcα2-6)GalNAcol, GlcNAcα1-4Galβ1-3[(6S)GlcNAcβ1-6]GalNAcol, GlcNAcα1-4(S)Galβ1-4GlcNAcβ1-3GalNAcol, GlcNAcβ1-3[Gal-(6S)GlcNAcβ1-6]GalNAcol, Galβ1-4GlcNAcβ1-3(6SGlcNAcβ1-6)GalNAcol, GlcNAcβ1-3[Galβ1-4(6S)GlcNAcβ1-6]GalNAcol, NeuAcα2-3(GalNAcβ1-4)Galβ1-3GalNAcol, GlcNAcα1-4Galβ1-3(NeuAcα2-6)GalNAcol, NeuAcα2-3Galβ1-3(GlcNAcβ1-6)GalNAcol, GalNAcβ1-4(NeuGcα2-3)Galβ1-3GalNAcol, NeuGcα2-3Galβ1-3(GlcNAcβ1-6)GalNAcol, Galβ1-3[Galβ1-3(Fucα1-4)GlcNAcβ1-6]GalNAcol, Galβ1-3(Fucα1-2Galβ1-4GlcNAcβ1-6)GalNAcol, Fucα1-2Galβ1-3(Galβ1-4GlcNAcβ1-6)GalNAcol, Galβ1-3(Galα1-3Galβ1-4GlcNAcβ1-6)GalNAcol, Galα1-3Galβ1-4GlcNAcβ1-3Galβ1-3GalNAcol, NeuGcα2-3(6S)Gal-(Fuc)GlcNAc, GlcNAcβ1-3[Gal-(Fuc)GlcNAcβ1-6]GalNAcol, Fucα1-2(GalNAcα1-3)Galβ1-4GlcNAcβ1-3GalNAcol, GlcNAcβ1-3(Fucα1-2Galβ1-4GlcNAcβ1-6)GalNAcol, 3SGalβ1-4GlcNAcβ1-3(6SGlcNAcβ1-6)GalNAcol, GlcNAcα1-4Galβ1-3(Galβ1-4GlcNAcβ1-6)GalNAcol, NeuAcα2-3Galβ1-3[(6S)GlcNAcβ1-6]GalNAcol, NeuAcα2-3Galβ1-3(NeuAcα2-6)GalNAcol, NeuGcα2-6Galβ1-3(6SGlcNAcβ1-6)GalNAcol, Galβ1-3[Galβ1-4(Fucα1-3)(6S)GlcNAcβ1-6]GalNAcol, Galβ1-3[Fucα1-2(6S)Galβ1-4GlcNAcβ1-6]GalNAcol, Gal(Fuc)(6S)GlcNAcβ1-3Galβ1-3GalNAcol, Galβ1-3[Fucα1-2Galβ1-4(6S)GlcNAcβ1-6]GalNAcol, Fucα1-2Galβ1-3[Galβ1-4(6S)GlcNAcβ1-6]GalNAcol, NeuAcα2-3Galβ1-3(NeuGcα2-6)GalNAcol, NeuGcα2-3Galβ1-3(NeuGcα2-6)GalNAcol, GlcNAcβ1-3[Galβ1-4(Fucα1-3)(6S)GlcNAcβ1-6]GalNAcol, Gal(Fuc)GlcNAcβ1-3(6SGlcNAcβ1-6)GalNAcol, GlcNAcβ1-3[SGalβ1-4(Fucα1-3)GlcNAcβ1-6]GalNAcol, Fucα1-2Galβ1-4GlcNAcβ-3(6SGlcNAcβ1-6)GalNAcol, GlcNAcβ1-3[Fucα1-2Galβ1-4(6S)GlcNAcβ1-6]GalNAcol, GalNAcα1-3(Fucα1-2)Galβ1-3(6SGlcNAcβ1-6)GalNAcol, Fucα1-2Galβ1-4(6S)GlcNAcβ1-3(GlcNAcβ1-6)GalNAcol, Fucα1-2Galβ1-4GlcNAcβ-3(6SGlcNAcβ1-6)GalNAcol, Fucα1-2Galβ1-3GlcNAcβ1-3(NeuAcα2-6)GalNAcol, Fucα1-2Galβ1-4GlcNAcβ1-3(NeuAcα2-6)GalNAcol, Galβ1-4GlcNAcβ1-3[Galβ1-4(6S)GlcNAcβ1-6]GalNAcol, Fucα1-2Galβ1-4GlcNAcβ1-3(NeuGcα2-6)GalNAcol, NeuAcα2-3Galβ1-3(Galβ1-4GlcNAcβ1-6)GalNAcol, Galβ1-3[Fucα1-2Gal(Fuc)GlcNAcβ1-6]GalNAcol, Fucα1-2Galβ1-3(Fucα1-2Galβ1-4GlcNAcβ1-6)GalNAcol, Galβ1-3[3SGal-(Fuc)(6S)GlcNAcβ1-6]GalNAcol, SGal-(Fuc)(6S)GlcNAc-Galβ1-3GalNAcol, NeuGcα2-3Galβ1-3(Galβ1-4GlcNAcβ1-6)GalNAcol, NeuAcα2-3(GalNAcβ1-4)Galβ1-3(GlcNAcβ1-6)GalNAcol, 6SGalβ1-(Fucα1-)GlcNAcβ1-3(6SGlcNAcβ1-6)GalNAcol, 6SGalβ1-(Fucα1-)(6S)GlcNAcβ1-3(GlcNAcβ1-6)GalNAcol, NeuGcα2-3(GalNAcβ1-4)Galβ1-3(GlcNAcβ1-6)GalNAcol, Galβ1-3[Fucα1-2(GalNAcα1-3)Galβ1-4GlcNAcβ1-6]GalNAcol, 3SGal-GlcNAcβ1-3(6SGlcNAcβ1-6)Galβ1-3GalNAcol, SGalβ1-4GlcNAcβ1-3(SGalβ1-3GlcNAcβ1-6)GalNAcol, NeuAcα2-3Galβ1-3[Galβ1-4(6S)GlcNAcβ1-6]GalNAcol, Galβ1-3[Fucα1-2Galβ1-(Fuca-)(6S)GlcNAcβ1-6]GalNAcol, Fucα1-2Galβ1-3[Gal-(Fuc)(6S)GlcNAcβ1-6]GalNAcol, Fucα1-2Galβ1-3[Fucα1-2Galβ1-4(6S)GlcNAcβ1-6]GalNAcol, NeuGcα2-3Galβ1-3[Galβ1-4(6S)GlcNAcβ1-6]GalNAcol, NeuGcα2-3Galβ1-3(3SGal-GlcNAcβ1-6)GalNAcol, GlcNAcβ1-3[GalNAcα1-3(Fucα1-2)Galβ1-3GlcNAcβ1-6]GalNAcol, NeuAcα2-3(GalNAcβ1-4)Galβ1-3(6SGlcNAcβ1-6)GalNAcol, GlcNAcβ1-3[Fucα1-2Galβ1-(Fucα1-)(6S)GlcNAcβ1-6]GalNAcol, 6SGlcNAcβ1-3[Fucα1-2Galβ1-(Fucα1-)GlcNAcβ1-6]GalNAcol, NeuAcα2-3(GalNAcβ1-4)Galβ1-3(NeuAcα2-6)GalNAcol, GlcNAcβ1-3[NeuGcα2-3Galβ1-4(6S)GlcNAcβ1-6]GalNAcol, Galβ1-3[GalNAcα1-3(Fucα1-2)Galβ1-4(6S)GlcNAcβ1-6]GalNAcol, Galβ1-3GlcNAcβ1-3[Fucα1-2Galβ1-4(6S)GlcNAcβ1-6]GalNAcol, Galβ1-4GlcNAcβ1-3[Fucα1-2Galβ1-3(6S)GlcNAcβ1-6]GalNAcol, Fucα1-2Galβ1-4GlcNAcβ1-3[Galβ1-4(6S)GlcNAcβ1-6]GalNAcol, NeuAcα2-3(GalNAcβ1-4)Galβ1-3(NeuGcα2-6)GalNAcol, NeuAcα2-3Galβ1-3(Fucα1-2Galβ1-4GlcNAcβ1-6)GalNAcol, NeuGcα2-3(GalNAcβ1-4)Galβ1-3(NeuGcα2-6)GalNAcol, Fucα1-2Galβ1-3[SGal-(Fuc)(6S)GlcNAcβ1-6]GalNAcol, NeuGcα2-3Galβ1-3 (Fucα1-2Galβ1-4GlcNAcβ1-6)GalNAcol, Galα1-3Galβ1-3(NeuGcα2-3Galβ1-4GlcNAcβ1-6)GalNAcol, GlcNAcβ1-3[GalNAcα1-3(Fucα1-2)Galβ1-(6S)GlcNAcβ1-6]GalNAcol, GalNAcα1-3(Fucα1-2)Galβ1-3GlcNAcβ1-3(6SGlcNAcβ1-6)GalNAcol, GlcNAcα1-4(Fucα1-2)Galβ1-4GlcNAcβ1-3(NeuAcα2-6)GalNAcol, NeuAcα2-3Galβ1-3[GlcNAcα1-4Galβ1-4GlcNAcβ1-6]GalNAcol, Galβ1-(Fuc)GlcNAcβ1-3(Fucα1-2Galβ1-4GlcNAcβ1-6)GalNAcol, Fucα1-2Galβ1-4GlcNAcβ1-3(Fucα1-2Galβ1-4GlcNAcβ1-6)GalNAcol, SGal-GlcNAcβ1-6[Fucα1-2Gal-(6S)GlcNAcβ1-6]GalNAcol, NeuGcα2-3Galβ1-3(GlcNAcα1-4Galβ1-4GlcNAcβ1-6)GalNAcol, NeuAcα2-3Galβ1-3[Fucα1-2Galβ1-4(6S)GlcNAcβ1-6]GalNAcol, SGal-GlcNAcβ1-3(GlcNAcβ1-6)Galβ1-3(6SGlcNAcβ1-6)GalNAcol, NeuAcα2-3(GalNAcβ1-4)Galβ1-3[Galβ1-4(6S)GlcNAcβ1-6]GalNAcol, Fucα1-2Galβ1-3[GalNAcα1-3(Fucα1-2)Galβ1-3(6S)GlcNAcβ1-6]GalNAcol, Fucα1-2Galβ1-4GlcNAcβ1-3[Fucα1-2Galβ1-4(6S)GlcNAcβ1-6]GalNAcol, Fucα1-2Galβ1-3[GalNAcα1-3(Fucα1-2)Galβ1-4(6S)GlcNAcβ1-6]GalNAcol, SGal-(Fuc)GlcNAcβ1-3[SGal-(Fuc)GlcNAcβ1-6]GalNAcol, SGal-(Fuc)(6S)GlcNAcβ1-3(3SGal-GlcNAcβ1-6)GalNAcol, Gal(Fuc)(6S)GlcNAcβ1-3(Fucα1-2)Galβ1-3(6SGlcNAcβ1-6)GalNAcol, SGal-(Fuc)GlcNAcβ1-3[Gal-(Fuc)(6S)GlcNAcβ1-6]GalNAcol, SGal-GlcNAcβ1-3[Fucα1-2Gal-(Fuc)(6S)GlcNAcβ1-6]GalNAcol, NeuGcα2-3(GlcNAcβ1-4)Galβ1-3 (Fucα1-2Galβ1-4GlcNAcβ1-6)GalNAcol, NeuGcα2-3Galβ1-3[Fucα1-2Galβ1-3(Fucα1-4)(6S)GlcNAcβ1-6]GalNAcol, SGal-GlcNAcβ1-3[GalNAcα1-3(Fucα1-2)Gal-(6S)GlcNAcβ1-6]GalNAcol, NeuAcα2-3Galβ1-3[GalNAcα1-3(Fucα1-2)Gal-(6S)GlcNAcβ1-6]GalNAcol, Fucα1-2Galβ1-4(6S)GlcNAcβ1-3[Fucα1-2Galβ1-3(Fucα1-4)GlcNAcβ1-6]GalNAcol, GlcNAcα1-4Galβ1-3[NeuGcα2-6Gal-(Fuc)(6S)GlcNAcβ1-6]GalNAcol, GlcNAcα1-4(Fucα1-2)GlcNAcβ1-3[Fucα1-2)Galβ1-4(6S)GlcNAcβ1-6]GalNAcol, GalNAcα1-3(Fucα1-2)Gal-GlcNAcβ1-3[Gal(Fuc)(6S)GlcNAcβ1-6]GalNAcol, Galβ1-3GlcNAcβ1-3[GalNAcα1-3(Fucα1-2)Gal-(Fucα1-2)(6S)GlcNAcβ1-6]GalNAcol, SGal-(Fuc)GlcNAcβ1-3[Fucα1-2Gal-(Fuc)(6S)GlcNAcβ1-6]GalNAcol, Fucα1-2Gal-(Fuc)GlcNAcβ1-3[SGal-(Fuc)(6S)GlcNAcβ1-6]GalNAcol, GalNAcα1-3(Fucα1-2)Gal-GlcNAcβ1-3(NeuAcα2-3Gal-GlcNAcβ1-6)GalNAcol, SGal-(Fuc)GlcNAcβ1-3[GalNAcα1-3(Fucα1-2)Gal-(6S)GlcNAcβ1-6]GalNAcol, Gal-(Fuc)(6S)GlcNAcβ1-3[GalNAcα1-3(Fucα1-2)Gal-(6S)GlcNAcβ1-6]GalNAcol, GalNAcα1-3(Fucα1-2)Gal-GlcNAcβ1-3(NeuGcα2-3Gal-GlcNAcβ1-6)GalNAcol, Fucα1-2Gal(Fuc)GlcNAcβ1-3[Fucα1-2Gal-(Fuc)(6S)GlcNAcβ1-6]GalNAcol, Gal-(6S)GlcNAcβ1-3[SGal-(Fuc)(6S)GlcNAcβ1-6]Galβ1-3(GlcNAcβ1-6)GalNAcol, Fucα1-2Gal(Fuc)GlcNAc-(6S)Galβ1-3[6SGal-(Fuc)GlcNAcβ1-6]GalNAcol, GalNAcα1-3(Fucα1-2)Galβ1-4GlcNAcβ1-3[GalNAcα1-3(Fucα1-2)Galβ1-4(6S)GlcNAcβ1-6]GalNAcol, GalNAcα1-3(Fucα1-2)Gal-GlcNAcβ1-3[GalNAcα1-3(Fucα1-2)Gal-(Fuc)(6S)GlcNAcβ1-6]GalNAcol, NeuAcα2-Galβ1-4GlcNAcβ1-2Manα1-3(Galβ1-4GlcNAcβ1-2Manα1-6)Manβ1-4GlcNAcβ1-4(Fucα1-6)GlcNAcol, and Fucα1-2Galβ1-3(Fucα1-4)GlcNAcβ1-3[NeuGcα2-6(3S)Galβ1-GlcNAcβ1-3(Fucα1-2)Gal-(Fuc)(6S)GlcNAcβ1-6]GalNAcol.

In some embodiments, the composition comprises at least 1, 2, 3, 4, 5, 6, 7, 8, 9, or all 10 sialylated glycopeptide-bound oligosaccharide selected from the following: NeuAcα2-6GalNAc, NeuGcα2-6GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, and Fucα1-2Galβ1-3(NeuAcβ2-6)GalNAc. In some embodiments, the composition comprises NeuAcα2-3Galβ1-3GalNAc (Sialyl-T antigen), NeuAcα2-6GalNAc (Sialyl Tn antigen), and NeuAcα2-3Galβ1-3(NeuAcα2-6)GalNAc (Disialyl T antigen).

In some embodiments, the composition has substantially no free glycans. In some embodiments, the composition has a pH of less than 7.5. In some embodiments, the composition is a powder. In some embodiments, the composition further comprises one or more excipients or carriers. In some embodiments, the composition is administered orally or rectally.

In some embodiments of the methods disclosed herein, the composition comprises at least one, at least two, at least three, at least four, at least five, or all six glycopeptide-bound oligosaccharide having a structure selected from Galβ1-3GalNAcol (T antigen), NeuAcα2-6GalNAcol (Sialyl Tn antigen), Galβ1-4(Fucα1-3)(6S)GlcNAcol (Lewis X), 6SGalβ1-3(Fucα1-4)GlcNAcol (Lewis A), NeuAcα2-3Galβ1-3(NeuAcα2-6)GalNAc (Disialyl T antigen), and NeuAcα2-3Galβ1-3GalNAcol (Sialyl T antigen).

In some embodiments of the methods disclosed herein, the composition is administered daily for 28 days or more.

In some embodiments of the methods disclosed herein, the composition is administered in an amount effective to increase one or more of Akkermansia spp., Butyricicoccus spp., Clostridium spp., or Parabacteroides spp in a subject's gut or a sample isolated from a subject's gut. In some embodiments, a sample isolated from a subject's gut is a fecal sample.

In some embodiments of the methods disclosed herein, the subject has a tumor that is refractory to or has developed resistance to an immune checkpoint inhibitor therapy.

In some embodiments of the methods disclosed herein, subject has a solid tumor, such as for example colorectal cancer (CRC), stage 3 or 4 melanoma, or metastatic CRC. In some embodiments, the subject has breast cancer or lung cancer. The cancer or tumor may be localized or metastatic, and any of Stage I to IV. In further embodiments, the subject has received a prior therapy such as for example a chemotherapy or immune therapy that comprises or consists essentially of, or consists of the administration of a checkpoint inhibitor. e.g., an anti-PD-1 or anti-PDL-1 therapy.

In some embodiments, oral administration of the composition results in an increased level or activity of CD8+ T cells cross-reactive to a glycan tumor antigen and a glycan present on a glycopeptide of the composition.

As provided in more detail below, Applicant has found that certain embodiments will delay or prevent the appearance of a tumor. Thus, in one aspect, the term “treat” intends the prevention or inhibition of the appearance of or progression of a tumor in a subject susceptible to cancer.

Also provided herein is a therapeutic composition comprising one or more of GCX or GBX, alone or in combination with a carrier, such as a pharmaceutically acceptable carrier. In one aspect, the GCX and GBX compositions are described in Example 13. The compositions can further contain a stabilizer, preservative, cryoprotective agent, or flavor. In another aspect, the composition further comprises an additional therapeutic agent, such as for example an anticancer therapy. Non-limiting examples of such include checkpoint inhibitors, such as an anti-PD-1 or anti-PDL-1 compound, composition or therapy. Examples of such are provided herein. The therapeutic compositions can be formulated for ease of use, direct or systemic, examples of such include intratumoral, intravenous, or oral.

The above discussed, and many other features and attendant advantages of the present inventions will become better understood by reference to the following detailed description of the invention.

### BRIEF DESCRIPTION OF THE DRAWINGS

FIG. 1. A schematic of diafiltration for the manufacture of the compositions comprising glycopeptides of the instant disclosure.

FIG. 2. An illustration of the protocol used to test administration of compositions comprising glycopeptides with and without ICI for the treatment of melanoma. The melanoma used in this protocol were immune checkpoint inhibitor (ICI)-resistant tumors. Tumor volume was measured every three days. Tumors were weighed at the end of the experiment.

FIGS. 3A-3B. Effect of compositions comprising glycopeptides against ICI resistant melanoma. (A) Representative photos of tumors excised at the end of the experiment. (B) Tumor volume measurements under different treatments. Orally administered compositions comprising glycopeptides induced a consistent systemic effect in reducing tumor growth (as a monotherapy or in combination with an immune checkpoint inhibitor (ICI)). Statistically significant tumor suppression was observed with GBX and an anti-PD-1 antibody combination in tumors that are resistant to ICI. Significant tumor suppression was observed using GCX as a monotherapy.

FIGS. 4A-4B. GBX in combination with anti-PD-1 reduce melanoma tumor volume by 50%. (A) Tumor volume and (B) tumor weight measurements under different treatments. Orally administered GBX glycopeptides in combination with ICI reduces tumor volume and weight by 50% while ICI alone does not. Effectiveness of oral administration on a distant organ demonstrates a systemic immune response targeted to the tumor.

FIGS. 5A-5B. GBX reduce melanoma tumor resistance to anti-PD-1 therapy. (A) Tumor volume and (B) tumor weight measurements under different treatments. Orally administered GBX glycopeptides turn a “cold” (i.e., resistant) tumor into a “hot” (i.e., not resistant) tumor as shown by better ICI efficacy in the combination treatment.

FIGS. 6A-6B. GCX as a monotherapy reduces melanoma tumor volume by 50%. (A) Tumor volume and (B) tumor weight measurements under different treatments. Orally administered GBX glycopeptides in combination with ICI reduces tumor volume and weight by 50% while ICI alone does not. GCX glycopeptides are effective in tumor volume reduction as a monotherapy.

FIG. 7. Possible methods of action for compositions comprising glycopeptides against ICI resistant melanoma.

FIG. 8. The immuno-oncology background for melanoma.

FIGS. 9A-9E. The effect of combination of GBX or GCX with anti-PD1 on tumor growth. C57BL/6 mice received GBX or GCX in the drinking water starting 14 days prior to subcutaneous (s.c.) injection of 200,000 YUMM1.7 melanoma cells. On day 6, 9 and 12 after melanoma cell injection, mice were injected with anti-PD1 or isotype control antibodies. (A) Experimental set-up, (B) representative pictures of tumors from the indicated groups, (C) tumor volume over time, (D) tumor volume and (E) tumor weight on day 15. *p<0.05, **p<0.01, ***p<0.001, 1-way ANOVA

FIGS. 10A-10D. The effect on myeloid cells upon GBX or GCX treatment. C57BL/6 mice received GBX or GCX in the drinking water starting 14 days prior to s.c. injection of 200′000 YUMM1.7 melanoma cells. On day 6, 9 and 12 after melanoma cell injection, mice were injected with anti-PD1 or isotype control antibodies. Tumor infiltrating immune cells ware analyzed on day 15 after tumor cell injection for the relative abundance of (A) dendritic cells (DC, characterized as CD11c+, MHCIIhigh), (B) neutrophils (characterized as Grl+), (C) macrophages (characterized as F4/80+), and (D) proportion of M1 (MHCIIhigh, CD206-macrophages) vs M2 (MHCIIlow, CD206+ macrophages). *p<0.05, 1-way ANOVA

FIGS. 11A-11L. GBX and GCX glycopeptides each promote anti-tumor CD8 T cell responses. C57BL/6 mice received GBX or GCX in the drinking water starting 14 days prior to s.c injection of 200,000 YUMM1.7 melanoma cells. On day 6, 9 and 12 after melanoma cell injection, mice were injected with anti-PD1 or isotype control antibodies. Tumor-infiltrating immune cells were analyzed on day 15 for (A) B cells (B220+CD3− cells), (B) T cells (CD3+, B220−, CD11b− cells), (C) CD4+ T cells, (D) CD8+ T cells, (E) Th1 cells (IFNg+ CD4+ T cells), (F) TNFa+ CD4+ T cells, (G) Th17 cells (IL-17+CD4+ T cells, (H) regulatory T cells (FoxP3+ CD4+ T cells), (I) IFNg+ CD8+ T cells, (J) TNFa+ CD8+ T cells, (K) Perforrin+ CD8+ T cells, (L) GranzymeB+ CD8+ T cells. *p<0.05, **p<0.01, ***p<0.001, 1-way ANOVA

FIGS. 12A-12F. GBX and GCX glycopeptides have some effect on T cell exhaustion markers. C57BL/6 mice received GBX or GCX in the drinking water starting 14 days prior to s.c injection of 200′000 YUMM1.7 melanoma cells. On day 6, 9 and 12 after melanoma cell injection, mice were injected with anti-PD1 or isotype control antibodies. (A)-(C) Tumor-infiltrating CD4+ and (D)-(F) CD8+ T cells were analyzed on day 15 for the exhaustion markers CTLA4 (FIGS. 12A and 12D), PD1 (FIGS. 12B and 12E), Tim3 (FIGS. 12C and 12F). *p<0.05, **p<0.01, ***p<0.001, 1-way ANOVA.

FIGS. 13A-13Q. GBX and GCX show moderate effects on serum cytokine levels. C57BL/6 mice received GBX or GCX in the drinking water starting 14 days prior to s.c injection of 200,000 YUMM1.7 melanoma cells. On day 6, 9 and 12 after melanoma cell injection, mice were injected with anti-PD1 or isotype control antibodies. Blood was collected on the day of tumor cell injection (d0) and 15 days after tumor cell injection (d15) and analyzed for the indicated cytokines. (A) Eotaxin, (B) G-CSF, (C) IL-5, (D) IL-10, (E) IL-17, (F) KC, (G) RANTES, (H) TNFα, (I) GM-CSF, (J) IFNγ, (K) IL-12p40, (L) IL-12p70, (M) MCP1, (N) MIP1α, (O) IL-1a, (P) IL-13, (Q) MIP1β. *p<0.05, **p<0.01, ***p<0.001, 1-way ANOVA.

FIGS. 14A-14C. Two-dimensional ordination of the microbial profiles by principal component analysis (PCA) showing ASVs correlating with the different sample sets. (A) Baseline (d-14) (B) After pre-feeding with test items but before tumor cell injection and aPD1 administration (d0) and (C) After tumor cell injection and 14 days of treatment phase (d14). Ctr group=mice that neither had test items supplementation in water, nor aPD1 administration throughout the study duration, Ctr−aPD1 group=mice that had aPD1 administration on Day 6 but did not have test items supplementation in water, GBX group=mice that did not have aPD1 administration but had GBX supplementation in water throughout the study, GBX−aPD1 group=mice that did have aPD1 administration on Day 6 and had GBX supplementation in water throughout the study, GCX group=mice that did not have aPD1 administration but had GCX supplementation in water throughout the study and GCX+aPD1 group=mice that did have aPD1 administration on Day 6 and had GCX supplementation in water throughout the study. Significant differences; p<0.05, PERMANOVA.

FIGS. 15A-15C. Diversity analysis of the microbial profiles after the pre-feeding phase but prior to tumor cell injection (d0) and after the tumor cell injection and 14 days of treatment (d14). (A) Observed ASVs (richness); (B) Shannon Diversity Index; (C) Evenness. Ctr group=mice that neither had test items supplementation in water, nor aPD1 administration throughout the study duration, Ctr−aPD1 group=mice that had aPD1 administration on Day 6 but didn't have test items supplementation in water, GBX group=mice that did not have aPD1 administration but had GBX supplementation in water throughout the study, GBX−aPD1 group=mice that did have aPD1 administration on Day 6 and GBX supplementation in water throughout the study, GCX group=mice that did not have aPD1 administration but had GCX supplementation in water throughout the study and GCX+aPD1 group=mice that did have aPD1 administration on Day 6 and GCX supplementation in water throughout the study. *Significant differences with baseline; ^{abcd} Significant differences between groups; p<0.05, Mann-Whitney test.

FIGS. 16A-16H. Percent change in relative abundance (% of sequences) of bacterial genera in mice fecal samples relative to the baseline after the pre-feeding phase but prior to tumor cell injection (d0) and after the tumor cell injection and 14 days of treatment (d14). (A) Akkermansia spp.; (B) Bacteroides spp.; (C) Barnesiella spp.; (D) Botryococcus spp.; (E) Clostridium IV; (F) Clostridium_XIVa; (G) Clostridium_XIVb; (H) Eubacterium spp. Ctr group=mice that neither had test items supplementation in water, nor aPD1 administration throughout the study duration, Ctr−aPD1 group=mice that had aPD1 administration on Day 6 but didn't have test items supplementation in water, GBX group=mice that did not have aPD1 administration but had GBX supplementation in water throughout the study, GBX−aPD1 group=mice that had aPD1 administration on Day 6 and GBX supplementation in water throughout the study, GCX group=mice that did not have aPD1 administration but had GCX supplementation in water throughout the study and GCX+aPD1 group=mice that had aPD1 administration on Day 6 and GCX supplementation in water throughout the study. *Significant differences with baseline. ^{abcd} Significant differences between groups; p<0.05, Mann-Whitney test.

FIGS. 17A-17H. Percent change in relative abundance (% of sequences) of bacterial genera in mice fecal samples relative to the baseline after the pre-feeding phase but prior to tumor cell injection (d0) and after the tumor cell injection and 14 days of treatment (d14). (A) Lactobacillus spp.; (B) Odoribacter spp.; (C) Oscillibacter spp.; (D) Parabacteroides spp.; (E) Prevotella spp; (F) Pseudoflavonifractor spp.; (G) Roseburia spp.; (H) Ruminococcus spp. Ctr group=mice that neither had test items supplementation in water, nor aPD1 administration throughout the study duration, Ctr−aPD1 group=mice that had aPD1 administration on Day 6 but didn't have test items supplementation in water, GBX group=mice that did not have aPD1 administration but had GBX supplementation in water throughout the study, GBX−aPD1 group=mice that had aPD1 administration on Day 6 and GBX supplementation in water throughout the study, GCX group=mice that did not have aPD1 administration but had GCX supplementation in water throughout the study and GCX+aPD1 group=mice that had aPD1 administration on Day 6 and GCX supplementation in water throughout the study. *Significant differences with baseline. ^{abcd} Significant differences between groups; p<0.05, Mann-Whitney test.

FIGS. 18A-18E. GBX and GCX reduce tumor growth. C57BL/6 mice received GBX or GCX in the drinking water starting 14 days prior to s.c injection of 200′000 MC38 CRC cells. On day 6, 9, 12 and 15 after cell injection, mice were injected with anti-PD1 or isotype control antibodies. (A) Experimental set-up, (B) representative pictures of tumors from the indicated groups, (C) tumor volume over time, (D) tumor volume and (E) tumor weight on day 15. *p<0.05, **p<0.01, ***p<0.001, 1-way ANOVA

FIGS. 19A-19D. Moderate effects on myeloid cells are observed upon GBX or GCX treatment. C57BL/6 mice received GBX or GCX in the drinking water starting 14 days prior to s.c injection of 200,000 MC38 CRC cells. On day 6, 9, 12 and 15 after CRC cell injection, mice were injected with anti-PD1 or isotype control antibodies. Tumor infiltrating immune cells ware analyzed on day 15 after tumor cell injection for the relative abundance of (A) dendritic cells (DC, characterized as CD11c+, MHCIIhigh), (B) neutrophils (characterized as Grl+), (C) macrophages (characterized as F4/80+), and (D) proportion of M1 (MHCIIhigh, CD206-macrophages) vs M2 (MHCIIlow, CD206+ macrophages). *p<0.05, 1-way ANOVA.

FIGS. 20A-20L, GBX and GCX promote anti-tumor T cell responses. C57BL/6 mice received GBX or GCX in the drinking water starting 14 days prior to s.c injection of 200′000 MC38 CRC cells. On day 6, 9, 12 and 15 after CRC cell injection, mice were injected with anti-PD1 or isotype control antibodies. Tumor-infiltrating immune cells were analyzed on day 15 for (A) B cells (B220+ CD3− cells), (B) T cells (CD3+, B220−, CD11b− cells), (C) CD4+ T cells, (D) CD8+ T cells, (E) Th1 cells (IFNg+ CD4+ T cells), (F) TNFa+ CD4+ T cells, (G) Th17 cells (IL-17+ CD4+ T cells, (H) regulatory T cells (FoxP3+ CD4+ T cells), (I) IFNg+ CD8+ T cells, (J) TNFa+CD8+ T cells, (K) Perforrin+ CD8+ T cells, (L) Granzyme B+CD8+ T cells. *p<0.05, **p<0.01, ***p<0.001, 1-way ANOVA.

FIGS. 21A-21F. GBX and GCX do not affect T cell exhaustion markers. C57BL/6 mice received GBX or GCX in the drinking water starting 14 days prior to s.c injection of 200′000 MC38 CRC cells. On day 6, 9, 12 and 15 after CRC cell injection, mice were injected with anti-PD1 or isotype control antibodies. Tumor-infiltrating CD4+ (A, C, E) and CD8+(B, D, F); T cells were analyzed on day 15 for the exhaustion markers (A and B) CTLA4, (C and D) PD1, (E and F) Tim3. *p<0.05, **p<0.01, ***p<0.001, 1-way ANOVA.

FIGS. 22A-22P. GBX and GCX effects on serum cytokine levels. C57BL/6 mice received GBX or GCX in the drinking water starting 14 days prior to s.c injection of 200′000 MC38 CRC cells. On day 6, 9, 12 and 15 after CRC cell injection, mice were injected with anti-PD1 or isotype control antibodies. Blood was collected on the day of tumor cell injection (d0) and 18 days after tumor cell injection (d18) and analyzed for the indicated cytokines. (A) Eotaxin, (B) G-CSF, (C) IL-5, (D) IL-10, (E) IL-17, (F) KC, (G) RANTES, (H) TNFα, (I) GM-CSF, (J) IFNγ, (K) IL-12p40, (L) IL-12p70, (M) MCP1, (N) MIP1α, (O) IL-1α, (P) MIP1β. *p<0.05, **p<0.01, ***p<0.001, 1-way ANOVA.

FIG. 23. The protocol for the colorectal cancer experiments provided in example 6. Tumor volume was measured every three days. Tumor weight was measured at the end of the experiment (D18 after subcutaneous injection of MC-38). Cytokine panel was measured from serum collected on DO and D18.

FIG. 24. Through molecular mimicry, glycopeptide containing compositions as provided herein can induce tumor-specific CD4+ and CD8+ T cells. The disclosed glycopeptides may share epitopes with cancer (neo)antigens through molecular mimicry. Glycosylation of tumor proteins generates neo-antigens that serve as targets for tumor-specific T cells. The disclosed glycopeptices can bind DC-SIGN, be internalized, and cross-presented as an antigen on the surface of the DC. In turn, the DC stimulates tumor-specific CD4+ and CD8+ T cells.

FIG. 25. Glycopeptide-containing compositions as provided herein have tumor-associated O-glycans that can aid in anti-tumor immunity. These tumor-associated O-glycans can be found on abnormal mucin-1 expressed by tumor. Mucin-1 (Muc1) and its special glycoforms are now an important target for CAR-T therapies in solid tumor.

FIG. 26. Glycopeptide-containing compositions as provided herein can modulate towards a Th1-like immune state favoring an anti-tumor response.

FIG. 27. Glycopeptide containing compositions as provided herein can modulate the microbiome toward a composition and function that favors an anti-tumor response.

FIG. 28. Glycopeptide containing compositions as provided herein can provide tumor-specific effector T cell stimulation against tumor cells.

FIG. 29. A schematic of the protocol described in Example 7.

FIG. 30. A graph that shows that mice previously treated with GBX or GCX have higher proportion of activated T cells vs untreated mice and have higher numbers of activated T cells that specifically recognize/cross-react to the antigen(s) present in GBX or GCX. See Example 7 for details.

FIGS. 31A-31B. (A) Different core structures of glycans found in the compositions of the disclosure. (B) Comparison of the distribution of core structures between two GCX and one GBX batches.

### DETAILED DESCRIPTION OF THE DISCLOSURE

### Definitions

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, or cell, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

As used herein, the term “excipient” refers to a natural or synthetic substance formulated alongside the active ingredient of a medication, included for the purpose of long-term stabilization, bulking up solid formulations, or to confer a therapeutic enhancement on the active ingredient in the final dosage form, such as facilitating drug absorption, reducing viscosity, or enhancing solubility.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

An “effective amount” is an amount necessary to achieve the desired therapeutic or diagnostic result and will vary with the disease and subject or patient to be treated. Effective amounts are determined using methods known in the art and as described herein.

The expression “gastrointestinal tract mucins” encompasses any natural source of mucin from which glycans and glycopeptides can be extracted, suitable for mammalian nutrition or pharmaceutical use. Typical sources of gastrointestinal tract mucins are extracts from gastrointestinal tract, in particular from porcine sources or from bovine sources. Commercial sources for gastrointestinal tract mucins include Biofac A/S (Kastrup, Denmark), Zhongshi Duqing (Heze, China), Shenzhen Taier Biotechnology Co., LTD (Shenzhen, China), and Dongying Tiandong Pharmaceutical Co. (Shandong, China). In some embodiments, the gastrointestinal tract mucins are from porcine gastric mucus.

The expression “subject” refers to mammals. For examples, mammals contemplated by the present invention include human, primates, domesticated animals such as cattle, sheep, pigs, horses, rodents, cats, dogs, and other pets. In some embodiments, the subject is a human. In some embodiments, the subject may be an infant (1 year old or less for a human), a toddler (3 years old or less for a human), a child, a young adult, an adult or a geriatric. In some embodiments, the infant is a newborn. The subject may be male or female. In some embodiments, the subject is female and of child-bearing age.

The term “glycoprotein” refers to proteins linked to oligosaccharides, e.g., proteins either N-linked or O-linked to oligosaccharides, and having a molecular weight of more than about 5 KDa.

The term “glycopeptide” refers to peptides linked to oligosaccharides, e.g. peptides either N-linked or O-linked to oligosaccharides, and having a molecular weight of less than about 5 KDa. Methods of determining molecular weight of glycopeptides and glycoproteins are known in the art and are not limited. In some embodiments, the molecular weight of glycopeptides and glycoproteins are determined by size exclusion chromatography.

The term “glycan” as used herein refers to an oligosaccharide. The term “free glycan” is synonymous with the term “free oligosaccharide,” as also used herein.

In some embodiments, peptides are defined as having a molecular weight of less than about 5 KDa. In some embodiments, the term peptides include glycopeptides. In some embodiments, proteins are defined as having a molecular weight of more than about 5 KDa. In some embodiments, the term proteins include glycoproteins.

As used herein, “a partially purified fraction” of gastrointestinal tract mucins comprises at least about 40%, at least about 50%, at least about 60%, at least about 70%, at least about 80%, at least about 90%, at least about 92.5%, at least about 95%, at least about 97.5%, at least about 98%, at least about 99%, or at least about 99.5% of the protein- and peptide-glycans present in un-purified gastrointestinal tract mucins. In some embodiments, the mucins or partially purified fraction thereof has been subject to an acid treatment.

The terms “treating” and “treatment” refer to administering to a subject an effective amount of a composition so that the subject experiences a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. In another aspect, the term “treatment” excludes prophylaxis. Alternatively, treatment is “effective” if the progression of a disease is reduced or halted. “Treatment” can also mean prolonging survival as compared to expected survival if not receiving treatment.

The term “chemotherapy” refers to administration of any genotoxic agent (e.g., DNA damaging agent), including conventional or non-conventional chemotherapeutic agents, for the treatment or prevention of cancer. Chemotherapeutic agents include agents that have been modified, (e.g., fused to antibodies or other targeting agents). Examples of chemotherapeutic agents include, but are not limited to, platinum compounds (e.g, cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, bendamustine, mitomycin C), antitumor antibiotics (e.g., daunorubicin, doxorubicin, idarubicin, epirubicin, mitoxantrone, bleomycin, plicamycin, dactinomycin), taxanes (e.g., paclitaxel, /raZ>-paclitaxel and docetaxel), antimetabolites (e.g:, 5-fluorouracil, cytarabine, premetrexed, thioguanine, floxuridine, capecitabine, and methotrexate), nucleoside analogues (e.g., fludarabine, clofarabine, cladribine, pentostatin, nelarabine, gemcitabine, 5-flurouracil), topoisomerase inhibitors (e.g., topotecan, irinotecan, SN-38, CPT-11), hypomethylating agents (e.g., azacitidine and decitabine), proteasome inhibitors (e.g., bortezomib), epipodophyllotoxins (e.g., etoposide and teniposide), DNA synthesis inhibitors (e.g., hydroxyurea), and vinca alkaloids (e.g., vincristine, vindesine, vinorelbine, and vinblastine). Chemotherapeutic agents include DNA intercalating agents (e.g, pyrrol obenzodiazepines).

The terms, “decrease”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two-standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

Several classes of checkpoint inhibitors/regulators (immune checkpoint inhibitors “ICI”) are known in the art, including lymphocyte activation gene-3 (LAG-3), T cell immunoglobulin and ITIM domain (TIGIT), T cell immunoglobulin and mucin-domain containing-3 (TIM-3), V-domain immunoglobulin suppressor of T cell activation (VISTA), B7 homolog 3 protein (B7-H3), inducible T cell costimulatory (ICOS) and B and T lymphocyte attenuator (BTLA), anti-cytotoxic T lymphocyte associated antigen-4 (CTLA-4) as well as inhibitors of CTLA-4, programmed death 1 (PD-1; also referred to herein as PD1), and programmed death ligand-1 (PD-L1).

The latter 3 classes of checkpoint inhibitors, CTLA-4, PD-1 and PD-L1 inhibitors, have contributed several medically relevant drugs such as monoclonal antibody (mAb) inhibitors. Example of anti-CTLA-4 inhibitory mAb is ipilimumab (approved globally). Example of anti-PD-L1 inhibitory mAbs are atezolizumab, avelumab and durvalumab (approved globally). Non-limiting examples of anti-PD1 inhibitor mAbs include pembrolizumab and nivolumab (approved globally); sintilimab, tislelizumab, toripalimab, and camrelizumab (approved in China); geptanolimab serplulimab zimberelimab cemiplimab, dostarlimab, prolgolimab, balstilimab, penpulimab, retifanlimab, cadonilimab, pucotenlimab, sasanlimab, and cetrelimab.

Checkpoint inhibitory mAbs of distinct subclasses can be combined in a distinct modality (e.g. ipilimumab+nivolumab), or combined individually with chemotherapy, biologic therapies, anti-angiogenic therapies such as VEGF inhibitors, anti-TGFβs, cell therapies, mRNA therapies and so on.

As used herein, the phrase “GBX composition” refers to a composition prepared by the methods described in Examples 1 and 2. Briefly, a GBX composition is produced by a method comprising: (a) dissolving gastrointestinal mucin from stomach of an animal in water comprising calcium hydroxide; (b) adding diatomaceous earth to (a) and filtering the resulting solution; (c) adding a cationic substance to (b); and (d) filtering and concentrating the solution from (c), thereby producing the composition. In some embodiments, the method further comprises adjusting the pH of the solution in (b) using carbon dioxide. In some embodiments, the dissolving is achieved at about 60° C. (e.g., 55° C., 56° C., 57° C., 58° C., 59° C., 60° C., 61° C., 62° C., 63° C., 64° C., or 65° C.). In some embodiments, the cationic substance comprises an ion exchange hydrogen form resin.

In some embodiments, the GBX composition comprises at least one glycopeptide-bound oligosaccharide having a general formulae selected from Hex1HexNAc1, Hex1HexNAc1deHex1, Hex1HexNAc1deHex1Sul1, Hex1HexNAc1deHex2, Hex1HexNAc1deHex2Sul1, Hex1HexNAc1Sul1, Hex1HexNAc2, Hex1HexNAc2deHex1, Hex1HexNAc2deHex1Sul1, Hex1HexNAc2deHex2, Hex1HexNAc2Sul1, Hex1HexNAc3, Hex1HexNAc3deHex1, Hex1HexNAc3deHex1Sul1, Hex1HexNAc3Sul1, Hex1HexNAc4, Hex1HexNAcSul1, Hex2HexNAc1, Hex2HexNAc1deHex1, Hex2HexNAc1deHex1Sul1, Hex2HexNAc2, Hex2HexNAc2deHex1, Hex2HexNAc2deHex1Sul1, Hex2HexNAc2deHex2, Hex2HexNAc2deHex2Sul1, Hex2HexNAc2Sul1, Hex2HexNAc3, Hex2HexNAc3deHex1, Hex2HexNAc3deHex1Sul1, Hex2HexNAc4, Hex2HexNAc4deHex1, Hex3HexNAc2deHex1, Hex3HexNAc2deHex1Sul1, Hex3HexNAc3deHex1, Hex3HexNAc3deHex1Sul1, Hex4HexNAc3deHex1Sul1, HexNAc1deHex1Sul1, HexNAc2, HexNAc2deHex1, HexNAc2Sul1, NeuAc1Hex1HexNAc1, NeuAc1Hex1HexNAc1deHex1, NeuAc1Hex1HexNAc2, NeuAc1Hex2HexNAc2, and NeuAc1HexNAc1.

In some embodiments, the GBX composition comprises glycopeptide-bound oligosaccharides having at least 10, at least 20, at least, 30, at least 40, at least 45, or all (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46) of the general formulae listed above. In some embodiments, the GBX composition comprises at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%,) of all glycopeptide-bound oligosaccharides in the composition.

In some embodiments, the GBX composition comprises at least 10 glycopeptide-bound oligosaccharides having the following formulae: Hex1HexNAc1deHex1, Hex1HexNAc3deHex1Sul1, Hex1NAc3Sul1, NeuAc1Hex1HexNAc1, Hex1HexNAc2deHex1, NeuAc1Hex1HexNAc1deHex1, Hex1HexNAc3deHex1Sul1, NeuAc1Hex1HexNAc1, Hex1HexNAc2deHex1Sul1, Hex1HexNAc2Sul1, Hex1HexNAc1, and Hex1HexNAc3Sul1. In some embodiments, each formula represents between about 3% and 10% (e.g., about 3%, 4%, 5%, 6%, 7%, 8%, 9%, or 10%) of all glycopeptide-bound oligosaccharides in the composition.

In some embodiments, the GBX composition comprises at least 10 different glycopeptide-bound oligosaccharides having a structure selected from Galβ1-3GalNAc, GlcNAcα1-4Gal, GlcNAcβ1-4Gal, Fucα1-2(GalNAcα1-3)Gal, Fucα1-2Galβ1-3GalNAc, Fucα1-2Galβ1-3GlcNAc, Fucα1-2Galβ1-4GlcNAc, Gal-GlcNAc-Fuc, 6SGalβ1-3(Fucα1-4)GlcNAc, Fucα1-2Galβ1-4(6S)GlcNAc, Galβ1-4(Fucα1-3)(6S)GlcNAc, Fucα1-2Galβ1-4(Fucα1-3)GlcNAc, Fucα1-2Gal(Fuc)(6S)GlcNAc, (S)Galβ1-GlcNAc, Galβ1-4(6S)GlcNAc, Galβ1-3(GlcNAcβ1-6)GalNAc, GlcNAcα1-4Galβ1-3GalNAc, GlcNAcα1-4Galβ1-4GlcNAc, Fucα1-2(GalNAcα1-3)Galβ1-3GalNAc, Fucα1-2(GalNAcα1-3)Galβ1-4GlcNAc, Fucα1-2Galβ1-3(GlcNAcβ1-6)GalNAc, GlcNAcα1-4Gal(Fuc)GlcNAc, Fucα1-2(GalNAcα1-3)Galβ1-4(6S)GlcNAc, Fucα1-2Galβ1-3(6S-GlcNAcβ1-6)GalNAc, Fucα1-2Galβ1-4(6S)GlcNAcβ1-3GalNAc, Fucα1-2(GalNAcα1-3)Gal-(Fuc)GlcNAc, Galβ1-3(6SGlcNAcβ1-6)GalNAc, GlcNAcα1-3Galβ1-4GlcNAcβ1-6GalNAc, GlcNAcα1-4Galβ1-3(GlcNAcβ1-6)GalNAc, Fucα1-2(GalNAcα1-3)Galβ1-4GlcNAcβ1-6GalNAc, Fucα1-2Galβ1-3(GalNAcβ1-4GlcNAcβ1-6)GalNAc, GalNAcα1-3(Fucα1-2)Galβ1-3(6SGlcNAcβ1-6)GalNAc, GlcNAcβ1-3[Fucα1-2Galβ1-4(6S)GlcNAcβ1-6]GalNAc, GlcNAcα1-4Galβ1-3[(6S)GlcNAcβ1-6]GalNAc, GlcNAcα1-4Galβ1-3(GalNAcβ1-4GlcNAcβ1-6)GalNAc, Galβ1-4(6S)GlcNAcβ1-6GalNAc, SGalβ1-3(GlcNAcβ1-6)GalNAc, Galβ1-4GlcNAcβ1-3Gal, Fucα1-2Galβ-4GlcNAcβ1-3Gal, Fucα1-2(S)Galβ1-4GlcNAcβ1-4Gal, Fucα1-2Galβ1-3(6S)GlcNAcβ1-4Gal, Fucα1-2Galβ1-4(6S)GlcNAcβ1-4Gal, Galβ1-3(Galβ1-4GlcNAcβ1-6)GalNAc, Galβ1-4GlcNAcβ1-3Galβ1-3GalNAc, GlcNAcα1-4Galβ1-4GlcNAcβ1-3Gal, Fucα1-2Galβ1-3(Galβ1-4GlcNAcβ1-6)GalNAc, Fucα1-2Galβ1-4GlcNAcβ1-3Galβ1-4GlcNAc, GalNAcα1-3(Fucα1-2)Galβ1-4GlcNAcβ1-3Gal, Galβ1-3(Fucα1-2Galβ1-4GlcNAcβ1-6)GalNAc, Galβ1-3(Fucα1-4)GlcNAcβ1-3Galβ1-3GalNAc, Galβ1-4(Fucα1-3)GlcNAcβ1-3Galβ1-3GalNAc, Fucα1-2Galβ1-3[Galβ1-4(6S)GlcNAcβ1-6]GalNAc, Fucα1-2Galβ1-4(6S)GlcNAcβ1-3Galβ1-3GalNAc, GalNAcα1-3(Fucα1-2)Galβ1-4(6S)GlcNAcβ1-3Gal, Fucα1-2Galβ1-3(Fucα1-2Galβ1-4GlcNAcβ1-6)GalNAc, Fucα1-2Galβ1-3[Fucα1-2Galβ1-4(6S)GlcNAcβ1-6]GalNAc, Galβ1-3[Galβ1-4(6S)GlcNAcβ1-6]GalNAc, Galβ1-4GlcNAcβ1-3(GlcNAcβ1-6)Galβ1-3GalNAc, GlcNAcα1-4Galβ1-3(Galβ1-4GlcNAcβ1-6)GalNAc, Fucα1-2Galβ1-3(GlcNAcα1-4Galβ1-4GlcNAcβ1-6)GalNAc, Galβ1-3[Fucα1-2(GalNAcα1-3)Galβ1-4GlcNAcβ1-6]GalNAc, GlcNAcα1-4Galβ1-3(Fucα1-2Galβ1-4GlcNAcβ1-6)GalNAc, GalNAcα1-3(Fucα1-2)Galβ1-4(6S)GlcNAcβ1-3Galβ1-3GalNAc, Gal-GlcNAcβ1-3(GlcNAcα1-4Galβ1-4GlcNAcβ1-6)GalNAc, GlcNAcα1-4Galβ1-3(GlcNAcα1-4Galβ1-4GlcNAcβ1-6)GalNAc, GlcNAcα1-4Galβ1-3[GalNAcα1-3(Fucα1-2)Galβ1-4GlcNAcβ1-6]GalNAc, Fucα1-2Galβ1-4GlcNAcβ1-3(Galβ1-4GlcNAcβ1-6)Gal, Fucα1-2Galβ1-4(6S)GlcNAcβ1-3(Galβ1-4GlcNAcβ1-6)Gal, Fucα1-2Galβ1-4GlcNAcβ1-3Galβ1-4GlcNAcβ1-3Galβ1-3GalNAc, Fucα1-2Galβ1-4(6S)GlcNAcβ1-3Galβ1-4GlcNAcβ1-3Galβ1-4GlcNAc, Fucα1-2Galβ1-4(6S)GlcNAcβ1-3Galβ1-4GlcNAcβ1-3(Galβ1-4GlcNAcβ1-6)Gal, 6SGlcNAc-Fuc, HexNAc-GlcA, GlcNAcβ1-6GalNAc, GlcNAc-GlcNAc-Fuc, 6SGlcNAcβ1-3GalNAc, 6SGlcNAcβ1-6GalNAc, Galβ1-3(NeuAcα2-6)GalNAc, NeuAcα2-3Galβ1-3GalNAc, Fucα1-2Galβ1-3(NeuAcα2-6)GalNAc, GlcNAcα1-4Galβ1-3(NeuAcα2-6)GalNAc, Galβ1-4GlcNAcβ1-3Galβ1-3(NeuAcα2-6)GalNAc, NeuAc-HexNAc, and NeuAcα2-6GalNAc.

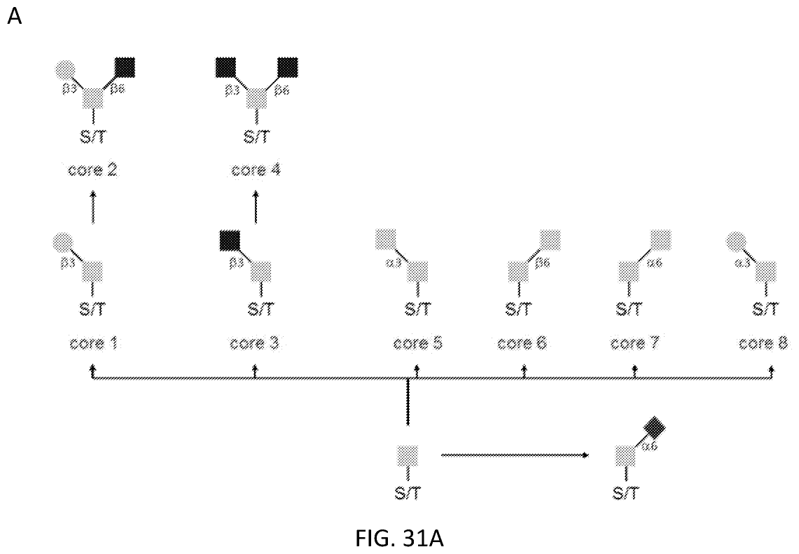

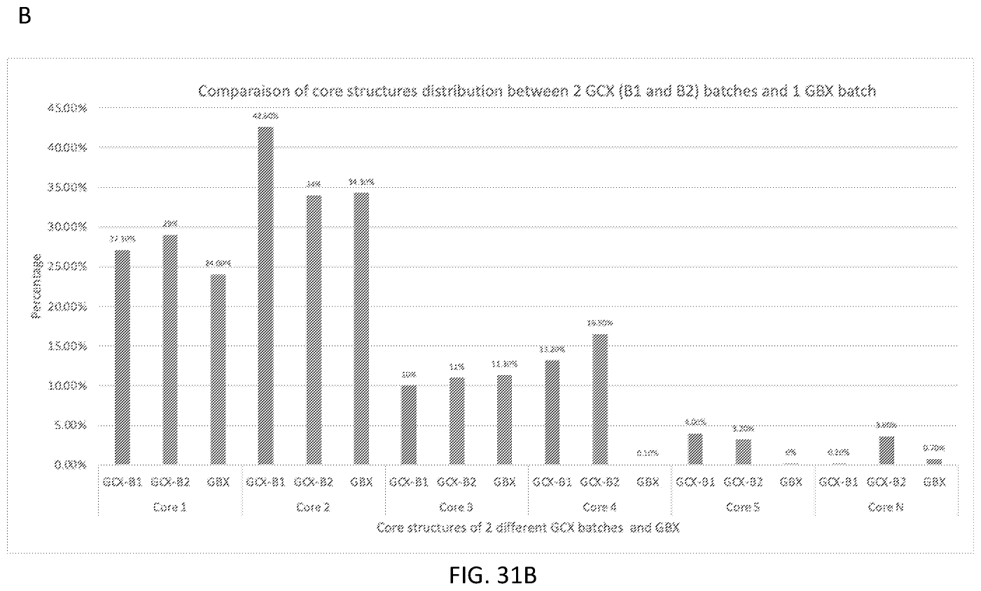

In some embodiments, the GBX composition comprises between 20% and 30% Core 1 type oligosaccharides, between 30% and 50% (e.g., about 30, 35, 40, 45, or 50%) Core 2 type oligosaccharides, between 7% and 12% (e.g., about 7, 8, 9, 10, 11 or 12%) Core 3 type oligosaccharides, between 0.1% and 2% (e.g., about 0.1, 0.2, 0.3, 0.4, 0.5, 0.8, 1, 1.2, 1.5, 1.8, 1.9, or 2%) Core 4 type oligosaccharides, no Core 5 type oligosaccharides, and between 0.1% and 2% (e.g., about 0.1, 0.5, 0.8, 1, 1.5, or 2%) Core N type oligosaccharides as measured by liquid chromatograph-electrospray ionization tandem mass spectrometry (LC-ESI/MS) and further explained in Example 13. Cores 1-5 and N are as shown in FIG. 31A.

The GBX composition can be combined with a carrier, such as a pharmaceutically acceptable carrier, that can optionally be combined with one or more of a preservative, a flavor agent, a cryoprotectant or a stabilizer. In one aspect, the composition is processed for the ease of storage, such as lyophilization.

As used herein, the phrase “GCX composition” refers to a composition prepared by the methods described in Example 3. Briefly, a GCX a composition is produced by a method comprising: (a) stabilizing gastrointestinal mucin from intestine of an animal at pH 5.0; (b) desalinating the stabilized mucin using dialysis; (c) concentrating the desalinated mucin; (d) subjecting the concentrate from (d) to diafiltration; thereby producing the composition. In some embodiments, the concentrating in (c) is achieved by evaporation with a rotary evaporator at least 80° C. In some embodiments, the effective amount of the composition is about 0.2-0.8 grams per kilogram of the subject. In some embodiments, the method further comprises administering an immune checkpoint inhibitor before, after, or simultaneously with the composition comprising glycopeptides.

In some embodiments, the GCX composition comprises at least one glycopeptide-bound oligosaccharide having a general formulae selected from Hex1HexNAc1, HexNAc2, Hex1HexNAc1Sul, Hex1NAc2Sul1, NeuAc1HexNAc1, NeuGc1HexNAc1, Hex1HexNAc1deHex1, Hex2HexNAc1, Hex1HexNAc2, HexNAc3, Hex1HexNAc2Sul1, NeuAc1Hex1HexNAc1, NeuGc1Hex1HexNAc1, Hex2HexNAc1deHex1, Hex1NAc3Sul1, NeuAc1HexNAc2, NeuGc1HexNAc2, Hex1HexNAc2deHex1, Hex2HexNAc2, Hex1HexNAc3, Hex1HexNAc2deHex1Sul1, NeuAc1Hex1HexNAc1deHex1, Hex2HexNAcSul1, NeuGc1Hex1HexNAc1deHex1, Hex1HexNAc3Sul1, NeuAc1Hex1HexNAc2, NeuGc1Hex1HexNAc2, Hex2HexNAc2deHex1, Hex3HexNAc2, Hex1HexNAc3deHex1, Hex2HexNAc3, NeuAc1Hex1HexNAc2Sul1, NeuAc2Hex1HexNAc1, NeuGc1Hex1HexNAc2Sul1, Hex2HexNAc2deHex1Sul1, NeuAc1NeuGc1Hex1HexNAc1, NeuGc2Hex1HexNAc1, Hex1HexNAc3deHex1Sul1, NeuAc1Hex1HexNAc2deHex1, Hex2HexNAc3Sul1, NeuGc1Hex1HexNAc2deHex1, NeuAc1Hex2HexNAc2, Hex2HexNAc2deHex2, NeuGc1Hex2HexNAc2, Hex2HexNAc3deHex1, NeuAc1Hex2HexNAc2Sul1, Hex2HexNAc2deHex2Sul1, NeuGc1Hex2HexNAc2Sul1, Hex1HexNAc4deHex1, NeuAc1Hex1HexNAc3Sul1, Hex1HexNAc3deHex2Sul1, NeuAc2Hex1HexNAc2, NeuGc1Hex1HexNAc3Sul1, Hex2HexNAc3deHex1Sul1, NeuAc1Hex2HexNAc2deHex1, NeuGc2Hex1HexNAc2, NeuGc1Hex2HexNAc2deHex1, NeuGc1Hex3HexNAc2, Hex1HexNAc4deHex1 Sul1, NeuAc1Hex1HexNAc3deHex1, Hex2HexNAc3deHex2 NeuGc1Hex2HexNAc3, NeuAc1Hex2HexNAc2deHex1Sul1, Hex2HexNAc3deHex2Sul1, NeuAc1Hex2HexNAc3deHex1Sul1, NeuGc1Hex2HexNAc3deHex1Sul1, Hex2HexNAc4deHex2Sul1, Hex2HexNAc5deHex2Sul1, NeuAc1Hex5HexNAc4deHex1, and NeuAc1Hex4HexNAc4deHex2Sul1.

In some embodiments, the GCX composition comprises glycopeptide-bound oligosaccharides having at least 10, at least 20, at least, 30, at least 40, at least 45, at least 50, at least 55, at least 60, at least 65, (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65) or all of the general formulae listed above. In some embodiments, the GCX composition comprises at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%,) of all glycopeptide-bound oligosaccharides in the composition.

In some embodiments, the GCX composition comprises at least 10 glycopeptide-bound oligosaccharides having a structure selected from Galβ1-3GalNAc, GalNAcα1-3GalNAc, GlcNAcβ1-6GalNAc, 3SGalβ1-3GalNAc, 6SGlcNAcβ1-3GalNAc, 6SGlcNAcβ1-6GalNAc, NeuAcα2-6GalNAc, NeuGcα2-6GalNAc, Fucα1-2(GalNAcα1-3)Gal, Fucα1-2Galβ1-3GalNAc, Fucα1-2Galβ1-4GlcNAc, Galβ1-4GlcNAcβ1-3Gal, Galβ1-3(GlcNAcβ1-6)GalNAc, Galβ1-4GlcNAcβ1-3GalNAc, GlcNAcβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Fucα1-2Galβ-4GlcNAcβ1-3Gal, 6SGlcNAcβ1-3(GlcNAcβ1-6)GalNAc, GlcNAcβ1-3(6S-GlcNAcβ1-6)GalNAc, GalNAcα1-3(NeuAcα2-6)GalNAc, GlcNAcβ1-3(NeuAcα2-6)GalNAc, GalNAcα1-3(NeuGcα2-6)GalNAc, GlcNAcβ1-3(NeuGcα2-6)GalNAc, Fucα1-2(GalNAcα1-3)Galβ1-3GalNAc, Fucα1-2Galβ1-3(GlcNAcβ1-6)GalNAc, Fucα1-2Galβ1-3GlcNAcβ1-3GalNAc, Galβ1-3(Galβ1-4GlcNAcβ1-6)GalNAc, Galβ1-3GlcNAcβ1-3Galβ1-3GalNAc, GlcNAcβ1-3(Galβ1-4GlcNAcβ1-6)GalNAc, Fucα1-2Galβ1-3(6S-GlcNAcβ1-6)GalNAc, Fucα1-2Galβ1-3(NeuAcα2-6)GalNAc, Galβ1-3[Galβ1-4(6S)GlcNAcβ1-6]GalNAc, Fucα1-2Galβ1-3(NeuGcα2-6)GalNAc, Galβ1-4GlcNAcβ1-3(6SGlcNAcβ1-6)GalNAc, GlcNAcα1-4Galβ1-3[(6S)GlcNAcβ1-6]GalNAc, GlcNAcβ1-3[Galβ1-4(6S)GlcNAcβ1-6]GalNAc, GlcNAcα1-4Galβ1-3(NeuAcα2-6)GalNAc, NeuAcα2-3(GalNAcβ1-4)Galβ1-3GalNAc, GalNAcβ1-4(NeuGcα2-3)Galβ1-3GalNAc, NeuGcα2-3Galβ1-3(GlcNAcβ1-6)GalNAc, Fucα1-2Galβ1-3(Galβ1-4GlcNAcβ1-6)GalNAc, Galβ1-3(Fucα1-2Galβ1-4GlcNAcβ1-6)GalNAc, Galα1-3Galβ1-4GlcNAcβ1-3Galβ1-3GalNAc, Galβ1-3(Galα1-3Galβ1-4GlcNAcβ1-6)GalNAc, Fucα1-2(GalNAcα1-3)Galβ1-4GlcNAcβ1-3GalNAc, GlcNAcβ1-3(Fucα1-2Galβ1-4GlcNAcβ1-6)GalNAc, GlcNAcα1-4Galβ1-3(Galβ1-4GlcNAcβ1-6)GalNAc, NeuAcα2-3Galβ1-3[(6S)GlcNAcβ1-6]GalNAc, NeuAcα2-3Galβ1-3(NeuAcα2-6)GalNAc, NeuGcα2-6Galβ1-3(6SGlcNAcβ1-6)GalNAc, Fucα1-2Galβ1-3[Gal 01-4(6S)GlcNAcβ1-6]GalNAc, Galβ1-3[Fucα1-2Galβ1-4(6S)GlcNAcβ1-6]GalNAc, NeuAcα2-3Galβ1-3(NeuGcα2-6)GalNAc, NeuGcα2-3Galβ1-3(NeuGcα2-6)GalNAc, Fucα1-2Galβ1-4(6S)GlcNAcβ1-3(GlcNAcβ1-6)GalNAc, Fucα1-2Galβ1-4GlcNAcβ-3(6SGlcNAcβ1-6)GalNAc, GalNAcα1-3(Fucα1-2)Galβ1-3(6SGlcNAcβ1-6)GalNAc, GlcNAcβ1-3[Fucα1-2Galβ1-4(6S)GlcNAcβ1-6]GalNAc, Fucα1-2Galβ1-4GlcNAcβ1-3(NeuAcα2-6)GalNAc, Galβ1-4GlcNAcβ1-3[Galβ1-4(6S)GlcNAcβ1-6]GalNAc, Fucα1-2Galβ1-4GlcNAcβ1-3(NeuGcα2-6)GalNAc, NeuAcα2-3Galβ1-3(Galβ1-4GlcNAcβ1-6)GalNAc, Fucα1-2Galβ1-3(Fucα1-2Galβ1-4GlcNAcβ1-6)GalNAc, NeuGcα2-3Galβ1-3(Galβ1-4GlcNAcβ1-6)GalNAc, Galβ1-3[Fucα1-2(GalNAcα1-3)Galβ1-4GlcNAcβ1-6]GalNAc, NeuAcα2-3Galβ1-3[Galβ1-4(6S)GlcNAcβ1-6]GalNAc, Fucα1-2Galβ1-3[Fucα1-2Galβ1-4(6S)GlcNAcβ1-6]GalNAc, NeuGcα2-3Galβ1-3[Galβ1-4(6S)GlcNAcβ1-6]GalNAc, GlcNAcβ1-3[GalNAcα1-3(Fucα1-2)Galβ1-3GlcNAcβ1-6]GalNAc, NeuAcα2-3(GalNAcβ1-4)Galβ1-3(6SGlcNAcβ1-6)GalNAc, 6SGlcNAcβ1-3[Fucα1-2Galβ1-(Fucα1-)GlcNAcβ1-6]GalNAc, NeuAcα2-3(GalNAcβ1-4)Galβ1-3(NeuAcα2-6)GalNAc, GlcNAcβ1-3[NeuGcα2-3Galβ1-4(6S)GlcNAcβ1-6]GalNAc, Fucα1-2Galβ1-4GlcNAcβ1-3[Galβ1-4(6S)GlcNAcβ1-6]GalNAc, Galβ1-3[GalNAcα1-3(Fucα1-2)Galβ1-4(6S)GlcNAcβ1-6]GalNAc, Galβ1-3GlcNAcβ1-3[Fucα1-2Galβ1-4(6S)GlcNAcβ1-6]GalNAc, Gal(31-4GlcNAcβ1-3[Fucα1-2Galβ1-3(6S)GlcNAcβ1-6]GalNAc, NeuAcα2-3Galβ1-3(Fucα1-2Galβ1-4GlcNAcβ1-6)GalNAc, NeuGcα2-3(GalNAcβ1-4)Galβ1-3(NeuGcα2-6)GalNAc, NeuGcα2-3Galβ1-3 (Fucα1-2Galβ1-4GlcNAcβ1-6)GalNAc, Galα1-3Galβ1-3(NeuGcα2-3Galβ1-4GlcNAcβ1-6)GalNAc, GalNAcα1-3(Fucα1-2)Galβ1-3GlcNAcβ1-3(6SGlcNAcβ1-6)GalNAc, GlcNAcβ1-3[GalNAcα1-3(Fucα1-2)Galβ1-(6S)GlcNAcβ1-6]GalNAc, GlcNAcα1-4(Fucα1-2)Galβ1-4GlcNAcβ1-3(NeuAcα2-6)GalNAc, Fucα1-2Galβ1-4GlcNAcβ1-3(Fucα1-2Galβ1-4GlcNAcβ1-6)GalNAc, Galβ1-(Fuc)GlcNAcβ1-3(Fucα1-2Galβ1-4GlcNAcβ1-6)GalNAc, NeuGcα2-3Galβ1-3(GlcNAcα1-4Galβ1-4GlcNAcβ1-6)GalNAc, NeuAcα2-3Galβ1-3[Fucα1-2Galβ1-4(6S)GlcNAcβ1-6]GalNAc, Fucα1-2Galβ1-3[GalNAcα1-3(Fucα1-2)Galβ1-3(6S)GlcNAcβ1-6]GalNAc, Fucα1-2Galβ1-3[GalNAcα1-3(Fucα1-2)Galβ1-4(6S)GlcNAcβ1-6]GalNAc, Fucα1-2Galβ1-4GlcNAcβ1-3[Fucα1-2Galβ1-4(6S)GlcNAcβ1-6]GalNAc, NeuAcα2-3Galβ1-3[GalNAcα1-3(Fucα1-2)Gal-(6S)GlcNAcβ1-6]GalNAc, GlcNAcα1-4Galβ1-3[NeuGcα2-6Gal-(Fuc)(6S)GlcNAcβ1-6]GalNAc, GlcNAcα1-4(Fucα1-2)GlcNAcβ1-3[Fucα1-2)Galβ1-4(6S)GlcNAcβ1-6]GalNAc, GalNAcα1-3(Fucα1-2)Galβ1-4GlcNAcβ1-3[GalNAcα1-3(Fucα1-2)Galβ1-4(6S)GlcNAcβ1-6]GalNAc, and NeuAcα2-Galβ1-4GlcNAcβ1-2Manα1-3(Galβ1-4GlcNAcβ1-2Manα1-6)Manβ1-4GlcNAcβ1-4(Fucα1-6)GlcNAc.

In some embodiments, the GCX composition comprises between 20% and 30% Core 1 type oligosaccharides, between 30% and 50% (e.g., about 30, 35, 40, 45, or 50%) Core 2 type oligosaccharides, between 7% and 12% (e.g., about 7, 8, 9, 10, 11 or 12%) Core 3 type oligosaccharides, between 8% and 20% (e.g., about 8, 9, 10, 11, 12, 13, 14, 15, 16, 17, 18, 19, or 20%) Core 4 type oligosaccharides, between 2% and 6% (e.g., about 2, 3, 4, 5, or 6%) Core 5 type oligosaccharides and between 0.1% and 5% (e.g., about 0.1, 0.5, 0.8, 1, 1.5, 2, 2.5, 3, 3.5, 4, 4.5, or 5%) Core N type oligosaccharides as measured by liquid chromatograph-electrospray ionization tandem mass spectrometry (LC-ESI/MS) and further explained in Example 13. Cores 1-5 and N are as shown in FIG. 31A.

The GCX composition can be combined with a carrier, such as a pharmaceutically acceptable carrier, that can optionally be combined with one or more of a preservative, a flavor agent, a cryoprotectant or a stabilizer. In one aspect, the composition is processed for the ease of storage, such as lyophilization.

### Treating or Preventing Tumors (Prophylaxys)

Some aspects of the present disclosure are directed to a method of treating a tumor in a subject in need thereof comprising administering to the subject an effective amount of a composition comprising glycopeptides obtained from gastrointestinal mucins, wherein the composition comprises less than about 25% (w/w) (e.g., less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or less) free glycans, and wherein the total protein content of the composition is 12% or less (w/w) (e.g., less than about 25%, 24%, 23%, 22%, 21%, 20%, 19%, 18%, 17%, 16%, 15%, 14%, 13%, 12%, 11%, 10%, 9%, 8%, 7%, 6%, 5% 4%, 3%, 2%, 1% or less), and wherein the composition comprises glycopeptides of multiple different oligosaccharide structures. In one aspect, the method further comprises identifying the subject for the therapy, by determining if the tumor or cancer is resistant to a checkpoint inhibitor. In some embodiments, the composition is administered as a monotherapy. In some embodiments, the composition is administered together with other cancer treatments, such as surgical resection, chemotherapy, immunotherapy (e.g., CAR-T, CAR-NK, or antibody-related therapies) or radiotherapy. In one aspect, the composition comprises, or consists essentially of, or consists of GBX or GCX. In one aspect, the subject has been diagnosed with cancer, in one aspect, a cancer that is resistant or can become resistant to an immune checkpoint inhibitor. In a further aspect, the subject diagnosed with the cancer is treated. Subjects not diagnosed with the cancer do not receive the composition.

Another aspect of the disclosure is directed to a method for enhancing an anti-tumor activity of a CAR-T therapy or an Immune Checkpoint Inhibitor (ICI) therapy against a tumor in a subject comprising administering to the subject a composition comprising glycopeptides obtained from gastrointestinal mucins, wherein the composition comprises less than about 25% (w/w) (e.g., less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or less) free glycans, and wherein the total protein content of the composition is 12% or less (w/w) (e.g., less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or less), and wherein the composition comprises glycopeptides of multiple different oligosaccharide structures. In one aspect, the composition comprises, or consists essentially of, or consists of GBX or GCX. In one aspect, the subject has been diagnosed with cancer, in one aspect, a cancer that is resistant or can become resistant to an immune checkpoint inhibitor. In a further aspect, the subject diagnosed with the cancer is treated. Subjects not diagnosed with the cancer do not receive the composition.

Another aspect of the disclosure is directed to a method for cancer prophylaxis (preventing formation of a tumor) in a subject comprising administering to the subject a composition comprising glycopeptides obtained from gastrointestinal mucins, wherein the composition comprises less than about 25% (w/w) (e.g., less than about 25%, 24%, 23%, 22%, 21%, 20%, 9%, 18%, 17%, 16%, 1%, 14%, 13%, %¹²%, 11%, 10%, 9%, 8%, 7%, 6%, 5%, 4%, 3%, 2%, 1% or less) free glycans, and wherein the total protein content of the composition is 12% or less (w/w) (e.g., less than about 25%, 24%, 23%, 22%, 21%, 20%, 9%, 18%, 17%, 16%, 1%, 14%, 13%, %¹²%, 11%, 10%, 9%, 8%, 7%, 6%, 5%, 4%, 3%, 2%, 1% or less), and wherein the composition comprises glycopeptides of multiple different oligosaccharide structures. In one aspect, the composition comprises, or consists essentially of, or consists of GBX or GCX. In one aspect, the subject has been diagnosed with cancer, in one aspect, a cancer that is resistant or can become resistant to an immune checkpoint inhibitor. In a further aspect, the subject diagnosed with the cancer is treated. Subjects not diagnosed with the cancer do not receive the composition.

The tumor is not limited and may be any kind of cancer, e.g., solid or blood cancer, e.g. carcinoma or sarcoma. In some embodiments, the cancer is ICI resistant. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the tumor is an immune checkpoint inhibitor (ICI)-resistant tumor. In some embodiments, the tumor is melanoma. In some embodiments, the tumor is ICI resistant melanoma. In some embodiments, the tumor is colorectal cancer (CRC). In some embodiments, the tumor is ICI resistant CRC. The cancer can be Stage I, Stage II, Stage III or Stage IV. The cancer can be localized or metastatic.

The subject can be any mammal, such as for example a canine, a feline or a human.

In some embodiments, the composition comprises less than about 0.1%, 0.5%, 1%, 5%, 10%, 12%, 14%, 15%, 6%1, 7%1, 8%1, 19%, 20%, 21%, 22%, 23%, or 24% free glycans (e.g., glycans not bond to peptides or proteins. In some embodiments, the composition comprises substantially no free glycans.

In some embodiments, the composition (e.g., a composition comprising sialylated glycopeptide-bound oligosaccharides) is administered (e.g., with or without a checkpoint inhibitor, e.g., an anti-PD-1 antibody, an anti-CTLA-4 antibody, an anti-PD-L1 antibody, or an anti-PD-L2 antibody) in an amount effective to: increase T cells expansion, preferably CD4 and CD8 T cell expansion and IFN-gamma-producing CD4 and CD8 T cell expansion; increase T cells trafficking and infiltration to the tumor, preferably CD4 and CD8 T cell, and IFN-gamma-producing CD4 and CD8 T cell; increase the level of perforin, increase the level or excretion activity of perforin secreting CD8+ T cells, preferably tumor infiltrating CD8+ T cells; increase the level or activity of granzyme B producing CD8+ T cells; instruct T cells to target antigens on the tumor cells using glycans on the glycopeptides as molecular mimicry of glycans on cancer cells (glycosylation of tumor proteins generates neo-antigens that can serve as targets for tumor-specific T cells and these neo-antigens may share epitopes with GNU glycans through molecular mimicry); increase the level or activity of TNF-producing CD8+ T cells, preferably tumor infiltrating TNFα-producing CD8+ T cells; increase the level or activity of TNFα-producing CD4+ T cells, preferably tumor infiltrating TNFα-producing CD4+ T cells; increase the level or activity of IL-17-producing CD4+ T cells (Th17), preferably tumor infiltrating IL-17-producing CD4+ T cells; increase and stimulate antigen-presenting cells (e.g., dendritic cells (DCs)) to prime and activate T cells; promote a Th1-like immune tone favoring an anti-tumor response by modulating the microbiome composition and function and modulating the phenotype of immune cells (glycans and glycopeptides and specific bacteria modulated by glycans and glycopeptides can mediate an adjuvant effect on dendritic cells and other immune cells to trigger a type 1 interferon2 or an IL-12 fingerprint, which is associated with protective type 1 T helper cell (TH1 cell) and CD8+ T cell antitumor immune responses), induce pro-inflammatory cytokines or other molecules (e.g., IFN-gamma and granzyme B); bind and stimulate Toll-like Receptors (TLR) and initiate T-cell immunity by inducing maturation of DCs and by acting as co-stimulatory receptors on T cells; reduce expression of immune checkpoint molecules on T-cells or cancer cells; reduce expression of PD1 receptor on T cells and/or PD1-Ligand on cancer cells or reduce or eliminate interactions between PD1 receptor and PD1-Ligand; reduce expression of CTLA4 on CD4+ T cells and/or CD8+ T cells, preferably tumor infiltrating CD4+ T cells and/or CD8+ T cells; reduce immunosuppressor cells (e.g., Treg cells), myeloid-derived suppressor cells, and/or anti-inflammatory M2 macrophages, preferably tumor infiltrating immunosuppressor cells (e.g., Treg cells, FoxP3+ CD4 T cells), myeloid-derived suppressor cells, and/or anti-inflammatory M2 macrophages; reduce a level or activity of macrophages, preferably tumor infiltrating macrophages; increase a level or activity of pro-inflammatory M1 macrophages, preferably tumor infiltrating pro-inflammatory M1 macrophages, or a ratio of M1 macrophages to M2 macrophages; reduce immune-suppressive IL-10 and/or TGF-beta production in a tumor micro-environment (TME) and/or in systemic circulation; reduce the level or activity of RANTES in a tumor micro-environment (TME) and/or in systemic circulation; reduce or eliminate interactions between tumor-associated glycan and inhibitory immune receptors (e.g. lectin); reduce or eliminate galectin shedding by the tumor in a tumor micro-environment (TME) or reduce or eliminate interactions of tumor-shed galectin and glycans present on immune cells; bind to Dendritic Cell-Specific Intercellular adhesion molecule-3-Grabbing Non-integrin (DC-SIGN) and increase internalization and presentation of antigen by APC cells; produce microbiota-derived metabolites (e.g., Inosine, short-chain fatty acids (SCFA) like butyrate, acetate, or propionate) and/or microbiota-derived structural components or fragments thereof that activate anti-tumor immune cells, influence their phenotype, or that directly affect tumor cells and the way they respond to T cells; and/or increase expression of chemokines, inflammasome-related genes, antigen presentation-related genes, and/or enhance the anti-tumor activity of a CAR-T therapy or the effectiveness of Immune Checkpoint Inhibitor (ICI) therapy.

Mucin 1 (MUC1) is commonly overexpressed in various epithelial adenocarcinomas such as lung, liver, colon, breast, pancreatic, and ovarian cancer. Mucin 1 (MUC-1) is aberrantly glycosylated in tumors. The aberrant glycans on MUC1 are oftentimes Tn, STn, sialyl-Lewisa and/or sialyl-Lewisx. Tn and sTn antigens are found in breast, prostate, colon, respiratory, pancreas, ovarian and gastric cancers. Tn antigen is expressed in >90% of breast cancers and 10-90% of other forms of cancers, and is prevalent in 25-70% of premalignant tissues in the colon. Many epithelial cancers (>80%) display sTn antigen and its expression is associated with decreased overall survival of patients. TF and sTF are both expressed on breast cancer, while TF is more prevalent in gastric, colon, pancreas, ovary, prostate, and stomach cancers and is also found in 90% of cancer types. Tn, sTn, and TF antigens are found co-expressed on tumor cells and their expression correlates to disease progression.

In addition to truncated O-glycans, the expression of specific sialyl and fucosyl transferases generates sialyl-Lewis antigens, NeuAcα2,3-Galβ1,3-(Fucα1,4)-GlcNAc-R (sLea, sialyl-Lewisa) and NeuAcα2,3-Galβ1,3-(Fucα1,3)-GlcNAc-R (sLex, sialyl-Lewisx). Sialyl-Lewisa structures are found in >50% of colon, stomach, and pancreas cancers, along with lung, liver, breast, and mesothelioma cancers. Whereas, sialyl-Lewisx structures are found in >90% of pancreas and stomach cancers, along with colon, esophagus, ovary, and breast cancers.

In some embodiments, the tumor has cells that overexpress MUC-1 (e.g., aberrantly glycosylated MUC-1) on their surface. In some embodiments, the tumor has cells with aberrantly glycosylated MUC-1 on their surface. In some embodiments, the aberrantly glycosylated MUC-1 comprises Tn, STn, sialyl-Lewisa and/or sialyl-Lewisx glycosylation. In some embodiments, the tumor has cells expressing one, two, or all three of Tn, sTn, and TF aberrant glycosylation. In some embodiments, the tumor is a Tn-MUC1 glycosylated tumor type). In some embodiments, the tumor is a lung, liver, colon, breast, pancreatic, prostate, gastric, mesothelioma, esophageal, or ovarian tumor. In some embodiments, the composition increases the effectiveness of Immune Checkpoint Inhibitor (ICI) therapy.

In some embodiments, the compositions disclosed herein enhance the anti-tumor activity of a CAR-T therapy by (1) increase the proportion of T cells overall and specific effector T cells in the tumor microenvironment (e.g., T cells cross-reactive with glycans in the composition and on the surface of the tumor) (2) decrease one or more immunosuppressant factors or conditions (e.g., serum IL-10, exhaustion marker CTLA4, and/or infiltrating macrophages). Combined, these effects allow for a better efficacy of engineered T cells (CAR-T). In some embodiments, the compositions disclosed herein enhance the anti-tumor activity of a CAR-T therapy in a solid tumor.

In some embodiments, the Car-T therapy is directed to the protein core MUC1. In some embodiments, the Car-T therapy is directed to specific truncated O-glycopeptide epitopes not expressed on normal tissues and shown to differentiate between wild-type and Tn-glycoforms of MUC1. In some embodiments, the Car-T therapy is directed to Tn (GalNAcα1-Ser/Thr) or sialyl-Tn (STn, NeuAcα2,6GalNAc-Ser/Thr). In some embodiments, the tumor is a lung, liver, colon, breast, pancreatic, prostate, gastric, mesothelioma, esophageal, or ovarian tumor. The tumor is not limited and may be any tumor described herein.

In some embodiments of the compositions and methods disclosed herein, the composition comprises the sialyl Tn antigen, the sialyl T antigen and the disialyl T antigen. In some embodiments, the composition comprising the sialyl Tn antigen, the Sialyl T antigen and the disialyl T antigen is orally administered to a subject and cross reacts with one or more of a sialyl Tn antigen, a Sialyl T antigen and a disialyl T antigen present on the surface of the tumor cells in the subject.

In some embodiments of the compositions and methods disclosed herein, the composition comprises the sialyl T antigen, the T antigen, the Lewis X antigen, and the Lewis A antigen. In some embodiments, the composition comprising the sialyl T antigen, the T antigen, the Lewis X antigen, and the Lewis A antigen is orally administered to a subject and cross reacts with one or more of a sialyl T antigen, a T antigen, a Lewis X antigen, and a Lewis A antigen present on the surface of the tumor cells in the subject.

In some specific embodiments, the composition (e.g., a composition comprising sialylated glycopeptide-bound oligosaccharides) is administered in an amount effective to increase IFN-gamma-producing CD4 cells (e.g., tumor infiltrating IFN-gamma-producing CD4 cells). IFN-gamma-producing CD4+ T cells are also known as Th1 cells. Th1 cells can improve ICI therapy. Th1 cells are responsible for activating and regulating the development and persistence of CD8 T cells. In addition, Th1 cells activate antigen-presenting cells (APC).

In some embodiments, the tumor is refractory to immune checkpoint inhibitor therapy. The immune checkpoint inhibitor is not limited. In some embodiments, the immune checkpoint inhibitor is an anti-PD-1 antibody, an anti-CTLA-4 antibody, an anti-PD-L1 antibody, or an anti-PD-L2 antibody. In some embodiments, the immune checkpoint inhibitor is nivolumab, pembrolizumab, atezolizumab, durvalumab, pidilizumab, PDR001, BMS-936559, avelumab, or SHR-1210. In some embodiments, the immune checkpoint inhibitor is nivolumab.

In some embodiments, the microenvironment of the tumor (TMI) is characterized by one or more of decreased T-cell, B-cell, and/or antigen-presenting cell function, decreased IL-2 production, generation of exhausted T cells, and increased circulating soluble IL-2 receptor, and the presence of one or more immunosuppressive cell populations (e.g., tumor-associated macrophages (TAMs), T-regulatory cells (Tregs) and/or myeloid-derived suppressor cells (MDSCs).

In some embodiments, the total glycoprotein content of the composition is 12% or less (w/w). In some embodiments, the total glycoprotein content of the composition (w/w) is less than about 11%, 10%, 9%, 8%, 7%, 6%, 5%, 4%, 3%, 2%, 1%, 0.5%, or 0.1%. In some embodiments, the composition comprises substantially no glycoproteins.

In some embodiments, the composition comprises at least 1, 2, 3, 4, 5, 6, 7, 8, 9, 10, 11, 12, 13, 14, 15, 16, 20, 25, 30, or more sialylated glycopeptide-bound oligosaccharides (i.e., 1, 2, 3, etc. different sialylated glycopeptide-bound oligosaccharide structures). In some embodiments, the sialic acid content of the composition is greater than 5%, 10%, 15%, 20%, 22.5%, 25%, 27.5%, 30%, 32.5%, 35%, or 40% (w/w).

Without being bound by theory, Applicants postulate that glycopeptides comprising sialic acid structures are similar to structures on the surface of tumor cells (e.g., melanoma tumor cells, CRC) and therefore activate the anti-cancer activity of immune cells (e.g., T cells) via binding to lectins on the immune cell surface (i.e., molecular mimicry) or via binding to and presentation by APCs to T-cells. Sialylated glycans may also block the interaction between lectins present on immune cells and glycans present on the tumor cells. Blocking this interaction is important to inhibit a tolerogenic tumor microenvironment. It is also postulated that sialylated glycans can block the interaction between galectin shed by the tumor and glycans present on immune cells. Again, here, blocking this interaction is important as otherwise the interaction contribute to the tolerogenic tumor microenvironment.

Sialylated glycans may also act as toll-like receptor ligands which may, upon binding to APCs, induce APC maturation, presentation of the sialylated glycans to T-cells, and activation of T-cells. Sialylated glycans may also directly bind to T-cell TLRs, increasing the proliferation of activated T-cells and reducing the immunosuppressive activity of Treg cells. See, Rodríguez, E., Schetters, S. & van Kooyk, Y. “The tumor glyco-code as a novel immune checkpoint for immunotherapy.” Nat Rev Immunol 18, 204-211 (2018), which is incorporated herein by reference.

It is also postulated that the sialylated glycans may cause tumor cells to change their glycosylation pattern and thereby reduce the interactions between immunosuppressive lectins and tumor-associated glycans and increase anti-tumor immune response. See, Chakraborty A, Dimitroff C J. Cancer immunotherapy needs to learn how to stick to its guns. J Clin Invest. 2019 Dec. 2; 129(12):5089-5091, which is incorporated herein by reference.

Finally and without being bound by theory, Applicant theorizes that sialylated glycans modulate the microbiome and metabolites produced by the microbiome in a direction that induces a more potent tumor immune response.

In some embodiments, administration of the composition comprising glycopeptides increases the sensitivity of the tumor to immune checkpoint-inhibitor therapy, decreases tumor weight, and/or decrease tumor volume. In one aspect, the composition comprises, or consists essentially of, or consists of GBX or GCX.

In some embodiments, the method further comprises administering an immune checkpoint inhibitor to the subject (e.g., human). The administration schedule for the composition and ICI are not limited. In some embodiments, the ICI is administered before, or before and simultaneously with, the composition comprising glycopeptides. In some embodiments, the composition is administered starting at least 2 days, at least 3 days, at least 4 days, at least 5 days, at least 6 days, at least a week, at least 2 weeks, or at least a month before the IC. In some embodiments, the composition is administered simultaneously with the ICI. In some embodiments, the composition is administered with the ICI or shortly after the ICI and administration is continued for at least 2 days, at least 3 days, at least 4 days, at least 5 days, at least 6 days, at least a week, at least 2 weeks, or at least a month. The immune checkpoint inhibitor (ICI) is not limited and may be any ICI described herein.

In some embodiments, the composition is administered in an effective amount to reduce or prevent a rise in the level of Monocyte Chemoattractant Protein-1 (MCP-1) caused by the immune checkpoint inhibitor in a tumor micro-environment (TME) and/or in systemic circulation.

The composition may be obtained from mucins from any suitable source. In some embodiments, the mucins are obtained from porcine gastrointestinal tract. In some embodiments, the mucins are obtained from porcine stomach. In some embodiments, the compositions are obtained by the methods provided in Examples 1-3 herein. In some embodiments, the composition is a composition comprising a mixture of glycopeptides or a composition obtained by a purification method provided in WO 2020/104486, published on May 28, 2020, which is incorporated herein by reference in its entirety. In some embodiments, the composition is a composition comprising a mixture of glycopeptides or a composition obtained by a purification method provided in WO 2020/157321, published on Aug. 6, 2020, which is incorporated herein by reference in its entirety.

In some embodiments, the composition is obtained from porcine gastric mucins, wherein: the composition is obtained without subjecting the mucins or a partially purified fraction thereof to conditions or reagents that cause complete release of oligosaccharides from glycopeptides; the total oligosaccharide content of the composition is >10% (w/w); the ratio of glycopeptides:free glycans is >4:1 (w/w); and the total glycoprotein content of the composition is 12% or less (w/w).

In some embodiments, the total oligosaccharide content of the composition (e.g., composition) is greater than about 10%, 12%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 50%, or 55% (w/w). As used herein, the total oligosaccharide content is the total weight of oligosaccharide in the composition. Such weight does not include the weight of proteins or peptides attached to the oligosaccharides. In some embodiments, the oligosaccharide content of the composition is greater than about 10% (w/w). In some embodiments, the oligosaccharide content of the composition is greater than or equal to about 15% (w/w). In some embodiments, the oligosaccharide content of the composition is greater than or equal to about 20% (w/w). In some embodiments, the oligosaccharide content comprises substantially all oligosaccharides bound to glycoprotein or glycopeptide without substantially any unbound oligosaccharides. Methods of determining oligosaccharide content are known in the art and are not limited. In some embodiments, oligosaccharide content is determined by HPAEC-PAD with an acid pre-treatment to hydrolyze the glycans into monosaccharides.

In some embodiments, the ratio of glycopeptides:free glycans is >4:1 (w/w). In some embodiments, the ratio of glycopeptides:free glycans (w/w) is about 4:1, about 5:1, about 6:1, about 7:1, about 8:1, about 9:1, about 10:1, about 11:1, about 12:1, about 13:1, about 14:1, about 15:1, about 20:1, or more.

In some embodiments, the total glycoprotein content of the composition is 12% or less (w/w). In some embodiments, the total glycoprotein content of the composition is 11%, 10%, 9%, 8%, 7%, 6%, 5%, 4%, 3%, 2%, 1%, or less (w/w).

In some embodiments, the composition comprises at least one glycopeptide-bound oligosaccharide having a general formulae selected from Hex1HexNAc1, HexNAc2, Hex1HexNAc1Sul1, HexNAc1deHex1Sul1, HexNAc2Sul1, NeuAc1HexNAc1, Hex2HexNAc1, Hex1HexNAc1deHex1, Hex1HexNAc2, HexNAc2deHex1, Hex1HexNAc1deHex1Sul1, Hex1HexNAcSul1, Hex2HexNAc1deHex1, NeuAc1Hex1HexNAc1, Hex1HexNAc1deHex2, Hex2HexNAc2, Hex1HexNAc2deHex1, Hex2HexNAc1deHex1Sul1, Hex1HexNAc1deHex2Sul1, Hex1HexNAc3, Hex1HexNAc2deHex1Sul1, NeuAc1Hex1HexNAc1deHex1, Hex2HexNAc2Sul1, NeuAc1Hex1HexNAc2, Hex1HexNAc3Sul1, Hex2HexNAc2deHex1, Hex1HexNAc2deHex2, Hex1HexNAc3deHex1, Hex2HexNAc3, Hex2HexNAc2deHex1Sul1, Hex1HexNAc4, Hex1HexNAc3deHex1Sul1, Hex3HexNAc2deHex1, NeuAc1Hex2HexNAc2, Hex2HexNAc2deHex2, Hex2HexNAc3deHex1, Hex3HexNAc2deHex1Sul1, Hex2HexNAc2deHex2Sul1, Hex2HexNAc4, Hex2HexNAc3deHex1Sul1, Hex3HexNAc3deHex1, Hex2HexNAc4deHex1, Hex3HexNAc3deHex1Sul1, and Hex4HexNAc3deHex1Sul1. In some embodiments, the composition comprises at least one or both of NeuAc1HexNAc1 and NeuAc1Hex1HexNAc1.

In some embodiments, the composition comprises glycopeptide-bound oligosaccharides having at least 10, at least 20, at least 30, at least 40, at least 44, or all of the general formulae provided above (e.g., the composition comprises at least 10, 20, 30, 40, 44 glycopeptides, wherein each of the glycopeptides comprises a different glycopeptide-bound oligosaccharide from the above list). In some embodiments, the composition comprises glycopeptide-bound oligosaccharides having at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or all of the general formulae provided above.

In some embodiments, the composition comprises at least one glycopeptide-bound oligosaccharide having a general formulae selected from Hex1HexNAc1, Hex1HexNAc1deHex1, Hex1HexNAc1deHex1Sul1, Hex1HexNAc1deHex2, Hex1HexNAc1deHex2Sul1, Hex1HexNAc1Sul1, Hex1HexNAc2, Hex1HexNAc2deHex1, Hex1HexNAc2deHex1Sul1, Hex1HexNAc2deHex2, Hex1HexNAc2Sul1, Hex1HexNAc3, Hex1HexNAc3deHex1, Hex1HexNAc3deHex1Sul1, Hex1HexNAc3Sul1, Hex1HexNAc4, Hex1HexNAcSul1, Hex2HexNAc1, Hex2HexNAc1deHex1, Hex2HexNAc1deHex1Sul1, Hex2HexNAc2, Hex2HexNAc2deHex1, Hex2HexNAc2deHex1Sul1, Hex2HexNAc2deHex2, Hex2HexNAc2deHex2Sul1, Hex2HexNAc2Sul1, Hex2HexNAc3, Hex2HexNAc3deHex1, Hex2HexNAc3deHex1Sul1, Hex2HexNAc4, Hex2HexNAc4deHex1, Hex3HexNAc2deHex1, Hex3HexNAc2deHex1Sul1, Hex3HexNAc3deHex1, Hex3HexNAc3deHex1Sul1, Hex4HexNAc3deHex1Sul1, HexNAc1deHex1Sul1, HexNAc2, HexNAc2deHex1, HexNAc2Sul1, NeuAc1Hex1HexNAc1, NeuAc1Hex1HexNAc1deHex1, NeuAc1Hex1HexNAc2, NeuAc1Hex2HexNAc2, and NeuAc1HexNAc1.

In some embodiments, the composition comprises glycopeptide-bound oligosaccharides having at least 10, at least 20, at least, 30, at least 40, at least 45, or all (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46) of the general formulae listed above. In some embodiments, the composition comprises at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%,) of all glycopeptide-bound oligosaccharides in the composition.

In some embodiments, the composition comprises at least one glycopeptide-bound oligosaccharide having a general formulae selected from Hex1HexNAc1, HexNAc2, Hex1HexNAc1 Sul, Hex1NAc2Sul1, NeuAc1HexNAc1, NeuGc1HexNAc1, Hex1HexNAc1deHex1, Hex2HexNAc1, Hex1HexNAc2, HexNAc3, Hex1HexNAc2Sul1, NeuAc1Hex1HexNAc1, NeuGc1Hex1HexNAc1, Hex2HexNAc1deHex1, Hex1NAc3Sul1, NeuAc1HexNAc2, NeuGc1HexNAc2, Hex1HexNAc2deHex1, Hex2HexNAc2, Hex1HexNAc3, Hex1HexNAc2deHex1Sul1, NeuAc1Hex1HexNAc1deHex1, Hex2HexNAcSul1, NeuGc1Hex1HexNAc1deHex1, Hex1HexNAc3Sul1, NeuAc1Hex1HexNAc2, NeuGc1Hex1HexNAc2, Hex2HexNAc2deHex1, Hex3HexNAc2, Hex1HexNAc3deHex1, Hex2HexNAc3, NeuAc1Hex1HexNAc2Sul1, NeuAc2Hex1HexNAc1, NeuGc1Hex1HexNAc2Sul1, Hex2HexNAc2deHex1Sul1, NeuAc1NeuGc1Hex1HexNAc1, NeuGc2Hex1HexNAc1, Hex1HexNAc3deHex1Sul1, NeuAc1Hex1HexNAc2deHex1, Hex2HexNAc3Sul1, NeuGc1Hex1HexNAc2deHex1, NeuAc1Hex2HexNAc2, Hex2HexNAc2deHex2, NeuGc1Hex2HexNAc2, Hex2HexNAc3deHex1, NeuAc1Hex2HexNAc2Sul1, Hex2HexNAc2deHex2Sul1, NeuGc1Hex2HexNAc2Sul1, Hex1HexNAc4deHex1, NeuAc1Hex1HexNAc3Sul1, Hex1HexNAc3deHex2Sul1, NeuAc2Hex1HexNAc2, NeuGc1Hex1HexNAc3Sul1, Hex2HexNAc3deHex1Sul1, NeuAc1Hex2HexNAc2deHex1, NeuGc2Hex1HexNAc2, NeuGc1Hex2HexNAc2deHex1, NeuGc1Hex3HexNAc2, Hex1HexNAc4deHex1 Sul1, NeuAc1Hex1HexNAc3deHex1, Hex2HexNAc3deHex2 NeuGc1Hex2HexNAc3, NeuAc1Hex2HexNAc2deHex1Sul1, Hex2HexNAc3deHex2Sul1, NeuAc1Hex2HexNAc3deHex1Sul1, NeuGc1Hex2HexNAc3deHex1Sul1, Hex2HexNAc4deHex2Sul1, Hex2HexNAc5deHex2Sul1, NeuAc1Hex5HexNAc4deHex1, and NeuAc1Hex4HexNAc4deHex2Sul1.

In some embodiments, the composition comprises glycopeptide-bound oligosaccharides having at least 10, at least 20, at least, 30, at least 40, at least 45, at least 50, at least 55, at least 60, at least 65, (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65) or all of the general formulae listed above. In some embodiments, the composition comprises at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%,) of all glycopeptide-bound oligosaccharides in the composition. In some embodiments, the composition comprises at least one glycopeptide-bound oligosaccharide having a structure selected from Galβ1-3GalNAcol, GlcNAcβ1-4Galol, GlcNAcα1-4Galol, HexNAc-GlcAol, GlcNAcβ1-6GalNAcol, Galβ1-4(6S)GlcNAcol, 6SGlcNAc-Fucol, Galβ1-4(6S)GlcNAcol, (S)Galβ1-GlcNAcol, (S)Galβ1-GlcNAcol, 6SGlcNAcβ1-6GalNAcol, 6SGlcNAcβ1-3GalNAcol, NeuAc-HexNAcol, NeuAcα2-6GalNAcol, Galβ1-4GlcNAcβ1-3Gal minus H₂O, Fucα1-2(GalNAcα1-3)Galol, Gal-GlcNAc-Fucol, Gal-GlcNAc-Fucol, Fucα1-2Galβ1-4GlcNAcol, Fucα1-2Galβ1-3GlcNAcol, Fucα1-2Galβ1-3GalNAcol, Galβ1-4GlcNAcβ1-3Gal, GlcNAcα1-4Galβ1-4GlcNAc minus H₂O, GlcNAc-GlcNAc-Fucol, Galβ1-3(GlcNAcβ1-6)GalNAcol, GlcNAcα1-4Galβ1-3GalNAcol, GlcNAcα1-4Galβ1-4GlcNAcol, Fucα1-2Galβ1-4(6S)GlcNAcol, Galβ1-4(Fucα1-3)(6S)GlcNAcol, 6SGalβ1-3(Fucα1-4)GlcNAcol, SGalβ1-3(GlcNAcβ1-6)GalNAcol, Galβ1-3(6SGlcNAcβ1-6)GalNAcol, Galβ1-4(6S)GlcNAcβ1-6GalNAcol, Fucα1-2Galβ-4GlcNAcβ1-3Gal minus H₂O, Galβ1-3(NeuAcα2-6)GalNAcol, NeuAcα2-3Galβ1-3GalNAcol, Fucα1-2Galβ1-4(Fucα1-3)GlcNAcol, Fucα1-2Galβ-4GlcNAcβ1-3Gal, GlcNAcα1-4Galβ1-4GlcNAcβ1-3Gal minus H₂O, Fucα1-2(GalNAcα1-3)Galβ1-4GlcNAcol, Fucα1-2(GalNAcα1-3)Galβ1-3GalNAcol, GlcNAcα1-4Gal(Fuc)GlcNAcol, GlcNAcα1-4Gal(Fuc)GlcNAcol, Fucα1-2Galβ1-3(GlcNAcβ1-6)GalNAcol, GlcNAcα1-4Galβ1-4GlcNAcβ1-3Gal, Galβ1-3(Galβ1-4GlcNAcβ1-6)GalNAcol, GlcNAcα1-4Galβ1-4GlcNAcβ1-3Galol, Galβ1-3(Galβ1-4GlcNAcβ1-6)GalNAcol, Galβ1-4GlcNAcβ1-3Galβ1-3GalNAcol, Fucα1-2Galβ1-4(6S)GlcNAcβ1-4Gal minus H₂O, Fucα1-2(S)Galβ1-4GlcNAcβ1-4Gal minus H2O, Fucα1-2Gal(Fuc)(6S)GlcNAcol, Fucα1-2Galβ1-3(6S)GlcNAcβ1-4Galol, GlcNAcα1-4Galβ1-3(GlcNAcβ1-6)GalNAcol, GlcNAcα1-3Galβ1-4GlcNAcβ1-6GalNAcol, Fucα1-2(GalNAcα1-3)Galβ1-4(6S)GlcNAcol, Fucα1-2Galβ1-4(6S)GlcNAcβ1-3GalNAcol, Fucα1-2Galβ1-3(6S-GlcNAcβ1-6)GalNAcol, Fucα1-2Galβ1-3(NeuAcα2-6)GalNAcol, Galβ1-3[Galβ1-4(6S)GlcNAcβ1-6]GalNAcol, GlcNAcα1-4Galβ1-3(NeuAcα2-6)GalNAcol, GlcNAcα1-4Galβ1-3[(6S)GlcNAcβ1-6]GalNAcol, GalNAcα1-3(Fucα1-2)Galβ1-4GlcNAcβ1-3Gal minus H₂O, GlcNAcα1-4Galβ1-3(NeuAcα2-6)GalNAcol, Fucα1-2(GalNAcα1-3)Gal-(Fuc)GlcNAcol, Galβ1-3(Fucα1-4)GlcNAcβ1-3Galβ1-3GalNAcol, Galβ1-4(Fucα1-3)GlcNAcβ1-3Galβ1-3GalNAcol, Fucα1-2Galβ1-4GlcNAcβ1-3Galβ1-4GlcNAcol, Galβ1-3(Fucα1-2Galβ1-4GlcNAcβ1-6)GalNAcol, Fucα1-2Galβ1-3(Galβ1-4GlcNAcβ1-6)GalNAcol, Fucα1-2(GalNAcα1-3)Galβ1-4GlcNAcβ1-6GalNAcol, Fucα1-2Galβ1-3(GalNAcβ1-4GlcNAcβ1-6)GalNAcol, Galβ1-4GlcNAcβ1-3(GlcNAcβ1-6)Galβ1-3GalNAcol, GlcNAcα1-4Galβ1-3(Galβ1-4GlcNAcβ1-6)GalNAcol, GalNAcα1-3(Fucα1-2)Galβ1-4(6S)GlcNAcβ1-3Gal minus H₂O, Fucα1-2Galβ1-4(6S)GlcNAcβ1-3Galβ1-3GalNAcol, Fucα1-2Galβ1-3[Galβ1-4(6S)GlcNAcβ1-6]GalNAcol, GlcNAcα1-4Galβ1-3(GalNAcβ1-4GlcNAcβ1-6)GalNAcol, GlcNAcβ1-3[Fucα1-2Galβ1-4(6S)GlcNAcβ1-6]GalNAcol, GalNAcα1-3(Fucα1-2)Galβ1-3(6SGlcNAcβ1-6)GalNAcol, Fucα1-2Galβ1-4GlcNAcβ1-3(Galβ1-4GlcNAcβ1-6)Gal, Fucα1-2Galβ1-4GlcNAcβ1-3(Galβ1-4GlcNAcβ1-6)Gal, Galβ1-4GlcNAcβ1-3Galβ1-3(NeuAcα2-6)GalNAcol, Fucα1-2Galβ1-3(Fucα1-2Galβ1-4GlcNAcβ1-6)GalNAcol, Galβ1-3[Fucα1-2(GalNAcα1-3)Galβ1-4GlcNAcβ1-6]GalNAcol, GlcNAcα1-4Galβ1-3(Fucα1-2Galβ1-4GlcNAcβ1-6)GalNAcol, Fucα1-2Galβ1-3(GlcNAcα1-4Galβ1-4GlcNAcβ1-6)GalNAcol, Fucα1-2Galβ1-4(6S)GlcNAcβ1-3(Galβ1-4GlcNAcβ1-6)Gal minus H₂O, Fucα1-2Galβ1-3[Fucα1-2Galβ1-4(6S)GlcNAcβ1-6]GalNAcol, GlcNAcα1-4Galβ1-3(GlcNAcα1-4Galβ1-4GlcNAcβ1-6)GalNAcol, Gal-GlcNAcβ1-3(GlcNAcα1-4Galβ1-4GlcNAcβ1-6)GalNAcol, GalNAcα1-3(Fucα1-2)Galβ1-4(6S)GlcNAcβ1-3Galβ1-3GalNAcol, Fucα1-2Galβ1-4GlcNAcβ1-3Galβ1-4GlcNAcβ1-3Galβ1-3GalNAcol, GlcNAcα1-4Galβ1-3[GalNAcα1-3(Fucα1-2)Galβ1-4GlcNAcβ1-6]GalNAcol, Fucα1-2Galβ1-4(6S)GlcNAcβ1-3Galβ1-4GlcNAcβ1-3Galβ1-4GlcNAcol, and Fucα1-2Galβ1-4(6S)GlcNAcβ1-3Galβ1-4GlcNAcβ1-3(Galβ1-4GlcNAcβ1-6)Gal minus H₂O. In some embodiments, the composition comprises Galβ1-3GalNAcol (T antigen) and NeuAcα2-3Galβ1-3GalNAcol (Sialyl T antigen). In some embodiments, the composition comprises Galβ1-3(Fucα1-4)GlcNAcβ1-3Galβ1-3GalNAcol (Lewis A) and Galβ1-4(Fucα1-3)GlcNAcβ1-3Galβ1-3GalNAcol (Lewis X).

In some embodiments, the composition comprises glycopeptide-bound oligosaccharides having at least 10, at least 20, at least 50, at least 75, or all of the structures provided above (e.g., the composition comprises at least 10, 20, 50, 75, or all of the glycopeptides, wherein each of the glycopeptides comprises a different glycopeptide-bound oligosaccharide from the above list). In some embodiments, the composition comprises glycopeptide-bound oligosaccharides having at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or all of the structures provided above.

In some embodiments, the composition comprises glycopeptide-bound oligosaccharides having at least 10, at least 20, at least 50, at least 75, or all of the structures provided above (e.g., the composition comprises at least 10, 20, 50, 75, or 99 glycopeptides, wherein each of the glycopeptides comprises a different glycopeptide-bound oligosaccharide from the above list).

In some embodiments, the composition has a salt content of less than between about 5% and 1%. In some embodiments, the composition has a salt content of less than about 2%. In some embodiments, the composition has a salt content of less than about 1%.

In some embodiments, the composition has a pH of less than 7.5.

In some embodiments, the composition does not comprise more than about 5%, more than about 4%, more than about 3%, more than about 2%, more than about 1%, more than about 0.5%, or more than about 0.1% free glycans by weight. In some embodiments, the composition has a free glycan content of less than 1% by weight. In some embodiments, the composition has a free glycan content of less than 0.1% by weight. In some embodiments, the composition has a free glycan content of substantially zero. The phrase “free glycans” refers to glycans that are not attached to a protein or polypeptide.

In some embodiments, the composition comprising a mixture of glycopeptides is obtained from porcine intestinal mucins or a partially purified fraction thereof, wherein: the composition is obtained without subjecting the mucins or the partially purified fraction thereof to conditions or reagents that release oligosaccharides from glycopeptides; the composition has a total glycan content of greater than 10%; the composition comprises glycopeptide-bound oligosaccharides having each of the following general formulae: Hex1HexNAc1, HexNAc2, NeuAc1HexNAc1, NeuGc1HexNAc1, Hex1HexNAc1Fuc1, Hex1HexNAc2, Hex1HexNAc2Sul1, NeuAc1Hex1HexNAc1, NeuGc1Hex1HexNAc1, NeuAc1HexNAc2, NeuGc1HexNAc2, Hex1HexNAc2Fuc1, Hex1HexNAc2Fuc1Sul1, NeuAc1Hex1HexNAc1Fuc1, Hex1HexNAc3Sul1, Hex2HexNAc2Fuc1, Hex1HexNAc3Fuc1Sul1, Hex2HexNAc2Fuc2Sul1, and the composition does not substantially contain insoluble particles having a diameter greater than 7 μm or compounds having a molecular weight of greater than about 3 kDa.

In some embodiments, the composition comprises glycopeptide-bound oligosaccharides having each of the following general formulae: Hex2HexNAc3deHex1Sul2, Hex2HexNAc3deHex1Sul2, NeuGc1Hex2HexNAc3, NeuAc1Hex2HexNAc2deHex1Sul1, Hex2HexNAc4Sul2, Hex2HexNAc4Sul2, NeuAc1Hex2HexNAc3Sul1, Hex2HexNAc3deHex2Sul1, Hex2HexNAc3deHex2Sul1, Hex2HexNAc3deHex2Sul1, Hex2HexNAc3deHex2sul1, Hex2HexNAc3deHex1Sul3, NeuGc1Hex2HexNAc3Sul1, NeuGc1Hex2HexNAc3Sul1, Hex2HexNAc3deHex2Sul2, Hex2HexNAc3deHex2Sul2, Hex2HexNAc3deHex2Sul2, Hex2HexNAc3deHex2Sul2, NeuGc1Hex2HexNAc3deHex1, NeuGc1Hex2HexNAc3Sul2, Hex3HexNAc3deHex1Sul2, NeuGc1Hex2HexNAc2deHex2Sul1, Hex2HexNAc4deHex1Sul2, NeuAc1Hex2HexNAc3deHex1Sul1, Hex2HexNAc3deHex3 Sul1, NeuGc1Hex2HexNAc3deHex1Sul1, NeuGc1Hex2HexNAc3deHex1Sul1, Hex3HexNAc3deHex2Sul1, NeuGc1Hex2HexNAc2deHex2Sul2, NeuGc1Hex3HexNAc2deHex1Sul2, Hex2HexNAc4deHex2Sul1, Hex2HexNAc4deHex2Sul1, Hex2HexNAc4deHex2Sul1, NeuGc1Hex2HexNAc4Sul1, Hex3Hex4deHex1Sul1, Hex2HexNAc3deHex3Sul2, Hex2HexNAc3deHex3Sul2, NeuGc1Hex2HexNAc3deHex1Sul2, NeuGc1Hex2HexNAc3deHex1Sul2, NeuGc1Hex2HexNAc3deHex1Sul2, NeuGc1Hex3HexNAc2deHex2Sul1, NeuAc1Hex2HexNAc4deHex1, Hex2HexNAc4deHex2Sul2, Hex2HexNAc4deHex2Sul2, NeuGc1Hex2HexNAc4deHex1, Hex2HexNAc3deHex4Sul1, Hex3HexNAc4deHex1Sul2, NeuGc1Hex2HexNAc3deHex2Sul1, NeuGc1Hex2HexNAc3deHex2Sul1, NeuGc1Hex3HexNAc3deHex1Sul1, NeuGc1Hex2HexNAc2deHex3 Sul2, Hex2HexNAc4deHex3 Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc3deHex2Sul2, Hex3HexNAc3deHex3 Sul2, NeuGc1Hex3HexNAc3deHex1Sul2, Hex2HexNAc5deHex2Sul1, Hex2HexNAc4deHex3Sul2, NeuGc1Hex2HexNAc4deHex2Sul1, NeuGc1Hex2HexNAc4deHex2Sul1, NeuGc1Hex3HexNAc4deHex1Sul1, Hex2HexNAc5deHex3 Sul1, NeuGc1Hex2HexNAc4deHex2Sul2, NeuGc1Hex2HexNAc4deHex2Sul2, NeuGc1Hex2HexNAc4deHex2Sul2, NeuGc1Hex3HexNAc4deHex1Sul2, Hex3HexNAc4deHex4Sul2, NeuAc1Hex5HexNAc4deHex1, NeuAc1Hex4HexNAc4deHex2Sul1, NeuGc1Hex3HexNAc4deHex4Sul1, NeuGc1Hex4HexNAc4deHex3Sul1, and NeuGc1Hex3HexNAc4deHex4Sul2. In some embodiments, the composition comprises at least one, two or all three of NeuAc1HexNAc1, NeuAc2Hex1HexNAc1, and NeuAc1Hex1HexNAc1.

In some embodiments, the sialic acid content of the composition is greater than 5%, 10%, 15%, 20%, 22.5%, 25%, 27.5%, 30%, 32.5%, 35%, or 40% (w/w). In some embodiments, the sialic acid content of the composition is greater than 25% (w/w) and less than 50% (e.g., 25, 30, 35, 40, 45, or 49%). In some embodiments, sialic acid content is determined by LC-MS/MS.

In some embodiments, the composition comprises glycopeptide-bound oligosaccharides having at least 7, 14, 21 or all 28 of the following structures: Galβ1-3GalNAc, GlcNAcβ1-6GalNAc, NeuAcα2-6GalNAc, NeuGcα2-6GalNAc, Fucα1-2Galβ1-3GalNAc, Gal+GlcNAcβ1-6GalNAc, Galβ1-3(GlcNAcβ1-6)GalNAc, Galβ1-3GlcNAcβ1-6GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, Fucα1-2(GalNAcα1-3)Galβ1-3GalNAc, Fucα1-2Galβ1-4GlcNAcβ1-6GalNAc, Fucα1-2Galβ1-3(GlcNAcβ1-6)GalNAc, Fucα1-2Galβ1-3(6S-GlcNAcβ1-6)GalNAc, Fucα1-2Galβ1-3(NeuAcβ2-6)GalNAc, GlcNAcβ1-3[Galβ1-4(6S)GlcNAcβ1-6]GalNAc, Galβ1-4GlcNAcβ1-3[(6S)GlcNAcβ1-6]GalNAc, Galβ1-3(Fucα1-2Galβ1-4GlcNAcβ1-6)GalNAc, Fucα1-2Galβ1-4(6S)GlcNAcβ1-6[GlcNAcβ1-3]GalNAc, GlcNAcβ1-3[Fucα1-2Galβ1-3(6S-)GlcNAcβ1-6]GalNAc, and Fucα1-2Galβ1-3[Fucα1-2Galβ1-4(6S)GlcNAcβ1-6]GalNAc.

In some embodiments, the composition comprises at least 1, 2, 3, 4, 5, 6, 7, 8, 9, or all 10 sialylated glycopeptide-bound oligosaccharide selected from the following: NeuAcα2-6GalNAc, NeuGcα2-6GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, and Fucα1-2Galβ1-3(NeuAcβ2-6)GalNAc. In some embodiments, the composition comprises NeuAcα2-3Galβ1-3GalNAc (Sialyl-T antigen), NeuAcα2-6GalNAc (Sialyl Tn antigen), and NeuAcα2-3Galβ1-3(NeuAcα2-6)GalNAc (Disialyl T antigen).

Another aspect of the disclosure is directed to a method for treating a tumor in a subject who has been diagnosed with cancer comprising administering to the subject an effective amount of a composition comprising glycopeptides produced by a method comprising: (a) dissolving gastrointestinal mucin in water comprising calcium hydroxide; (b) adding diatomaceous earth to (a) and filtering the resulting solution; (c) adding a cationic substance to (b); and (d) filtering and concentrating the solution from (c), thereby producing the composition.

In some embodiments, the method further comprises adjusting the pH of the solution in (b) using carbon dioxide.

In some embodiments, the dissolving is achieved at about 60° C.

In some embodiments, the cationic substance comprises an ion exchange hydrogen form resin.

Another aspect of the disclosure is directed to a method for treating a tumor in a subject who has been diagnosed with cancer comprising administering to the subject an effective amount of a composition comprising glycopeptides produced by a method comprising: (a) stabilizing gastrointestinal mucin at pH 5.0; (b) desalinating the stabilized mucin using dialysis; (c) concentrating the desalinated mucin; (d) subjecting the concentrate from (d) to diafiltration; thereby producing the composition. In one aspect, the subject has been diagnosed a cancer that is resistant or can become resistant to an immune checkpoint inhibitor. In a further aspect, the subject diagnosed with the cancer is treated. Subjects not diagnosed with the cancer do not receive the composition.

In some embodiments, the concentrating in (c) is achieved by evaporation with a rotary evaporator at least 80° C.

In some embodiments, the effective amount of the composition is about 0.2-0.8 grams per kilogram of the subject.

In some embodiments, the method further comprises administering a chemotherapeutic agent before, after, or simultaneously with the composition comprising glycopeptides. In some embodiments, the chemotherapeutic agent is an immune checkpoint inhibitor.

In some embodiments, the composition has substantially no free glycans. In some embodiments, the composition has a pH of less than 7.5.

In some embodiments, the composition is a powder. In some embodiments, the composition further comprises one or more excipients or carriers. In some embodiments, the composition is administered orally or rectally.

The composition may take the form of a slurry, powder, or liquid. In some embodiments, the composition is a powder.

In some embodiments of the methods disclosed herein, the composition comprises at least one, at least two, at least three, at least four, at least five, or all six glycopeptide-bound oligosaccharide having a structure selected from Galβ1-3GalNAcol (T antigen), NeuAcα2-6GalNAcol (Sialyl Tn antigen), Galβ1-4(Fucα1-3)(6S)GlcNAcol (Lewis X), 6SGalβ1-3(Fucα1-4)GlcNAcol (Lewis A), NeuAcα2-3Galβ1-3(NeuAcα2-6)GalNAc (Disialyl T antigen), and NeuAcα2-3Galβ1-3GalNAcol (Sialyl T antigen).

Routes of administering the composition are not limited and may be any suitable route. In some embodiments, the composition is administered orally, parenteral, or rectally.

In some embodiments, the composition is in the form of a pharmaceutical composition. In some embodiments, the composition further comprises one or more excipients or carriers. In some embodiments, the composition comprises a pharmaceutically-acceptable carrier. The term “pharmaceutically-acceptable carrier”, as used herein, means one or more compatible solid or liquid vehicles, fillers, diluents, or encapsulating substances which are suitable for administration to a human or non-human animal. In preferred embodiments, a pharmaceutically-acceptable carrier is a non-toxic material that does not interfere with the effectiveness of the biological activity of the active ingredients (i.e., the glycopeptides). The term “compatible”, as used herein, means that the components of the pharmaceutical compositions are capable of being comingled with an agent (i.e., the glycopeptides), and with each other, in a manner such that there is no interaction which would substantially reduce the pharmaceutical efficacy of the pharmaceutical composition under ordinary use situations. Pharmaceutically-acceptable carriers should be of sufficiently high purity and sufficiently low toxicity to render them suitable for administration to the human or non-human animal being treated.

Some examples of substances which can serve as pharmaceutically-acceptable carriers are pyrogen-free water; isotonic saline; phosphate buffer solutions; sugars such as lactose, glucose, and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobrama; polyols such as propylene glycol, glycerin, sorbitol, mannitol, and polyethylene glycol; sugar; alginic acid; cocoa butter (suppository base); emulsifiers, such as the Tweens; as well as other non-toxic compatible substances used in pharmaceutical formulation. Wetting agents and lubricants such as sodium lauryl sulfate, as well as coloring agents, flavoring agents, excipients, tableting agents, stabilizers, antioxidants, and preservatives, can also be present. It will be appreciated that a pharmaceutical composition can contain multiple different pharmaceutically acceptable carriers.

In some embodiments, the composition is administered for at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days. In some embodiments, the composition is administered daily for 28 days or more.

In some embodiments, the composition is administered in an amount effective to increase one or more of Akkermansia spp., Butyricicoccus spp., Clostridium spp., or Parabacteroides spp.

In some embodiments, the composition is administered to a human subject orally or via suppository in an effective amount of about 10-80 grams per day. In some embodiments, the composition is administered to a human subject orally or via suppository in an effective amount of about 0.2 to 0.8 (e.g., 0.2, 0.3, 0.4, 0.5, 0.6, 0.7, or 0.8) grams per kg of the subject's weight. In some embodiments, the composition is administered to a human subject orally or via suppository in an effective amount of about 0.4 to 0.6 (e.g., 0.4, 0.5, or 0.6) grams per kg of the subject's weight. In some embodiments, the composition is administered to a human subject orally or via suppository in an effective amount of about 0.5 to 0.6 (0.5 or 0.6) grams per kg of the subject's weight. In some embodiments, the amount per day is divided into 1, 2, 3, 4, 5, or 6 doses per day.

In some embodiments, the subject has a tumor that is refractory to or has developed resistance to an immune checkpoint inhibitor therapy.

In some embodiments, the subject has stage 3 or 4 melanoma, CRC, or metastatic CRC. In some embodiments, the subject has breast cancer or lung cancer.

In some embodiments, administration of the composition results in an increased level or activity of T cells cross-reactive to a glycan tumor antigen and a glycan present on a glycopeptide of the composition. In some embodiments, the T cells are CD8+ T cells. In some embodiments, the T cells are CD4+ T cells. In some embodiments, the composition is administered orally or via a suppository.

Another aspect of the disclosure is directed to a method for treating a tumor in a subject who has been diagnosed with cancer comprising administering to the subject an effective amount of a composition comprising glycopeptides produced by a method comprising: (a) dissolving gastrointestinal mucin from stomach of an animal in water comprising calcium hydroxide; (b) adding diatomaceous earth to (a) and filtering the resulting solution; (c) adding a cationic substance to (b); and (d) filtering and concentrating the solution from (c), thereby producing the composition. In some embodiments, the method further comprises adjusting the pH of the solution in (b) using carbon dioxide. In some embodiments, the dissolving is achieved at about 60° C. (e.g., 55° C., 56° C., 57° C., 58° C., 59° C., 60° C., 61° C., 62° C., 63° C., 64° C., or 65° C.). In some embodiments, the cationic substance comprises an ion exchange hydrogen form resin.

Another aspect of the disclosure is directed to a method for treating a tumor in a subject who has been diagnosed with cancer comprising administering to the subject an effective amount of a composition comprising glycopeptides produced by a method comprising: (a) stabilizing gastrointestinal mucin at pH 5.0; (b) desalinating the stabilized mucin using dialysis; (c) concentrating the desalinated mucin; (d) subjecting the concentrate from (d) to diafiltration; thereby producing the composition. In some embodiments, the concentrating in (c) is achieved by evaporation with a rotary evaporator at least 80° C. In some embodiments, the effective amount of the composition is about 0.2-0.8 grams per kilogram of the subject. In some embodiments, the method further comprises administering an immune checkpoint inhibitor before, after, or simultaneously with the composition comprising glycopeptides.

### Compositions

Another aspect of the disclosure is directed to a “GCX composition” as prepared by the methods described in Example 3. In some embodiments, a GCX composition is produced by a method comprising: (a) stabilizing gastrointestinal mucin from intestine of an animal at pH 5.0; (b) desalinating the stabilized mucin using dialysis; (c) concentrating the desalinated mucin; (d) subjecting the concentrate from (d) to diafiltration; thereby producing the composition. In some embodiments, the concentrating in (c) is achieved by evaporation with a rotary evaporator at least 80° C. In some embodiments, the effective amount of the composition is about 0.2-0.8 grams per kilogram of the subject. In some embodiments, the method further comprises administering an immune checkpoint inhibitor before, after, or simultaneously with the composition comprising glycopeptides.

In some embodiments, the GCX composition comprises at least one glycopeptide-bound oligosaccharide having a general formulae selected from Hex1HexNAc1, HexNAc2, Hex1HexNAc1Sul, Hex1NAc2Sul1, NeuAc1HexNAc1, NeuGc1HexNAc1, Hex1HexNAc1deHex1, Hex2HexNAc1, Hex1HexNAc2, HexNAc3, Hex1HexNAc2Sul1, NeuAc1Hex1HexNAc1, NeuGc1Hex1HexNAc1, Hex2HexNAc1deHex1, Hex1NAc3Sul1, NeuAc1HexNAc2, NeuGc1HexNAc2, Hex1HexNAc2deHex1, Hex2HexNAc2, Hex1HexNAc3, Hex1HexNAc2deHex1Sul1, NeuAc1Hex1HexNAc1deHex1, Hex2HexNAcSul1, NeuGc1Hex1HexNAc1deHex1, Hex1HexNAc3Sul1, NeuAc1Hex1HexNAc2, NeuGc1Hex1HexNAc2, Hex2HexNAc2deHex1, Hex3HexNAc2, Hex1HexNAc3deHex1, Hex2HexNAc3, NeuAc1Hex1HexNAc2Sul1, NeuAc2Hex1HexNAc1, NeuGc1Hex1HexNAc2Sul1, Hex2HexNAc2deHex1Sul1, NeuAc1NeuGc1Hex1HexNAc1, NeuGc2Hex1HexNAc1, Hex1HexNAc3deHex1Sul1, NeuAc1Hex1HexNAc2deHex1, Hex2HexNAc3Sul1, NeuGc1Hex1HexNAc2deHex1, NeuAc1Hex2HexNAc2, Hex2HexNAc2deHex2, NeuGc1Hex2HexNAc2, Hex2HexNAc3deHex1, NeuAc1Hex2HexNAc2Sul1, Hex2HexNAc2deHex2Sul1, NeuGc1Hex2HexNAc2Sul1, Hex1HexNAc4deHex1, NeuAc1Hex1HexNAc3Sul1, Hex1HexNAc3deHex2Sul1, NeuAc2Hex1HexNAc2, NeuGc1Hex1HexNAc3Sul1, Hex2HexNAc3deHex1Sul1, NeuAc1Hex2HexNAc2deHex1, NeuGc2Hex1HexNAc2, NeuGc1Hex2HexNAc2deHex1, NeuGc1Hex3HexNAc2, Hex1HexNAc4deHex1 Sul1, NeuAc1Hex1HexNAc3deHex1, Hex2HexNAc3deHex2 NeuGc1Hex2HexNAc3, NeuAc1Hex2HexNAc2deHex1Sul1, Hex2HexNAc3deHex2Sul1, NeuAc1Hex2HexNAc3deHex1Sul1, NeuGc1Hex2HexNAc3deHex1Sul1, Hex2HexNAc4deHex2Sul1, Hex2HexNAc5deHex2Sul1, NeuAc1Hex5HexNAc4deHex1, and NeuAc1Hex4HexNAc4deHex2Sul1.

In some embodiments, the composition comprises glycopeptide-bound oligosaccharides having at least 10, at least 20, at least, 30, at least 40, at least 45, at least 50, at least 55, at least 60, at least 65, (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65) or all of the general formulae listed above. In some embodiments, the composition comprises at least 10 of the general formulae listed above, and each formula is present in the composition between 0.1% and 10% (e.g., 0.1%, 0.2%, 0.3%, 0.4%, 0.5%, 0.6%, 0.7%, 0.8%, 0.9% 1.5%, 2%, 2.5%, 3%, 3.5%, 4%, 4.5%, 5%, 5.5%, 6%, 6.5%, 7%, 7.5%, 8%, 8.5%, 9%, 9.5%, 10%,) of all glycopeptide-bound oligosaccharides in the composition.

In some embodiments, the composition comprises at least 10 glycopeptide-bound oligosaccharides having a structure selected from Galβ1-3GalNAc, GalNAcα1-3GalNAc, GlcNAcβ1-6GalNAc, 3SGalβ1-3GalNAc, 6SGlcNAcβ1-3GalNAc, 6SGlcNAcβ1-6GalNAc, NeuAcα2-6GalNAc, NeuGcα2-6GalNAc, Fucα1-2(GalNAcα1-3)Gal, Fucα1-2Galβ1-3GalNAc, Fucα1-2Galβ1-4GlcNAc, Galβ1-4GlcNAcβ1-3Gal, Galβ1-3(GlcNAcβ1-6)GalNAc, Galβ1-4GlcNAcβ1-3GalNAc, GlcNAcβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Fucα1-2Galβ-4GlcNAcβ1-3Gal, 6SGlcNAcβ1-3(GlcNAcβ1-6)GalNAc, GlcNAcβ1-3(6S-GlcNAcβ1-6)GalNAc, GalNAcα1-3(NeuAcα2-6)GalNAc, GlcNAcβ1-3(NeuAcα2-6)GalNAc, GalNAcα1-3(NeuGcα2-6)GalNAc, GlcNAcβ1-3(NeuGcα2-6)GalNAc, Fucα1-2(GalNAcα1-3)Galβ1-3GalNAc, Fucα1-2Galβ1-3(GlcNAcβ1-6)GalNAc, Fucα1-2Galβ1-3GlcNAcβ1-3GalNAc, Galβ1-3(Galβ1-4GlcNAcβ1-6)GalNAc, Galβ1-3GlcNAcβ1-3Galβ1-3GalNAc, GlcNAcβ1-3(Galβ1-4GlcNAcβ1-6)GalNAc, Fucα1-2Galβ1-3(6S-GlcNAcβ1-6)GalNAc, Fucα1-2Galβ1-3(NeuAcα2-6)GalNAc, Galβ1-3[Galβ1-4(6S)GlcNAcβ1-6]GalNAc, Fucα1-2Galβ1-3(NeuGcα2-6)GalNAc, Galβ1-4GlcNAcβ1-3(6SGlcNAcβ1-6)GalNAc, GlcNAcα1-4Galβ1-3[(6S)GlcNAcβ1-6]GalNAc, GlcNAcβ1-3[Galβ1-4(6S)GlcNAcβ1-6]GalNAc, GlcNAcα1-4Galβ1-3(NeuAcα2-6)GalNAc, NeuAcα2-3(GalNAcβ1-4)Galβ1-3GalNAc, GalNAcβ1-4(NeuGcα2-3)Galβ1-3GalNAc, NeuGcα2-3Galβ1-3(GlcNAcβ1-6)GalNAc, Fucα1-2Galβ1-3(Galβ1-4GlcNAcβ1-6)GalNAc, Galβ1-3(Fucα1-2Galβ1-4GlcNAcβ1-6)GalNAc, Galα1-3Galβ1-4GlcNAcβ1-3Galβ1-3GalNAc, Galβ1-3(Galα1-3Galβ1-4GlcNAcβ1-6)GalNAc, Fucα1-2(GalNAcα1-3)Galβ1-4GlcNAcβ1-3GalNAc, GlcNAcβ1-3(Fucα1-2Galβ1-4GlcNAcβ1-6)GalNAc, GlcNAcα1-4Galβ1-3(Galβ1-4GlcNAcβ1-6)GalNAc, NeuAcα2-3Galβ1-3[(6S)GlcNAcβ1-6]GalNAc, NeuAcα2-3Galβ1-3(NeuAcα2-6)GalNAc, NeuGcα2-6Galβ1-3(6SGlcNAcβ1-6)GalNAc, Fucα1-2Galβ1-3[Gal 01-4(6S)GlcNAcβ1-6]GalNAc, Galβ1-3[Fucα1-2Galβ1-4(6S)GlcNAcβ1-6]GalNAc, NeuAcα2-3Galβ1-3(NeuGcα2-6)GalNAc, NeuGcα2-3Galβ1-3(NeuGcα2-6)GalNAc, Fucα1-2Galβ1-4(6S)GlcNAcβ1-3(GlcNAcβ1-6)GalNAc, Fucα1-2Galβ1-4GlcNAcβ-3(6SGlcNAcβ1-6)GalNAc, GalNAcα1-3(Fucα1-2)Galβ1-3(6SGlcNAcβ1-6)GalNAc, GlcNAcβ1-3[Fucα1-2Galβ1-4(6S)GlcNAcβ1-6]GalNAc, Fucα1-2Galβ1-4GlcNAcβ1-3(NeuAcα2-6)GalNAc, Galβ1-4GlcNAcβ1-3[Galβ1-4(6S)GlcNAcβ1-6]GalNAc, Fucα1-2Galβ1-4GlcNAcβ1-3(NeuGcα2-6)GalNAc, NeuAcα2-3Galβ1-3(Galβ1-4GlcNAcβ1-6)GalNAc, Fucα1-2Galβ1-3(Fucα1-2Galβ1-4GlcNAcβ1-6)GalNAc, NeuGcα2-3Galβ1-3(Galβ1-4GlcNAcβ1-6)GalNAc, Galβ1-3[Fucα1-2(GalNAcα1-3)Galβ1-4GlcNAcβ1-6]GalNAc, NeuAcα2-3Galβ1-3[Galβ1-4(6S)GlcNAcβ1-6]GalNAc, Fucα1-2Galβ1-3[Fucα1-2Galβ1-4(6S)GlcNAcβ1-6]GalNAc, NeuGcα2-3Galβ1-3[Galβ1-4(6S)GlcNAcβ1-6]GalNAc, GlcNAcβ1-3[GalNAcα1-3(Fucα1-2)Galβ1-3GlcNAcβ1-6]GalNAc, NeuAcα2-3(GalNAcβ1-4)Galβ1-3(6SGlcNAcβ1-6)GalNAc, 6SGlcNAcβ1-3[Fucα1-2Galβ1-(Fucα1-)GlcNAcβ1-6]GalNAc, NeuAcα2-3(GalNAcβ1-4)Galβ1-3(NeuAcα2-6)GalNAc, GlcNAcβ1-3[NeuGcα2-3Galβ1-4(6S)GlcNAcβ1-6]GalNAc, Fucα1-2Galβ1-4GlcNAcβ1-3[Galβ1-4(6S)GlcNAcβ1-6]GalNAc, Galβ1-3[GalNAcα1-3(Fucα1-2)Galβ1-4(6S)GlcNAcβ1-6]GalNAc, Galβ1-3GlcNAcβ1-3[Fucα1-2Galβ1-4(6S)GlcNAcβ1-6]GalNAc, Galβ1-4GlcNAcβ1-3[Fucα1-2Galβ1-3(6S)GlcNAcβ1-6]GalNAc, NeuAcα2-3Galβ1-3(Fucα1-2Galβ1-4GlcNAcβ1-6)GalNAc, NeuGcα2-3(GalNAcβ1-4)Galβ1-3(NeuGcα2-6)GalNAc, NeuGcα2-3Galβ1-3 (Fucα1-2Galβ1-4GlcNAcβ1-6)GalNAc, Galα1-3Galβ1-3(NeuGcα2-3Galβ1-4GlcNAcβ1-6)GalNAc, GalNAcα1-3(Fucα1-2)Galβ1-3GlcNAcβ1-3(6SGlcNAcβ1-6)GalNAc, GlcNAcβ1-3[GalNAcα1-3(Fucα1-2)Galβ1-(6S)GlcNAcβ1-6]GalNAc, GlcNAcα1-4(Fucα1-2)Galβ1-4GlcNAcβ1-3(NeuAcα2-6)GalNAc, Fucα1-2Galβ1-4GlcNAcβ1-3(Fucα1-2Galβ1-4GlcNAcβ1-6)GalNAc, Galβ1-(Fuc)GlcNAcβ1-3(Fucα1-2Galβ1-4GlcNAcβ1-6)GalNAc, NeuGcα2-3Galβ1-3(GlcNAcα1-4Galβ1-4GlcNAcβ1-6)GalNAc, NeuAcα2-3Galβ1-3[Fucα1-2Galβ1-4(6S)GlcNAcβ1-6]GalNAc, Fucα1-2Galβ1-3[GalNAcα1-3(Fucα1-2)Galβ1-3(6S)GlcNAcβ1-6]GalNAc, Fucα1-2Galβ1-3[GalNAcα1-3(Fucα1-2)Galβ1-4(6S)GlcNAcβ1-6]GalNAc, Fucα1-2Galβ1-4GlcNAcβ1-3[Fucα1-2Galβ1-4(6S)GlcNAcβ1-6]GalNAc, NeuAcα2-3Galβ1-3[GalNAcα1-3(Fucα1-2)Gal-(6S)GlcNAcβ1-6]GalNAc, GlcNAcα1-4Galβ1-3[NeuGcα2-6Gal-(Fuc)(6S)GlcNAcβ1-6]GalNAc, GlcNAcα1-4(Fucα1-2)GlcNAcβ1-3[Fucα1-2)Galβ1-4(6S)GlcNAcβ1-6]GalNAc, GalNAcα1-3(Fucα1-2)Galβ1-4GlcNAcβ1-3[GalNAcα1-3(Fucα1-2)Galβ1-4(6S)GlcNAcβ1-6]GalNAc, and NeuAcα2-Galβ1-4GlcNAcβ1-2Manα1-3(Galβ1-4GlcNAcβ1-2Manα1-6)Manβ1-4GlcNAcβ1-4(Fucα1-6)GlcNAc.

In some embodiments, the composition comprises glycopeptide-bound oligosaccharides having at least 10, at least 20, at least 50, at least 75, or all of the structures provided above (e.g., the composition comprises at least 10, 20, 50, 75, or 99 glycopeptides, wherein each of the glycopeptides comprises a different glycopeptide-bound oligosaccharide from the above list).

In some embodiments, the composition has a salt content of less than about 2%. In some embodiments, the composition has a pH of less than 7.5 (e.g., pH 7.4, 7.3, 7.2, 7.1, 7, 6.9, 6.8, 6.7, 6.6, 6.5, 6.4, 6.3, 6.2, 6.1, or 6). In some embodiments, the composition has a free glycan content of less than 0.1% by weight.

In some embodiments, the composition comprising a mixture of glycopeptides is obtained from porcine intestinal mucins or a partially purified fraction thereof, wherein: the composition is obtained without subjecting the mucins or the partially purified fraction thereof to conditions or reagents that release oligosaccharides from glycopeptides; the composition has a total glycan content of greater than about 10%; the composition comprises glycopeptide-bound oligosaccharides having each of the following general formulae: Hex1HexNAc1, HexNAc2, NeuAc1HexNAc1, NeuGc1HexNAc1, Hex1HexNAc1Fuc1, Hex1HexNAc2, Hex1HexNAc2Sul1, NeuAc1Hex1HexNAc1, NeuGc1Hex1HexNAc1, NeuAc1HexNAc2, NeuGc1HexNAc2, Hex1HexNAc2Fuc1, Hex1HexNAc2Fuc1Sul1, NeuAc1Hex1HexNAc1Fuc1, Hex1HexNAc3Sul1, Hex2HexNAc2Fuc1, Hex1HexNAc3Fuc1Sul1, Hex2HexNAc2Fuc2Sul1, and the composition does not substantially contain insoluble particles having a diameter greater than 7 μm or compounds having a molecular weight of greater than about 3 kDa. In some embodiments, the composition comprises glycopeptide-bound oligosaccharides having each of the following general formulae: Hex2HexNAc3deHex1Sul2, Hex2HexNAc3deHex1Sul2, NeuGc1Hex2HexNAc3, NeuAc1Hex2HexNAc2deHex1Sul1, Hex2HexNAc4Sul2, Hex2HexNAc4Sul2, NeuAc1Hex2HexNAc3Sul1, Hex2HexNAc3deHex2Sul1, Hex2HexNAc3deHex2Sul1, Hex2HexNAc3deHex2Sul1, Hex2HexNAc3deHex2sul1, Hex2HexNAc3deHex1Sul3, NeuGc1Hex2HexNAc3Sul1, NeuGc1Hex2HexNAc3Sul1, Hex2HexNAc3deHex2Sul2, Hex2HexNAc3deHex2Sul2, Hex2HexNAc3deHex2Sul2, Hex2HexNAc3deHex2Sul2, NeuGc1Hex2HexNAc3deHex1, NeuGc1Hex2HexNAc3Sul2, Hex3HexNAc3deHex1Sul2, NeuGc1Hex2HexNAc2deHex2Sul1, Hex2HexNAc4deHex1Sul2, NeuAc1Hex2HexNAc3deHex1Sul1, Hex2HexNAc3deHex3 Sul1, NeuGc1Hex2HexNAc3deHex1Sul1, NeuGc1Hex2HexNAc3deHex1Sul1, Hex3HexNAc3deHex2Sul1, NeuGc1Hex2HexNAc2deHex2Sul2, NeuGc1Hex3HexNAc2deHex1Sul2, Hex2HexNAc4deHex2Sul1, Hex2HexNAc4deHex2Sul1, Hex2HexNAc4deHex2Sul1, NeuGc1Hex2HexNAc4Sul1, Hex3Hex4deHex1Sul1, Hex2HexNAc3deHex3Sul2, Hex2HexNAc3deHex3Sul2, NeuGc1Hex2HexNAc3deHex1Sul2, NeuGc1Hex2HexNAc3deHex1Sul2, NeuGc1Hex2HexNAc3deHex1Sul2, NeuGc1Hex3HexNAc2deHex2Sul1, NeuAc1Hex2HexNAc4deHex1, Hex2HexNAc4deHex2Sul2, Hex2HexNAc4deHex2Sul2, NeuGc1Hex2HexNAc4deHex1, Hex2HexNAc3deHex4Sul1, Hex3HexNAc4deHex1Sul2, NeuGc1Hex2HexNAc3deHex2Sul1, NeuGc1Hex2HexNAc3deHex2Sul1, NeuGc1Hex3HexNAc3deHex1Sul1, NeuGc1Hex2HexNAc2deHex3 Sul2, Hex2HexNAc4deHex3 Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc3deHex2Sul2, Hex3HexNAc3deHex3 Sul2, NeuGc1Hex3HexNAc3deHex1Sul2, Hex2HexNAc5deHex2Sul1, Hex2HexNAc4deHex3Sul2, NeuGc1Hex2HexNAc4deHex2Sul1, NeuGc1Hex2HexNAc4deHex2Sul1, NeuGc1Hex3HexNAc4deHex1Sul1, Hex2HexNAc5deHex3 Sul1, NeuGc1Hex2HexNAc4deHex2Sul2, NeuGc1Hex2HexNAc4deHex2Sul2, NeuGc1Hex2HexNAc4deHex2Sul2, NeuGc1Hex3HexNAc4deHex1Sul2, Hex3HexNAc4deHex4Sul2, NeuAc1Hex5HexNAc4deHex1, NeuAc1Hex4HexNAc4deHex2Sul1, NeuGc1Hex3HexNAc4deHex4Sul1, NeuGc1Hex4HexNAc4deHex3Sul1, and NeuGc1Hex3HexNAc4deHex4Sul2. In some embodiments, the composition comprises at least one, two or all three of NeuAc1HexNAc1, NeuAc2Hex1HexNAc1, and NeuAc1Hex1HexNAc1.

In some embodiments, the sialic acid content of the composition is greater than 25% and less than 50% (e.g., 25, 30, 35, 40, 45, or 49%).

In some embodiments, the composition comprises glycopeptide-bound oligosaccharides having at least 7, 14, 21 or all 28 of the following structures: Galβ1-3GalNAc, GlcNAcβ1-6GalNAc, NeuAcα2-6GalNAc, NeuGcα2-6GalNAc, Fucα1-2Galβ1-3GalNAc, Gal+GlcNAcβ1-6GalNAc, Galβ1-3(GlcNAcβ1-6)GalNAc, Galβ1-3GlcNAcβ1-6GalNAc, Galβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, Fucα1-2(GalNAcα1-3)Galβ1-3GalNAc, Fucα1-2Galβ1-4GlcNAcβ1-6GalNAc, Fucα1-2Galβ1-3(GlcNAcβ1-6)GalNAc, Fucα1-2Galβ1-3(6S-GlcNAcβ1-6)GalNAc, Fucα1-2Galβ1-3(NeuAcβ2-6)GalNAc, GlcNAcβ1-3[Galβ1-4(6S)GlcNAcβ1-6]GalNAc, Galβ1-4GlcNAcβ1-3[(6S)GlcNAcβ1-6]GalNAc, Galβ1-3(Fucα1-2Galβ1-4GlcNAcβ1-6)GalNAc, Fucα1-2Galβ1-4(6S)GlcNAcβ1-6[GlcNAcβ1-3]GalNAc, GlcNAcβ1-3[Fucα1-2Galβ1-3(6S-)GlcNAcβ1-6]GalNAc, Fucα1-2Galβ1-3[Fucα1-2Galβ1-4(6S)GlcNAcβ1-6]GalNAc, and NeuAcα2-3Galβ1-3(NeuAcα2-6)GalNAc.

In some embodiments, the composition comprises glycopeptide-bound oligosaccharides having at least 10, 20, 30, 40, 50, 60, 70, 80, 90, 100, 110, 120, 130, 140, 150, 160, or all of the following structures: Galβ1-3GalNAcol, GlcNAcβ1-3GalNAcol, GalNAcα1-3GalNAcol, SGalβ1-GlcNAc, 3SGalβ1-3GalNAcol, 6SGlcNAcβ1-6GalNAcol, 6SGlcNAcβ1-3GalNAcol, NeuAcα2-6GalNAcol, NeuGcα2-6GalNAcol, Fucα1-2(GalNAcα1-3)Galol, Fucα1-2Galβ1-4GlcNAcol, Fucα1-2Galβ1-3GalNAcol, Galβ1-4GlcNAcβ1-3Galol, Galβ1-3(GlcNAcβ1-6)GalNAcol, Galβ1-4GlcNAcβ1-3GalNAcol, 6SGal(Fuc)GlcNAc, 3SGal-GlcNAcβ1-3Galol, GlcNAcβ1-3(GlcNAcβ1-6)GalNAcol, Galβ1-3(6SGlcNAcβ1-6)GalNAcol, 3SGalβ1-3GlcNAcβ1-3GalNAcol, Galβ1-3(NeuAcα2-6)GalNAcol, NeuAcα2-3Galβ1-3GalNAcol, Galβ1-3(NeuGcα2-6)GalNAcol, NeuGcα2-3Galβ1-3GalNAcol, Fucα1-2Galβ-4GlcNAcβ1-3Galol, 6SGlcNAcβ1-3(GlcNAcβ1-6)GalNAcol, GlcNAcβ1-3(6S-GlcNAcβ1-6)GalNAcol, GlcNAcβ1-3(NeuAcα2-6)GalNAcol, GalNAcα1-3(NeuAcα2-6)GalNAcol, GlcNAcβ1-3(NeuGcα2-6)GalNAcol, GalNAcα1-3(NeuGcα2-6)GalNAcol, Galβ1-3(Fucα1-4)GlcNAcβ1-3GalNAcol, Fucα1-2(GalNAcα1-3)Galβ1-3GalNAcol, Fucα1-2Galβ1-3GlcNAcβ1-3GalNAcol, Fucα1-2Galβ1-3(GlcNAcβ1-6)GalNAcol, Galβ1-3(Galβ1-4GlcNAcβ1-6)GalNAcol, Galβ1-3GlcNAcβ1-3Galβ1-3GalNAcol, Fucα1-2Gal(Fuc)(6S)GlcNAc, 6SGalβ1-4(Fucα1-3)GlcNAcβ1-3Galol, GlcNAcβ1-3(Galβ1-4GlcNAcβ1-6)GalNAcol, 6SGalβ1-4(Fucα1-3)GlcNAcβ1-3GalNAcol, Fucα1-2Galβ1-3(6S-GlcNAcβ1-6)GalNAcol, Fucα1-2Galβ1-3(NeuAcα2-6)GalNAcol, Galβ1-3[Galβ1-4(6S)GlcNAcβ1-6]GalNAcol, 3SGalβ1-3GlcNAcβ1-3Galβ1-3GalNAcol, Fucα1-2Galβ1-3(NeuGcα2-6)GalNAcol, GlcNAcα1-4Galβ1-3[(6S)GlcNAcβ1-6]GalNAcol, GlcNAcα1-4(S)Galβ1-4GlcNAcβ1-3GalNAcol, GlcNAcβ1-3[Gal-(6S)GlcNAcβ1-6]GalNAcol, Galβ1-4GlcNAcβ1-3(6SGlcNAcβ1-6)GalNAcol, GlcNAcβ1-3[Galβ1-4(6S)GlcNAcβ1-6]GalNAcol, NeuAcα2-3(GalNAcβ1-4)Galβ1-3GalNAcol, GlcNAcα1-4Galβ1-3(NeuAcα2-6)GalNAcol, NeuAcα2-3Galβ1-3(GlcNAcβ1-6)GalNAcol, GalNAcβ1-4(NeuGcα2-3)Galβ1-3GalNAcol, NeuGcα2-3Galβ1-3(GlcNAcβ1-6)GalNAcol, Galβ1-3[Galβ1-3(Fucα1-4)GlcNAcβ1-6]GalNAcol, Galβ1-3(Fucα1-2Galβ1-4GlcNAcβ1-6)GalNAcol, Fucα1-2Galβ1-3(Galβ1-4GlcNAcβ1-6)GalNAcol, Galβ1-3(Galα1-3Galβ1-4GlcNAcβ1-6)GalNAcol, Galα1-3Galβ1-4GlcNAcβ1-3Galβ1-3GalNAcol, NeuGcα2-3(6S)Gal-(Fuc)GlcNAc, GlcNAcβ1-3[Gal-(Fuc)GlcNAcβ1-6]GalNAcol, Fucα1-2(GalNAcα1-3)Galβ1-4GlcNAcβ1-3GalNAcol, GlcNAcβ1-3(Fucα1-2Galβ1-4GlcNAcβ1-6)GalNAcol, 3SGalβ1-4GlcNAcβ1-3(6SGlcNAcβ1-6)GalNAcol, GlcNAcα1-4Galβ1-3(Galβ1-4GlcNAcβ1-6)GalNAcol, NeuAcα2-3Galβ1-3[(6S)GlcNAcβ1-6]GalNAcol, NeuAcα2-3Galβ1-3(NeuAcα2-6)GalNAcol, NeuGcα2-6Galβ1-3(6SGlcNAcβ1-6)GalNAcol, Galβ1-3[Galβ1-4(Fucα1-3)(6S)GlcNAcβ1-6]GalNAcol, Galβ1-3[Fucα1-2(6S)Galβ1-4GlcNAcβ1-6]GalNAcol, Gal(Fuc)(6S)GlcNAcβ1-3Galβ1-3GalNAcol, Galβ1-3[Fucα1-2Galβ1-4(6S)GlcNAcβ1-6]GalNAcol, Fucα1-2Galβ1-3[Galβ1-4(6S)GlcNAcβ1-6]GalNAcol, NeuAcα2-3Galβ1-3(NeuGcα2-6)GalNAcol, NeuGcα2-3Galβ1-3(NeuGcα2-6)GalNAcol, GlcNAcβ1-3[Galβ1-4(Fucα1-3)(6S)GlcNAcβ1-6]GalNAcol, Gal(Fuc)GlcNAcβ1-3(6SGlcNAcβ1-6)GalNAcol, GlcNAcβ1-3[SGalβ1-4(Fucα1-3)GlcNAcβ1-6]GalNAcol, Fucα1-2Galβ1-4GlcNAcβ-3(6SGlcNAcβ1-6)GalNAcol, GlcNAcβ1-3[Fucα1-2Galβ1-4(6S)GlcNAcβ1-6]GalNAcol, GalNAcα1-3(Fucα1-2)Galβ1-3(6SGlcNAcβ1-6)GalNAcol, Fucα1-2Galβ1-4(6S)GlcNAcβ1-3(GlcNAcβ1-6)GalNAcol, Fucα1-2Galβ1-4GlcNAcβ-3(6SGlcNAcβ1-6)GalNAcol, Fucα1-2Galβ1-3GlcNAcβ1-3(NeuAcα2-6)GalNAcol, Fucα1-2Galβ1-4GlcNAcβ1-3(NeuAcα2-6)GalNAcol, Galβ1-4GlcNAcβ1-3[Galβ1-4(6S)GlcNAcβ1-6]GalNAcol, Fucα1-2Galβ1-4GlcNAcβ1-3(NeuGcα2-6)GalNAcol, NeuAcα2-3Galβ1-3(Galβ1-4GlcNAcβ1-6)GalNAcol, Galβ1-3[Fucα1-2Gal(Fuc)GlcNAcβ1-6]GalNAcol, Fucα1-2Galβ1-3(Fucα1-2Galβ1-4GlcNAcβ1-6)GalNAcol, Galβ1-3[3SGal-(Fuc)(6S)GlcNAcβ1-6]GalNAcol, SGal-(Fuc)(6S)GlcNAc-Galβ1-3GalNAcol, NeuGcα2-3Galβ1-3(Galβ1-4GlcNAcβ1-6)GalNAcol, NeuAcα2-3(GalNAcβ1-4)Galβ1-3(GlcNAcβ1-6)GalNAcol, 6SGalβ1-(Fucα1-)GlcNAcβ1-3(6SGlcNAcβ1-6)GalNAcol, 6SGalβ1-(Fucα1-)(6S)GlcNAcβ1-3(GlcNAcβ1-6)GalNAcol, NeuGcα2-3(GalNAcβ1-4)Galβ1-3(GlcNAcβ1-6)GalNAcol, Galβ1-3[Fucα1-2(GalNAcα1-3)Galβ1-4GlcNAcβ1-6]GalNAcol, 3SGal-GlcNAcβ1-3(6SGlcNAcβ1-6)Galβ1-3GalNAcol, SGalβ1-4GlcNAcβ1-3(SGalβ1-3GlcNAcβ1-6)GalNAcol, NeuAcα2-3Galβ1-3[Galβ1-4(6S)GlcNAcβ1-6]GalNAcol, Galβ1-3[Fucα1-2Galβ1-(Fucα-)(6S)GlcNAcβ1-6]GalNAcol, Fucα1-2Galβ1-3[Gal-(Fuc)(6S)GlcNAcβ1-6]GalNAcol, Fucα1-2Galβ1-3[Fucα1-2Galβ1-4(6S)GlcNAcβ1-6]GalNAcol, NeuGcα2-3Galβ1-3[Galβ1-4(6S)GlcNAcβ1-6]GalNAcol, NeuGcα2-3Galβ1-3(3SGal-GlcNAcβ1-6)GalNAcol, GlcNAcβ1-3[GalNAcα1-3(Fucα1-2)Galβ1-3GlcNAcβ1-6]GalNAcol, NeuAcα2-3(GalNAcβ1-4)Galβ1-3(6SGlcNAcβ1-6)GalNAcol, GlcNAcβ1-3[Fucα1-2Galβ1-(Fucα1-)(6S)GlcNAcβ1-6]GalNAcol, 6SGlcNAcβ1-3[Fucα1-2Galβ1-(Fucα1-)GlcNAcβ1-6]GalNAcol, NeuAcα2-3(GalNAcβ1-4)Galβ1-3(NeuAcα2-6)GalNAcol, GlcNAcβ1-3[NeuGcα2-3Galβ1-4(6S)GlcNAcβ1-6]GalNAcol, Galβ1-3[GalNAcα1-3(Fucα1-2)Galβ1-4(6S)GlcNAcβ1-6]GalNAcol, Galβ1-3GlcNAcβ1-3[Fucα1-2Galβ1-4(6S)GlcNAcβ1-6]GalNAcol, Galβ1-4GlcNAcβ1-3[Fucα1-2Galβ1-3(6S)GlcNAcβ1-6]GalNAcol, Fucα1-2Galβ1-4GlcNAcβ1-3[Galβ1-4(6S)GlcNAcβ1-6]GalNAcol, NeuAcα2-3(GalNAcβ1-4)Galβ1-3(NeuGcα2-6)GalNAcol, NeuAcα2-3Galβ1-3(Fucα1-2Galβ1-4GlcNAcβ1-6)GalNAcol, NeuGcα2-3(GalNAcβ1-4)Galβ1-3(NeuGcα2-6)GalNAcol, Fucα1-2Galβ1-3[SGal-(Fuc)(6S)GlcNAcβ1-6]GalNAcol, NeuGcα2-3Galβ1-3 (Fucα1-2Galβ1-4GlcNAcβ1-6)GalNAcol, Galα1-3Galβ1-3(NeuGcα2-3Galβ1-4GlcNAcβ1-6)GalNAcol, GlcNAcβ1-3[GalNAcα1-3(Fucα1-2)Galβ1-(6S)GlcNAcβ1-6]GalNAcol, GalNAcα1-3(Fucα1-2)Galβ1-3GlcNAcβ1-3(6SGlcNAcβ1-6)GalNAcol, GlcNAcα1-4(Fucα1-2)Galβ1-4GlcNAcβ1-3(NeuAcα2-6)GalNAcol, NeuAcα2-3Galβ1-3[GlcNAcα1-4Galβ1-4GlcNAcβ1-6]GalNAcol, Galβ1-(Fuc)GlcNAcβ1-3(Fucα1-2Galβ1-4GlcNAcβ1-6)GalNAcol, Fucα1-2Galβ1-4GlcNAcβ1-3(Fucα1-2Galβ1-4GlcNAcβ1-6)GalNAcol, SGal-GlcNAcβ1-6[Fucα1-2Gal-(6S)GlcNAcβ1-6]GalNAcol, NeuGcα2-3Galβ1-3(GlcNAcα1-4Galβ1-4GlcNAcβ1-6)GalNAcol, NeuAcα2-3Galβ1-3[Fucα1-2Galβ1-4(6S)GlcNAcβ1-6]GalNAcol, SGal-GlcNAcβ1-3(GlcNAcβ1-6)Galβ1-3(6SGlcNAcβ1-6)GalNAcol, NeuAcα2-3(GalNAcβ 1-4)Galβ1-3[Galβ1-4(6S)GlcNAcβ1-6]GalNAcol, Fucα1-2Galβ1-3[GalNAcα1-3(Fucα1-2)Galβ1-3(6S)GlcNAcβ1-6]GalNAcol, Fucα1-2Galβ1-4GlcNAcβ1-3[Fucα1-2Galβ1-4(6S)GlcNAcβ1-6]GalNAcol, Fucα1-2Galβ1-3[GalNAcα1-3(Fucα1-2)Galβ1-4(6S)GlcNAcβ1-6]GalNAcol, SGal-(Fuc)GlcNAcβ1-3[SGal-(Fuc)GlcNAcβ1-6]GalNAcol, SGal-(Fuc)(6S)GlcNAcβ1-3(3SGal-GlcNAcβ1-6)GalNAcol, Gal(Fuc)(6S)GlcNAcβ1-3(Fucα1-2)Galβ1-3(6SGlcNAcβ1-6)GalNAcol, SGal-(Fuc)GlcNAcβ1-3[Gal-(Fuc)(6S)GlcNAcβ1-6]GalNAcol, SGal-GlcNAcβ1-3[Fucα1-2Gal-(Fuc)(6S)GlcNAcβ1-6]GalNAcol, NeuGcα2-3(GlcNAcβ1-4)Galβ1-3 (Fucα1-2Galβ1-4GlcNAcβ1-6)GalNAcol, NeuGcα2-3Galβ1-3[Fucα1-2Galβ1-3(Fucα1-4)(6S)GlcNAcβ1-6]GalNAcol, SGal-GlcNAcβ1-3[GalNAcα1-3(Fucα1-2)Gal-(6S)GlcNAcβ1-6]GalNAcol, NeuAcα2-3Galβ1-3[GalNAcα1-3(Fucα1-2)Gal-(6S)GlcNAcβ1-6]GalNAcol, Fucα1-2Galβ1-4(6S)GlcNAcβ1-3[Fucα1-2Galβ1-3(Fucα1-4)GlcNAcβ1-6]GalNAcol, GlcNAcα1-4Galβ1-3[NeuGcα2-6Gal-(Fuc)(6S)GlcNAcβ1-6]GalNAcol, GlcNAcα1-4(Fucα1-2)GlcNAcβ1-3[Fucα1-2)Galβ1-4(6S)GlcNAcβ1-6]GalNAcol, GalNAcα1-3(Fucα1-2)Gal-GlcNAcβ1-3[Gal(Fuc)(6S)GlcNAcβ1-6]GalNAcol, Galβ1-3GlcNAcβ1-3[GalNAcα1-3(Fucα1-2)Gal-(Fucα1-2)(6S)GlcNAcβ1-6]GalNAcol, SGal-(Fuc)GlcNAcβ1-3[Fucα1-2Gal-(Fuc)(6S)GlcNAcβ1-6]GalNAcol, Fucα1-2Gal-(Fuc)GlcNAcβ1-3[SGal-(Fuc)(6S)GlcNAcβ1-6]GalNAcol, GalNAcα1-3(Fucα1-2)Gal-GlcNAcβ1-3(NeuAcα2-3Gal-GlcNAcβ1-6)GalNAcol, SGal-(Fuc)GlcNAcβ1-3[GalNAcα1-3(Fucα1-2)Gal-(6S)GlcNAcβ1-6]GalNAcol, Gal-(Fuc)(6S)GlcNAcβ1-3[GalNAcα1-3(Fucα1-2)Gal-(6S)GlcNAcβ1-6]GalNAcol, GalNAcα1-3(Fucα1-2)Gal-GlcNAcβ1-3(NeuGcα2-3Gal-GlcNAcβ1-6)GalNAcol, Fucα1-2Gal(Fuc)GlcNAcβ1-3[Fucα1-2Gal-(Fuc)(6S)GlcNAcβ1-6]GalNAcol, Gal-(6S)GlcNAcβ1-3[SGal-(Fuc)(6S)GlcNAcβ1-6]Galβ1-3(GlcNAcβ1-6)GalNAcol, Fucα1-2Gal(Fuc)GlcNAc-(6S)Galβ1-3[6SGal-(Fuc)GlcNAcβ1-6]GalNAcol, GalNAcα1-3(Fucα1-2)Galβ1-4GlcNAcβ1-3[GalNAcα1-3(Fucα1-2)Galβ1-4(6S)GlcNAcβ1-6]GalNAcol, GalNAcα1-3(Fucα1-2)Gal-GlcNAcβ1-3[GalNAcα1-3(Fucα1-2)Gal-(Fuc)(6S)GlcNAcβ1-6]GalNAcol, NeuAcα2-Galβ1-4GlcNAcβ1-2Manα1-3(Galβ1-4GlcNAcβ1-2Manα1-6)Manβ1-4GlcNAcβ1-4(Fucα1-6)GlcNAcol, and Fucα1-2Galβ1-3(Fucα1-4)GlcNAcβ1-3[NeuGcα2-6(3S)Galβ1-GlcNAcβ1-3(Fucα1-2)Gal-(Fuc)(6S)GlcNAcβ1-6]GalNAcol.

In some embodiments, the composition comprises at least 1, 2, 3, 4, 5, 6, 7, 8, 9, or all 10 sialylated glycopeptide-bound oligosaccharide selected from the following: NeuAcα2-6GalNAc, NeuGcα2-6GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, and Fucα1-2Galβ1-3(NeuAcβ2-6)GalNAc. In some embodiments, the composition comprises NeuAcα2-3Galβ1-3GalNAc (Sialyl-T antigen), NeuAcα2-6GalNAc (Sialyl Tn antigen), and NeuAcα2-3Galβ1-3(NeuAcα2-6)GalNAc (Disialyl T antigen).

In some embodiments, the composition has substantially no free glycans. In some embodiments, the composition has a pH of less than 7.5. In some embodiments, the composition is a powder. In some embodiments, the composition further comprises one or more excipients or carriers. In some embodiments, the composition is administered orally or rectally.

In some embodiments of the methods disclosed herein, the composition comprises at least one, at least two, at least three, at least four, at least five, or all six glycopeptide-bound oligosaccharide having a structure selected from Galβ1-3GalNAcol (T antigen), NeuAcα2-6GalNAcol (Sialyl Tn antigen), Galβ1-4(Fucα1-3)(6S)GlcNAcol (Lewis X), 6SGalβ1-3(Fucα1-4)GlcNAcol (Lewis A), NeuAcα2-3Galβ1-3(NeuAcα2-6)GalNAc (Disialyl T antigen), and NeuAcα2-3Galβ1-3GalNAcol (Sialyl T antigen).

In some embodiments, the GCX composition comprises between 20% and 30% Core 1 type oligosaccharides, between 30% and 50% (e.g., about 30, 35, 40, 45, or 50%) Core 2 type oligosaccharides, between 7% and 12% (e.g., about 7, 8, 9, 10, 11 or 12%) Core 3 type oligosaccharides, between 8% and 20% (e.g., about 8, 9, 10, 11, 12, 13, 14, 15, 16, 17, 18, 19, or 20%) Core 4 type oligosaccharides, between 2% and 6% (e.g., about 2, 3, 4, 5, or 6%) Core 5 type oligosaccharides and between 0.1% and 5% (e.g., about 0.1, 0.5, 0.8, 1, 1.5, 2, 2.5, 3, 3.5, 4, 4.5, or 5%) Core N type oligosaccharides as measured by liquid chromatograph-electrospray ionization tandem mass spectrometry (LC-ESI/MS) and further explained in Example 13. Cores 1-5 and N are as shown in FIG. 31A.

### EXAMPLES

### Example 1—Preparation of GBX

### Protocol 1

300 ml D.I. water was introduced to a 500 ml beaker. Then, under magnetic stirring (500 rpm), 3.2 g of calcium hydroxide was added to the beaker. The suspension solution was kept at room temperature (about 25° C.) and under magnetic stirring (500 rpm) for 10 minutes.

Under magnetic stirring (800 rpm) and at room temperature (about 25° C.), 20 g of mucin was added to the suspension. The suspension was kept at room temperature (about 25° C.) and under magnetic stirring (800 rpm) for 30 minutes. The temperature was brought up to 60° C. using a heating plate. The reaction mixture was kept at 60° C. and under magnetic stirring (500 rpm) for 3 hours.

The reaction mixture was cooled down to room temperature. Then, about 3 g of Celite (diatomaceous earth) was added to the mixture under magnetic stirring (500 rpm) at room temperature (about 25° C.) for 10 minutes. The reaction mixture was then filtered under vacuum on a Buchner funnel with a paper filter (Whatman). The filtrate was neutralized to pH under 7.5 using C02. Then 1 to 2 g of celite was added to the filtrate. The suspension solution was stirred magnetically at 500 rpm for 5 minutes.

The suspension was filtered again under vacuum on a Buchner funnel with a paper filter (Whatman). Dowex ion exchange hydrogen form resin was added to adjust the pH to 6.5 to 7. Dowex ion exchange hydrogen form resin was filtered out under vacuum using a Buchner funnel with a paper filter (Whatman).

Finally, the filtrate was concentrated under rotary evaporator and then spray dried.

GBX was collected with a yield of about 50%.

The resulting GBX composition was found to have a total oligosaccharide content of >10% (w/w) with a ratio of glycopeptides:free glycans of greater than 4:1 (w/w). The total glycoprotein of the composition was 12% or less (w/w).

The bound glycans and the free glycans in the GBX composition were found to have the following general formulae:
Hex1HexNAc1, HexNAc2, Hex1HexNAc1Sul1, HexNAc1deHex1Sul1, HexNAc2Sul1, NeuAc1HexNAc1, Hex2HexNAc1, Hex1HexNAc1deHex1, Hex1HexNAc2, HexNAc2deHex1, Hex1HexNAc1deHex1Sul1, Hex1HexNAcSul1, Hex2HexNAc1deHex1, NeuAc1Hex1HexNAc1, Hex1HexNAc1deHex2, Hex2HexNAc2, Hex1HexNAc2deHex1, Hex2HexNAc1deHex1Sul1, Hex1HexNAc1deHex2Sul1, Hex1HexNAc3, Hex1HexNAc2deHex1Sul1, NeuAc1Hex1HexNAc1deHex1, Hex2HexNAc2Sul1, NeuAc1Hex1HexNAc2, Hex1HexNAc3Sul1, Hex2HexNAc2deHex1, Hex1HexNAc2deHex2, Hex1HexNAc3deHex1, Hex2HexNAc3, Hex2HexNAc2deHex1Sul1, Hex1HexNAc4, Hex1HexNAc3deHex1Sul1, Hex3HexNAc2deHex1, NeuAc1Hex2HexNAc2, Hex2HexNAc2deHex2, Hex2HexNAc3deHex1, Hex3HexNAc2deHex1Sul1, Hex2HexNAc2deHex2Sul1, Hex2HexNAc4, Hex2HexNAc3deHex1Sul1, Hex3HexNAc3deHex1, Hex2HexNAc4deHex1, Hex3HexNAc3deHex1Sul1, and Hex4HexNAc3deHex1Sul1.

In some embodiments, the glycans in the GBX composition were found to have the following general formulae: Hex1HexNAc1, Hex1HexNAc1deHex1, Hex1HexNAc1deHex1Sul1, Hex1HexNAc1deHex2, Hex1HexNAc1deHex2Sul1, Hex1HexNAc1Sul1, Hex1HexNAc2, Hex1HexNAc2deHex1, Hex1HexNAc2deHex1Sul1, Hex1HexNAc2deHex2, Hex1HexNAc2Sul1, Hex1HexNAc3, Hex1HexNAc3deHex1, Hex1HexNAc3deHex1Sul1, Hex1HexNAc3Sul1, Hex1HexNAc4, Hex1HexNAcSul1, Hex2HexNAc1, Hex2HexNAc1deHex1, Hex2HexNAc1deHex1Sul1, Hex2HexNAc2, Hex2HexNAc2deHex1, Hex2HexNAc2deHex1Sul1, Hex2HexNAc2deHex2, Hex2HexNAc2deHex2Sul1, Hex2HexNAc2Sul1, Hex2HexNAc3, Hex2HexNAc3deHex1, Hex2HexNAc3deHex1Sul1, Hex2HexNAc4, Hex2HexNAc4deHex1, Hex3HexNAc2deHex1, Hex3HexNAc2deHex1Sul1, Hex3HexNAc3deHex1, Hex3HexNAc3deHex1Sul1, Hex4HexNAc3deHex1Sul1, HexNAc1deHex1Sul1, HexNAc2, HexNAc2deHex1, HexNAc2Sul1, NeuAc1Hex1HexNAc1, NeuAc1Hex1HexNAc1deHex1, NeuAc1Hex1HexNAc2, NeuAc1Hex2HexNAc2, and NeuAc1HexNAc1.

In some embodiments, the GBX composition comprises glycopeptide-bound oligosaccharides having at least 10, at least 20, at least, 30, at least 40, at least 45, or all (e.g., at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46) of the general formulae listed above. In some embodiments, in the GBX composition, there are at least 10 glycopeptide-bound oligosaccharides having different formula, and each formula is present in the composition makes up between 0.1% and 10% (e.g., 0.1%, 0.2%, 0.3%, 0.4%, 0.5%, 0.6%, 0.7%, 0.8%, 0.9% 1.5%, 2%, 2.5%, 3%, 3.5%, 4%, 4.5%, 5%, 5.5%, 6%, 6.5%, 7%, 7.5%, 8%, 8.5%, 9%, 9.5%, 10%,) of all glycopeptide-bound oligosaccharides in the composition.

It was found that the following glycan formulae were the most abundant in the composition, each having between 3% and 10% representation of all glycopeptide-bound oligosaccharides in the composition: Hex1HexNAc1deHex1, Hex1HexNAc3deHex1Sul1, Hex1NAc3Sul1, NeuAc1Hex1HexNAc1, Hex1HexNAc2deHex1, NeuAc1Hex1HexNAc1deHex1, Hex1HexNAc3deHex1Sul1, NeuAc1Hex1HexNAc1, Hex1HexNAc2deHex1Sul1, Hex1HexNAc2Sul1, Hex1HexNAc1, and Hex1HexNAc3Sul1.

It was found that the glycopeptide-bound oligosaccharides in the GBX composition have at least 10 of the following glycan structures: Galβ1-3GalNAcol, GlcNAcβ1-4Galol, GlcNAcα1-4Galol, HexNAc-GlcAol, GlcNAcβ1-6GalNAcol, Galβ1-4(6S)GlcNAcol, 6SGlcNAc-Fucol, Galβ1-4(6S)GlcNAcol, (S)Galβ1-GlcNAcol, (S)Galβ1-GlcNAcol, 6SGlcNAcβ1-6GalNAcol, 6SGlcNAcβ1-3GalNAcol, NeuAc-HexNAcol, NeuAcα2-6GalNAcol, Galβ1-4GlcNAcβ1-3Gal minus H₂O, Fucα1-2(GalNAcα1-3)Galol, Gal-GlcNAc-Fucol, Gal-GlcNAc-Fucol, Fucα1-2Galβ1-4GlcNAcol, Fucα1-2Galβ1-3GlcNAcol, Fucα1-2Galβ1-3GalNAcol, Galβ1-4GlcNAcβ1-3Gal, GlcNAcα1-4Galβ1-4GlcNAc minus H₂O, GlcNAc-GlcNAc-Fucol, Galβ1-3(GlcNAcβ1-6)GalNAcol, GlcNAcα1-4Galβ1-3GalNAcol, GlcNAcα1-4Galβ1-4GlcNAcol, Fucα1-2Galβ1-4(6S)GlcNAcol, Galβ1-4(Fucα1-3)(6S)GlcNAcol, 6SGalβ1-3(Fucα1-4)GlcNAcol, SGalβ1-3(GlcNAcβ1-6)GalNAcol, Galβ1-3(6SGlcNAcβ1-6)GalNAcol, Galβ1-4(6S)GlcNAcβ1-6GalNAcol, Fucα1-2Galβ-4GlcNAcβ1-3Gal minus H₂O, Galβ1-3(NeuAcα2-6)GalNAcol, NeuAcα2-3Galβ1-3GalNAcol, Fucα1-2Galβ1-4(Fucα1-3)GlcNAcol, Fucα1-2Galβ-4GlcNAcβ1-3Gal, GlcNAcα1-4Galβ1-4GlcNAcβ1-3Gal minus H₂O, Fucα1-2(GalNAcα1-3)Galβ1-4GlcNAcol, Fucα1-2(GalNAcα1-3)Galβ1-3GalNAcol, GlcNAcα1-4Gal(Fuc)GlcNAcol, GlcNAcα1-4Gal(Fuc)GlcNAcol, Fucα1-2Galβ1-3(GlcNAcβ1-6)GalNAcol, GlcNAcα1-4Galβ1-4GlcNAcβ1-3Gal, Galβ1-3(Galβ1-4GlcNAcβ1-6)GalNAcol, GlcNAcα1-4Galβ1-4GlcNAcβ1-3Galol, Galβ1-3(Galβ1-4GlcNAcβ1-6)GalNAcol, Galβ1-4GlcNAcβ1-3Galβ1-3GalNAcol, Fucα1-2Galβ1-4(6S)GlcNAcβ1-4Gal minus H₂O, Fucα1-2(S)Galβ1-4GlcNAcβ1-4Gal minus H₂O, Fucα1-2Gal(Fuc)(6S)GlcNAcol, Fucα1-2Galβ1-3(6S)GlcNAcβ1-4Galol, GlcNAcα1-4Galβ1-3(GlcNAcβ1-6)GalNAcol, GlcNAcα1-3Galβ1-4GlcNAcβ1-6GalNAcol, Fucα1-2(GalNAcα1-3)Galβ1-4(6S)GlcNAcol, Fucα1-2Galβ1-4(6S)GlcNAcβ1-3GalNAcol, Fucα1-2Galβ1-3(6S-GlcNAcβ1-6)GalNAcol, Fucα1-2Galβ1-3(NeuAcα2-6)GalNAcol, Galβ1-3[Galβ1-4(6S)GlcNAcβ1-6]GalNAcol, GlcNAcα1-4Galβ1-3(NeuAcα2-6)GalNAcol, GlcNAcα1-4Galβ1-3[(6S)GlcNAcβ1-6]GalNAcol, GalNAcα1-3(Fucα1-2)Galβ1-4GlcNAcβ1-3Gal minus H₂O, GlcNAcα1-4Galβ1-3(NeuAcα2-6)GalNAcol, Fucα1-2(GalNAcα1-3)Gal-(Fuc)GlcNAcol, Galβ1-3(Fucα1-4)GlcNAcβ1-3Galβ1-3GalNAcol, Galβ1-4(Fucα1-3)GlcNAcβ1-3Galβ1-3GalNAcol, Fucα1-2Galβ1-4GlcNAcβ1-3Galβ1-4GlcNAcol, Galβ1-3(Fucα1-2Galβ1-4GlcNAcβ1-6)GalNAcol, Fucα1-2Galβ1-3(Galβ1-4GlcNAcβ1-6)GalNAcol, Fucα1-2(GalNAcα1-3)Galβ1-4GlcNAcβ1-6GalNAcol, Fucα1-2Galβ1-3(GalNAcβ1-4GlcNAcβ1-6)GalNAcol, Galβ1-4GlcNAcβ1-3(GlcNAcβ1-6)Galβ1-3GalNAcol, GlcNAcα1-4Galβ1-3(Galβ1-4GlcNAcβ1-6)GalNAcol, GalNAcα1-3(Fucα1-2)Galβ1-4(6S)GlcNAcβ1-3Gal minus H₂O, Fucα1-2Galβ1-4(6S)GlcNAcβ1-3Galβ1-3GalNAcol, Fucα1-2Galβ1-3[Galβ1-4(6S)GlcNAcβ1-6]GalNAcol, GlcNAcα1-4Galβ1-3(GalNAcβ1-4GlcNAcβ1-6)GalNAcol, GlcNAcβ1-3[Fucα1-2Galβ1-4(6S)GlcNAcβ1-6]GalNAcol, GalNAcα1-3(Fucα1-2)Galβ1-3(6SGlcNAcβ1-6)GalNAcol, Fucα1-2Galβ1-4GlcNAcβ1-3(Galβ1-4GlcNAcβ1-6)Gal, Fucα1-2Galβ1-4GlcNAcβ1-3(Galβ1-4GlcNAcβ1-6)Gal, Galβ1-4GlcNAcβ1-3Galβ1-3(NeuAcα2-6)GalNAcol, Fucα1-2Galβ1-3(Fucα1-2Galβ1-4GlcNAcβ1-6)GalNAcol, Galβ1-3[Fucα1-2(GalNAcα1-3)Galβ1-4GlcNAcβ1-6]GalNAcol, GlcNAcα1-4Galβ1-3(Fucα1-2Galβ1-4GlcNAcβ1-6)GalNAcol, Fucα1-2Galβ1-3(GlcNAcα1-4Galβ1-4GlcNAcβ1-6)GalNAcol, Fucα1-2Galβ1-4(6S)GlcNAcβ1-3(Galβ1-4GlcNAcβ1-6)Gal minus H₂O, Fucα1-2Galβ1-3[Fucα1-2Galβ1-4(6S)GlcNAcβ1-6]GalNAcol, GlcNAcα1-4Galβ1-3(GlcNAcα1-4Galβ1-4GlcNAcβ1-6)GalNAcol, Gal-GlcNAcβ1-3(GlcNAcα1-4Galβ1-4GlcNAcβ1-6)GalNAcol, GalNAcα1-3(Fucα1-2)Galβ1-4(6S)GlcNAcβ1-3Galβ1-3GalNAcol, Fucα1-2Galβ1-4GlcNAcβ1-3Galβ1-4GlcNAcβ1-3Galβ1-3GalNAcol, GlcNAcα1-4Galβ1-3[GalNAcα1-3(Fucα1-2)Galβ1-4GlcNAcβ1-6]GalNAcol, Fucα1-2Galβ1-4(6S)GlcNAcβ1-3Galβ1-4GlcNAcβ1-3Galβ1-4GlcNAcol, and Fucα1-2Galβ1-4(6S)GlcNAcβ1-3Galβ1-4GlcNAcβ1-3(Galβ1-4GlcNAcβ1-6)Gal minus H₂O.

It was found that the glycopeptide-bound oligosaccharides in the GBX composition have at least 10 of the following glycan structures: Galβ1-3GalNAc, GlcNAcα1-4Gal, GlcNAcβ1-4Gal, Fucα1-2(GalNAcα1-3)Gal, Fucα1-2Galβ1-3GalNAc, Fucα1-2Galβ1-3GlcNAc, Fucα1-2Galβ1-4GlcNAc, Gal-GlcNAc-Fuc, 6SGalβ1-3(Fucα1-4)GlcNAc, Fucα1-2Galβ1-4(6S)GlcNAc, Galβ1-4(Fucα1-3)(6S)GlcNAc, Fucα1-2Galβ1-4(Fucα1-3)GlcNAc, Fucα1-2Gal(Fuc)(6S)GlcNAc, (S)Galβ1-GlcNAc, Galβ1-4(6S)GlcNAc, Galβ1-3(GlcNAcβ1-6)GalNAc, GlcNAcα1-4Galβ1-3GalNAc, GlcNAcα1-4Galβ1-4GlcNAc, Fucα1-2(GalNAcα1-3)Galβ1-3GalNAc, Fucα1-2(GalNAcα1-3)Galβ1-4GlcNAc, Fucα1-2Galβ1-3(GlcNAcβ1-6)GalNAc, GlcNAcα1-4Gal(Fuc)GlcNAc, Fucα1-2(GalNAcα1-3)Galβ1-4(6S)GlcNAc, Fucα1-2Galβ1-3(6S-GlcNAcβ1-6)GalNAc, Fucα1-2Galβ1-4(6S)GlcNAcβ1-3GalNAc, Fucα1-2(GalNAcα1-3)Gal-(Fuc)GlcNAc, Galβ1-3(6SGlcNAcβ1-6)GalNAc, GlcNAcα1-3Galβ1-4GlcNAcβ1-6GalNAc, GlcNAcα1-4Galβ1-3(GlcNAcβ1-6)GalNAc, Fucα1-2(GalNAcα1-3)Galβ1-4GlcNAcβ1-6GalNAc, Fucα1-2Galβ1-3(GalNAcβ1-4GlcNAcβ1-6)GalNAc, GalNAcα1-3(Fucα1-2)Galβ1-3(6SGlcNAcβ1-6)GalNAc, GlcNAcβ1-3[Fucα1-2Galβ1-4(6S)GlcNAcβ1-6]GalNAc, GlcNAcα1-4Galβ1-3[(6S)GlcNAcβ1-6]GalNAc, GlcNAcα1-4Galβ1-3(GalNAcβ1-4GlcNAcβ1-6)GalNAc, Galβ1-4(6S)GlcNAcβ1-6GalNAc, SGalβ1-3(GlcNAcβ1-6)GalNAc, Galβ1-4GlcNAcβ1-3Gal, Fucα1-2Galβ-4GlcNAcβ1-3Gal, Fucα1-2(S)Galβ1-4GlcNAcβ1-4Gal, Fucα1-2Galβ1-3(6S)GlcNAcβ1-4Gal, Fucα1-2Galβ1-4(6S)GlcNAcβ1-4Gal, Galβ1-3(Galβ1-4GlcNAcβ1-6)GalNAc, Galβ1-4GlcNAcβ1-3Galβ1-3GalNAc, GlcNAcα1-4Galβ1-4GlcNAcβ1-3Gal, Fucα1-2Galβ1-3(Galβ1-4GlcNAcβ1-6)GalNAc, Fucα1-2Galβ1-4GlcNAcβ1-3Galβ1-4GlcNAc, GalNAcα1-3(Fucα1-2)Galβ1-4GlcNAcβ1-3Gal, Galβ1-3(Fucα1-2Galβ1-4GlcNAcβ1-6)GalNAc, Galβ1-3(Fucα1-4)GlcNAcβ1-3Galβ1-3GalNAc, Galβ1-4(Fucα1-3)GlcNAcβ1-3Galβ1-3GalNAc, Fucα1-2Galβ1-3[Galβ1-4(6S)GlcNAcβ1-6]GalNAc, Fucα1-2Galβ1-4(6S)GlcNAcβ1-3Galβ1-3GalNAc, GalNAcα1-3(Fucα1-2)Galβ1-4(6S)GlcNAcβ1-3Gal, Fucα1-2Galβ1-3(Fucα1-2Galβ1-4GlcNAcβ1-6)GalNAc, Fucα1-2Galβ1-3[Fucα1-2Galβ1-4(6S)GlcNAcβ1-6]GalNAc, Galβ1-3[Galβ1-4(6S)GlcNAcβ1-6]GalNAc, Galβ1-4GlcNAcβ1-3(GlcNAcβ1-6)Galβ1-3GalNAc, GlcNAcα1-4Galβ1-3(Galβ1-4GlcNAcβ1-6)GalNAc, Fucα1-2Galβ1-3(GlcNAcα1-4Galβ1-4GlcNAcβ1-6)GalNAc, Galβ1-3[Fucα1-2(GalNAcα1-3)Galβ1-4GlcNAcβ1-6]GalNAc, GlcNAcα1-4Galβ1-3(Fucα1-2Galβ1-4GlcNAcβ1-6)GalNAc, GalNAcα1-3(Fucα1-2)Galβ1-4(6S)GlcNAcβ1-3Galβ1-3GalNAc, Gal-GlcNAcβ1-3(GlcNAcα1-4Galβ1-4GlcNAcβ1-6)GalNAc, GlcNAcα1-4Galβ1-3(GlcNAcα1-4Galβ1-4GlcNAcβ1-6)GalNAc, GlcNAcα1-4Galβ1-3[GalNAcα1-3(Fucα1-2)Galβ1-4GlcNAcβ1-6]GalNAc, Fucα1-2Galβ1-4GlcNAcβ1-3(Galβ1-4GlcNAcβ1-6)Gal, Fucα1-2Galβ1-4(6S)GlcNAcβ1-3(Galβ1-4GlcNAcβ1-6)Gal, Fucα1-2Galβ1-4GlcNAcβ1-3Galβ1-4GlcNAcβ1-3Galβ1-3GalNAc, Fucα1-2Galβ1-4(6S)GlcNAcβ1-3Galβ1-4GlcNAcβ1-3Galβ1-4GlcNAc, Fucα1-2Galβ1-4(6S)GlcNAcβ1-3Galβ1-4GlcNAcβ1-3(Galβ1-4GlcNAcβ1-6)Gal, 6SGlcNAc-Fuc, HexNAc-GlcA, GlcNAcβ1-6GalNAc, GlcNAc-GlcNAc-Fuc, 6SGlcNAcβ1-3GalNAc, 6SGlcNAcβ1-6GalNAc, Galβ1-3(NeuAcα2-6)GalNAc, NeuAcα2-3Galβ1-3GalNAc, Fucα1-2Galβ1-3(NeuAcα2-6)GalNAc, GlcNAcα1-4Galβ1-3(NeuAcα2-6)GalNAc, Galβ1-4GlcNAcβ1-3Galβ1-3(NeuAcα2-6)GalNAc, NeuAc-HexNAc, and NeuAcα2-6GalNAc.

### Example 2—Alternate GBX Purification Protocol

### Protocol 2 (CO₂ Neutralization Omitted as Dowex Ion Exchange Hydrogen Resin can Also Remove the Calcium)

300 ml D.I. water was introduced to a 500 ml beaker. Then, under magnetic stirring (500 rpm), 3.2 g of calcium hydroxide was added to the beaker. The suspension solution was kept at room temperature (about 25° C.) and under magnetic stirring (500 rpm) for 10 minutes.

Under magnetic stirring (800 rpm) and at room temperature (about 25° C.), 20 g of ali mucin (Porcine Gastric Mucin) was added to the suspension. The suspension was kept at room temperature (about 25° C.) and under magnetic stirring (800 rpm) for 30 minutes. The temperature was brought up to 60° C. using a heating plate. The reaction mixture was kept at 60° C. and under magnetic stirring (500 rpm) for 3 hours.

The reaction mixture was cooled down to room temperature. Then, about 3 g of Celite was added to the mixture under magnetic stirring (500 rpm) at room temperature (about 25° C.) for 10 minutes. The reaction mixture was then filtered under vacuum on a Buchner funnel with a paper filter (Whatman).

Dowex ion exchange hydrogen form resin was added to adjust the pH to 6.5 to 7. Dowex ion exchange hydrogen form resin was filtered out under vacuum using a Buchner funnel with a paper filter (Whatman).

Finally, the filtrate was concentrated under rotary evaporator and then spray dried.

GBX was collected with a yield of about 50%.

### Example 3—GCX Preparation

A 3 kDa spiral wound ultrafiltration (UF) membrane module with filtration surface area 2.7 m² was selected and was operated in a pilot installation. Such surface area allowed for a relatively high permeate flow (63-100 L/h) and shortened the experimental running time. The feed solution was made by mixing the powder GNU100 with demineralized water (DW), concentration before diafiltration were 5%, 5.5% and 7.5% (w/v) respectively in three different trials. GNU100 was prepared by the methods disclosed in Examples 1-3 of WO 2020/104486, published May 28, 2020, which is incorporated herein by reference in its entirety.

Briefly, GNU100 preparation included the following: Partially purified hydrolyzed porcine gastrointestinal tract mucins were obtained from commercial sources and stabilized at a pH of 5.0 using sulfuric acid or sodium hydroxide, as appropriate. The stabilized mucins were then centrifuged at low speed (500 to 10,000 Xg) to remove large insoluble particles, fats, and lipids. The mucins were then desalinated using a dialysis membrane (Slide-A-Lyzer Dialysis Flask (2K MWCO) ThermoFisher Scientific) and then concentrated by evaporation with a rotary evaporator (Fisher Scientific) with heating to at least 80° C., forming a slurry. The slurry was further processed by partially filtering through a 0.2 mM Polyethersulfone (PES) filter (Millipore Sigma) to remove some amino acids and salts, and the retentate collected

One hundred ml of the retentate was filtrated on Whatman filter paper (diameter 110 mm, pore size 4-7 inn) by suction using a Buchner funnel. About 100 ml of filtrate (brown liquid) was obtained. The solid residue was discarded, and the filtrate dried under rotary evaporator at 50 mbar and 50° C. m=31.8 g Total yield=31.8%. Dry substance yield=88% to yield a powder composition called GNU 100. The powder composition was white to yellow with a neutral or slight amino acid smell and had a 2-5% moisture content. The water solubility of the powder was 80 to 120 g/L at 25° C.

Alternatively, GNU100 preparation included the following: Partially purified hydrolyzed porcine gastrointestinal tract mucins were obtained from commercial sources and stabilized at a pH of 5.0 using sulfuric acid or sodium hydroxide, as appropriate. The stabilized mucins were then centrifuged at low speed (500 to 10,000 Xg) to remove large insoluble particles, fats, and lipids. The mucins were desalinated using a dialysis membrane (Slide-A-Lyzer Dialysis Flask (2K MWCO) ThermoFisher Scientific) and then concentrated by evaporation with a rotary evaporator (Fisher Scientific) with heating to at least 80° C., forming a slurry. The slurry was further processed by partially filtering through a 0.2 mM Polyethersulfone (PES) filter (Millipore Sigma) to remove some salts and amino acids, and the retentate collected.

One hundred ml of the retentate was filtrated on Whatman filter paper (diameter 110 mm, pore size 4-7 mm) by suction using a Buchner funnel. 100 ml of filtrate (brown liquid) was obtained. The filtrate was then ultra-filtrated though a PES membrane having a molecular weight cutoff (MWCO) of 2 kDa (Millipore Sigma) and the retentate collected. The retentate was then dried under rotary evaporator at 50 mbar and 50° C.

Alternatively, GNU100 preparation alternatively included the following: Partially purified hydrolyzed porcine gastrointestinal tract mucins were obtained from commercial sources and stabilized at a pH of 5.0 using sulfuric acid or sodium hydroxide, as appropriate. The stabilized mucins were then centrifuged at low speed (5,000 to 10,000×g) to remove large insoluble particles and lipids. The mucins were then desalinated using a dialysis membrane (Slide-A-Lyzer Dialysis Flask (2K MWCO) ThermoFisher Scientific) and then concentrated by evaporation with a rotary evaporator (Fisher Scientific) with heating to at least 80° C., forming a slurry. The slurry was further purified by filtering through a 0.2 mM Polyethersulfone (PES) filter (Millipore Sigma) and the retentate collected.

One hundred ml of the retentate was filtrated on Whatman filter paper (diameter 110 mm, pore size 4-7 mm) by suction using a Buchner funnel. 100 ml of filtrate (brown liquid) was obtained. The filtrate was then concentrated.

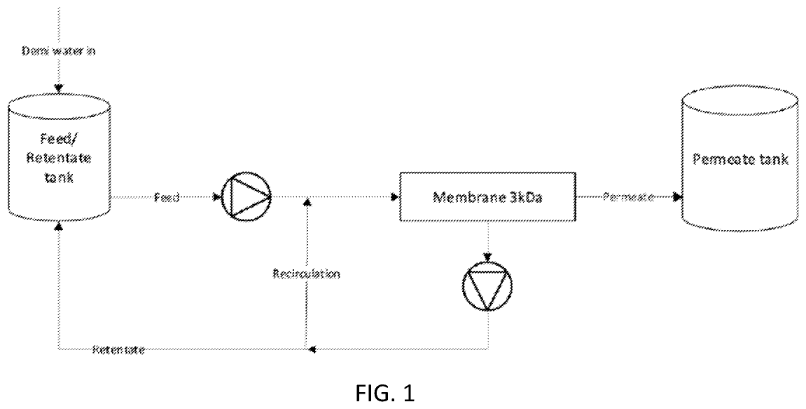

GNU100 was further processed as follows: Operating parameters were kept the same for each trial, in which feed flowrate was 450 L/h at a feed pressure fixed at 3 bar. The system is equipped with an internal recirculation pump which can provide 1.05 m³/h crossflow through the membrane surface for less membrane fouling. Retentate was returned to the feed tank for further processing. DW was continuously added into the feed tank with the same flowrate as the permeate. This diafiltration (DF) concept facilitates to ‘wash out’ the unwanted small MW ions and substances for purifying the target proteins, as well as to maintain a stable permeate flow within a trial. Up to 10 cycles of DF was performed for each trial, the conductivity was reduced from 10.47 mS/cm (based on a 7.5% w/v diafiltration starting concentration) to 0.57 mS/cm in the 80 L feed/retentate tank. Afterwards, filtration continued without adding DW in, for a purpose of concentrating the retentate. The trial with highest diafiltration starting concentration 7.5% (w/v) demonstrated the highest product yield, in the end of this trial, approximately 6 L retentate with conductivity of 2.14 mS/cm was obtained, which has a dry product content of 240 g (on a basis of 6 kg powder as feed). The protocol for diafiltration is shown in FIG. 1.

The GCX was found to have the following structures:

| Mass^{[1]} | Composition^{[2]} | Putative structures^{[3]} |
|---|---|---|
| 384 | Hex1HexNAc1 | Galβ1-3GalNAcol |
| 425-1 | HexNAc2 | GlcNAcβ1-3GalNAcol |
| 425-2 | HexNAc2 | GalNAcα1-3GalNAcol |
| 462 | Hex1HexNAc1Sul | SGalβ1-GlcNAc |
| 464 | Hex1HexNAc1Sul | 3SGalβ1-3GalNAcol |
| 505-1 | HexNAc2Sul1 | 6SGlcNAcβ1-6GalNAcol |
| 505-2 | HexNAc2Sul1 | 6SGlcNAcβ1-3GalNAcol |
| 513 | NeuAc1HexNAc1 | NeuAcα2-6GalNAcol |
| 529 | NeuGc1HexNAc1 | NeuGcα2-6GalNAcol |
| 530-1 | Hex1HexNAc1deHex1 | Fucα1-2(GalNAcα1-3)Galol |
| 530-2 | Hex1HexNAc1deHex1 | Fucα1-2Galβ1-4GlcNAcol |
| 530-3 | Hex1HexNAc1deHex1 | Fucα1-2Galβ1-3GalNAcol |
| 546 | Hex2HexNAc1 | Galβ1-4GlcNAcβ1-3Galol |
| 587-1 | Hex1HexNAc2 | Galβ1-3(GlcNAcβ1- |
| | | 6)GalNAcol |
| 587-2 | Hex1HexNAc2 | Galβ1-4GlcNAcβ1- |
| | | 3GalNAcol |
| 608 | Hex1HexNAc1deHex1Sul1 | 6SGal(Fuc)GlcNAc |
| 626 | Hex2HexNAc1Sul1 | 3SGal-GlcNAcβ1-3Galol |
| 628 | HexNAc3 | GlcNAcβ1-3(GlcNAcβ1- |
| | | 6)GalNAcol |
| 667-1 | Hex1HexNAc2Sul1 | Galβ1-3(6SGlcNAcβ1- |
| | | 6)GalNAcol |
| 667-2 | Hex1HexNAc2Sul1 | 3SGalβ1-3GlcNAcβ1- |
| | | 3GalNAcol |
| 675-1 | NeuAc1Hex1HexNAc1 | Galβ1-3(NeuAcα2- |
| | | 6)GalNAcol |
| 675-2 | NeuAc1Hex1HexNAc1 | NeuAcα2-3Galβ1-3GalNAcol |
| 691-1 | NeuGc1Hex1HexNAc1 | Galβ1-3(NeuGcα2- |
| | | 6)GalNAcol |
| 691-2 | NeuGc1Hex1HexNAc1 | NeuGcα2-3Galβ1-3GalNAcol |
| 692 | Hex2HexNAc1deHex1 | Fucα1-2Galβ-4GlcNAcβ1- |
| | | 3Galol |
| 708-1 | HexNAc3Sul1 | 6SGlcNAcβ1-3(GlcNAcβ1- |
| | | 6)GalNAcol |
| 708-2 | HexNAc3Sul1 | GlcNAcβ1-3(6S-GlcNAcβ1- |
| | | 6)GalNAcol |
| 716-1 | NeuAc1HexNAc2 | GlcNAcβ1-3(NeuAcα2- |
| | | 6)GalNAcol |
| 716-2 | NeuAc1HexNAc2 | GalNAcα1-3(NeuAcα2- |
| | | 6)GalNAcol |
| 732-1 | NeuGc1HexNAc2 | GlcNAcβ1-3(NeuGcα2- |
| | | 6)GalNAcol |
| 732-2 | NeuGc1HexNAc2 | GalNAcα1-3(NeuGcα2- |
| | | 6)GalNAcol |
| 733-1 | Hex1HexNAc2deHex1 | Galβ1-3(Fucα1-4)GlcNAcβ1- |
| | | 3GalNAcol |
| 733-2 | Hex1HexNAc2deHex1 | Fucα1-2(GalNAcα1-3)Galβ1- |
| | | 3GalNAcol |
| 733-3 | Hex1HexNAc2deHex1 | Fucα1-2Galβ1-3GlcNAcβ1- |
| | | 3GalNAcol |
| 733-4 | Hex1HexNAc2deHex1 | Fucα1-2Galβ1-3(GlcNAcβ1- |
| | | 6)GalNAcol |
| 749-1 | Hex2HexNAc2 | Galβ1-3(Galβ1-4GlcNAcβ1- |
| | | 6)GalNAcol |
| 749-2 | Hex2HexNAc2 | Galβ1-3GlcNAcβ1-3Galβ1- |
| | | 3GalNAcol |
| 754 | Hex1HexNAc1deHex2Sul1 | Fucα1-2Gal(Fuc)(6S)GlcNAc |
| 772 | Hex2HexNAc1deHex1Sul1 | 6SGalβ1-4(Fucα1- |
| | | 3)GlcNAcβ1-3Galol |
| 790 | Hex1HexNAc3 | GlcNAcβ1-3(Galβ1- |
| | | 4GlcNAcβ1-6)GalNAcol |
| 813-1 | Hex1HexNAc2deHex1Sul1 | 6SGalβ1-4(Fucα1- |
| | | 3)GlcNAcβ1-3GalNAcol |
| 813-2 | Hex1HexNAc2deHex1Sul1 | Fucα1-2Galβ1-3(6S- |
| | | GlcNAcβ1-6)GalNAcol |
| 821 | NeuAc1Hex1HexNAc1deHex1 | Fucα1-2Galβ1-3(NeuAcα2- |
| | | 6)GalNAcol |
| 829-1 | Hex2HexNAcSul1 | Galβ1-3[Galβ1- |
| | | 4(6S)GlcNAcβ1-6]GalNAcol |
| 829-2 | Hex2HexNAcSul1 | 3SGalβ1-3GlcNAcβ1-3Galβ1- |
| | | 3GalNAcol |
| 837 | NeuGc1Hex1HexNAc1deHex1 | Fucα1-2Galβ1-3(NeuGcα2- |
| | | 6)GalNAcol |
| 870-1 | Hex1HexNAc3Sul1 | GlcNAcα1-4Galβ1- |
| | | 3[(6S)GlcNAcβ1-6]GalNAcol |
| 870-2 | Hex1HexNAc3Sul1 | GlcNAcα1-4(S)Galβ1- |
| | | 4GlcNAcβ1-3GalNAcol |
| 870-3 | Hex1HexNAc3Sul1 | GlcNAcβ1-3[Gal- |
| | | (6S)GlcNAcβ1-6]GalNAcol |
| 870-4 | Hex1HexNAc3Sul1 | Galβ1-4GlcNAcβ1- |
| | | 3(6SGlcNAcβ1-6)GalNAcol |
| 870-5 | Hex1HexNAc3Sul1 | GlcNAcβ1-3[Galβ1- |
| | | 4(6S)GlcNAcβ1-6]GalNAcol |
| 878-1 | NeuAc1Hex1HexNAc2 | NeuAcα2-3(GalNAcβ1- |
| | | 4)Galβ1-3GalNAcol |
| 878-2 | NeuAc1Hex1HexNAc2 | GlcNAcα1-4Galβ1- |
| | | 3(NeuAcα2-6)GalNAcol |
| 878-3 | NeuAc1Hex1HexNAc2 | NeuAcα2-3Galβ1- |
| | | 3(GlcNAcβ1-6)GalNAcol |
| 894-1 | NeuGc1Hex1HexNAc2 | GalNAcβ1-4(NeuGcα2- |
| | | 3)Galβ1-3GalNAcol |
| 894-2 | NeuGc1Hex1HexNAc2 | NeuGcα2-3Galβ1- |
| | | 3(GlcNAcβ1-6)GalNAcol |
| 895-1 | Hex2HexNAc2deHex1 | Galβ1-3[Galβ1-3(Fucα1- |
| | | 4)GlcNAcβ1-6]GalNAcol |
| 895-2 | Hex2HexNAc2deHex1 | Galβ1-3(Fucα1-2Galβ1- |
| | | 4GlcNAcβ1-6)GalNAcol |
| 895-3 | Hex2HexNAc2deHex1 | Fucα1-2Galβ1-3(Galβ1- |
| | | 4GlcNAcβ1-6)GalNAcol |
| 911-1 | Hex3HexNAc2 | Galβ1-3(Galα1-3Galβ1- |
| | | 4GlcNAcβ1-6)GalNAcol |
| 911-2 | Hex3HexNAc2 | Galα1-3Galβ1-4GlcNAcβ1- |
| | | 3Galβ1-3GalNAcol |
| 915 | NeuGc1Hex1HexNAc1deHex1Sul1 | NeuGcα2-3(6S)Gal- |
| | | (Fuc)GlcNAc |
| 936-1 | Hex1HexNAc3deHex1 | GlcNAcβ1-3[Gal- |
| | | (Fuc)GlcNAcβ1-6]GalNAcol |
| 936-2 | Hex1HexNAc3deHex1 | Fucα1-2(GalNAcα1-3)Galβ1- |
| | | 4GlcNAcβ1-3GalNAcol |
| 936-3 | Hex1HexNAc3deHex1 | GlcNAcβ1-3(Fucα1-2Galβ1- |
| | | 4GlcNAcβ1-6)GalNAcol |
| 950 | Hex1HexNAc3Sul2 | 3SGalβ1-4GlcNAcβ1- |
| | | 3(6SGlcNAcβ1-6)GalNAcol |
| 952 | Hex2HexNAc3 | GlcNAcα1-4Galβ1-3(Galβ1- |
| | | 4GlcNAcβ1-6)GalNAcol |
| 958 | NeuAc1Hex1HexNAc2Sul1 | NeuAcα2-3Galβ1- |
| | | 3[(6S)GlcNAcβ1-6]GalNAcol |
| 966 | NeuAc2Hex1HexNAc1 | NeuAcα2-3Galβ1- |
| | | 3(NeuAcα2-6)GalNAcol |
| 974 | NeuGc1Hex1HexNAc2Sul1 | NeuGcα2-6Galβ1- |
| | | 3(6SGlcNAcβ1-6)GalNAcol |
| 975-1 | Hex2HexNAc2deHex1Sul1 | Galβ1-3[Galβ1-4(Fucα1- |
| | | 3)(6S)GlcNAcβ1-6]GalNAcol |
| 975-2 | Hex2HexNAc2deHex1Sul1 | Galβ1-3[Fucα1-2(6S)Galβ1- |
| | | 4GlcNAcβ1-6]GalNAcol |
| 975-3 | Hex2HexNAc2deHex1Sul1 | Gal(Fuc)(6S)GlcNAcβ1- |
| | | 3Galβ1-3GalNAcol |
| 975-4 | Hex2HexNAc2deHex1Sul1 | Galβ1-3[Fucα1-2Galβ1- |
| | | 4(6S)GlcNAcβ1-6]GalNAcol |
| 975-5 | Hex2HexNAc2deHex1Sul1 | Fucα1-2Galβ1-3[Galβ1- |
| | | 4(6S)GlcNAcβ1-6]GalNAcol |
| 982 | NeuAc1NeuGc1Hex1HexNAc1 | NeuAcα2-3Galβ1- |
| | | 3(NeuGcα2-6)GalNAcol |
| 998 | NeuGc2Hex1HexNAc1 | NeuGcα2-3Galβ1- |
| | | 3(NeuGcα2-6)GalNAcol |
| 1016-1 | Hex1HexNAc3deHex1Sul1 | GlcNAcβ1-3[Galβ1-4(Fucα1- |
| | | 3)(6S)GlcNAcβ1-6]GalNAcol |
| 1016-2 | Hex1HexNAc3deHex1Sul1 | Gal(Fuc)GlcNAcβ1- |
| | | 3(6SGlcNAcβ1-6)GalNAcol |
| 1016-3 | Hex1HexNAc3deHex1Sul1 | GlcNAcβ1-3[SGalβ1- |
| | | 4(Fucα1-3)GlcNAcβ1- |
| | | 6]GalNAcol |
| 1016-4 | Hex1HexNAc3deHex1Sul1 | Fucα1-2Galβ1-4GlcNAcβ- |
| | | 3(6SGlcNAcβ1-6)GalNAcol |
| 1016-5 | Hex1HexNAc3deHex1Sul1 | GlcNAcβ1-3[Fucα1-2Galβ1- |
| | | 4(6S)GlcNAcβ1-6]GalNAcol |
| 1016-6 | Hex1HexNAc3deHex1Sul1 | GalNAcα1-3(Fucα1-2)Galβ1- |
| | | 3(6SGlcNAcβ1-6)GalNAcol |
| 1016-7 | Hex1HexNAc3deHex1Sul1 | Fucα1-2Galβ1- |
| | | 4(6S)GlcNAcβ1-3(GlcNAcβ1- |
| | | 6)GalNAcol |
| 1016-8 | Hex1HexNAc3deHex1Sul1 | Fucα1-2Galβ1-4GlcNAcβ- |
| | | 3(6SGlcNAcβ1-6)GalNAcol |
| 1024-1 | NeuAc1Hex1HexNAc2deHex1 | Fucα1-2Galβ1-3GlcNAcβ1- |
| | | 3(NeuAcα2-6)GalNAcol |
| 1024-2 | NeuAc1Hex1HexNAc2deHex1 | Fucα1-2Galβ1-4GlcNAcβ1- |
| | | 3(NeuAcα2-6)GalNAcol |
| 1032 | Hex2HexNAc3Sul1 | Galβ1-4GlcNAcβ1-3[Galβ1- |
| | | 4(6S)GlcNAcβ1-6]GalNAcol |
| 1040 | NeuGc1Hex1HexNAc2deHex1 | Fucα1-2Galβ1-4GlcNAcβ1- |
| | | 3(NeuGcα2-6)GalNAcol |
| 1040 | NeuAc1Hex2HexNAc2 | NeuAcα2-3Galβ1-3(Galβ1- |
| | | 4GlcNAcβ1-6)GalNAcol |
| 1041-1 | Hex2HexNAc2deHex2 | Galβ1-3[Fucα1- |
| | | 2Gal(Fuc)GlcNAcβ1- |
| | | 6]GalNAcol |
| 1041-2 | Hex2HexNAc2deHex2 | Fucα1-2Galβ1-3(Fucα1- |
| | | 2Galβ1-4GlcNAcβ1- |
| | | 6)GalNAcol |
| 1055-1 | Hex2HexNAc2deHex1Sul2 | Galβ1-3[3SGal- |
| | | (Fuc)(6S)GlcNAcβ1- |
| | | 6]GalNAcol |
| 1055-2 | Hex2HexNAc2deHex1Sul2 | SGal-(Fuc)(6S)GlcNAc- |
| | | Galβ1-3GalNAcol |
| 1056 | NeuGc1Hex2HexNAc2 | NeuGcα2-3Galβ1-3(Galβ1- |
| | | 4GlcNAcβ1-6)GalNAcol |
| 1079 | NeuGc1Hex2HexNAc1deHex1Sul1 | |
| 1081 | NeuAc1Hex1HexNAc3 | NeuAcα2-3(GalNAcβ1- |
| | | 4)Galβ1-3(GlcNAcβ1- |
| | | 6)GalNAcol |
| 1096-1 | Hex1HexNAc3deHex1Sul2 | 6SGalβ1-(Fucα1-)GlcNAcβ1- |
| | | 3(6SGlcNAcβ1-6)GalNAcol |
| 1096-2 | Hex1HexNAc3deHex1Sul2 | 6SGalβ1-(Fucα1-) |
| | | (6S)GlcNAcβ1-3(GlcNAcβ1- |
| | | 6)GalNAcol |
| 1097 | NeuGc1Hex1HexNAc3 | NeuGcα2-3(GalNAcβ1- |
| | | 4)Galβ1-3(GlcNAcβ1- |
| | | 6)GalNAcol |
| 1098 | Hex2HexNAc3deHex1 | Galβ1-3[Fucα1-2(GalNAcα1- |
| | | 3)Galβ1-4GlcNAcβ1- |
| | | 6]GalNAcol |
| 1105 | Hex1HexNAc2deHex3Sul1 | |
| 1112-1 | Hex2HexNAc3Sul2 | 3SGal-GlcNAcβ1- |
| | | 3(6SGlcNAcβ1-6)Galβ1- |
| | | 3GalNAcol |
| 1112-2 | Hex2HexNAc3Sul2 | SGalβ1-4GlcNAcβ1- |
| | | 3(SGalβ1-3GlcNAcβ1- |
| | | 6)GalNAcol |
| 1120 | NeuAc1Hex2HexNAc2Sul1 | NeuAcα2-3Galβ1-3[Galβ1- |
| | | 4(6S)GlcNAcβ1-6]GalNAcol |
| 1121-1 | Hex2HexNAc2deHex2Sul1 | Galβ1-3[Fucα1-2Galβ1-(Fucα-) |
| | | (6S)GlcNAcβ1-6]GalNAcol |
| 1121-2 | Hex2HexNAc2deHex2Sul1 | Fucα1-2Galβ1-3[Gal- |
| | | (Fuc)(6S)GlcNAcβ1- |
| | | 6]GalNAcol |
| 1121-3 | Hex2HexNAc2deHex2Sul1 | Fucα1-2Galβ1-3[Fucα1- |
| | | 2Galβ1-4(6S)GlcNAcβ1- |
| | | 6]GalNAcol |
| 1136-1 | NeuGc1Hex2HexNAc2Sul1 | NeuGcα2-3Galβ1-3[Galβ1- |
| | | 4(6S)GlcNAcβ1-6]GalNAcol |
| 1136-2 | NeuGc1Hex2HexNAc2Sul1 | NeuGcα2-3Galβ1-3(3SGal- |
| | | GlcNAcβ1-6)GalNAcol |
| 1139 | Hex1HexNAc4deHex1 | GlcNAcβ1-3[GalNAcα1- |
| | | 3(Fucα1-2)Galβ1- |
| | | 3GlcNAcβ1-6]GalNAcol |
| 1161 | NeuAc1Hex1HexNAc3Sul1 | NeuAcα2-3(GalNAcβ1- |
| | | 4)Galβ1-3(6SGlcNAcβ1- |
| | | 6)GalNAcol |
| 1162-1 | Hex1HexNAc3deHex2Sul1 | GlcNAcβ1-3[Fucα1-2Galβ1- |
| | | (Fucα1-)(6S)GlcNAcβ1- |
| | | 6]GalNAcol |
| 1162-2 | Hex1HexNAc3deHex2Sul1 | 6SGlcNAcβ1-3[Fucα1- |
| | | 2Galβ1-(Fucα1-)GlcNAcβ1- |
| | | 6]GalNAcol |
| 1169 | NeuAc2Hex1HexNAc2 | NeuAcα2-3(GalNAcβ1- |
| | | 4)Galβ1-3(NeuAcα2- |
| | | 6)GalNAcol |
| 1177 | NeuGc1Hex1HexNAc3Sul1 | GlcNAcβ1-3[NeuGcα2- |
| | | 3Galβ1-4(6S)GlcNAcβ1- |
| | | 6]GalNAcol |
| 1178-1 | Hex2HexNAc3deHex1Sul1 | Galβ1-3[GalNAcα1-3(Fucα1- |
| | | 2)Galβ1-4(6S)GlcNAcβ1- |
| | | 6]GalNAcol |
| 1178-2 | Hex2HexNAc3deHex1Sul1 | Galβ1-3GlcNAcβ1-3[Fucα1- |
| | | 2Galβ1-4(6S)GlcNAcβ1- |
| | | 6]GalNAcol |
| 1178-3 | Hex2HexNAc3deHex1Sul1 | Galβ1-4GlcNAcβ1-3[Fucα1- |
| | | 2Galβ1-3(6S)GlcNAcβ1- |
| | | 6]GalNAcol |
| 1178-4 | Hex2HexNAc3deHex1Sul1 | Fucα1-2Galβ1-4GlcNAcβ1- |
| | | 3[Galβ1-4(6S)GlcNAcβ1- |
| | | 6]GalNAcol |
| 1185 | NeuAc1NeuGc1Hex1HexNAc2 | NeuAcα2-3(GalNAcβ1- |
| | | 4)Galβ1-3(NeuGcα2- |
| | | 6)GalNAcol |
| 1186 | NeuAc1Hex2HexNAc2deHex1 | NeuAcα2-3Galβ1-3(Fucα1- |
| | | 2Galβ1-4GlcNAcβ1- |
| | | 6)GalNAcol |
| 1201 | NeuGc2Hex1HexNAc2 | NeuGcα2-3(GalNAcβ1- |
| | | 4)Galβ1-3(NeuGcα2- |
| | | 6)GalNAcol |
| 1201-1 | Hex2HexNAc2deHex2Sul1 | |
| 1201-2 | Hex2HexNAc2deHex2Sul1 | Fucα1-2Galβ1-3[SGal- |
| | | (Fuc)(6S)GlcNAcβ1- |
| | | 6]GalNAcol |
| 1202 | NeuGc1Hex2HexNAc2deHex1 | NeuGcα2-3Galβ1-3(Fucα1- |
| | | 2Galβ1-4GlcNAcβ1- |
| | | 6)GalNAcol |
| 1218 | NeuGc1Hex3HexNAc2 | Galα1-3Galβ1-3(NeuGcα2- |
| | | 3Galβ1-4GlcNAcβ1- |
| | | 6)GalNAcol |
| 1219-1 | Hex1HexNAc4deHex1Sul1 | GlcNAcβ1-3[GalNAcα1- |
| | | 3(Fucα1-2)Galβ1- |
| | | (6S)GlcNAcβ1-6]GalNAcol |
| 1219-2 | Hex1HexNAc4deHex1Sul1 | GalNAcα1-3(Fucα1-2)Galβ1- |
| | | 3GlcNAcβ1-3(6SGlcNAcβ1- |
| | | 6)GalNAcol |
| 1227 | NeuAc1Hex1HexNAc3deHex1 | GlcNAcα1-4(Fucα1-2)Galβ1- |
| | | 4GlcNAcβ1-3(NeuAcα2- |
| | | 6)GalNAcol |
| 1243 | NeuAc1Hex2HexNAc3 | NeuAcα2-3Galβ1- |
| | | 3[GlcNAcα1-4Galβ1- |
| | | 4GlcNAcβ1-6]GalNAcol |
| 1244-1 | Hex2HexNAc3deHex2 | Galβ1-(Fuc)GlcNAcβ1- |
| | | 3(Fucα1-2Galβ1-4GlcNAcβ1- |
| | | 6)GalNAcol |
| 1244-2 | Hex2HexNAc3deHex2 | Fucα1-2Galβ1-4GlcNAcβ1- |
| | | 3(Fucα1-2Galβ1-4GlcNAcβ1- |
| | | 6)GalNAcol |
| 1258-1 | Hex2HexNAc3deHex1Sul2 | |
| 1258-2 | Hex2HexNAc3deHex1Sul2 | |
| 1258-3 | Hex2HexNAc3deHex1Sul2 | SGal-GlcNAcβ1-6[Fucα1- |
| | | 2Gal-(6S)GlcNAcβ1- |
| | | 6]GalNAcol |
| 1258-4 | Hex2HexNAc3deHex1Sul2 | |
| 1259 | NeuGc1Hex2HexNAc3 | NeuGcα2-3Galβ1- |
| | | 3(GlcNAcα1-4Galβ1- |
| | | 4GlcNAcβ1-6)GalNAcol |
| 1266 | NeuAc1Hex2HexNAc2deHex1Sul1 | NeuAcα2-3Galβ1-3[Fucα1- |
| | | 2Galβ1-4(6S)GlcNAcβ1- |
| | | 6]GalNAcol |
| 1315-1 | Hex2HexNAc4Sul2 | SGal-GlcNAcβ1- |
| | | 3(GlcNAcβ1-6)Galβ1- |
| | | 3(6SGlcNAcβ1-6)GalNAcol |
| 1315-2 | Hex2HexNAc4Sul2 | |
| 1323 | NeuAc1Hex2HexNAc3Sul1 | NeuAcα2-3(GalNAcβ1- |
| | | 4)Galβ1-3[Galβ1- |
| | | 4(6S)GlcNAcβ1-6]GalNAcol |
| 1324-1 | Hex2HexNAc3deHex2Sul1 | Fucα1-2Galβ1-3[GalNAcα1- |
| | | 3(Fucα1-2)Galβ1- |
| | | 3(6S)GlcNAcβ1-6]GalNAcol |
| 1324-2 | Hex2HexNAc3deHex2Sul1 | Fucα1-2Galβ1-4GlcNAcβ1- |
| | | 3[Fucα1-2Galβ1- |
| | | 4(6S)GlcNAcβ1-6]GalNAcol |
| 1324-3 | Hex2HexNAc3deHex2Sul1 | Fucα1-2Galβ1-3[GalNAcα1- |
| | | 3(Fucα1-2)Galβ1- |
| | | 4(6S)GlcNAcβ1-6]GalNAcol |
| 1324-4 | Hex2HexNAc3deHex2sul1 | SGal-(Fuc)GlcNAcβ1-3[SGal- |
| | | (Fuc)GlcNAcβ1-6]GalNAcol |
| 1338 | Hex2HexNAc3deHex1Sul3 | SGal-(Fuc)(6S)GlcNAcβ1- |
| | | 3(3SGal-GlcNAcβ1- |
| | | 6)GalNAcol |
| 1339-1 | NeuGc1Hex2HexNAc3Sul1 | |
| 1339-2 | NeuGc1Hex2HexNAc3Sul1 | |
| 1404-1 | Hex2HexNAc3deHex2Sul2 | Gal(Fuc)(6S)GlcNAcβ1- |
| | | 3(Fucα1-2)Galβ1- |
| | | 3(6SGlcNAcβ1-6)GalNAcol |
| 1404-2 | Hex2HexNAc3deHex2Sul2 | SGal-(Fuc)GlcNAcβ1-3[Gal- |
| | | (Fuc)(6S)GlcNAcβ1- |
| | | 6]GalNAcol |
| 1404-3 | Hex2HexNAc3deHex2Sul2 | SGal-GlcNAcβ1-3[Fucα1- |
| | | 2Gal-(Fuc)(6S)GlcNAcβ1- |
| | | 6]GalNAcol |
| 1404-4 | Hex2HexNAc3deHex2Sul2 | |
| 1405 | NeuGc1Hex2HexNAc3deHex1 | NeuGcα2-3(GlcNAcβ1- |
| | | 4)Galβ1-3(Fucα1-2Galβ1- |
| | | 4GlcNAcβ1-6)GalNAcol |
| 1419 | NeuGc1Hex2HexNAc3Sul2 | |
| 1420 | Hex3HexNAc3deHex1Sul2 | |
| 1428 | NeuGc1Hex2HexNAc2deHex2Sul1 | NeuGcα2-3Galβ1-3[Fucα1- |
| | | 2Galβ1-3(Fucα1- |
| | | 4)(6S)GlcNAcβ1-6]GalNAcol |
| 1461 | Hex2HexNAc4deHex1Sul2 | SGal-GlcNAcβ1- |
| | | 3[GalNAcα1-3(Fucα1-2)Gal- |
| | | (6S)GlcNAcβ1-6]GalNAcol |
| 1469 | NeuAc1Hex2HexNAc3deHex1Sul1 | NeuAcα2-3Galβ1- |
| | | 3[GalNAcα1-3(Fucα1-2)Gal- |
| | | (6S)GlcNAcβ1-6]GalNAcol |
| 1470-3 | Hex2HexNAc3deHex3Sul1 | Fucα1-2Galβ1- |
| | | 4(6S)GlcNAcβ1-3[Fucα1- |
| | | 2Galβ1-3(Fucα1- |
| | | 4)GlcNAcβ1-6]GalNAcol |
| 1485-1 | NeuGc1Hex2HexNAc3deHex1Sul1 | |
| 1485-2 | NeuGc1Hex2HexNAc3deHex1Sul1 | GlcNAcα1-4Galβ1- |
| | | 3[NeuGcα2-6Gal- |
| | | (Fuc)(6S)GlcNAcβ1- |
| | | 6]GalNAcol |
| 1486 | Hex3HexNAc3deHex2Sul1 | |
| 1508 | NeuGc1Hex2HexNAc2deHex2Sul2 | |
| 1524 | NeuGc1Hex3HexNAc2deHex1Sul2 | |
| 1527-1 | Hex2HexNAc4deHex2Sul1 | GlcNAcα1-4(Fucα1- |
| | | 2)GlcNAcβ1-3[Fucα1- |
| | | 2)Galβ1-4(6S)GlcNAcβ1- |
| | | 6]GalNAcol |
| 1527-2 | Hex2HexNAc4deHex2Sul1 | GalNAcα1-3(Fucα1-2)Gal- |
| | | GlcNAcβ1- |
| | | 3[Gal(Fuc)(6S)GlcNAcβ1- |
| | | 6]GalNAcol |
| 1527-3 | Hex2HexNAc4deHex2Sul1 | Galβ1-3GlcNAcβ1- |
| | | 3[GalNAcα1-3(Fucα1-2)Gal- |
| | | (Fucα1-2)(6S)GlcNAcβ1- |
| | | 6]GalNAcol |
| 1542 | NeuGc1Hex2HexNAc4Sul1 | |
| 1543 | Hex3Hex4deHex1Sul1 | |
| 1550-1 | Hex2HexNAc3deHex3Sul2 | SGal-(Fuc)GlcNAcβ1- |
| | | 3[Fucα1-2Gal- |
| | | (Fuc)(6S)GlcNAcβ1- |
| | | 6]GalNAcol |
| 1550-2 | Hex2HexNAc3deHex3Sul2 | Fucα1-2Gal-(Fuc)GlcNAcβ1- |
| | | 3[SGal-(Fuc)(6S)GlcNAcβ1- |
| | | 6]GalNAcol |
| 1565-4 | NeuGc1Hex2HexNAc3deHex1Sul2 | |
| 1565-5 | NeuGc1Hex2HexNAc3deHex1Sul2 | |
| 1565-6 | NeuGc1Hex2HexNAc3deHex1Sul2 | |
| 1590-2 | NeuGc1Hex3HexNAc2deHex2Sul1 | |
| 1592 | NeuAc1Hex2HexNAc4deHex1 | GalNAcα1-3(Fucα1-2)Gal- |
| | | GlcNAcβ1-3(NeuAcα2-3Gal- |
| | | GlcNAcβ1-6)GalNAcol |
| 1607-1 | Hex2HexNAc4deHex2Sul2 | SGal-(Fuc)GlcNAcβ1- |
| | | 3[GalNAcα1-3(Fucα1-2)Gal- |
| | | (6S)GlcNAcβ1-6]GalNAcol |
| 1607-2 | Hex2HexNAc4deHex2Sul2 | Gal-(Fuc)(6S)GlcNAcβ1- |
| | | 3[GalNAcα1-3(Fucα1-2)Gal- |
| | | (6S)GlcNAcβ1-6]GalNAcol |
| 1608 | NeuGc1Hex2HexNAc4deHex1 | GalNAcα1-3(Fucα1-2)Gal- |
| | | GlcNAcβ1-3(NeuGcα2-3Gal- |
| | | GlcNAcβ1-6)GalNAcol |
| 1616 | Hex2HexNAc3deHex4Sul1 | Fucα1-2Gal(Fuc)GlcNAcβ1- |
| | | 3[Fucα1-2Gal- |
| | | (Fuc)(6S)GlcNAcβ1- |
| | | 6]GalNAcol |
| 1623-2 | Hex3HexNAc4deHex1Sul2 | Gal-(6S)GlcNAcβ1-3[SGal- |
| | | (Fuc)(6S)GlcNAcβ1-6]Galβ1- |
| | | 3(GlcNAcβ1-6)GalNAcol |
| 1631-1 | NeuGc1Hex2HexNAc3deHex2Sul1 | |
| 1631-2 | NeuGc1Hex2HexNAc3deHex2Sul1 | |
| 1647-2 | NeuGc1Hex3HexNAc3deHex1Sul1 | |
| 1654 | NeuGc1Hex2HexNAc2deHex3Sul2 | |
| 1673-2 | Hex2HexNAc4deHex3Sul1 | |
| 1688-1 | NeuGc1Hex2HexNAc4deHex1Sul1 | |
| 1688-2 | NeuGc1Hex2HexNAc4deHex1Sul1 | |
| 1688-3 | NeuGc1Hex2HexNAc4deHex1Sul1 | |
| 1688-4 | NeuGc1Hex2HexNAc4deHex1Sul1 | |
| 1711 | NeuGc1Hex2HexNAc3deHex2Sul2 | |
| 1712 | Hex3HexNAc3deHex3Sul2 | Fucα1-2Gal(Fuc)GlcNAc- |
| | | (6S)Galβ1-3[6SGal- |
| | | (Fuc)GlcNAcβ1-6]GalNAcol |
| 1727 | NeuGc1Hex3HexNAc3deHex1Sul2 | |
| 1730 | Hex2HexNAc5deHex2Sul1 | GalNAcα1-3(Fucα1-2)Galβ1- |
| | | 4GlcNAcβ1-3[GalNAcα1- |
| | | 3(Fucα1-2)Galβ1- |
| | | 4(6S)GlcNAcβ1-6]GalNAcol |
| 1753 | Hex2HexNAc4deHex3Sul2 | |
| 1834-1 | NeuGc1Hex2HexNAc4deHex2Sul1 | |
| 1834-2 | NeuGc1Hex2HexNAc4deHex2Sul1 | |
| 1850 | NeuGc1Hex3HexNAc4deHex1Sul1 | |
| 1876 | Hex2HexNAc5deHex3Sul1 | GalNAcα1-3(Fucα1-2)Gal- |
| | | GlcNAcβ1-3[GalNAcα1- |
| | | 3(Fucα1-2)Gal- |
| | | (Fuc)(6S)GlcNAcβ1- |
| | | 6]GalNAcol |
| 1914-1 | NeuGc1Hex2HexNAc4deHex2Sul2 | |
| 1914-2 | NeuGc1Hex2HexNAc4deHex2Sul2 | |
| 1914-3 | NeuGc1Hex2HexNAc4deHex2Sul2 | |
| 1930 | NeuGc1Hex3HexNAc4deHex1Sul2 | |
| 2061 | Hex3HexNAc4deHex4Sul2 | |
| 2078 | NeuAc1Hex5HexNAc4deHex1 | NeuAcα2-Galβ1-4GlcNAcβ1- |
| | | 2Mana1-3(Galβ1- |
| | | 4GlcNAcβ1-2Manα1- |
| | | 6)Manβ1-4GlcNAcβ1- |
| | | 4(Fucα1-6)GlcNAcol |
| 2142 | NeuAc1Hex4HexNAc4deHex2Sul1 | |
| 2288 | NeuGc1Hex3HexNAc4deHex4Sul1 | |
| 2304-2 | NeuGc1Hex4HexNAc4deHex3Sul1 | |
| 2368 | NeuGc1Hex3HexNAc4deHex4Sul2 | Fucα1-2Galβ1-3(Fucα1- |
| | | 4)GlcNAcβ1-3[NeuGcα2- |
| | | 6(3S)Galβ1-GlcNAcβ1- |
| | | 3(Fucα1-2)Gal- |
| | | (Fuc)(6S)GlcNAcβ1- |
| | | 6]GalNAcol |

^{[1]}Mass of structure in negative ion mode; mass with hyphens indicates the isomeric structures.
^{[2]}Hex, hexose; HexNAc, N-acetylhexosamine; deHex, fucose; NeuAc, N-acetylneuraminic acid; NeuGc, N-acetyl glycolylneuraminic acid; S, sulphate.
^{[3]} Structures are given in the text according following rules: the structure is described clockwise and left-to-right where reducing end locates rightmost side; “+” is used for uncertain location.

In some embodiments, the oligosaccharides/glycans in the GCX composition was found to comprise the following formulae: Hex1HexNAc1, HexNAc2, Hex1HexNAc1Sul, Hex1NAc2Sul1, NeuAc1HexNAc1, NeuGc1HexNAc1, Hex1HexNAc1deHex1, Hex2HexNAc1, Hex1HexNAc2, HexNAc3, Hex1HexNAc2Sul1, NeuAc1Hex1HexNAc1, NeuGc1Hex1HexNAc1, Hex2HexNAc1deHex1, Hex1NAc3Sul1, NeuAc1HexNAc2, NeuGc1HexNAc2, Hex1HexNAc2deHex1, Hex2HexNAc2, Hex1HexNAc3, Hex1HexNAc2deHex1Sul1, NeuAc1Hex1HexNAc1deHex1, Hex2HexNAcSul1, NeuGc1Hex1HexNAc1deHex1, Hex1HexNAc3Sul1, NeuAc1Hex1HexNAc2, NeuGc1Hex1HexNAc2, Hex2HexNAc2deHex1, Hex3HexNAc2, Hex1HexNAc3deHex1, Hex2HexNAc3, NeuAc1Hex1HexNAc2Sul1, NeuAc2Hex1HexNAc1, NeuGc1Hex1HexNAc2Sul1, Hex2HexNAc2deHex1Sul1, NeuAc1NeuGc1Hex1HexNAc1, NeuGc2Hex1HexNAc1, Hex1HexNAc3deHex1Sul1, NeuAc1Hex1HexNAc2deHex1, Hex2HexNAc3Sul1, NeuGc1Hex1HexNAc2deHex1, NeuAc1Hex2HexNAc2, Hex2HexNAc2deHex2, NeuGc1Hex2HexNAc2, Hex2HexNAc3deHex1, NeuAc1Hex2HexNAc2Sul1, Hex2HexNAc2deHex2Sul1, NeuGc1Hex2HexNAc2Sul1, Hex1HexNAc4deHex1, NeuAc1Hex1HexNAc3Sul1, Hex1HexNAc3deHex2Sul1, NeuAc2Hex1HexNAc2, NeuGc1Hex1HexNAc3Sul1, Hex2HexNAc3deHex1Sul1, NeuAc1Hex2HexNAc2deHex1, NeuGc2Hex1HexNAc2, NeuGc1Hex2HexNAc2deHex1, NeuGc1Hex3HexNAc2, Hex1HexNAc4deHex1 Sul1, NeuAc1Hex1HexNAc3deHex1, Hex2HexNAc3deHex2 NeuGc1Hex2HexNAc3, NeuAc1Hex2HexNAc2deHex1Sul1, Hex2HexNAc3deHex2Sul1, NeuAc1Hex2HexNAc3deHex1Sul1, NeuGc1Hex2HexNAc3deHex1Sul1, Hex2HexNAc4deHex2Sul1, Hex2HexNAc5deHex2Sul1, NeuAc1Hex5HexNAc4deHex1, and NeuAc1Hex4HexNAc4deHex2Sul1.

### Example 4—Melanoma Study 1

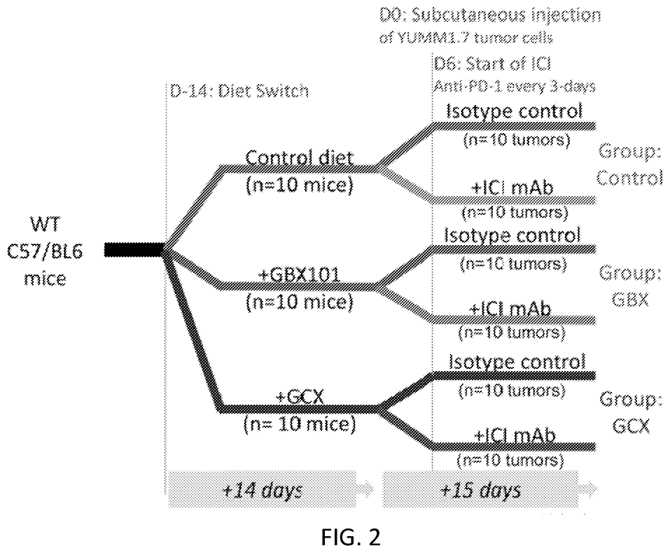

C57BL/6 mice (N=10/group) were treated either with normal drinking water or 3% test item (GBX or GCX as provided in Examples 1-3 above) throughout the experiment. After 14 days of pre-treatment, mice were injected subcutaneously with YUMM1.7 melanoma tumor cells (YUMM1.7 (ATCC #CRL-3362) is an ephithelial-like mouse melanoma cell line was developed as an inmmunocompetent cell line that recapitulates genetic drivers found in a significant proportion of human mnelanomas). Starting on day 6 after tumor inoculation, mice were injected with 200 ug/mouse of the immune checkpoint inhibitor anti-PD-1 (anti-PD-1(CD279) antibody (BioXCell; clone 29F.1A12), a mice equivalent of nivolumab) or control isotype at 3-day intervals. At the last day of the experiment, mice were euthanized and tumors weighed at the time of excision. The study protocol is provided in FIG. 2.

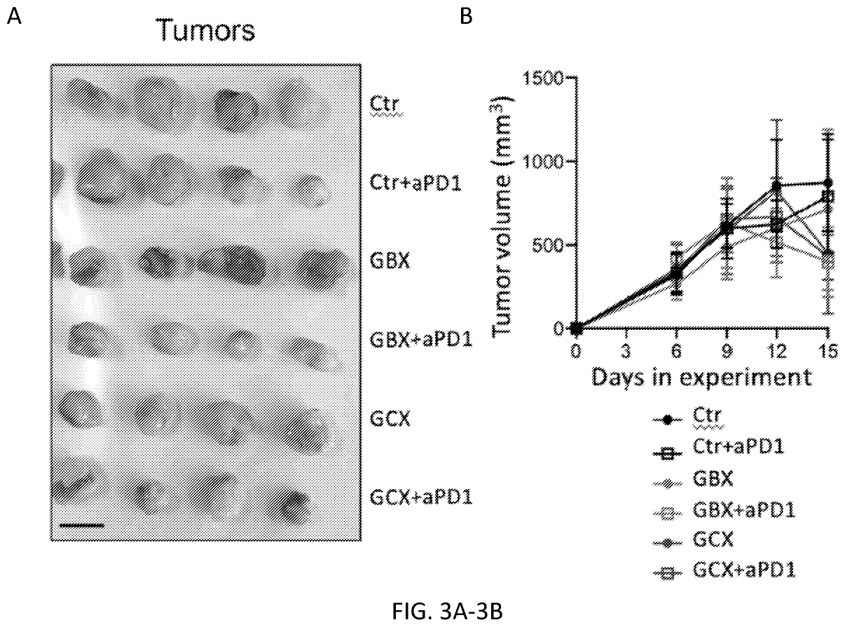

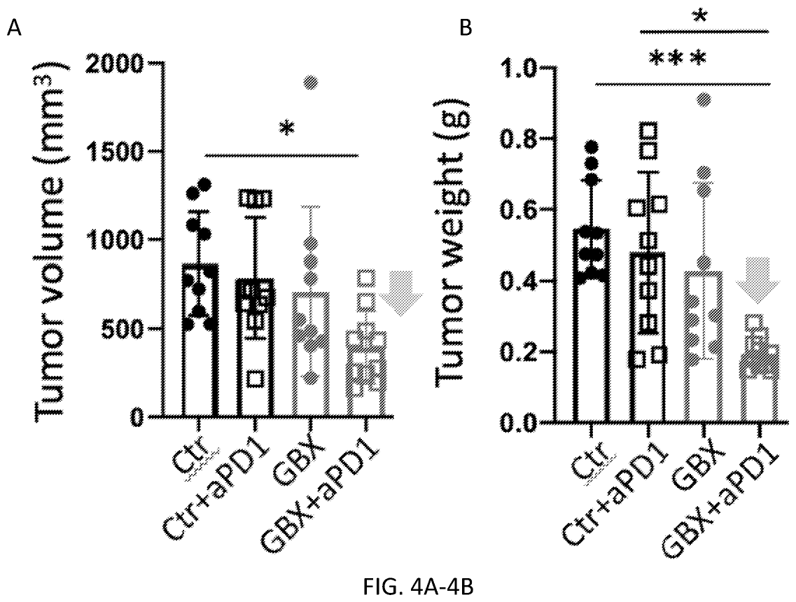

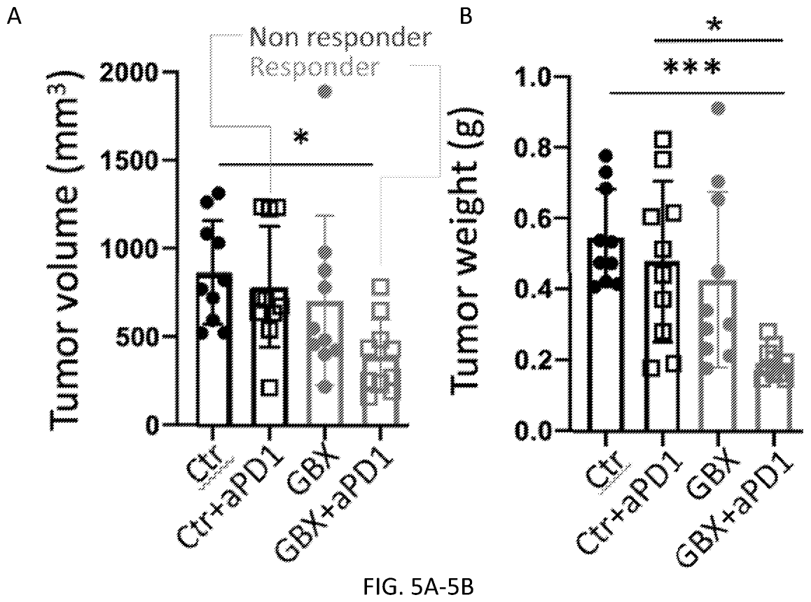

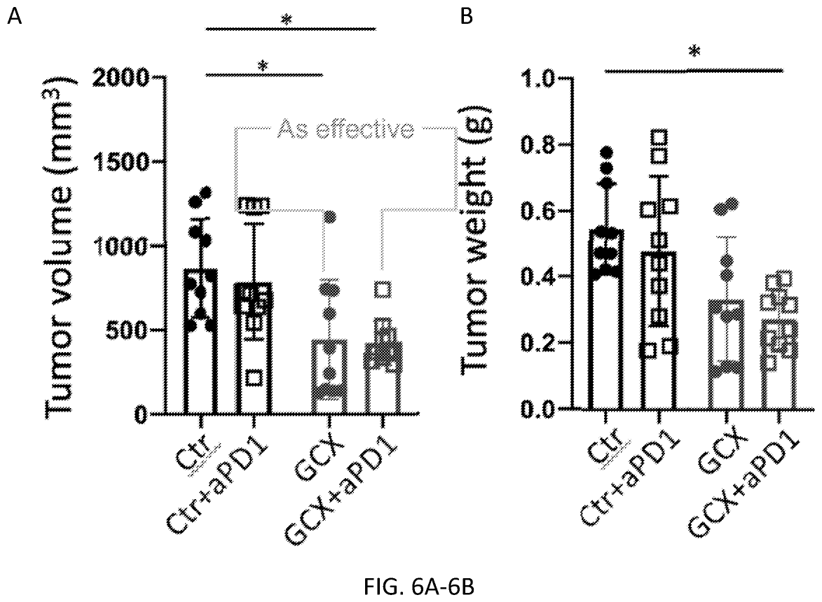

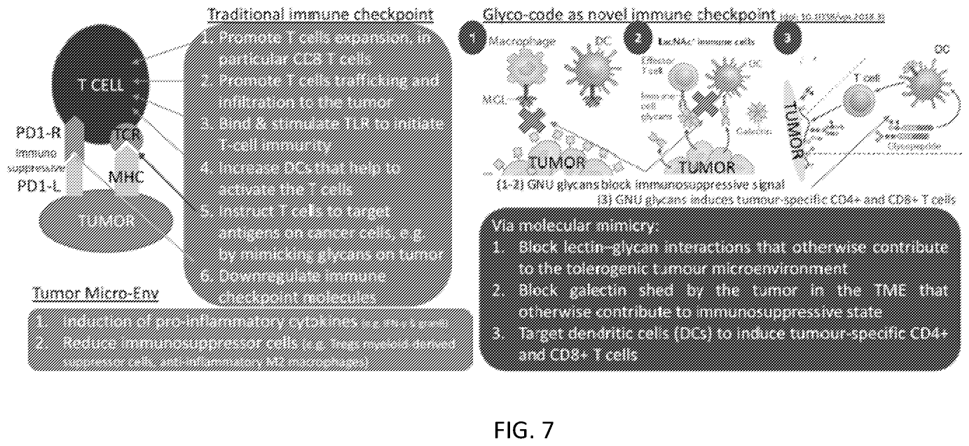

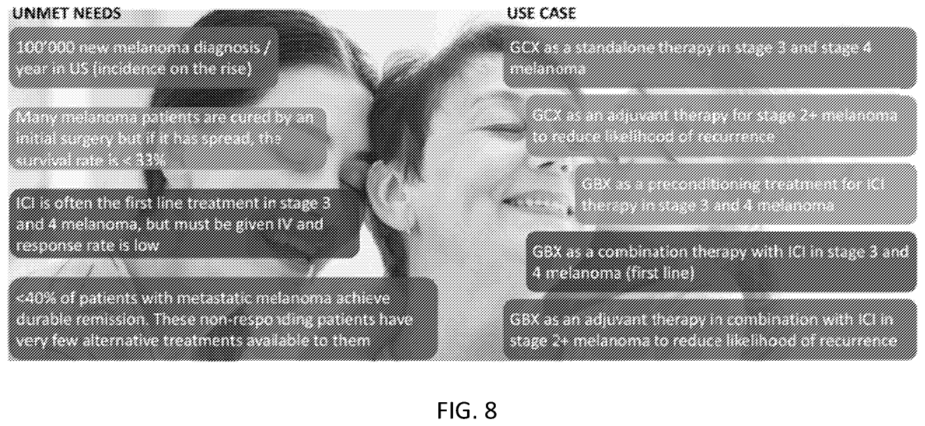

Tumor size was routinely monitored every 3 days. Addition of GBX and GCX to the drinking water in combination with the immune checkpoint inhibitor limited tumor volume and weight (reduction of ˜50%), while anti-PD-1 alone did not (reduction of ˜13% without statistical significance). GCX as a monotherapy attenuated tumor volume (reduction of ˜50%). The results of the study are shown in FIGS. 3-6.

### Example 5—Melanoma Study 2

### Study 1

Applicant evaluated the efficacy of GBX and GCX in reducing tumor size when administered alone or in combination with immune checkpoint inhibitors (ICI) in mice subcutaneously injected with melanoma tumor cells. Applicant also studied the impact of the treatment on (i) immune cell populations infiltrating the tumor tissue, (ii) inflammatory markers and metabolites & (iii) gut microbial diversity & composition. To evaluate these effects, oral administration with test items provided in drinking water at 3% w/v was tested in this study.

To test the effect of the compositions identified herein as “GBX” and “GCX” on melanoma growth and on PD1-inhibition-mediated anti-tumor effects, C57/BL6 mice (n=5/group) were injected subcutaneously with 200,000 YUMM1.7 melanoma cells in each flank. 10 mice received 3% GBX and 10 mice 3% GCX in the drinking water starting 14 days prior to tumor cell injection, while 10 mice received normal drinking water (control, Ctr). Since the compositons promote bacterial growth, the drinking water was filtered or replaced with new product every day. On day 6, day 9 and day 12 after tumor cell injection, 5 mice in each group received 200 g anti-PD1 antibody (BioXCell #PB0273; clone 29F.1A12) via intraperitoneal injection or control isotype (BioXCell #PB0089). Tumor size was measured every 3rd day using an electronic caliper and tumor volumes calculated using the ellipsoid formula 4/3*π*a*b2 where a is the length and b the width of the tumor to monitor differences in growth. On day 15 after tumor cell injection, the mice were sacrificed to analyze tumor weight, tumor infiltrating immune cells, and cytokine levels in the serum.

| | | | |
|---|---|---|---|
| D-14 | D0 | D6 | D15 |

MAJOR STUDY EVENTS

| | | | | |
|---|---|---|---|---|
| Randomization | X | | | |
| (to different diet | | | | |
| groups) | | | | |
| Tumor injection | | X | | |
| ICI treatment start | | | X | |
| Study end | | | | X |

COLLECTION

| | | | |
|---|---|---|---|
| Tumor | | | X |
| Serum | | X | X |
| Stool | X | X | X |
| Cecum & colon | | | X |
| content | | | |
| Colon tissue | | | X |

END-POINTS

| Tumor volume | | Every 3 days | |
|---|---|---|---|
| Tumor weights | | | X |
| TILs | | | X |
| 16S | (OPTIONAL) | X | X |
| Cytokine panel | | X | X |
| Untargeted | | X | X |
| metabolomic | | | |

END-POINTS

| END-POINT | DESCRIPTION | TIMEPOINT |
|---|---|---|
| Tumor volume | PRIMARY EP: To assess impact on | Every 3 days, |
| | tumor evolution | starting on D0 |
| Tumor weights | PRIMARY EP: To assess impact on | D15 (end of |
| | tumor evolution | exp) |
| TILs & subsets | To characterize anti-tumor immunity | D15 (end of |
| (from tumor | | exp) |
| sample) | | |
| 16S | To assess microbiome modulation | D0 |
| (from fecal | activity & link to anti-tumor | D15 (end of |
| samples) | immunity | exp) |
| | | OPTIONAL: |
| | | D-14 |
| | | (baseline) |
| Cytokine panel | To characterize systemic | D0 |
| (from serum) | inflammation-promoting effect | D15 |
| Untargeted | e.g. to look for inosine | D0 |
| metabolomics | | D15 |
| (from serum) | | |

### Study 2

### Animal Models and Husbandry Conditions

Female C57BL/6J mice at an age of 9-12 weeks were used for all experiments. The mice were housed in an SPF facility with food and water ad libitum and a 12 h light-dark cycle.

### Dose Formulation

Mice were treated with normal drinking water or supplemented with 3% w/v of test item throughout the experiment. To prevent bacterial growth, the drinking water/drinking water with test item was filtered or replaced with new product every day.

### Tumor Models & Treatments

The GBX and GCX compositions of this disclosure were dissolved in drinking water at a concentration of 3% (w/v) and sterile filtered using 0.22 um filters from Merck-Millipore. To prevent bacterial growth, the drinking water was filtered again 24 h after supplementation and replaced with fresh drinking water after 48 h. The mice received water with test compounds starting 14 days prior to tumor cell injection. 200,000 YUMM1.7 (ATCC #CRL-3362) melanoma cells were injected subcutaneously into the left and right flank (100 ul of a 1:1 culture medium:Matrigel mixture). Starting on day 6 after tumor cell injection, the mice were injected with 200 μg/mouse anti-PD1 (BioXcell #PB0273) or isotype control (BioXCell, #PB0089) every three days until the end of the experiment. Tumors were measured every 3 days using an electronic caliper and tumor volumes calculated using the ellipsoid formula 4/3*π*a*b2 where a is the length and b the width of the tumor.

### Tumor Cell Lines Culture

YUMM1.7 cells were purchased from ATCC and maintained in DMEM/F12 medium (Thermo Fisher Scientific #CRL-3362) with 10% FCS in a 37° C. 100% humidity cell culture incubator 10% C02. Cells were kept at a confluence below 70% at all times. For passaging, the cells were rinsed with PBS, detached with trypsin (1%) for 5 minutes, supplemented with growth medium and used for experiments or seeded at a lower density into new cell culture flasks. For experiments, the cells were centrifuged (1200 rpm, 5 min, 4° C.) and re-suspended in growth medium and mixed 1:1 with matrigel (Corning, #FAL354263)

### Flow Cytometry

For Flow cytometry, tumors were cut to approximately 0.5 mm³ pieces and digested in 6 mL RPMI containing 0.5 mg/mL collagenase type IV (Sigma Aldrich) and 0.05 mg/mL DNAse I (Roche) solution for 10 minutes on a shaker (300 rpm) at 37° C. Cells were homogenized passing through 18G1.5 syringe and centrifuged for 10 mins, 4° C., 1500 rpm. Single cell suspensions were centrifuged, washed in PBS and resuspended in antibody solution. For cytokine and cytotoxic factors, the cells were restimulated for 4 h in RPMI, 10% FCS supplemented with Ionomycin (1 ug/ml, Sigma-Aldrich, #I3909), PMA (1 ug/ml, Sigma-Aldrich, #1585) and Brefeldin A (50 ng/ml, Sigma-Aldrich, #B6542). After restimulation, the cells were stained for surface markers, washed in PBS, permeabilized using the FoxP3 staining kit from eBioscience (#00-5523-00), stained for intracellular molecules, washed and resuspended in PBS. Cells were acquired using a BD FACS Symphony analyzer.

### Cytokine Analysis

Serum cytokine levels were measured using the mouse 23-plex kit from Bio-rad (#M60009RDPD) according to the manufacturer's instructions. The serum was diluted 1:4 in assay buffer for analysis.

### Analysis of 16S Amplicon Sequencing Data

For 16S RNA sequencing, DNA was extracted from the stool using the DNA Soil kit from Qiagen following the manufacturer's instructions. To sequence the V3-V4 regions of the bacterial 16S rRNA gene, two-step PCR libraries using the primer pairs 341f_ill and 802r_ill were created. Subsequently, the Illumina MiSeq platform and a v2 500 cycle kit were used to sequence the PCR libraries. The produced paired-end reads that passed Illumina's chastity filter were subjected to demultiplexing and trimming of Illumina adaptor residuals (no further refinement or selection) using a publicly available Divisive Amplicon Denoising Algorithm (DADA2) pipeline. The resulting data were clustered to form amplicon sequence variants (ASVs) while discarding singletons and chimeras in the process. ASVs were aligned against the Ribosomal Database Project's Training Set 16, and taxonomy was predicted. All data were analyzed using Mann-Whitney U test in PAST; v3.12.

Microbiota data ordination was done using a principal coordinates analysis (PCA) with assessment of differences among microbial profiles of the different groups being done by one-way PERMANOVA (Bray-Curtis dissimilarity distance) using paleontological Statistics (PAST; v3.12) software. Alpha-diversity analyses, including richness (observed ASVs), Shannon diversity index (H), and evenness (e\hat{ }H/S) were calculated, with significant differences being calculated using Mann-Whitney U test in PAST; v3.12. Differences were considered significant with P<0.05.

### Data Visualization and Statistics

Flow cytometry, tumor growth/weight, and cytokine data were visualized and analyzed using GraphPad Prism version 8.4.3. One- or two-way ANOVA with Dunnet's correction for multiple testing was used. Corrected p-values below 0.05 were considered significant.

### Results & Discussions

GBX and GCX Reduce Tumor Growth in Combination with Anti-PD1

The compositions GBX and GCX supplementation in the drinking water was well tolerated and no differences were observed in water consumption and no abnormal behavior was detected that would indicate adverse effects of the products.

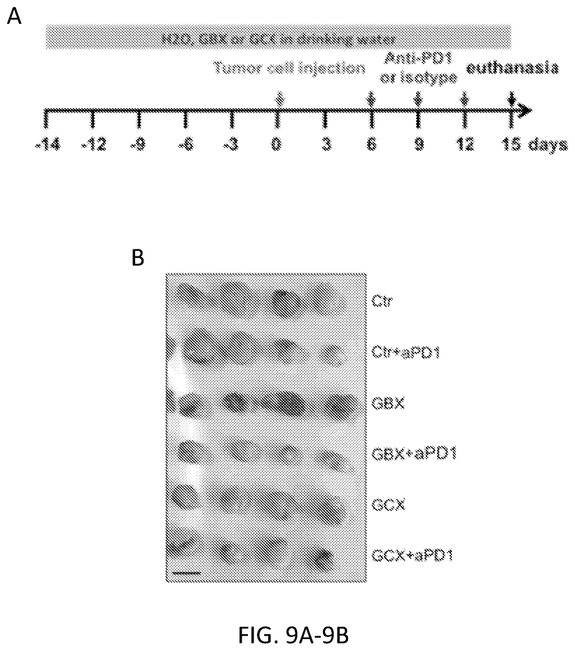

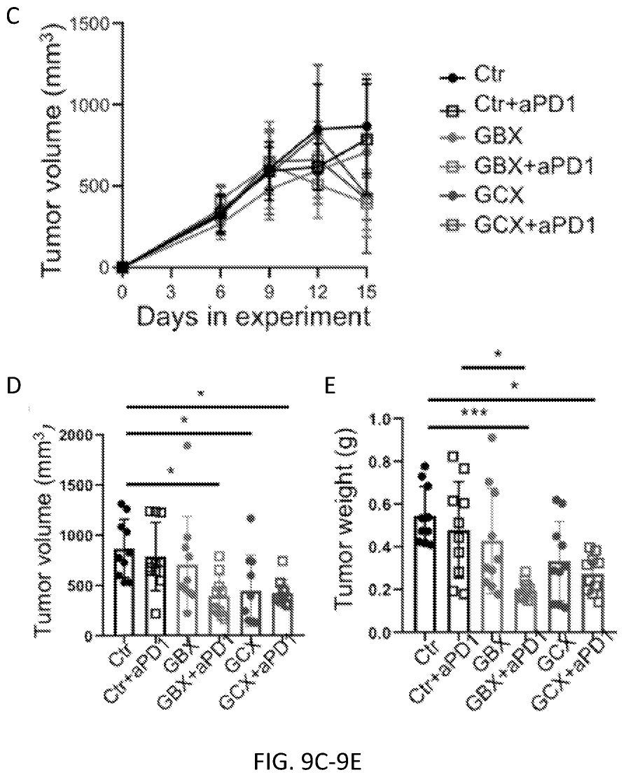

Up until day 9, no differences in tumor growth were observed in any of the groups, but starting on day 12, tumors in the GBX+anti-PD1, GCX and GCX+anti-PD1-treated groups started to shrink, resulting in a significantly reduced tumor volume in these three groups on day 15 after tumor cell injection (FIGS. 9B-D). Similar effects were observed in tumor weight, although this was not significant in the mice treated with GCX without anti-PD1 (FIG. 9E). Notably, anti-PD1-treatment on its own had no effect on tumor growth or tumor weight, indicating that anti-PD1 treatment alone was not sufficient to induce a strong anti-tumor immune reaction and additional immune cell activation was necessary to achieve an effect on tumor growth (FIGS. 9A-9E).

GBX and GCX in Combination with Anti-PD1 Promote Anti-Tumor T Cell Responses

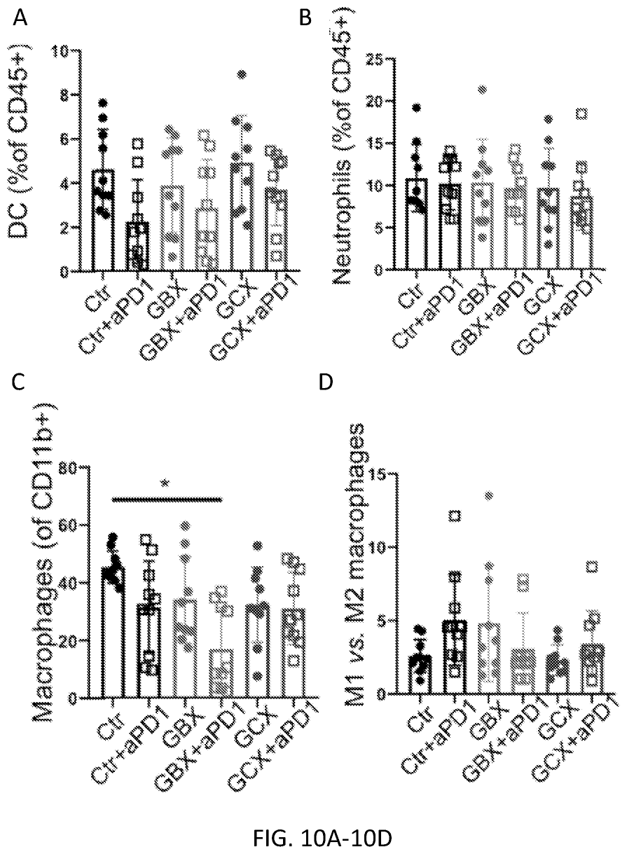

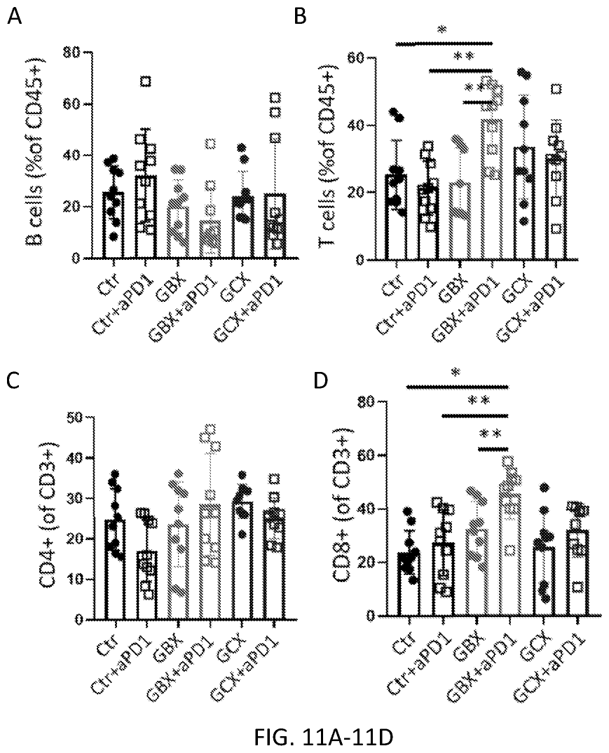

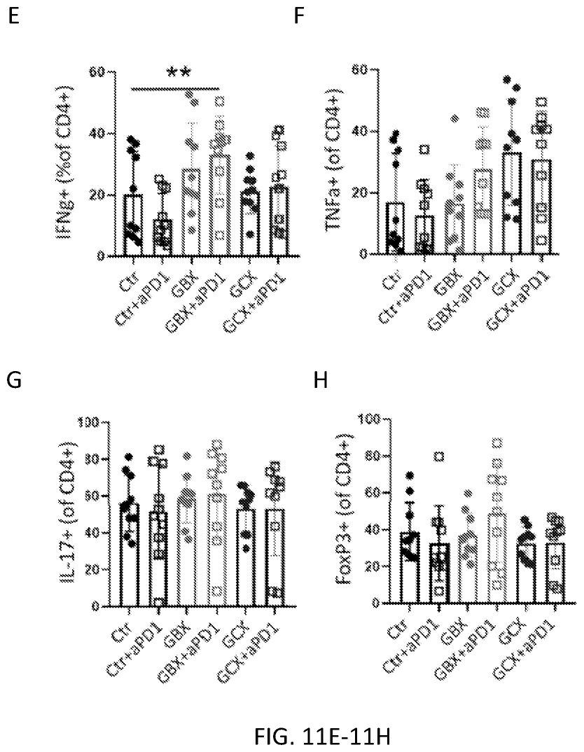

To assess the anti-tumor immune response, Applicant performed flow cytometry on tumor-infiltrating immune cells. When analyzing myeloid immune cells, the effects of GBX and GCX were very moderate, and no differences were observed in the proportions of neutrophils or dendritic cells (FIGS. 10A+B). Notably, the overall proportion of macrophages was decreased in mice receiving GBX in combination with anti-PD1 (FIG. 10C), but although there was a clear trend towards increased proportions of classically activated (M1) macrophages while alternatively activated (M2) macrophages tended to decrease upon anti-PD1 treatment (FIGS. 10C+D). Relative abundance of B cells was not significantly affected in any of the treatment groups (FIG. 11A).

In contrast to these minor effects on myeloid cells, there were clear effects on anti-tumor T cell responses upon administration of GBX and GCX. In combination with anti-PD1, GBX supplementation promoted the overall abundance of T cells (FIG. 11B), and among those the proportion of CD8+ T cells (FIG. 11D) and IFNγ producing CD4+ T cells (FIG. 11E), while with GBX alone there is a trend towards elevated numbers of IFNγ producing CD4+ T cells (FIG. 11E). Other T helper cell subsets (Th17, Treg) were not changed (FIGS. 11G+H). Furthermore, GBX promoted the number of IFNγ producing CD8+ T cells even in absence of anti-PD1 treatment (FIG. 11I), indicating that GBX did not only promote tumor infiltration but also activation of CD8+ T cells, which are known to directly kill tumor cells. Notably, these effects of GBX on CD8+ T cells were further increased in mice that additionally received anti-PD1, resulting in significantly increased levels of Perforrin and TNFα producing CD8+ T cells, while granzymeB expression was not altered (FIGS. 11I-L). The effects of GCX were less pronounced but there was a trend towards elevated numbers of TNFα+CD4 T cells (FIG. 11F), CD8+ T cells (FIG. 11D) and among those significantly elevated proportions of IFNγ+ and TNFα+CD8+ T cells in mice that received anti-PD1 in combination with GCX (FIGS. 11 I and J). These data clearly show that GBX and GCX either alone or in combination with anti-PD1 promote anti-tumor T cell responses.

Anti-PD1 Treatment Induces a Reduction of T Cell Exhaustion Markers Expression (CTLA4, PD1) while PD1 Expression is not Altered Upon GBX or GCX Administration

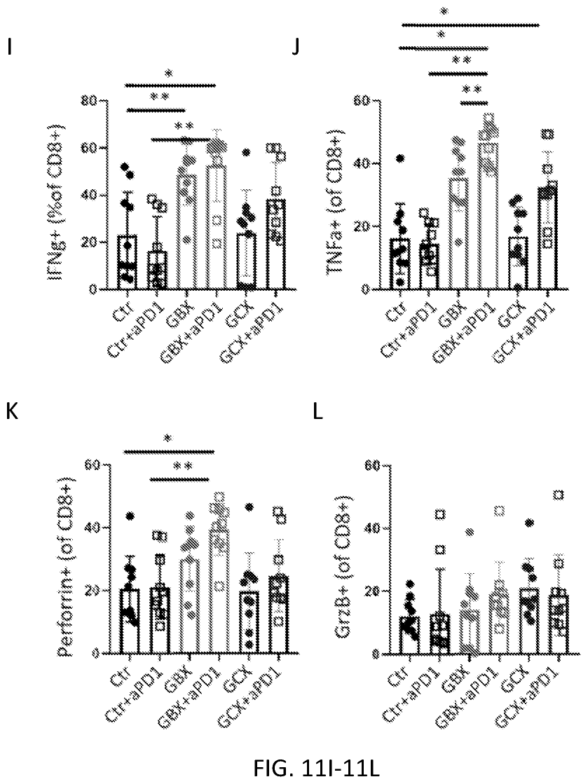

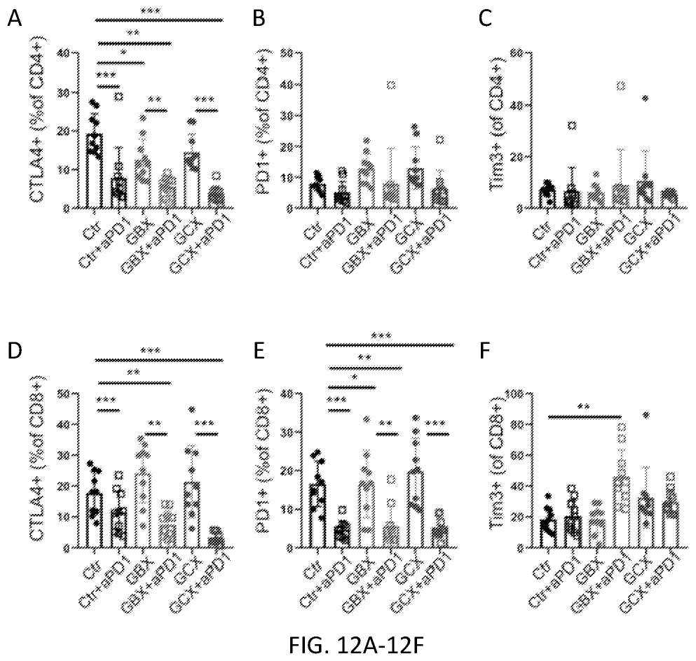

A critical mechanism how antigen-specific T cells are rendered anergic to prevent excessive immune response is the expression of exhaustion markers on T cells upon prolonged T cell receptor stimulation. Exhaustion molecules are surface receptors that are upregulated late during immune responses and mediate cell death or suppression of the activity to terminate no longer needed immune responses. Many cancers express ligands to those exhaustion markers and in this way promote T cell anergy and/or death leading to immune escape. In addition, the cytokine milieu in tumors often favors expression of exhaustion markers on T cells. In line with an inhibitory effect on T cell anergy/T cell exhaustion, anti-PD1 treatment alone or in combination resulted in reduced expression of the exhaustion marker CTLA4, but there was no significant difference between mice that received normal drinking water and those that received GBX or GCX, excepted that GBX-treatment on its own resulted in a reduction of CTLA4 expression on CD4+ T cells, but not on CD8+ T cells (FIGS. 12A and B). Further, anti-PD1 treatment induced a reduction in PD1 expression on tumor-associated T cells, however this only reached statistical significance in CD8+ T cells but not in CD4+ T cells (FIGS. 12C and D) and there were no effects observed on the exhaustion marker Tim3 (FIGS. 12E and F). Taken together, this indicates that in line with its mode of action, anti-PD1 treatment reduced T cell exhaustion, but there was no significant effect of the two test compounds.

### Mixed Effects on Serum Cytokine Levels

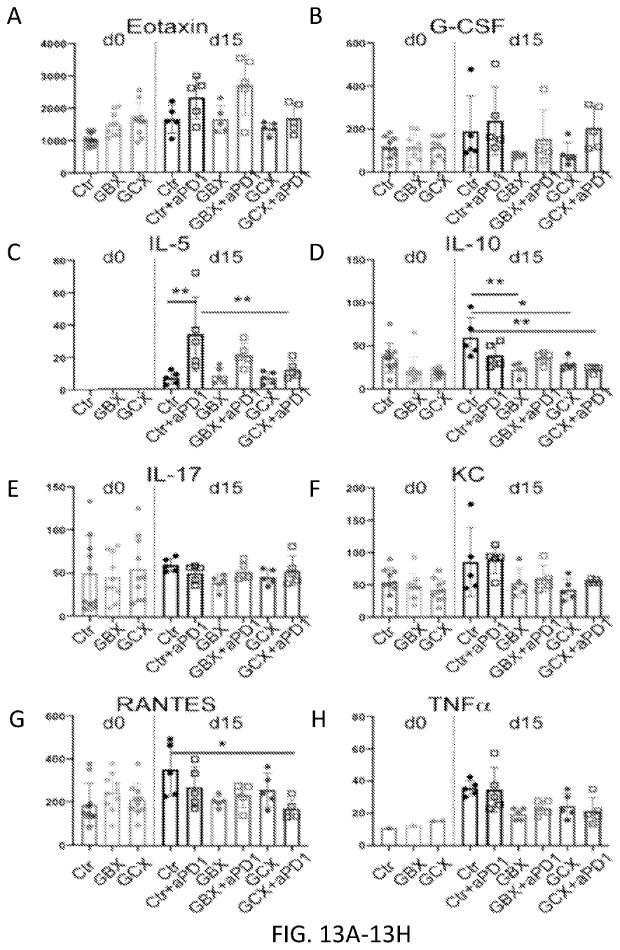

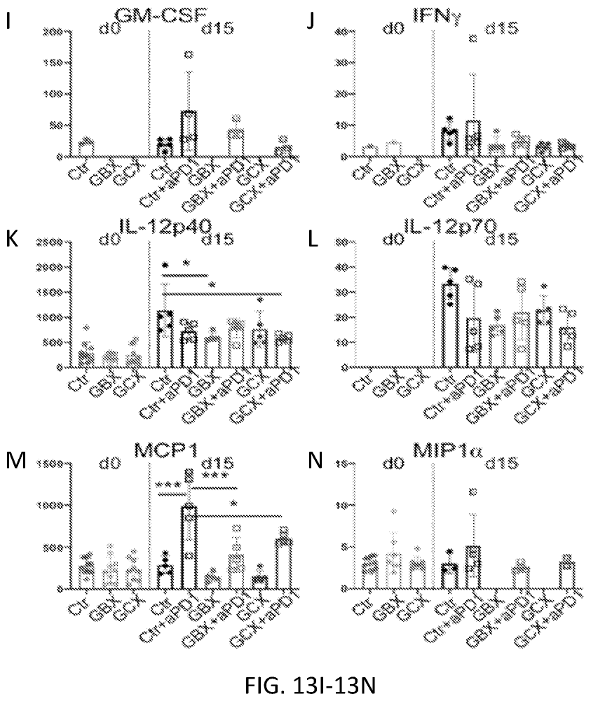

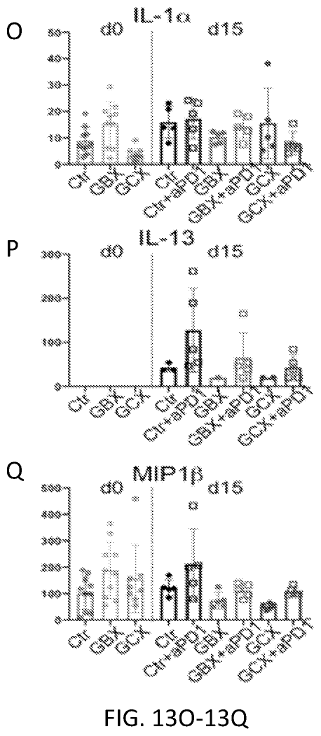

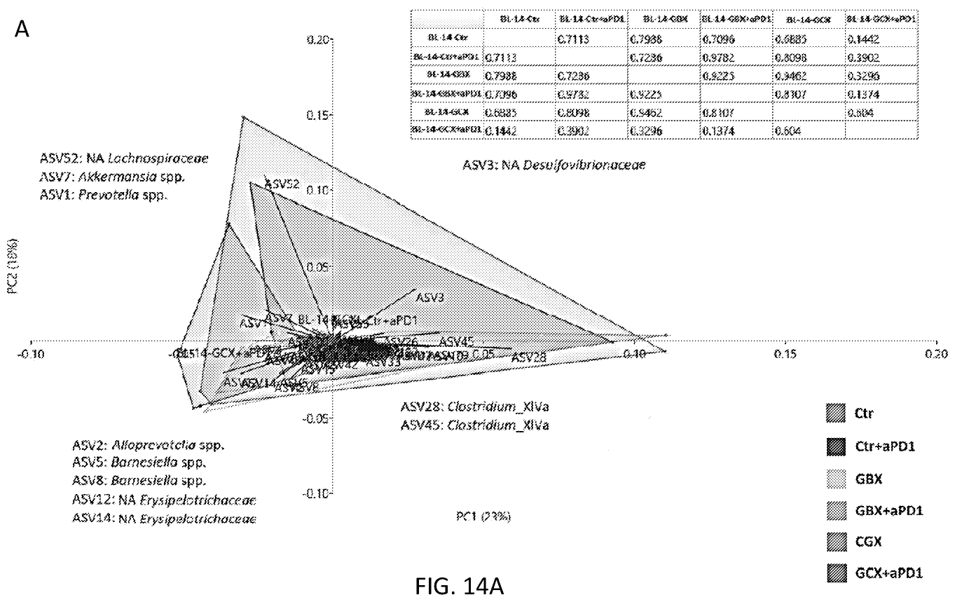

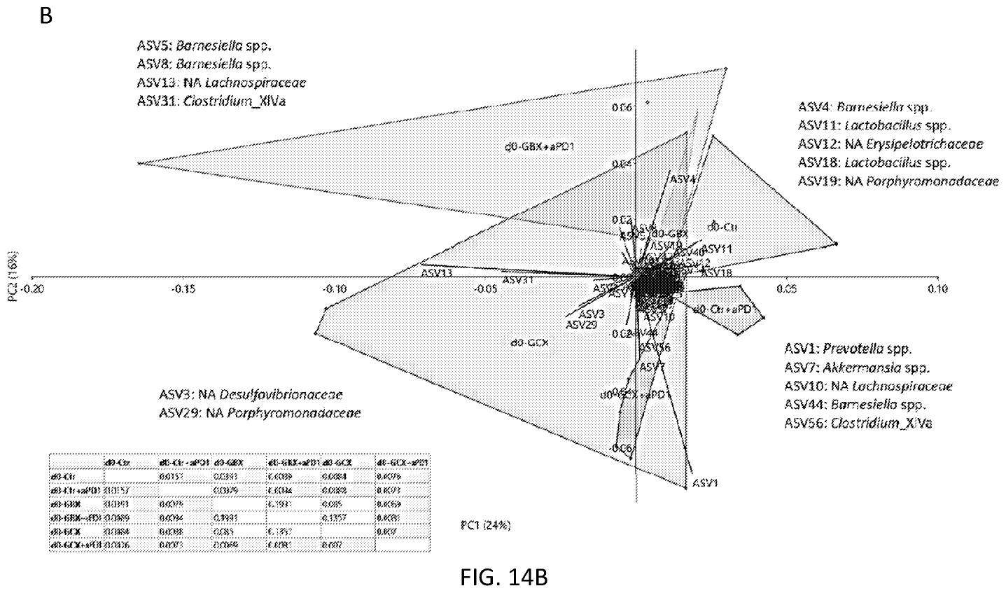

In contrast to the effects on tumor infiltrating immune cells, serum cytokine profiles did not show a signature that could be clearly correlated with the anti-tumor effects of the two compounds (FIG. 13). However, and quite notably, GBX and GCX administration resulted in reduced levels of IL-10, a molecule that is produced by a variety of cells, including tumor-infiltrating immune-suppressive myeloid cells/macrophages. In addition, GCX administration reduced levels of RANTES, a molecule responsible for macrophage chemoattraction to inflammatory sites, however this effect was only significant in combination with anti-PD1. In addition, both, GBX and GCX prevented the anti-PD1-induced rise in MCP1 (FIG. 13), which is another monocyte/macrophage-attracting and activating molecule that might promote the activation and function of tumor-infiltrating macrophages. Depending on the microenvironment, tumor-infiltrating macrophages can either promote tumor eradication by activating cytotoxic cells, or they suppress anti-tumor immune responses quite effectively (i.e. via production of IL-10 and TGFb). Given the mixed results in the serum cytokine profile, it might be of interest to investigate the cytokine milieu directly within the tumor tissue.

### Effects of Test Items Supplementation on Fecal Microbiota

### Sequencing Overview & PCA Analysis

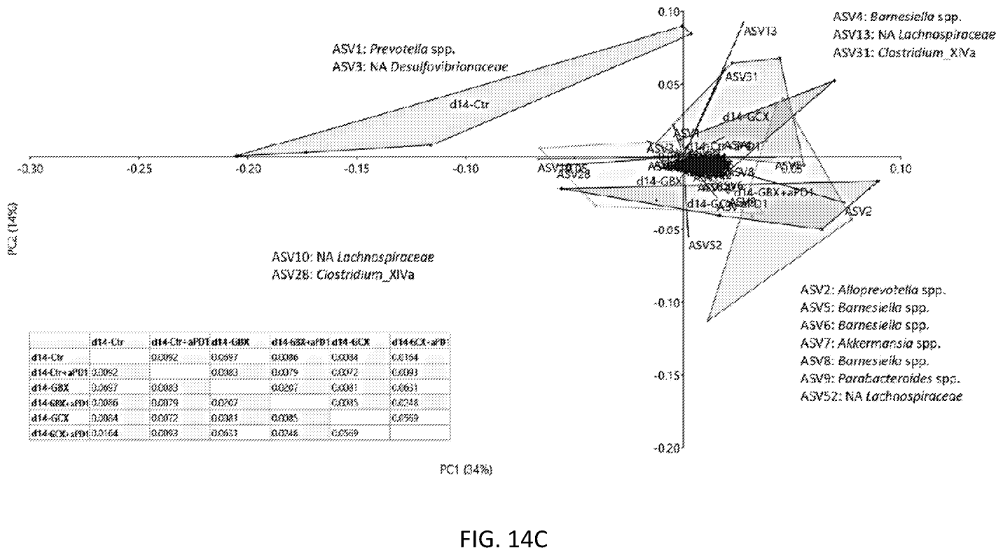

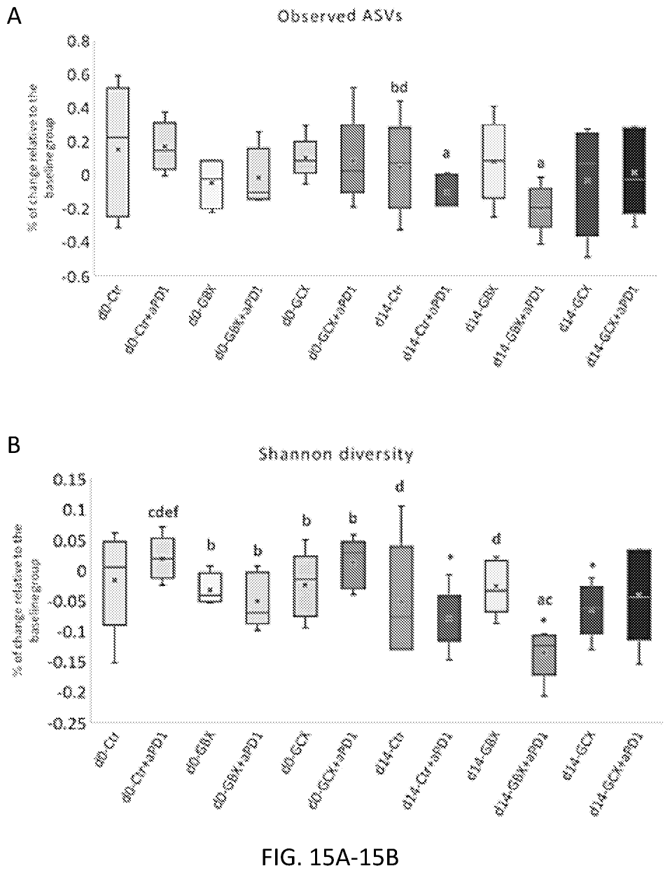

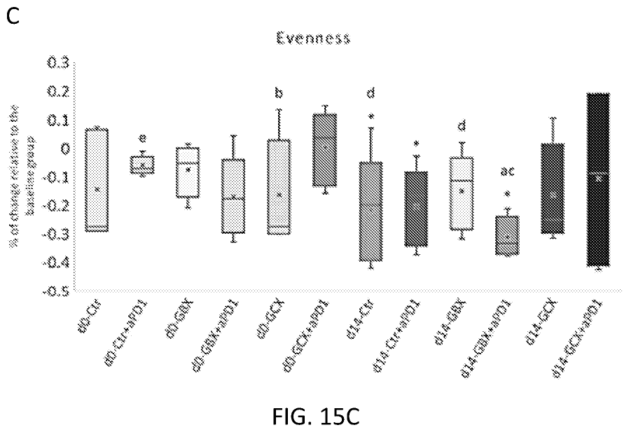

A total of 89 samples from mice stool were analyzed to investigate the composition of the bacterial microbiota (no stool was collected from a mouse in the GBX group on day 0). Samples were used to generate deep V3-V4 16S rRNA gene profiles. A total of 8,923,896 high-quality reads were obtained, with an average of 44,619 sequences per sample. The overall number of ASVs detected was 3300. The number of reads per sample ranged from 19,032 to 132,458. After the calculation of relative abundance per sample, further analyses were performed. Principal component analysis (PCA) of the microbial communities showed that all the mice started with a similar microbial profile with no significant clustering by group in the baseline pre-phase (d-14). Following 14 days of pre-feeding with the test items and prior to the tumor cell injection (d0), significant clustering was found between groups which highlights the specific modulation induced by GBX and GCX on the microbiota. Moreover, internal control groups (i.e. the mice that received aPD1 administration on day 6) also significantly clustered, which could be explained by the fact that the animals were divided in cages from the beginning of the experiment, which can lead to microbiome differences (Laukens et al., 2016). After melanoma cell injection and following 14 days of treatment (d14), significant clustering was found in all the groups, except between GBX group and GCX+aPD1 group and between GCX group and GCX+aPD1 group (FIGS. 14A-14C).

### Microbial Diversity

Alpha-diversity measures showed a significant decrease in Observed ASVs in Ctr−aPD1 group (with tumor cell injection and aPD1 administration) and in GBX−aPD1 group (with tumor cell injection, aPD1 administration and GBX supplementation) in comparison with the control group. These results were not observed in group GCX−aPD1. Moreover, a significant decreased was observed in Shannon diversity and Evenness in Ctr−aPD1 group and in GBX−aPD1 group in comparison with the baseline and with the control group. It has been previously shown that a higher diversity in the microbiome is correlated with a higher response to immunotherapy treatment (Gopalakrishnan et al., 2018; Zheng et al., 2019).

### Changes in Relative Abundance of Various Genera

At the genus level, 101 taxa were observed in the samples. The most predominant genera in all the samples were Barnesiella (30.4% of the total number of reads), followed by Prevotella (8.35%) and Clostridium_XlVa (7.97%). The three most abundant genus per group are summarized in Table 1.

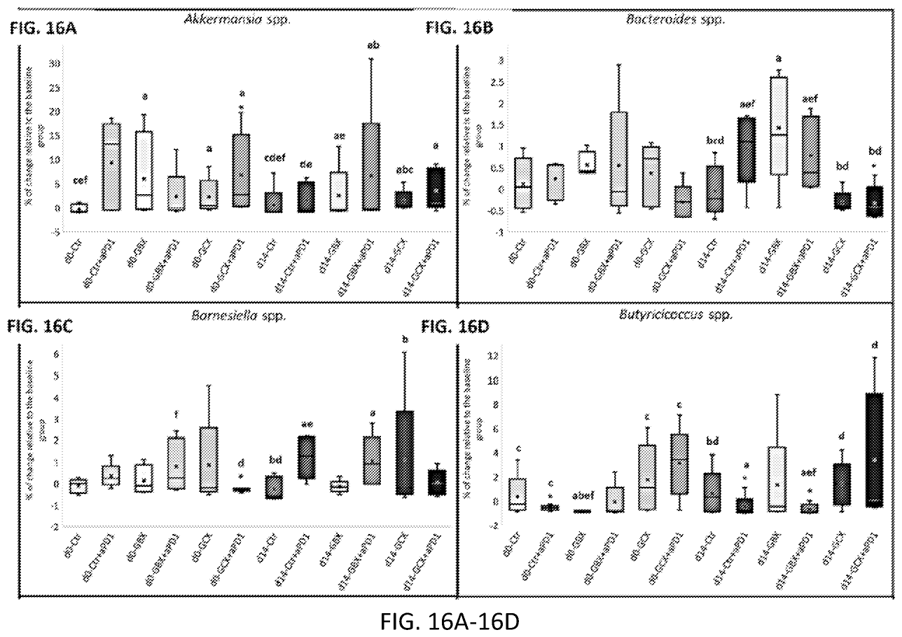

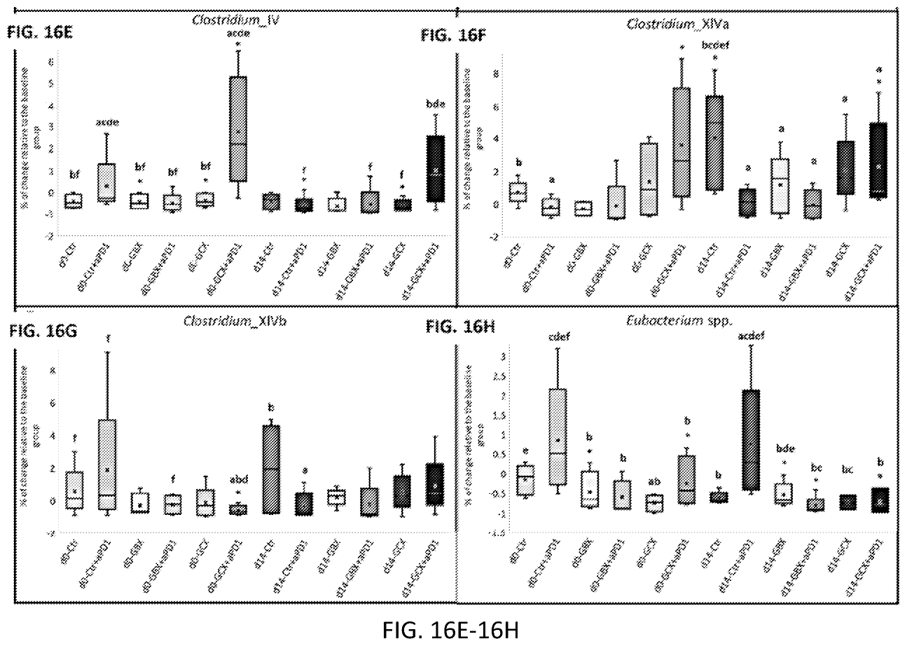

Akkermansia spp. showed a significant increase before tumor cell injection (d0) in mice receiving GBX and GCX in comparison with the control group. Moreover, this genus increased significantly after tumor cell injection (d14) in groups receiving GBX and GCX supplementation with and without aPD1 administration in comparison with the control group (FIGS. 16A-16H). These results agree with previous studies correlating anti-tumor properties to Akkermansia muciniphila (Routy et al., 2018; Li et al., 2020).

The genera Bacteroides and Barnesiella increased significantly in the control group receiving aPD1 after tumor cell injection (d14). There were no changes associated to GBX and GCX administration (FIGS. 16A-16H).

The genera Butyricicoccus and Clostridium_IV showed a significant decrease after tumor cell injection (d14) in control group with aPD1 administration (no supplementation) and GBX group with aPD1 administration while the GCX group with aPD1 administration suffered an increase (FIGS. 16A-16H). GCX seems to counteract the effect of aPD1. Recently, it was shown that Butyricicoccus pullicaecorum acted as a probiotic with anti-colorectal cancer properties (Chang et al., 2020). Moreover, a previous study showed a significant decrease of Clostridium IV in patients during cancer immunotherapy (Routy et al., 2018).

Clostridium_XIVa and Clostridium_XIVb did not show differences associated to the administration of GBX and GCX (FIGS. 16A-16H).

Eubacterium spp. suffered an imbalance when aPD1 was applied in the control group. The significant increased was not observed when GBX and GCX was applied suggesting the supplementation of both products was counteracting the effect of aPD1 (FIGS. 16A-16H).

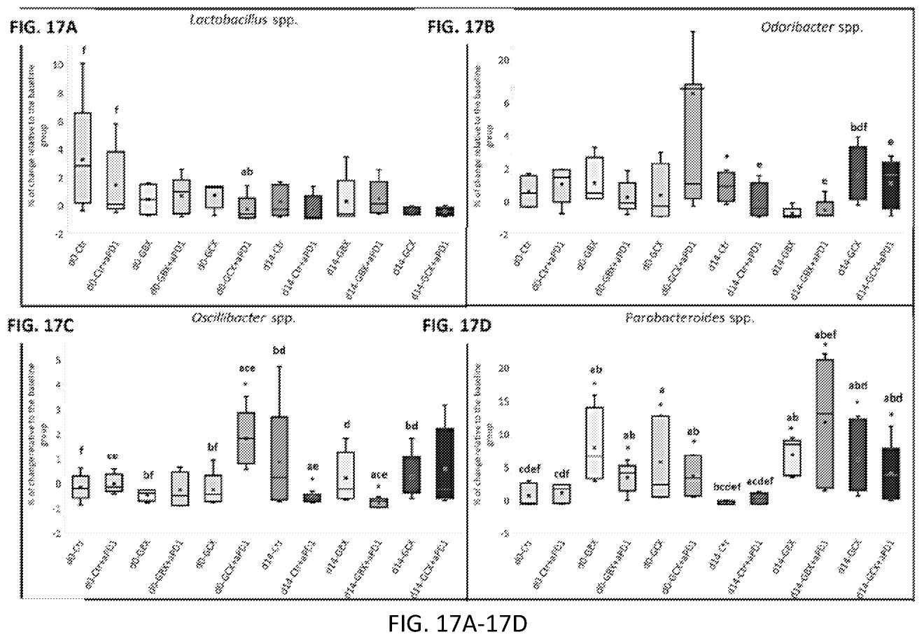

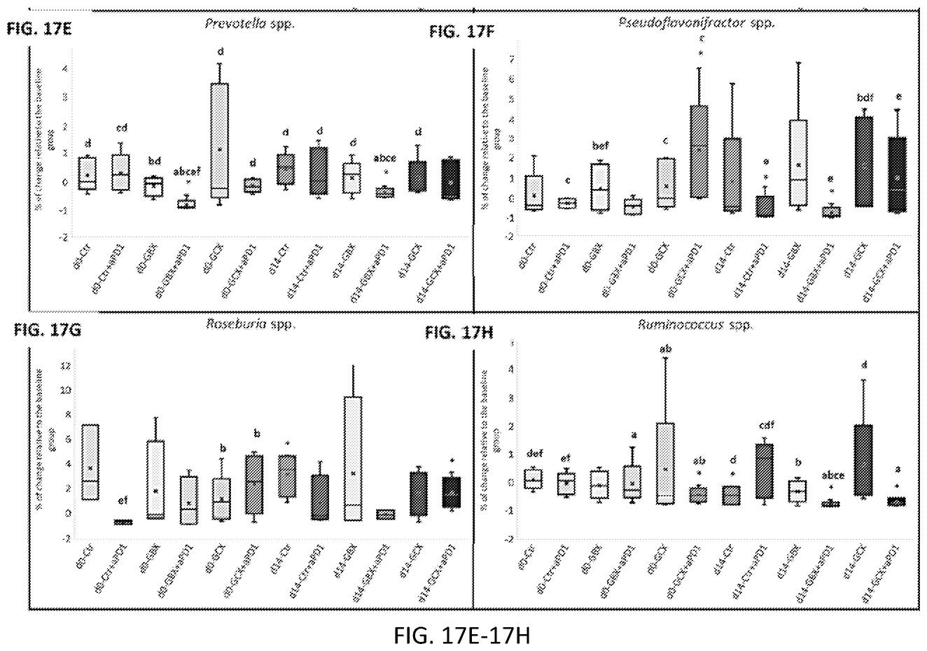

Lactobacillus spp. and Roseburia spp. did not show differences associated to the administration of GBX and GCX (FIGS. 17A-17H).

The administration of aPD1 negatively affected the genera Odoribacter, Oscillibacter and Pseudoflavonfractor when applied alone or supplemented with GBX. However, when applied together with GCX there was no negative effect (FIGS. 17A-17H).

Table 1. Mean relative abundance (% of sequences) of the three predominant bacterial genera in mice fecal samples after the pre-feeding phase but prior to tumor cell injection (d0) and after the tumor cell injection and 14 days of treatment (d14). Ctr group=mice that neither had test items supplementation in water, nor aPD1 administration throughout the study duration, Ctr−aPD1 group=mice that had aPD1 administration on Day 6 but not test items supplementation in water, GBX group=mice that did not have aPD1 administration but had GBX supplementation in water throughout the study, GBX−aPD1 group=mice that had aPD1 administration on Day 6 and GBX supplementation in water throughout the study, GCX group=mice that did not have aPD1 administration but had GCX supplementation in water throughout the study and GCX+aPD1 group=mice that had aPD1 administration on Day 6 and GCX supplementation in water throughout the study.

TABLE 1

| Time | Treatment | | | |
|---|---|---|---|---|
| point | group | | Genus | % |
| BL | Control | 1 | Barnesiella | 20.33 |
| | | 2 | Prevotella | 4.91 |
| | | 3 | Alistipes | 4.75 |
| | Control + aPD1 | 1 | Barnesiella | 14.60 |
| | | 2 | Prevotella | 6.54 |
| | | 3 | Clostridium_XIVa | 6.20 |
| | GBX | 1 | Barnesiella | 20.83 |
| | | 2 | Clostridium_XIVa | 5.93 |
| | | 3 | Alistipes | 5.09 |
| | GBX + aPD1 | 1 | Barnesiella | 16.84 |
| | | 2 | Clostridium_XIVa | 7.71 |
| | | 3 | Prevotella | 5.24 |
| | GCX | 1 | Barnesiella | 17.51 |
| | | 2 | Prevotella | 5.03 |
| | | 3 | Bacteroides | 3.93 |
| | GCX + aPD1 | 1 | Barnesiella | 21.84 |
| | | 2 | Prevotella | 7.43 |
| | | 3 | Lactobacillus | 6.04 |
| d0 | Control | 1 | Barnesiella | 17.99 |
| | | 2 | Lactobacillus | 9.27 |
| | | 3 | Prevotella | 5.40 |
| | Control + aPD1 | 1 | Barnesiella | 17.27 |
| | | 2 | Prevotella | 6.82 |
| | | 3 | Lactobacillus | 5.35 |
| | GBX | 1 | Barnesiella | 20.42 |
| | | 2 | Alistipes | 7.22 |
| | | 3 | Parabacteroides | 4.68 |
| | GBX + aPD1 | 1 | Barnesiella | 22.09 |
| | | 2 | Alistipes | 6.28 |
| | | 3 | Alloprevotella | 5.96 |
| | GCX | 1 | Barnesiella | 17.64 |
| | | 2 | Prevotella | 7.27 |
| | | 3 | Alistipes | 5.003 |
| | GCX + aPD1 | 1 | Barnesiella | 15.56 |
| | | 2 | Alistipes | 6.52 |
| | | 3 | Clostridium_XIVa | 6.44 |
| d14 | Control | 1 | Clostridium_XIVa | 13.94 |
| | | 2 | Barnesiella | 13.72 |
| | | 3 | Prevotella | 6.52 |
| | Control + aPD1 | 1 | Barnesiella | 27.82 |
| | | 2 | Alloprevotella | 9.005 |
| | | 3 | Bacteroides | 6.78 |
| | GBX | 1 | Barnesiella | 17.5 |
| | | 2 | Alloprevotella | 7.84 |
| | | 3 | Bacteroides | 6.81 |
| | GBX + aPD1 | 1 | Barnesiella | 26.72 |
| | | 2 | Alloprevotella | 9.28 |
| | | 3 | Bacteroides | 6.33 |
| | GCX | 1 | Barnesiella | 17.64 |
| | | 2 | Alloprevotella | 11.78 |
| | | 3 | Clostridium_XIVa | 6.79 |
| | GCX + aPD1 | 1 | Barnesiella | 20.98 |
| | | 2 | Alloprevotella | 12.98 |
| | | 3 | Prevotella | 5.46 |

Parabacteroides spp. showed a significant increase before tumor cell injection (d0) in mice receiving GBX and GCX in comparison with the control group. Moreover, this genus decreased significantly after tumor cell injection (d14) in control group with and without aPD1 and increased significantly in all groups receiving GBX and GCX supplementation with and without aPD1 administration (FIG. 17D). Parabacteroides spp. has been positively correlated with a reduction of tumor size after mucin treatment (Li et al., 2020).

### Conclusion

In conclusion, GBX and GCX were both able to boost an anti-tumor immune response upon PD1 inhibition. Furthermore, GCX has anti-tumor effects on its own.

### Discussion

In mice subcutaneously injected with YUMM1.7 melanoma tumor cells:
orally administered GBX in combination with anti-PD-1 reduces tumor volume & weight by ˜50% while anti-PD-1 or GBX alone does not.
orally administered GCX in combination with anti-PD-1 reduces tumor volume and weight by ˜50% while anti-PD-1 alone does not.
orally administered GCX is effective in tumor volume reduction independently of anti-PD-1 (as a monotherapy).

Considering GBX is enhancing the efficacy of the immune checkpoint inhibitor (ICI) but does not induce anti-tumor immunity in the absence of ICI, GBX is believed to promote the expansion and infiltration of T cells within the tumor microenvironment, in particular the effector CD8 T cells, but the ICI therapy is required to unleash the anti-tumor potential of CD8 T cells by blocking the PD1R-PD1L interaction that is otherwise immunosuppressive. Alternatively, GBX induces the expansion of T cells locally, and restricted to the gut-associated lymphoid tissue (GALT), while the ICI therapy helps to achieve a systemic activation, possibly via the gastrointestinal inflammation and alteration in the gut barrier integrity induced by the ICI.

With regards to GCX, it is hypothesized that GCX is both promoting the expansion and infiltration of effector T cells within the tumor microenvironment (e.g. by up-regulating pro-inflammatory cytokines and chemokines produced by epithelial cells which help recruit CD8 T cell to the colon) and reducing immunosuppressive responses at play (e.g. by downregulating the expression of PD1R on T cells, blocking other immunosuppressive interactions).

Without being bound by a particular theory, Applicant proposes two major axes with regards to the modes of action: (i) Microbiome and microbiome-derived immunostimulatory metabolites, (ii) Glycopeptide-driven molecular mimicry and cellular signaling.

### Microbiome & Microbiome-Derived Immunostimulatory Metabolites

GBX and GCX have been demonstrated to promote the growth of bacteria with therapeutical potential to regulate anti-tumor immunity. These bacteria are for example Akkermansia muciniphila, Parabacteroides spp., and Bacteroides thetaiotaomicron. Monocolonization of these bacteria in GF mice have demonstrated a modulation of the immune cell populations in the tumor microenvironment and expression of various cytokines and chemokines. Administration of A. muciniphila was associated with increased intra-tumoral immune infiltrates, mediated by the recruitment of CCR9+CXCR3+CD4+ T cells into the tumor bed and an increased ratio of CD4+ T cells to CD4+FoxP3+ T cells (Tregs) in response to PD-1 blockade. Bacteroides thetaiotaomicron and B. fragilis mediate Toll-like receptor 4 (TLR4)- and IL-12-dependent TH1 responses and therapeutic efficacy.

Important microbiome-derived metabolites or structural components have been shown to induce an anti-tumor immunity. Inosine, a metabolite produced by B. pseudolongum, but also by Akkermansia muciniphila, was shown to promote Th1 cell activation through T cell specific A_{2A} receptor (Inosine binds to adenosine A2A receptor on T cells, which enhance Th1 differentiation and activation when T-cells are co-stimulated by DC). The breaks down of peptidoglycans, components of the bacterial cell wall, by Enterococcus species (E. faecium, E. durans, E. hirae, E. mundtii) results in the release of muramyl peptide fragments which stimulates the innate immune sensor protein NOD2 and improves immunotherapy responses (NOD2 is a key pattern recognition receptor for muropeptides. NOD2 signaling generates a proinflammatory cascade through activation of transcription factor NF-kB and mitogen activated protein kinase (MAPK) phosphorylation cascade. NOD2 stimulation has been shown to generate conventional type 1 dendritic cells).

Considering these examples, GCX and GBX appear to induce specific modulations of the microbiome composition and function to trigger an anti-tumor response.

### Glycopeptide-Driven Molecular Mimicry and Cellular Signaling.

Given their specific chemical structures, GBX and GCX can act as:

Inducer of tumor-specific CD4+ and CD8+ T cells. Cancer (neo)antigens share epitopes with GBX and GCX through molecular mimicry. GBX and GCX can bind to DC-SIGN, be internalized and then cross-presented as an antigen on the surface of the dendritic cell, which would then migrate and stimulate tumor-specific CD4+ and CD8+ T cells. An improved tumor-specific T cell response will support a long-term tumor regression.

Activator of innate immune response via Toll-like receptor (TLR). Toll-like receptor (TLR) ligands have long been used as adjuvants for traditional vaccines and it seems they may also play a role enhancing efficiency of tumor immunotherapy. Their efficiencies as immunotherapeutic agents rely mostly on the initiation of T-cell immunity: antigen uptake, processing and presentation, maturation of dendritic cells, and activation of T cells. It is expected that GBX and GCX will bind to TLR on immature antigen presenting cells (APCs) like dendritic cells and induce their maturation to professional APCs that can present antigens on major histocompatibility complex 1 (MHC 1). Antigens are then presented to T-cells via T-cell receptor (TCR)-MHC 1 binding in the presence of co-stimulatory molecules such as cluster of differentiation 80 (CD80), CD86 (on antigen-presenting cells (APCs) binding CD28 (on T cells).

Beside expression on professional APCs, TLRs can also be expressed in T cells where they act as co-stimulatory receptors that complement TCR-induced signaling to enhance T cell proliferation and cytokine production. Additionally TLR stimulation of T regulatory cells may revert their immunosuppressive capabilities. This is particularly interesting for cancer research due to high levels of Tregs and their immunosuppressive activity in tumor microenvironment.

Blocker of immunosuppressive mechanisms. Several studies have shown that lectin receptors (for example, sialic acid-binding immunoglobulin-like lectins (SIGLECs), macrophage galactose-specific lectin (MGL) and dendritic cell (DC)-specific ICAM-3-grabbing non-integrin 1 (DC-SIGN; also known as CD209)) expressed by immune cells mediate immune suppression by responding to the tumor glyco-code. For example, DC-SIGN triggering by fucose-containing structures results in upregulation of the anti-inflammatory cytokines IL-10 and IL-27 and in the induction of T helper 2 (TH2), T follicular helper (TFH) or regulatory T (Treg) cells.

In addition to aberrant glycan expression, cancer cells may also display altered expression of glycan-binding lectins. Galectins, a family of soluble lectins, can be secreted by a wide range of tumors and are able to impair T cell effector function, instruct the differentiation of suppressive myeloid cells and modulate NK cell activity by binding to specific glycans expressed on these immune cell populations. For example, Galectin 1 (Gall) contributes to immune evasion through several mechanisms, including differentiation of tolerogenic DCs and the induction of apoptosis in TH1 and TH17 cells. Its blockade in the tumor microenvironment will augment the effector functions of CD4+ and CD8+ T cells.

Preventing the interaction of tumor-associated glycans with inhibitory immune receptors is a promising antitumor therapy. GBX and GCX are believed to enhance T cell-mediated antitumor responses and enhance NK cell activity by blocking glycan-lectin interactions.

SUMMARY TABLE:

| Block | Effect | MoA (mechanism of action) |
|---|---|---|
| Microbiome | Favor a T cell | Microbiota can modulate the peripheral immune |
| & | inflamed tumor | system and its diversity plays a crucial role in the |
| microbiome-derived | microenvironment | maturation, development and function of both |
| immunostimulatory | (hot tumor) | the innate and the adaptive immune systems. |
| metabolites | | Therefore, the modulation of specific bacteria |
| | | can promote the expansion and recruitment of |
| | | CD8 T cells in the tumor bed which carry out |
| | | direct cytotoxic reactions that kill cancer cells. |
| | | GBX and GCX promote the growth of bacteria |
| | | with therapeutical potential to regulate anti-tumor |
| | | immunity. |
| | | A. muciniphila is associated with increased intra- |
| | | tumoral immune infiltrates, mediated by the |
| | | recruitment of CD4 T cells into the tumor bed and |
| | | an increased ratio of CD4 T cells to Tregs in |
| | | response to PD-1 blockade. |
| | | Bacteroides thetaiotaomicron and B. fragilis |
| | | mediate Toll-like receptor 4 (TLR4)- and IL-12- |
| | | dependent Th1 responses and therapeutic |
| | | efficacy. |
| | | Akkermansia muciniphila and B. pseudolongum |
| | | produce inosine, a metabolite shown to promote |
| | | Th1 cell differentiation and activation through T |
| | | cell specific adenosine A2A receptor (upon co- |
| | | stimulation by DC). |
| | | Enterococcus species break down |
| | | peptidoglycans, resulting in the release of |
| | | muramyl peptide fragments which stimulate the |
| | | innate immune sensor protein NOD2 and |
| | | improves immunotherapy responses. NOD2 is a |
| | | key pattern recognition receptor present inside |
| | | DCs. NOD2 signaling generates a |
| | | proinflammatory cascade and has been shown to |
| | | generate conventional type 1 dendritic cells, |
| | | which excel at inducing anti-tumoral CD8 T cell |
| | | response. |
| Glycopeptide- | Remove | Tumor can evade the immune system by |
| driven | immunosuppressive | exploiting inhibitory signals such as the PD1 |
| molecular | signaling | pathway. Glycans can induce immunosuppressive |
| | | signaling as well through lectins (which are |
| | | glycan-binding receptors), making glycans a |
| | | novel type of immune checkpoint. |
| | | GBX and GCX may competitively bind to lectins |
| | | present on DCs and other immune cells. This |
| | | competitive binding may prevent the binding of |
| | | these lectins to glycans present on the tumor |
| | | which would result otherwise in an |
| | | immunosuppressive environment (production of |
| | | anti-inflammatory cytokines like IL-10 and IL- |
| | | 27, induction of Treg cells). |
| | | GBX and GCX may competitively bind to |
| | | Galectins shed by tumors. This competitive |
| | | binding may prevent the binding of these |
| | | Galectins to glycans presents on T cells and DCs |
| | | which would result otherwise an |
| | | immunosuppressive environment (induction of |
| | | apoptosis of T cells, differentiation in tolerogenic |
| | | DCs). |
| | Activate innate | The maturation and activation of APCs like DCs |
| | immune system for | is a critical step for activating an efficient T cell |
| | stimulating an | response (adaptive immunity). DCs can be |
| | efficient T cell | directly activated and become competent to |
| | response | prime T cell when their TLRs are stimulated. |
| | | GBX and GCX may stimulate Toll-like receptors |
| | | (TLRs) on DCs, turning immature DCs in |
| | | mature professional DCs that can present |
| | | antigens to T cells. |
| | | Note: TLRs can also be expressed in T cells |
| | | where they act as co-stimulatory receptors to |
| | | enhance T cell proliferation and cytokine |
| | | production. |
| | Induce tumor- | DCs capture, process, and (cross-) present |
| | specific CD4+ and | antigens to naive CD4+ and CD8+ T cells and are, |
| | CD8+ T cells | therefore, the main instigators in initiating |
| | | adaptive immunity. |
| | | GBX and GCX may share epitopes with cancer |
| | | (neo)antigens through molecular mimicry. |
| | | Indeed, glycosylation of tumor proteins |
| | | generates neo-antigens that can serve as targets |
| | | for tumor-specific T cells. GBX and GCX can |
| | | bind to DC-SIGN, be internalized and then |
| | | cross-presented as an antigen on the surface of |
| | | the DC, which would then migrate and stimulate |
| | | tumor-specific CD4+ and CD8+ T cells. An |
| | | improved tumor-specific T cell response will |
| | | support a long-term tumor regression. |

### Example 6

Applicant evaluated the efficacy of GBX and GCX in reducing tumor size when administered alone or in combination with immune checkpoint inhibitors (ICI) in mice subcutaneously injected with colorectal cancer (CRC) tumor cells. Applicant also evaluated the impact of the treatment on (i) immune cell populations infiltrating the tumor tissue, (ii) inflammatory markers and metabolites, and (iii) gut microbial diversity & composition. To evaluate these effects, oral administration with test items provided in drinking water at 3% w/v was tested in this study.

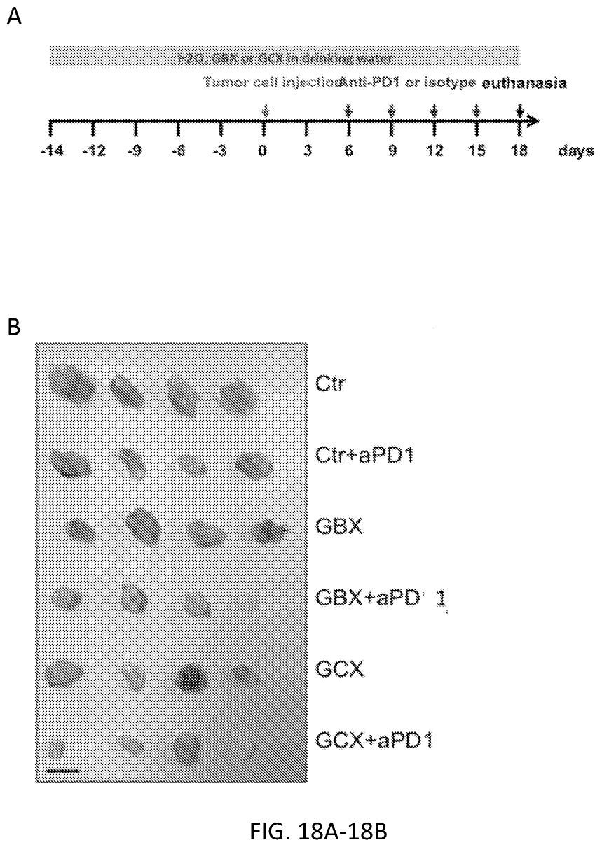

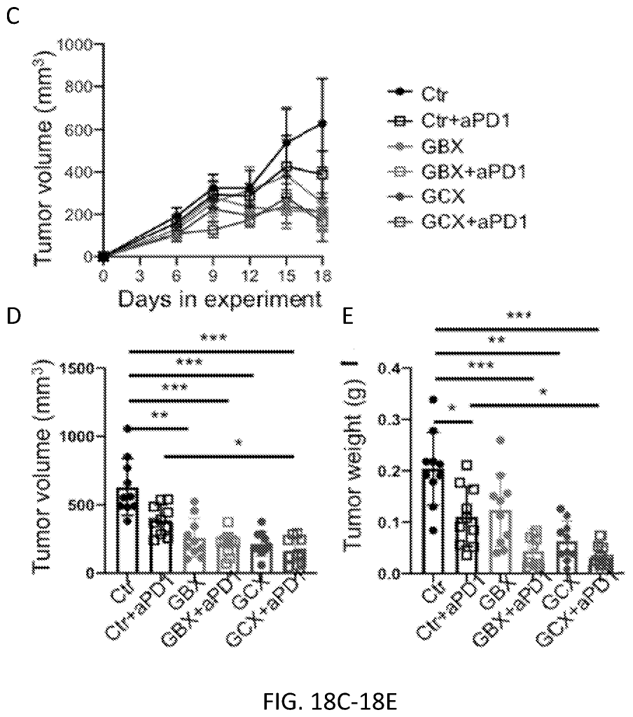

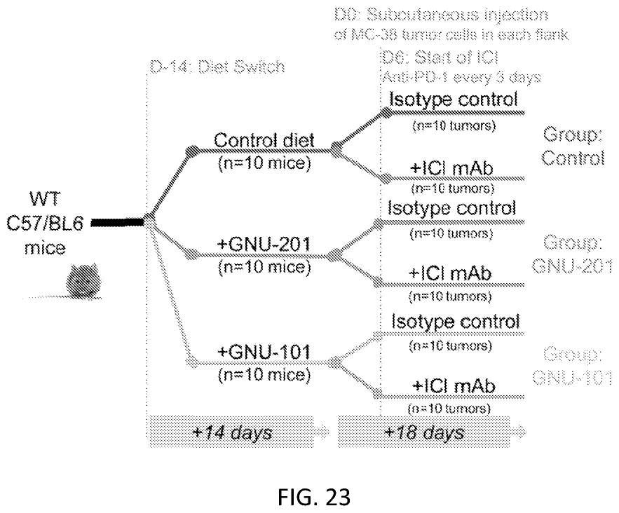

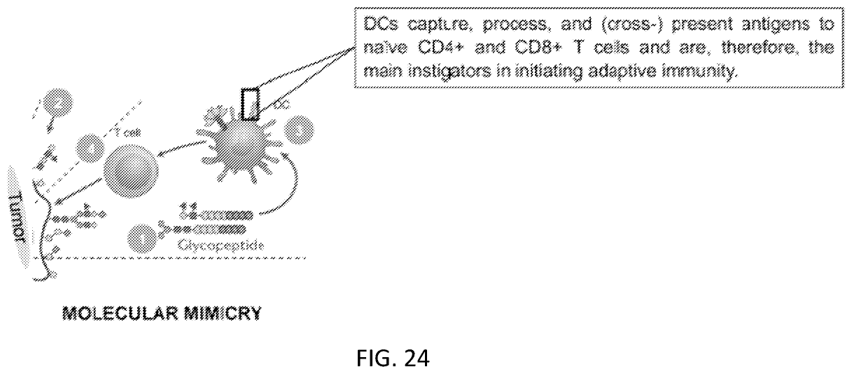

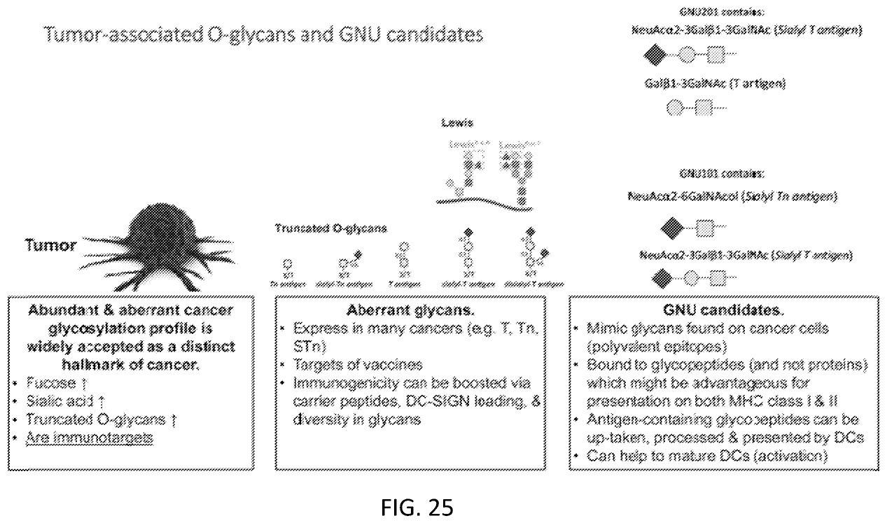

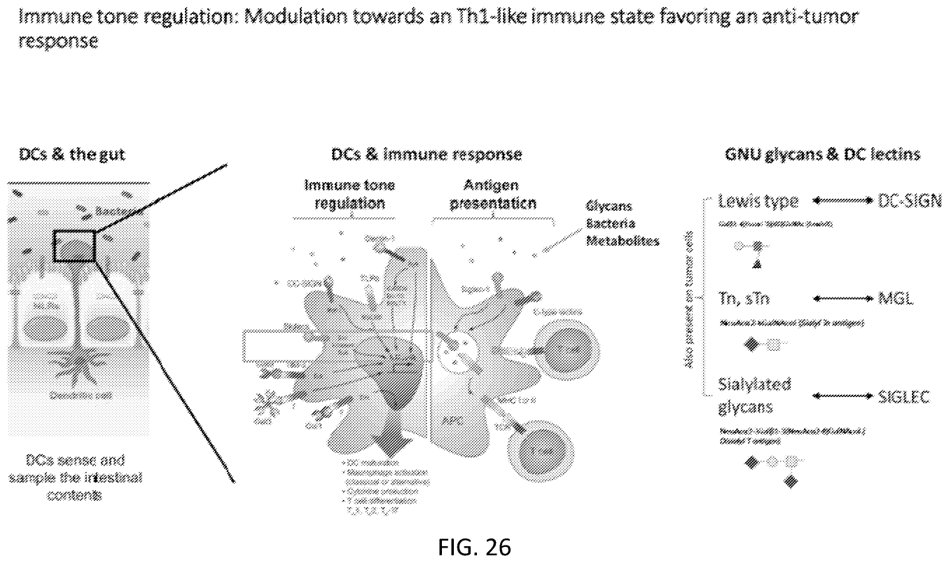

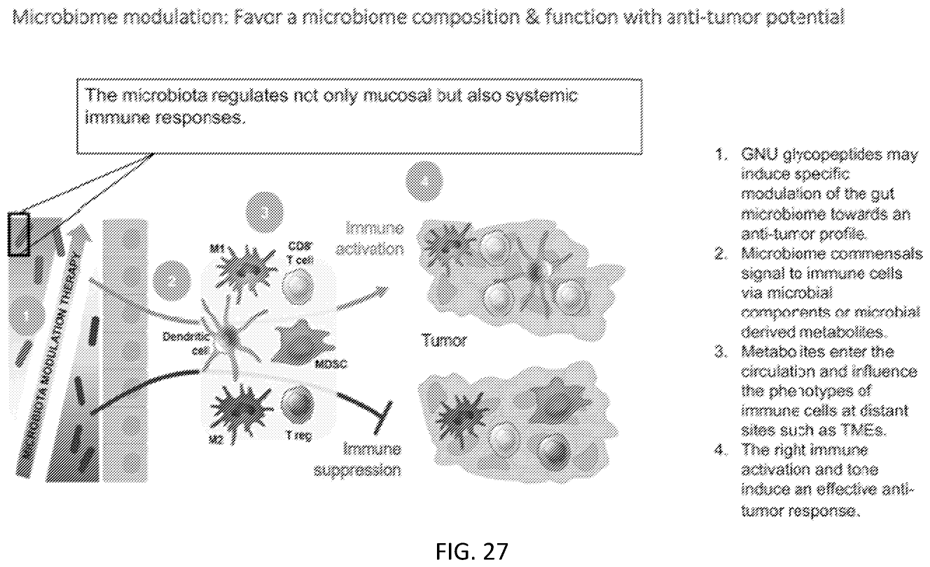

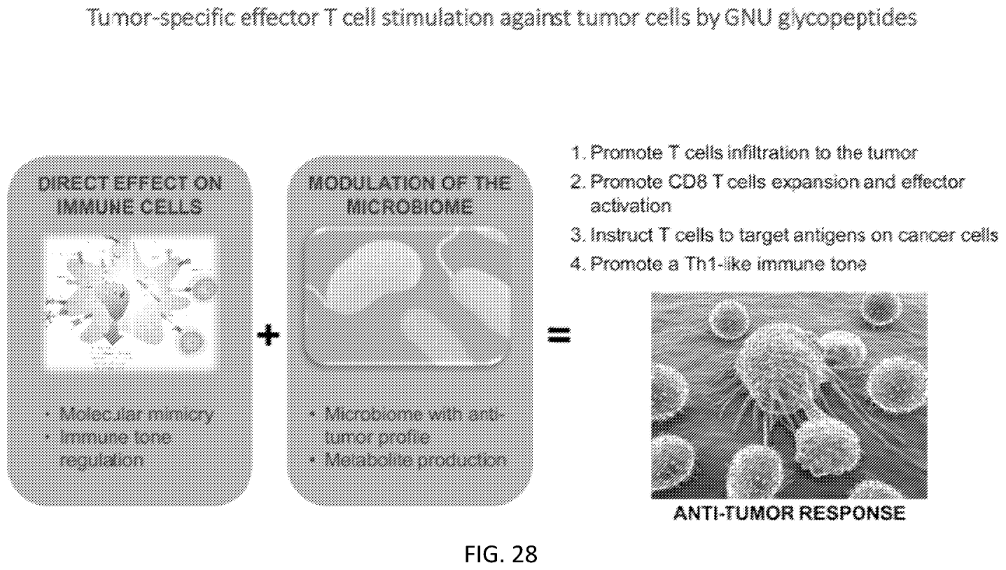

To test the effect of the test products “GBX” and “GCX” on CRC tumor growth and on PD1-inhibition-mediated anti-tumor effects, C57/BL6 mice (n=5/group) were injected subcutaneously with 200,000 MC38 cells in each flank. 10 mice received 3% GBX and 10 mice 3% GCX in the drinking water starting 14 days prior to tumor cell injection, while 10 mice received normal drinking water (control, Ctr). Since the test compounds promote bacterial growth, the drinking water was filtered or replaced with new product every day. On day 6, day 9, day 12 and day 15 after tumor cell injection, 5 mice in each group received 200 μg anti-PD1 antibody (BioXCell; clone 29F.1A12) via intraperitoneal injection (FIG. 18A, experimental overview) or control isotype (BioXCell). Tumor size was measured every 3rd day to monitor differences in growth. On day 18 after tumor cell injection, the mice were sacrificed to analyze tumor weight, tumor infiltrating immune cells, and cytokine levels in the serum. This study protocol is provided in FIG. 23.

| | | | |
|---|---|---|---|
| D-14 | D0 | D6 | D18 |

MAJOR STUDY EVENTS

| | | | | |
|---|---|---|---|---|
| Randomization | X | | | |
| (to different diet | | | | |
| groups) | | | | |
| Tumor injection | | X | | |
| ICI treatment start | | | X | |
| Study end | | | | X |

COLLECTION

| | | | |
|---|---|---|---|
| Tumor | | | X |
| Serum | | X | X |
| Stool | X | X | X |
| Cecum & colon | | | X |
| content | | | |
| Colon tissue | | | X |

END-POINTS

| Tumor volume | | Every 3 days | |
|---|---|---|---|
| Tumor weights | | | X |
| TILs | | | X |
| 16S | (OPTIONAL) | X | X |
| Cytokine panel | | X | X |
| Untargeted | | X | X |
| metabolomic | | | |

END-POINTS

| END-POINT | DESCRIPTION | TIMEPOINT |
|---|---|---|
| Tumor volume | PRIMARY EP: To assess impact on | Every 3 days, |
| | tumor evolution | starting on D0 |
| Tumor weights | PRIMARY EP: To assess impact on | D18 (end of exp) |
| | tumor evolution | |
| TILs & subsets | To characterize anti-tumor immunity | D18 (end of exp) |
| (from tumor sample) | | |
| 16S | To assess microbiome modulation | D0 |
| (from fecal samples) | activity & link to anti-tumor | D18 (end of exp) |
| | immunity | OPTIONAL: |
| | | D-14 (baseline) |
| Cytokine panel | To characterize systemic | D0 |
| (from serum) | inflammation-promoting effect | D18 |
| Untargeted | e.g. to look for inosine | D0 |
| metabolomics | | D18 |
| from serum) | | |

### Materials & Methods

### Animal Models and Husbandry Conditions

Female C57BL/6J mice at an age of 9-12 weeks were used for all experiments. The mice were housed in an SPF facility with food and water ad libitum and a 12 h light-dark cycle.

### Dose Formulation

Mice were treated with normal drinking water or supplemented with 300 w/v of test item (GBX or GCX) throughout the experiment. To prevent bacterial growth, the drinking water/drinking water with test item was filtered or replaced with new product every day.

### Tumor Models and Treatments

The test compounds were dissolved in drinking water at a concentration of 3% (w/v) and sterile filtered using 0.22 um filters from Merck-Millipore. To prevent bacterial growth, the drinking water was filtered again 24 h after supplementation and replaced with fresh drinking water after 48 h. The mice received water with test compounds starting 14 days prior to tumor cell injection. 200′000 MC38 CRC cells were injected subcutaneously into the left and right flank (100 ul of a 1:1 culture medium:Matrigel mixture). Starting on day 6 after tumor cell injection, the mice were injected with 200 μg/mouse anti-PD1 (BioXcell #PB0273) or isotype control (BioXCell, #PB0089) every three days until the end of the experiment. Tumors were measured every 3 days using an electronic caliper and tumor volumes calculated using the ellipsoid formula 4/3*π*a*b² where a is the length and b the width of the tumor.

### Tumor Cell Lines Culture

MC38 cells were a gift from Prof. Borsig (Institute of Physiology, University of Zurich) and maintained in DMEM medium (Thermo Fisher Scientific #11885-084) with 10% FCS in a 37° C. 100% humidity cell culture incubator at 10% C02. Cells were kept at a confluence below 70% at all times. For passaging, the cells were rinsed with PBS, detached with trypsin (1%) for 5 minutes, supplemented with growth medium and used for experiments or seeded at a lower density into new cell culture flasks. For experiments, the cells were centrifuged (1200 rpm, 5 min, 4° C.), re-suspended in growth medium and mixed 1:1 with matrigel (Corning, #FAL354263)

### Flow Cytometry

For flow cytometry, tumors were cut to approximately 0.5 mm³ pieces and digested in 6 mL RPMI containing 0.5 mg/mL collagenase type IV (Sigma Aldrich) and 0.05 mg/mL DNAse I (Roche) solution for 10 minutes on a shaker (300 rpm) at 37° C. Cells were homogenized passing through 18G1.5 syringe and centrifuged for 10 mins, 4° C., 1500 rpm. Single cell suspensions were centrifuged, washed in PBS and re suspended in antibody solution. For cytokine and cytotoxic factors, the cells were restimulated for 4 h in RPMI, 10% FCS supplemented with Ionomycin (1 ug/ml, Sigma-Aldrich, #I3909), PMA (1 ug/ml, Sigma-Aldrich, #1585) and Brefeldin A (50 ng/ml, Sigma-Aldrich, #B6542). After restimulation, the cells were stained for surface markers, washed in PBS, permeabilized using the FoxP3 staining kit from eBioscience (#00-5523-00), stained for intracellular molecules, washed and resuspended in PBS. Cells were acquired using a BD FACS Symphony analyzer.

### Cytokine Analysis

Serum cytokine levels were measured using the mouse 23-plex kit from Bio-rad (#M60009RDPD) according to the manufacturer's instructions. The serum was diluted 1:4 in assay buffer for analysis.

### Analysis of 16S Amplicon Sequencing Data

For 16S RNA sequencing, DNA was extracted from the stool using the DNA Soil kit from Qiagen following the manufacturer's instructions.

### Data Visualization and Statistics

Flow cytometry, tumor growth/weight, and cytokine data were visualized and analyzed using GraphPad Prism version 8.4.3. One- or two-way ANOVA with Dunnet's correction for multiple testing was used. Corrected p-values below 0.05 were considered significant.

### Results & Discussions

GBX and GCX Reduce Tumor Growth in Combination with Anti-PD1

GBX and GCX supplementation in the drinking water was well tolerated and no differences were observed in water consumption and no abnormal behavior was detected that would indicate adverse effects of the products.

Up until day 12, no significant differences in tumor growth were observed in any of the groups, but starting on day 15, it became obvious that the tumors treated with GBX, GCX, anti-PD1 or a combination of the products with anti-PD1 showed reduced growth, resulting in a significantly reduced tumor volume vs control in the groups GBX, GCX, GBX+anti-PD1 and GCX+anti-PD1 on day 18 after tumor cell injection (FIGS. 18B-D). Although the anti-PD1 therapy showed a reduction in tumor weight (FIG. 18E) and a numerical decrease in tumor volume (FIG. 18D), it showed a rather limited anti-tumor efficacy. In contrast, GBX and GCX treatment alone significantly reduced tumor volume compared with control and led to a numerically higher decrease than anti-PD1 alone, suggesting a higher efficacy (FIG. 18D). Moreover, combination of GCX with anti-PD1 showed significant reduction in tumor volume compared with anti-PD1 alone (a similar trend was observed with GBX). Similar effects were observed in tumor weight, although GBX treatment alone did not have an effect on tumor weight when compared with anti-PD1 (FIG. 18E). In summary, the data demonstrate that GBX and GCX as standalone treatment induce a strong anti-tumor response that is as good as or even better than anti-PD1 alone. Combination therapy should bring some additional therapeutic benefit for GBX.

### GBX and GCX Promote Anti-Tumor T Cell Responses

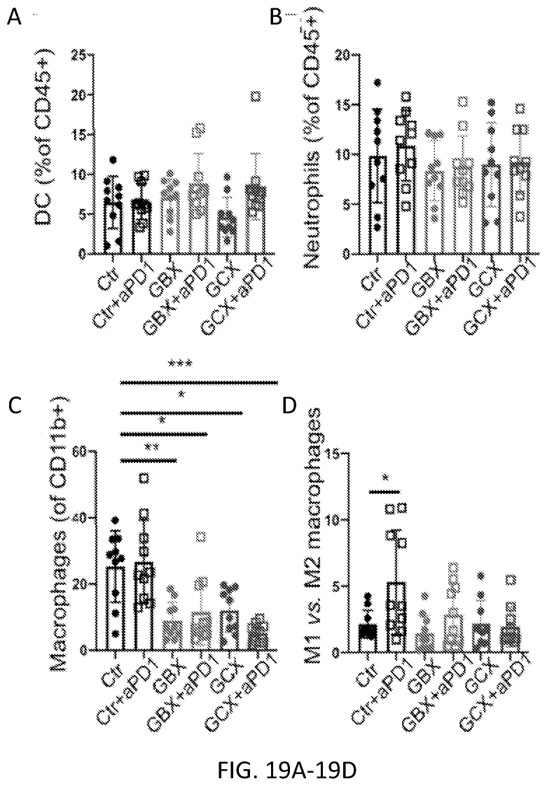

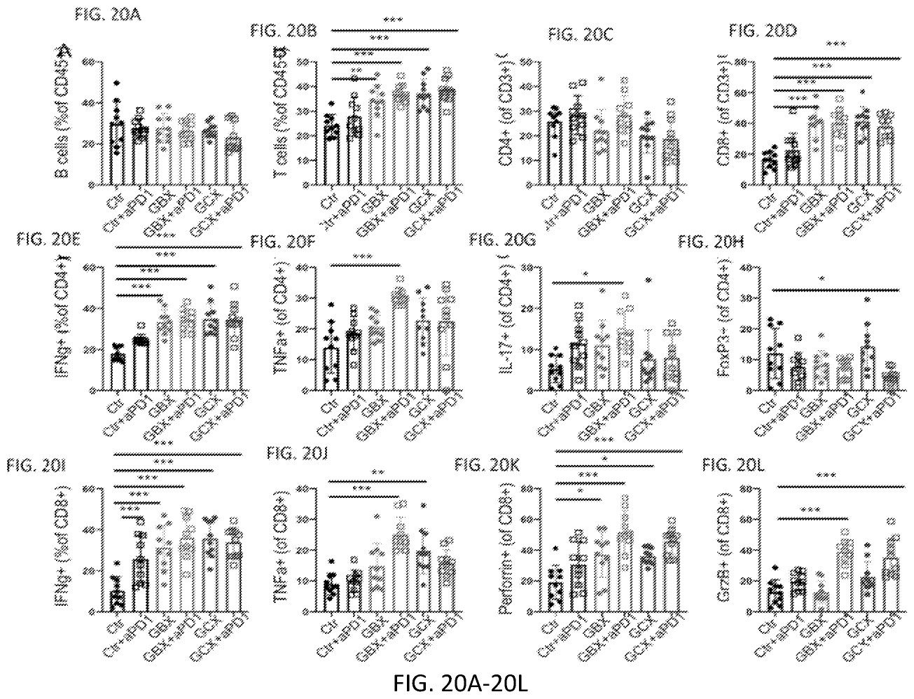

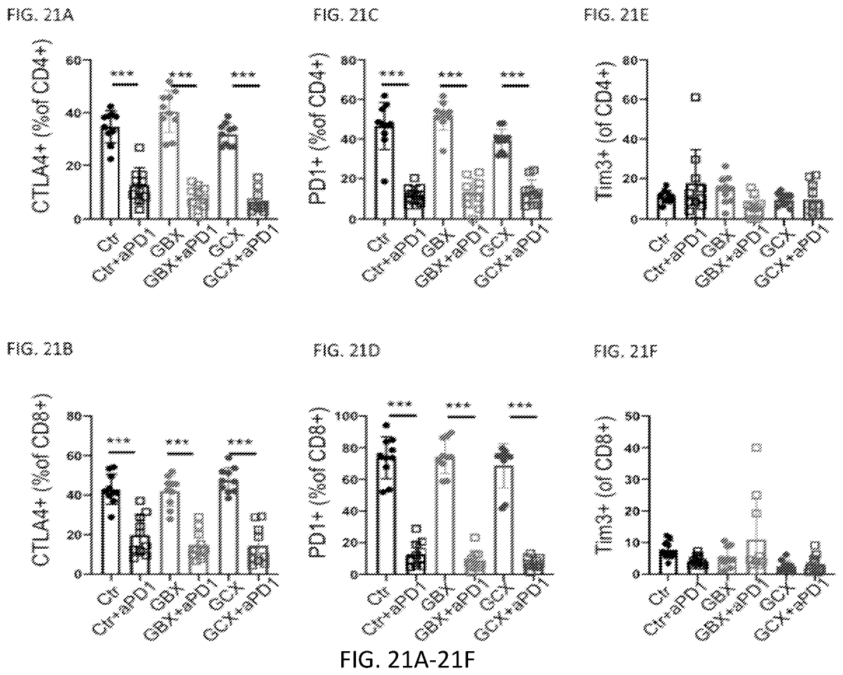

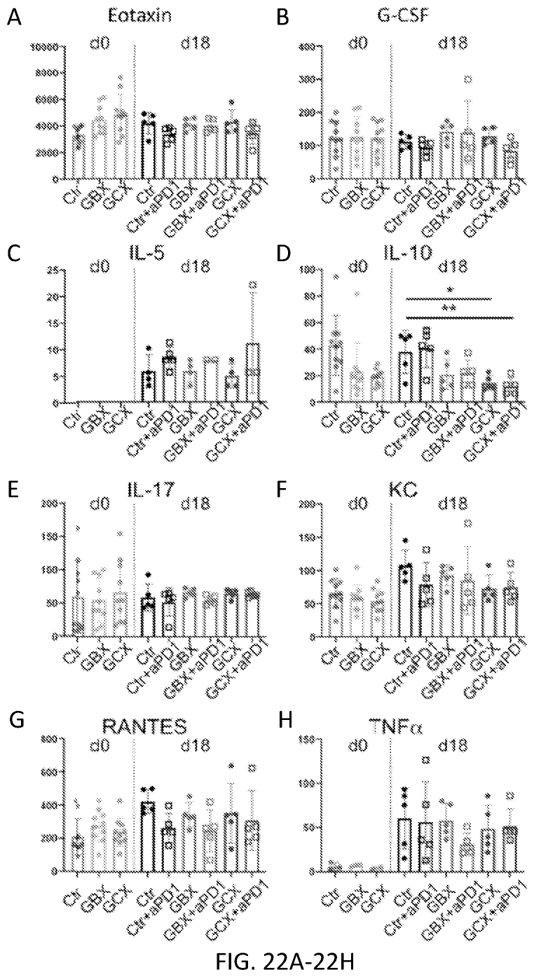

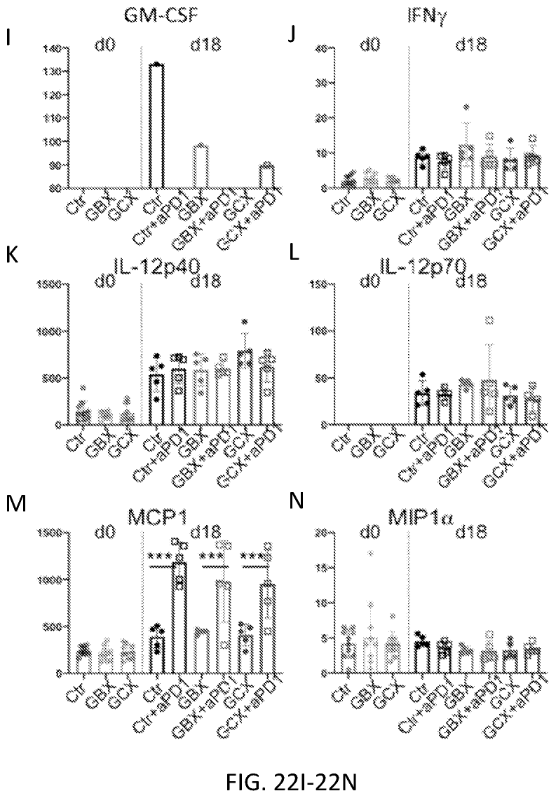

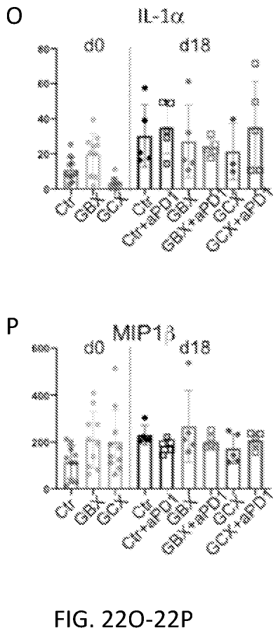

To assess the anti-tumor immune response, flow cytometry was performed on tumor-infiltrating immune cells. When analyzing myeloid immune cells, the effects of the test compounds were moderate, and no differences were observed in the proportions of neutrophils or dendritic cells (FIGS. 19A-19B). Notably, the overall proportion of macrophages was decreased in mice receiving GBX or GCX, both as treatment alone or in combination with anti-PD1 (FIG. 19C), but although there was a clear trend towards increased proportions of classically activated (M1) macrophages while alternatively activated (M2) macrophages tended to decrease upon anti-PD1 treatment, this only reached statistical significance in the control animals without GBX or GCX treatment (FIG. 19D). Likewise, relative abundance of B cells was not significantly affected in any of the treatment groups (FIG. 20A).

In contrast to these moderate effects on myeloid cells, there were clear effects on anti-tumor T cell responses upon administration of the two test compounds. GBX and GCX supplementation promoted the overall abundance of T cells (FIG. 20B), and among those the proportion of CD8+ T cells (FIG. 20D) and IFNγ producing CD4+ T cells (FIG. 20E). In addition, GBX treatment in combination with anti-PD1 resulted in increased levels of TNFα+ and IL-17+CD4+ T cells, and GCX treatment in combination with anti-PD1 reduced the abundance of FoxP3+ regulatory T cells (FIGS. 20F-20H). Regarding CD8+ T cells, anti-PD1 treatment alone promoted the number of IFNγ+ cells, while other activation markers were not affected. In contrast, treatment with GBX and GCX, alone or in combination with anti-PD1 promoted the proportion of IFNγ+ and perforrin+CD8+ T cells, and combination of GBX with anti-PD1 promoted the abundance of TNFα+ and granzyme B+CD8+ T cells (FIGS. 20I-20L). GCX treatment alone promoted the abundance of TNFα+CD8+ T cells while the combination of GCX with anti-PD1 promoted the abundance of granzyme B+CD8+ T cells (FIG. 20L). These data clearly show that GBX and GCX either alone or in combination with anti-PD1 promote anti-tumor T cell responses.

Anti-PD1 treatment induces a reduction of T cell exhaustion markers expression (CTLA4, PD1) while Tim3 expression is not affected.

A critical mechanism how antigen-specific T cells are rendered anergic to prevent excessive immune response is the expression of exhaustion markers on T cells upon prolonged T cell receptor stimulation. Exhaustion molecules are surface receptors that are upregulated late during immune responses and mediate cell death or suppression of the activity to terminate no longer needed immune responses. Many cancers express ligands to those exhaustion markers and in this way promote T cell anergy and/or death leading to immune escape. In addition, the cytokine milieu in tumors often favors expression of exhaustion markers on T cells. In line with an inhibitory effect on T cell anergy/T cell exhaustion, anti-PD1 treatment alone or in combination resulted in reduced expression of the exhaustion markers CTLA4 and PD1 (combination with GCX led to a numerically higher decrease in CTLA4 expression than anti-PD1 alone), but there was no significant difference between mice that received normal drinking water and those that received GBX or GCX (FIGS. 20A-20D). In contrast to these pronounced effects on CTLA4 and PD1, there were no effects observed on the exhaustion marker Tim3 (FIGS. 20E and 20F). Taken together, this indicates that in line with its mode of action, anti-PD1 treatment reduced T cell exhaustion, but there was no significant effect of the two test compounds.

### Minimal Effect of GBX and GCX on Serum Cytokine Levels

In contrast to the effects on tumor infiltrating immune cells, serum cytokine profiles did not show a signature that could be correlated with the anti-tumor effects of the two test compounds (FIG. 22). However, GCX administration resulted in reduced levels of IL-10 (a similar trend was observed for GBX), a molecule that is produced by a variety of cells, including tumor-infiltrating immune-suppressive myeloid cells/macrophages. Furthermore, anti-PD1 treatment resulted in elevated levels of MCP1, but the test compounds had no effect on this (FIG. 22). Given the overall minimal results in the serum cytokine profile, it might be of more interest to investigate the cytokine milieu directly within the tumor tissue.

### Conclusions

In conclusion, GBX and GCX were both able to boost an anti-tumor immune response for CRC, both, as stand-alone as well as in combination with PD1 inhibition. These effects were mirrored by strong effects on tumor-infiltrating T cells and elevated numbers of anti-tumor CD8 T cells might be responsible for the observed effects.

Overall, this experiment as well as the experiments using melanoma cells, and without being bound by theory suggest the following conclusions. First, a direct impact of GCX on the cancer or immune cells was observed. This would suggest that the compounds can directly interact with either the cancer or the immune cells or both. One must note that the compounds were applied orally and the tumors in both models were located subcutaneously. This makes a direct interaction with the cancer cells somehow unlikely. A potential mechanism of action (MoA) nevertheless might be that the compounds modulate glycosylation pattern of cells in the intestinal mucosa/submucosa which then modulates immune cells.

Secondly, the compounds might modulate the intestinal microbiome and the resulting changes in microbiome composition and metabolic activity might exert the observed antitumor effects. Evidence for such mechanisms comes e.g. from a paper from Montalban-Arques et al. Cell Host&Microbe 2021 or Tanoue et al Nature 2019 or from two human FMT studies published early 2021 in Science (Dawar et al and Baruch et al). One would then anticipate that the compounds induce such an alteration in the intestinal microbiome composition and metabolic activity that the “new” microbiome is able to activate anti-tumor immune cells or inactivate immunoregulatory cells.

### Example 7

In this study, Applicant examined the following:
1—Tested the capacity of GBX or GCX to elicit CD8+ T cells that specifically recognize/cross-react to antigen(s) present in GBX or GCX.
2—Evaluated GBX or GCX priming of the host to recognize such antigens and elicit a memory T cell response.
3—Evaluated whether via molecular mimicry, the T cells elicited by GBX or GCX will recognize/cross-react with cancer antigens.

### Materials & Methods

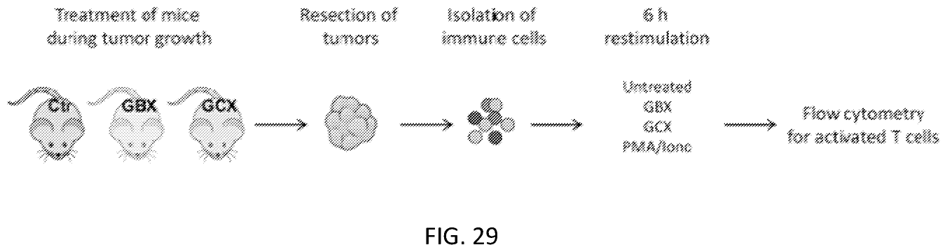

As part of the subcutaneous CRC mouse model, tumor infiltrating immune cells were isolated from the resected tumors originating from control mice (mice not treated with test items), as well as the mice previously treated with GBX or GCX in Example 6 above. Immune cells were then re-stimulated for 6 hours with GBX, GCX and PMA/ionomycin (positive control) or left untreated (negative control). Flow cytometry was used to measure the proportion of activated CD8 T cells using the activation marker CD69. Protocol is illustrated in FIG. 29.

### Results and Discussion

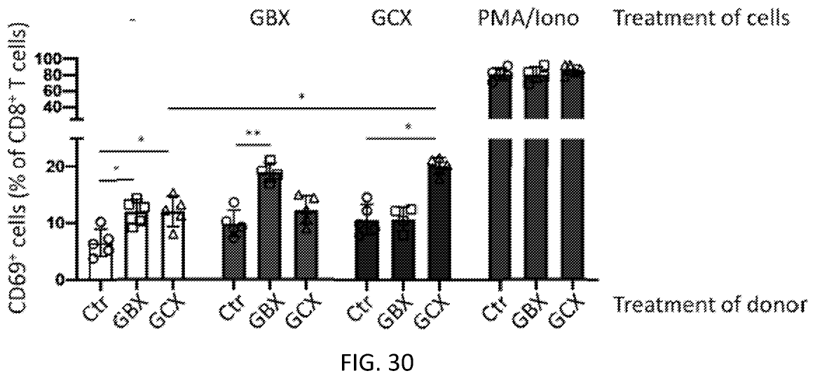

The results are shown in FIG. 30.

CD69 is a biomarker of T-cell activation status. One of the earliest cell surface antigens expressed by T cells following activation. CD69 is also preferentially expressed on cells that activate the memory phenotype.

From the black group (no re-stimulation) in FIG. 30, mice previously treated with GBX or GCX have higher proportion of activated T cells vs untreated mice. Indeed, Applicant observed that activation signals are higher in GBX- or GCX-treated mice vs control. Analyzing only this group cannot inform about the specificity/cross-reactivity of these activated T cells.

From the green and blue group (re-stimulation) in FIG. 30, Applicant observed that mice previously treated with GBX or GCX have higher numbers of activated T cells that specifically recognize/cross-react to the antigen(s) present in GBX or GCX. Indeed, the activation signal upon re-stimulation is stronger in mice previously treated with the same test item vs control.

In conclusion, GBX (GNU201) or GCX (GNU101) treatment induces in the recipient the activation of T cells with specificity/cross-reactivity to the antigen(s) present in GBX or GCX. GBX and GCX have thus the capacity to prime the host to respond to these antigens. Applicant expected the presence of antigens in GBX or GCX mimicking tumor antigens. Thus GBX or GCX induce a memory CD8+ T cell response, which, in turn, may cross-react with cancer antigens.

### Example 8

In this example, Applicant tests the efficacy of GBX and GCX in a therapeutic setting using CRC, and/or melanoma, and/or breast and/or lung mouse models.

Applicant evaluates GBX and GCX efficacy in decreasing tumor burden when GBX and GCX are applied post tumor injection. Applicant also evaluates GBX and GCX efficacy against a broad range of solid tumors, thus demonstrating a widespread anti-tumor mechanism of glycopeptides supplementation therapy. Using a subcutaneous CRC, and/or melanoma, and/or breast and/or lung mouse models, Applicant tests whether GBX and GCX reduce tumor burden when orally administered alone or in combination with ICI several days following tumor injections. The results provide information on (i) optimal treatment conditions (as stand-alone therapeutics or in combination with ICI mAb), (ii) pharmacokinetic and pharmacodynamic properties.

The CRC, melanoma, breast and lung mouse models are well-established. See J. Clin. Invest. 2021; 131(1):e140281. doi: 10.1172/JCI140281./Cell Host & Microbe (2021), 29, 1-16, https://doi.org/10.1016/j.chom.2021.08.001, incorporated herein by reference.

The following models are examined:
2.1: Subcutaneous, heterotopic MC38 cell injection model
2.2: Subcutaneous, orthotopic B16-F1 cell injection model
2.3: Subcutaneous, heterotopic 4T1 cell injection model
2.4: Subcutaneous, heterotopic LLC1.1 cell injection model

### Materials and Methods

### 2.1: Subcutaneous, Heterotopic MC38 Cell Injection Model.

GBX and GCX are used to address (i) efficacy at controlling tumor growth and (ii) efficacy at enhancing ICI therapy after administration of test compound through oral route with food/water. Treatment is performed in a therapeutical way (start of treatment 6 days post tumor cell injection) in absence/presence of ICI mAb. 5-fluorouracil (5-FU) is used as positive control, since 5-FU is a clinically well-established therapy for CRC. Applicant uses anti-PD1 antibody nivolumab as immune checkpoint inhibitor (ICI).

The following animal groups are compared (n=5 mice per group):

Groups
Negative control (control mAb isotype)
Positive control (5-FU)
ICI mAb*
GNU candidate 1 (GBX)
GNU candidate 2 (GCX)
GNU candidate 1 + ICI mAb
GNU candidate 2 + ICI mAb
*Anti-PD1 antibody nivolumab is used as immune checkpoint inhibitor (ICI)

### 2.2: Subcutaneous, Orthotopic B16-F1 Cell Injection Model.

B16-F1 melanoma cells have only very low metastatic potential and are therefore useful to study the impact of the instant compositions on the primary tumor. See, Cancer Growth Metastasis. 2015; 8(Suppl 1): 81-94. doi: 10.4137/CGM.S21214, incorporated herein by reference. The subcutaneous B16-F1 melanoma cell injection model follows the same study design as described above in 2.1.

The following animal groups are compared (n=5 mice per group):

Groups
Negative control (control mAb isotype)
ICI mAb*
GNU candidate 1 (GBX)
GNU candidate 2 (GCX)
GNU candidate 1 + ICI mAb
GNU candidate 2 + ICI mAb
*The anti-PD1 antibody nivolumab is used as immune checkpoint inhibitor (ICI).

### 2.3: Subcutaneous, Heterotopic 4T1 Cell Injection Model.

To validate the effect of the instant compositions in breast cancer mouse model, heterotopic tumour cell injection of 4T1 breast cancer cells is performed, a model that is well established. Treatment is performed to mimic cancer therapy (start of treatment 6 days post tumor cell injection) in absence/presence of ICI mAb. The subcutaneous 4T1 breast cancer cell injection model follows the same study design as described above in 2.1.

The following animal groups are compared (n=5 mice per group):

Groups
Negative control (control mAb isotype)
ICI mAb*
GNU candidate 1 (GBX)
GNU candidate 2 (GCX)
GNU candidate 1 + ICI mAb
GNU candidate 2 + ICI mAb
The anti-PD1 antibody nivolumab is used as immune checkpoint inhibitor (ICI).

### 2.4: Subcutaneous, Heterotopic LLC1.1 Cell Injection Model.

To validate the effect of GBX and GCX in lung cancer mouse model, heterotopic tumour cell injection of LLC1.1 lung cancer cells is performed, a model that is well established. Treatment is performed to mimic cancer therapy (start of treatment 6 days post tumor cell injection) in absence/presence of ICI mAb. The subcutaneous LLC1.1 lung cancer cell injection model follows the same study design as described above in 2.1.

The following animal groups are compared (n=5 mice per group):

Groups
Negative control (control mAb isotype)
ICI mAb*
GNU candidate 1 (GBX)
GNU candidate 2 (GCX)
GNU candidate 1 + ICI mAb
GNU candidate 2 + ICI mAb
The anti-PD1 antibody nivolumab is used as immune checkpoint inhibitor (ICI).

### Materials and Methods for 2.1, 2.2, 2.3 and 2.4:

Conventionally housed C57BL/6 mice are used for all experiments. For all experiments, only littermate breeding is used. Mice are housed under specific pathogen-free conditions with free access to water and food ad libitum. All procedures and experiments are approved by the local animal welfare committees.

GBX and GCX are administered orally (with or without ICI mAb) and compared to negative control (control mAb isotype), positive controls and ICI therapy. The concentration range is selected based on currently available preliminary results. CRC or melanoma tumor cells is suspended in culture medium, mixed 1:1 with Matrigel, and injected subcutaneously into each of the flanks of the C57BL/6 mice. Mice is euthanized 2-3 weeks after injection. At the end of each experiment, tumor and colon tissue are collected for histologic preparation by H&E staining and immunohistochemistry, mRNA and protein level assessment as well as FACS analysis of immune cells and spleen (systemic level, both models). Additionally, mouse plasma/serum are sampled and stored for further analyses.

| | D-14 | D0 | D6 | D18 |
|---|---|---|---|---|
| MAJOR STUDY EVENTS | | | | |
| Acclimatation to | X | | | |
| base diet start | | | | |
| Tumor injection | | X | | |
| Treatment start | | | X | |
| Study end | | | | X |
| COLLECTION | | | | |
| Tumor | | | | X |
| Serum | | X | | X |
| Stool | X | X | | X |
| Cecum & colon | | | | X |
| content | | | | |
| Colon tissue | | | | X |
| Colon lamina | | | | X |
| propria | | | | |
| Spleen | | | | X |
| Lymph nodes | | | | X |
| END-POINTS | | | | |
| Mice healthy status | Daily | | | |
| Tumor volume | | Every 3 days | | |
| Tumor weights | | | | X |
| FC of TILs | | | | X |
| FC of immune cells | | | | X |
| in other sites | | | | |
| Histology | | | | X |
| RT-PCR/WB | | | | X |
| 16S | X | X | | X |
| Cytokine panel | | | | X |
| Untargeted | | X | | X |
| metabolomic | | | | |

### Experimental Read-Outs for 2.1 and 2.2:

### Tumor Volume (Timepoint: Every 3 Days):

Primary endpoint is tumour volume following oral administration of GBX and GCX at compared to negative control (control mAb isotype) and positive control. Tumor development is measured every 3 days using a digital caliper. Tumor volume is calculated using the ellipsoid formula: 4/3*3.14*Length/2*(Width/2)² where the shorter dimension is used as width and depth. Mice are euthanized when the volume reached 1 cm³ or the length reached 2 cm.

### Mice Healthy Status (Timepoint Daily):

Mice are monitored daily with respect to body weight and clinical parameters and as requested per animal welfare conditions.

### Histology (Timepoint: End of Experiment):

Tumor, tumor-adjacent tissue and colon are used for H&E staining and IHC. H&E is performed according to standard procedures. Tissues for IHC are deparaffinized and antigens retrieved using citrate buffer. Ki67, cleaved caspase 3 and activated/cleaved MMP-9 will be analyzed.

### Flow Cytometry Analysis (Timepoint: End of Experiment):

Spleen, lymph nodes, colon lamina propria, and tumor tissue/cells are used for flow cytometry analysis. Single cell suspensions are prepared, stained and re-stimulated.

### Real-Time RT-qPCR and Western Blot Analysis or RNAseq (Timepoint: End of Experiment):

Tumor, tumor-adjacent and colon tissue/cells from mice are used for real-time RT-qPCR and/or Western Blot or RNAseq analysis performed according to standard procedures. The following genes/proteins expression are evaluated.

Barrier function: claudin-1, claudin-2, claudin-4, claudin-8, occludin, ZO-1 and MUC-2.

Cytokine and chemokine: pro-inflammatory: TNF-alpha, IFN-gamma, IL-1alpha, IL-1beta, IL-6, IL-8, GRO-alpha, MCP-1 and the ones classified as anti-inflammatory: IFN-alpha, IL-4, IL-10, TGF-beta-1-3.

### RNAseq: To Assess Effect on Tumor Cells Gene Expression (Timepoint: End of Experiment):

Tumor tissue/cells from mice are used for RNAseq analysis performed according to standard procedures. This analysis provised a comprehensive view about the modulation of the gene expression levels of the tumor cells in response to treatment with the compositions of the disclosure.

TLRs & Microbial recognition: TLR1, TLR2, TLR4, TLR5, TLR7, TLR9.

### Tumor Microenvironment Cytokine: To Assess Inflammatory Status (Timepoint: End of Experiment):

Multiplex ELISA will be performed detecting cytokines and growth factors in the tumor microenvironment of the mice. This analysis provides a comprehensive overview about the modulation of the immune tone of the tumor microenvironment in response to GBX and/or GCX candidate treatment.

### Serum/Cecal Metabolomics: To Assess Indirect Mode of Action of Gnubiotics' Candidate Mediated by the Microbiome Metabolic Activity (Timepoint: At Baseline, at the End of Experiment):

Metabolomics analyses are carried out with a high-resolution mass spectrometer (Agilent QTOF 6550). Each individual sample is normalized by scaling to the average of the log 10 of the intensities of all annotated features. Significance analysis is done by heteroscedastic (two tail, unequal variance) t-test. In addition, p-values are adjusted according to Benjamini-Hochberg, and q-values according to Storey and Tibshirani.

### Microbiota Analysis (16S): To Assess Microbiome Modulatory Activity of Gnubiotics' Candidate (Timepointat Baseline, at the End of Experiment):

Fecal samples are stored at ˜80° C. until further processing. For DNA isolation, ˜0.25 g of feces is processed using the DNeasy® Power Soil® Kit (Qiagen) following the manufacturer's instructions. DNA concentration is determined spectrophotometrically using a NanoDrop ND-1000. A concentration of 20 ng/μl per sample is sequenced. In short, a targeted polymerase chain reaction-based sequencing approach is used, where unique DNA regions of the 16S rRNA gene will be targeted to generate amplicons. Amplicon library is sequenced using MiSeq Illumina platform. Two negative control reactions without the DNA template and two negative control reactions without the amplification primers are included in the run. Fastq files generated by the sequencer are used to de-multiplex and analyze the raw reads, using a publicly available Divisive Amplicon Denoising Algorithm (DADA2) pipeline. Read counts generated by the pipeline are divided by the total sample read count to obtain relative abundances.

Remarks: The measurements of the secondary end-points “Tumor microenvironment cytokine”, “serum/cecal metabolomics”, “Microbiota analysis (16S)” is only executed after completion of an initial analysis of the primary end-point “Tumor volume” to guide the selection of the groups that are analyzed.

### Example 9

Dose Range Finding (DRF) Studies in CRC and/or Melanoma and/or Breast and/or Lung Mouse Models

### Description of Study

Applicant evaluates the lowest dose with an anti-tumor efficacy. Using a subcutaneous CRC and/or melanoma and/or breast and/or lung mouse models, Applicant tests different dosages of GBX or GCX applied within the regular mouse food or water and record (i) anti-tumor efficacy, (ii) anti-tumor immune response and (iii) toxicologic read-outs (e.g. liver and kidney serum parameters, histology).

The following tasks are performed:
3.1: Subcutaneous, heterotopic MC38 cell injection model
3.2: Subcutaneous, orthotopic B16-F1 cell injection model
3.3: Subcutaneous, heterotopic 4T1 cell injection model
3.4: Subcutaneous, heterotopic LLC1.1 cell injection model

### Materials and Methods

### 3.1: Subcutaneous, Heterotopic MC38 Cell Injection Model.

A dose-response curve is generated to address (i) local and systemic toxicity, (ii) efficacy at controlling tumor growth and driving an immune response. Treatment is performed in a therapeutical way (start of treatment 6 days post tumor cell injection) in absence/presence of ICI mAb.

The following animal groups are compared (n=5 mice per group):

Groups
Negative control (control mAb isotype)
Positive control (5-FU)
ICI mAb*
Low dose of GBX or GCX (+ ICI)
Medium dose of GBX or GCX (+ ICI)
High dose of GBX or GCX (+ ICI)
*The anti-PD1 antibody nivolumab is used as immune checkpoint inhibitor (ICI).

### Task 3.2: Subcutaneous, Orthotopic B16-F1Cell Injection Model.

The DRF study with melanoma cells will follow the same study design as described above in 3.1.

The following animal groups are compared (n=5 mice per group):

Groups
Negative control (control mAb isotype)
ICI mAb*
Low dose of GBX or GCX (+ICI)
Medium dose of GBX or GCX (+ICI)
High dose of GBX or GCX (+ICI)
*The anti-PD1 antibody nivolumab is used as immune checkpoint inhibitor (ICI).

### 3.3: Subcutaneous, Heterotopic 4T1 Cell Injection Model

The DRF study with breast tumor cells will follow the same study design as described above in 3.1.

The following animal groups are compared (n=5 mice per group):

Groups
Negative control (control mAb isotype)
ICI mAb*
Low dose of GBX or GCX (+ICI)
Medium dose of GBX or GCX (+ICI)
High dose of GBX or GCX (+ICI)
*The anti-PD1 antibody nivolumab is used as immune checkpoint inhibitor (ICI)

### 3.4: Subcutaneous, Heterotopic LLC1.1 Cell Injection Model

The DRF study with lung tumor cells follows the same study design as described above in 3.1.

The following animal groups are compared (n=5 mice per group):

Groups
Negative control (control mAb isotype)
ICI mAb*
Low dose of GBX or GCX (+ICI)
Medium dose of GBX or GCX (+ICI)
High dose of GBX or GCX (+ICI)
*The anti-PD1 antibody nivolumab is used as immune checkpoint inhibitor (ICI).

Method overview for 3.1, 3.2, 3.3 and 3.4:

The method is the same as in Example 8 except the additional organs lung, liver and kidneys will be collected for safety parameter analysis.

| | | | |
|---|---|---|---|
| D-14 | D0 | D6 | D18 |

MAJOR STUDY EVENTS

| | | | | |
|---|---|---|---|---|
| Acclimatation to | X | | | |
| base diet start | | | | |
| Tumor injection | | X | | |
| Treatment start | | | X | |
| Study end | | | | X |

COLLECTION

| | | |
|---|---|---|
| Tumor | | X |
| Serum | X | X |
| Cecum & colon | | X |
| content | | |
| Colon tissue | | X |
| Colon lamina | | X |
| propria | | |
| Spleen | | X |
| Lymph nodes | | X |
| Organ collection | | X |
| (colon, liver, | | |
| kidney, lung and | | |
| heart) | | |

END-POINTS

| Mice healthy | Daily | | |
|---|---|---|---|
| status | | | |
| Tumor volume | | Every 3 days | |
| Tumor weights | | | X |
| FC of TILs | | | X |
| FC of immune | | | X |
| cells in other | | | |
| sites | | | |
| Safety parameters | | X | X |

Experimental read-outs for 3.1, 3.2, 3.3 and 3.4:
Tumor volume (timepoint: every 3 days): as in Example 8
Mice healthy status (timepoint daily): as in Example 8
Flow cytometry analysis (timepoint: end of experiment): as in Example 8

Safety parameters (timepoint: just before tumor cell injection, at the end of experiment):

Blood samples are taken for differential blood-cell counts, hematologic analysis, serum biochemistry and potential bacterial translocation. Additional indicators are checked, such as spleen-weight index, hepatic enzymes (e.g. ALAT, ASAT and bilirubin), kidney values (e.g. serum creatinine and urea), serum lactate and serum glucose. Histology is performed from colon, liver, kidney, lung and heart at the end of the experiment.

### Example 10

Test Tumor Eradication and Immune Memory in CRC and/or Melanoma and/or Breast and/or Lung Mouse Models

### Description of Study

Having validated the potency of GBX and GCX in the control of tumor growth in previous studies, this study aims to demonstrate significant enhanced overall survival and complete tumor regression, defined as no or negligible detectable tumors at the end of the study period in mice treated with the lead candidate. Using a subcutaneous CRC and/or melanoma and/or breast and/or lung mouse models, Applicant tests GBX and GCX applied within the regular mouse food or water and record tumor growth until study termination criteria are reached (e.g., tumor volume, body weight loss, health status). Mice with complete regression are rechallenged with tumor cells to demonstrate the development of immunologic memory.

The following are performed:
4.1: Tumor eradication and survival
4.2: Rechallenge of mice with complete regression
4.3: Rechallenge of mice receiving allogeneic transplant

### Description of Activities

### 4.1: Tumor Eradication and Survival.

Survival and tumor eradication are assessed in mice receiving the anti-tumor treatment. Treatment is performed in a therapeutical way (start of treatment 6 days post tumor cell injection) in absence/presence of ICI mAb.

The following animal groups are compared (n=15 mice per group):

Groups
Negative control (control mAb isotype)
GBX or GCX (+ ICI)
* The anti-PD1 antibody nivolumab is used as immune checkpoint inhibitor (ICI).

4.2: Rechallenge of Mice with Complete Regression.

For the tumor rechallenge study, mice that were tumor-free after treatment in 4.1 receive an injection of tumor cells 14 days after the last treatment day. These mice are compared to tumor-naive and treatment-naive control mice and to tumor-naïve mice that had previously received the treatment. The number of mice included into the rechallenge studies is dependent on the number of complete responders for each individual task (control mice n=10).

Groups

Tumor-naive and treatment-naive mice
(negative control) − n = 10
Tumor-naive mice that had previously
received the treatment
(negative control) − n = 10
Mice with complete regression

### 4.3: Rechallenge of Mice Receiving Allogeneic Transplant.

In this rechallenge study, only RAG−/− mice are used (do not contain mature B and T lymphocytes). Tumor-naive and treatment-naive mice receive an allogeneic transplant of CD8 T cells isolated from tumors and lymph nodes from responding mice. Control groups consist in tumor-naive and treatment-naive control mice and tumor-naive and treatment-naive control mice receiving CD8 T cells isolated from tumors and lymph nodes from treatment naive mice.

Groups

Tumor-naive and treatment-naive mice
(negative control) − n = 10
Tumor-naive and treatment-naive mice + CD8 T cells
from treatment-naive mice (negative control) − n = 10
Tumor-naive and treatment-naive mice + CD8 T
cells isolated from responding mice − n = 10

### Experimental Read-Outs for 4.1, 4.2 and 4.3:

Kaplan-Meier survival curve (timepoint: every day): the effect of the treatment is assessed by measuring the number of subjects survived or saved after the treatment over a period of time.
Tumor volume (timepoint: every 3 days): as in Example 8
Mice healthy status (timepoint daily): as in Example 8

### Example 11

Evaluate the effect of lead candidate in orthotopic and metastatic CRC mouse models.

### Description of Study

The aim of this study is to test GBX or GCX efficacy in a relevant environment and evaluate efficacy in preclinical tumor models that mimic the disease process in humans and specific clinical situations. Therefore, Applicant focuses in in this study on a cecum injection mouse model and on a model developing metastases of CRC.

Applicant tests the effect of GBX or GCX applied in regular mouse food or water with the appropriate dose concentration determined in the DRF studies of Example 9 in the following well-established mouse model of CRC: (1) orthotopic MC38 cell injection model in the cecum, and (2) intrasplenic MC-38 tumour cell injection representing a model for metastasizing colon tumours.

The following tasks are performed:
5.1: Orthotopic MC38 cell injection model in the cecum
5.2: Intrasplenic MC-38 tumour cell injection representing a model for metastasizing colon tumours

### Materials and Methods

### 5.1: Orthotopic MC38 Cell Injection Model in the Cecum.

Here, Applicant uses the orthotopic MC38 cell injection model in the cecum. The advantage of this model is that the colon tumors are located in situ in their conventional environment. This is important with respect to the clinical application of GBX and GCX in human patients later on (clinical phase). The cecum injection model follows the same study design and approach as in 2.1 above. Treatment is performed in a therapeutical way (start of treatment 6 days post tumor cell injection) in absence/presence of ICI mAb.

The following animal groups are compared (n=5 mice per group):

Groups

Negative control (control mAb isotype)
Positive control (5-FU)
ICI* alone
GBX or GCX
(best dose from the DRF study of Example 9)
GBX or GCX + 5-FU
GBX or GCX + ICI*

*The anti-PD1 antibody nivolumab is used as immune checkpoint inhibitor (ICI).

### 5.2: Intrasplenic MC-38 Tumour Cell Injection Representing a Model for Metastasizing Colon Tumours

To validate the effect of GBX or GCX in a stage IV colon cancer mouse model, the intrasplenic tumour cell injection model is performed using luciferase-tagged murine MC-38 colon adenocarcinoma cells derived from C57/B6 background. For applying this model, 5×10⁵ MC-38 cells are injected into the spleen (or the tail vein, both possible). Treatment is performed in a therapeutical way (start of treatment 6 days post tumor cell injection) in absence/presence of ICI mAb. The MC38 cell injection CRC model is well established.

The following animal groups are compared (n=5 mice per group):

Groups

Negative control (control mAb isotype)
Positive control (5-FU)
ICI* alone
GBX or GCX
(best dose from the DRF study Example 9)
GBX or GCX + 5-FU
GBX or GCX + ICI*

*The anti-PD1 antibody nivolumab is used as immune checkpoint inhibitor (ICI).

### Methods and Experimental Read-Outs for 5.1 and 5.2:

At the end of the experiment, mice are sacrificed, and tumour development will be studied by histological approaches. Tumour tissue as well as tumour adjacent tissue, liver, lung, kidney, spleen, peritoneum and mesenterial lymph nodes are analysed by histology (H&E staining and specific IHC) and collected for mRNA and protein analysis as well as for FACS analysis of immune cells. Luciferase-tagged MC-38 cells in primary tumour and tumour surrounding tissue as well as in liver metastases are studied by fluorescence imaging approaches. Tumour and non-tumour tissue will be collected separately whenever possible and stored for further studies.

Furthermore, FACS analysis is performed on immune cells from the tumor, spleen, lymph nodes, colon lamina propria. Transcriptomic analysis is done using tumor tissue/cells and colonic cells. Finally, assessment of cytokine levels in the tumor microenvironment and metabolite profiles in the serum and cecum are also done.

### Example 12

### Evaluate Anti-Tumor Response in Additional Mouse Models of Solid Tumors

### Materials and Methods

Applicant evaluates the anti-tumor efficacy of GBX or GCX in additional solid tumors, thus validating the compounds and compositions as therapeutic options not only for melanoma and CRC, but also for a broad range of solid tumors. A widespread anti-tumor mechanism of glycopeptides is evaluated as supplementation therapy. Applicant tests GBX or GCX with the appropriate dose concentration determined in the DRF studies of Example 9 in preclinical mouse models of lung and breast.

The following are performed:
6.1: Subcutaneous, heterotopic 4T1 cell injection model
6.2: Subcutaneous, heterotopic LLC1.1 cell injection model

### Materials and Methods

6.1: Subcutaneous, heterotopic 4T1 cell injection model.

To validate the effect of GBX or GCX in breast cancer mouse model, heterotopic tumour cell injection of 4T1 breast cancer cells is performed, a model that is well established. Treatment is performed in a therapeutical way (start of treatment 6 days post tumor cell injection) in absence/presence of ICI mAb.

The following animal groups are compared (n=6 mice per group):

Groups

Negative control (control mAb isotype)
ICI* alone
GBX or GCX
(best dose from the DRF study of Example 9)
GBX or GCX + ICI*

*The anti-PD1 antibody nivolumab is used as immune checkpoint inhibitor (ICI).

### 6.2: Subcutaneous, Heterotopic LLC1.1 Cell Injection Model.

To validate the effect of GBX or GCX in lung cancer mouse model, heterotopic tumour cell injection of LLC1.1 lung cancer cells are performed, a model that is well established. Treatment is performed in a therapeutical way (start of treatment 6 days post tumor cell injection) in absence/presence of ICI mAb.

The following animal groups are compared (n=6 mice per group):

Groups
Negative control (control mAb isotype)
ICI* alone
GBX or GCX (best dose from the
DRF study in Example 9)
GBX or GCX + ICI*
*The anti-PD1 antibody nivolumab is used as immune checkpoint inhibitor (ICI)

### Experimental Read-Outs for 6.1 and 6.2:

Tumor volume (timepoint: every 3 days): as in Example 8
Mice healthy status (timepoint daily): as in Example 8
Flow cytometry analysis (timepoint: end of experiment): as in Example 8.

### Example 13: O-Glycan core structures in GBX and GCX compositions

FIG. 31A shows different core structures (Core 1-5 and N) of glycans found in the compositions of the disclosure. FIG. 31B and the table below shows the comparison of the distribution of core structures between two GCX and one GBX batches as measured by liquid chromatograph-electrospray ionization tandem mass spectrometry (LC-ESI/MS).

| | | |
|---|---|---|
| Core 1 | GCX-B1 | 27.10% |
| | GCX-B2 | 29% |
| | GBX | 24.00% |
| Core 2 | GCX-B1 | 42.60% |
| | GCX-B2 | 34% |
| | GBX | 34.30% |
| Core 3 | GCX-B1 | 10% |
| | GCX-B2 | 11% |
| | GBX | 11.30% |
| Core 4 | GCX-B1 | 13.20% |
| | GCX-B2 | 16.50% |
| | GBX | 0.10% |
| Core 5 | GCX-B1 | 4.00% |
| | GCX-B2 | 3.20% |
| | GBX | 0% |
| Core N | GCX-B1 | 0.20% |
| | GCX-B2 | 3.60% |
| | GBX | 0.70% |

It was observed that, a majority (about 70-80%) of the olgisaccharides/glycans in the compositions had Core 1 or Core 2 structure. About 10% to 20% of the glycans in the compositions had Core 3 structure. Interestingly, Core 4 and Core 5 structures were enriched only in GCX compositions and were virtually absent from GBX. GBX composition was also very low on Core N structures.

### Materials and Methods: O-glycan Analysis

The glycans were desalted using 1 ml Dowex (AG50W-X8), which were packed to a SPE cartridge (Strata X), pre-washed with 2×1 ml methanol, 2×1 ml 1 M HCl and 2×1 ml water. After applying the sample, both the flowthrough and 3×0.5 ml water washout were selected and freeze-dried. The borate was removed by evaporation with 5×200 ul methanol.

O-glycans were released by beta-elimination. Two different ways to remove salts were used: dialysis and Dowex ion-exchange chromatography. Dialysis result was only to give an estimate amount of released glycans.

After desalting, the sample was re-constituted in 50 ul of water and analyzed by LC-MS/MS. Glycans were analysed by liquid chromatograph-electrospray ionization tandem mass spectrometry (LC-ESI/MS). The oligosaccharides were separated on a column (10 cm×250 μm) packed in-house with 5 μm porous graphite particles (Hypercarb, Thermo-Hypersil, Runcorn, UK). The oligosaccharides were injected on to the column and eluted with an acetonitrile gradient (Buffer A, 10 mM ammonium bicarbonate; Buffer B, 10 mM ammonium bicarbonate in 80% acetonitrile). The gradient (0-45% Buffer B) was eluted for 46 min, followed by a wash step with 100% Buffer B, and equilibrated with Buffer A in next 24 min. A 40 cm×50 μm i.d. fused silica capillary was used as transfer line to the ion source.

Dowex right after beta-elimination without dialysis. Dowex AG50W-X8 is a cation exchange resin which is used to remove sodium in the reaction buffer. The borate is removed by repetitive evaporation with methanol. PGM O-glycans are most neutral and some negatively charged. So, they do not bind to Dowex.

The samples were analyzed in negative ion mode on a LTQ linear ion trap mass spectrometer (Thermo Electron, San Jose, CA), with an IonMax standard ESI source equipped with a stainless steel needle kept at −3.5 kV. Compressed air was used as nebulizer gas. The heated capillary was kept at 270° C., and the capillary voltage was −50 kV. In order to reach the number of glycans (>100), two mass ranges (m/z 380-1190 and m/z 1190-2000) were performed (two microscan, maximum 100 ms, target value of 30,000) followed by data-dependent MS2 scans (two microscans, maximum 100 ms, target value of 10,000) with normalized collision energy of 35%, isolation window of 2.5 units, activation q=0.25 and activation time 30 ms). The threshold for MS2 was set to 200 counts. Data acquisition and processing were conducted with Xcalibur software (Version 2.0.7). The LC-MS/MS data was processed using Progenesis QI (Nonlinear Dynamics, Waters).

### EQUIVALENTS

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control. Other embodiments are set forth within the following claims.

1. A method for one or more of in a subject in need thereof:
treating a tumor in a subject who has been diagnosed with cancer;
enhancing an anti-tumor activity of a CAR-T therapy;
enhancing an Immune Checkpoint Inhibitor (ICI) therapy against a tumor;
or cancer prophylaxis,
the method comprising administering to the subject an effective amount of a composition comprising glycopeptides obtained from gastrointestinal mucins, wherein the composition comprises less than about 25% (w/w) free glycans, and wherein the total protein content of the composition is 12% or less (w/w), and wherein the composition comprises glycopeptides of multiple different oligosaccharide structures, and wherein the composition is obtained from porcine intestinal mucins or a partially purified fraction thereof, wherein:
a) the composition is obtained without subjecting the mucins or the partially purified fraction thereof to conditions or reagents that release oligosaccharides from glycopeptides:
b) the composition has a total glycan content of greater than 10%;
c) the composition comprises glycopeptide-bound oligosaccharides having each of the following general formulae:
i. Hex1HexNAc1
ii. HexNAc2
iii. NeuAc1HexNAc1
iv. NeuGc1HexNAc1
v. Hex1HexNAc1Fuc1
vi. Hex1HexNAc2
vii. Hex1HexNAc2Sul1
viii. NeuAc1Hex1HexNAc1
ix. NeuGc1Hex1HexNAc1
x. NeuAc1HexNAc2
xi. NeuGc1HexNAc2
xii. Hex1HexNAc2Fuc1
xiii. Hex1HexNAc2Fuc1Sul1
xiv. NeuAc1Hex1HexNAc1Fuc1
xv. Hex1HexNAc3Sul1
xvi. Hex2HexNAc2Fuc1
xvii. Hex1HexNAc3Fuc1Sul1
xviii. Hex2HexNAc2Fuc2Sul1, and
the composition does not substantially contain insoluble particles having a diameter greater than 7 μm or substances having a molecular weight of greater than 3 kDa.

2. (canceled)

3. The method of claim 1, wherein the tumor is refractory to immune checkpoint inhibitor therapy.

4.-5. (canceled)

6. The method of claim 1, wherein the composition comprises at least one sialylated glycopeptide-bound oligosaccharide, or
wherein the composition comprises at least 1, 2, 3, 4, 5, 6, 7, 8, 9, or all 10 sialylated glycopeptide-bound oligosaccharide selected from the following a) through k):
a) NeuAcα2-6GalNAc
b) NeuGcα2-6GalNAc
c) Galβ1-3(NeuAcα2-6)GalNAc
d) NeuAcα2-3Galβ1-3GalNAc
e) Galβ1-3(NeuGcα2-6)GalNAc
f) NeuGcα2-3Galβ1-3GalNAc
g) GlcNAc-(NeuAcα2-6)GalNAc
i) GalNAc-(NeuAcα2-6)GalNAc
j) HexNAc-(NeuGcα2-6)GalNAc
k) Fucα1-2Galβ1-3(NeuAcβ2-6)GalNAc,
or wherein the composition comprises NeuAcα2-3Galβ1-3GalNAc (Sialyl-T antigen), NeuAcα2-6GalNAc (Sialyl Tn antigen), and NeuAcα2-3Galβ1-3(NeuAcα2-6)GalNAc (Disialyl T antigen).

7. (canceled)

8. The method of claim 1, further comprising administering an immune checkpoint inhibitor before, after, or simultaneously with the composition comprising glycopeptides, wherein the composition is administered in an effective amount to reduce or prevent a rise in the level of Monocyte Chemoattractant Protein-1 (MCP-1) caused by the immune checkpoint inhibitor in a tumor micro-environment (TME) and/or in systemic circulation.

9.-10. (canceled)

11. The method of claim 1, wherein the composition comprises at least 30 glycopeptide-bound oligosaccharide having a general formulae selected from Hex1HexNAc1, HexNAc2, Hex1HexNAc1Sul1, HexNAc1deHex1Sul1, HexNAc2Sul1, NeuAc1HexNAc1, Hex2HexNAc1, Hex1HexNAc1deHex1, Hex1HexNAc2, HexNAc2deHex1, Hex1HexNAc1deHex1Sul1, Hex1HexNAcSul1, Hex2HexNAc1deHex1, NeuAc1Hex1HexNAc1, Hex1HexNAc1deHex2, Hex2HexNAc2, Hex1HexNAc2deHex1, Hex2HexNAc1deHex1Sul1, Hex1HexNAc1deHex2Sul1, Hex1HexNAc3, Hex1HexNAc2deHex1Sul1, NeuAc1Hex1HexNAc1deHex1, Hex2HexNAc2Sul1, NeuAc1Hex1HexNAc2, Hex1HexNAc3Sul1, Hex2HexNAc2deHex1, Hex1HexNAc2deHex2, Hex1HexNAc3deHex1, Hex2HexNAc3, Hex2HexNAc2deHex1Sul1, Hex1HexNAc4, Hex1HexNAc3deHex1Sul1, Hex3HexNAc2deHex1, NeuAc1Hex2HexNAc2, Hex2HexNAc2deHex2, Hex2HexNAc3deHex1, Hex3HexNAc2deHex1Sul1, Hex2HexNAc2deHex2Sul1, Hex2HexNAc4, Hex2HexNAc3deHex1Sul1, Hex3HexNAc3deHex1, Hex2HexNAc4deHex1, Hex3HexNAc3deHex1Sul1, and Hex4HexNAc3deHex1Sul1; or
wherein the composition comprises at least 30 glycopeptide-bound oligosaccharide having a general formulae selected from Hex1HexNAc1, Hex1HexNAc1deHex1, Hex1HexNAc1deHex1Sul1, Hex1HexNAc1deHex2, Hex1HexNAc1deHex2Sul1, Hex1HexNAc1Sul1, Hex1HexNAc2, Hex1HexNAc2deHex1, Hex1HexNAc2deHex1Sul1, Hex1HexNAc2deHex2, Hex1HexNAc2Sul1, Hex1HexNAc3, Hex1HexNAc3deHex1, Hex1HexNAc3deHex1Sul1, Hex1HexNAc3Sul1, Hex1HexNAc4, Hex1HexNAcSul1, Hex2HexNAc1, Hex2HexNAc1deHex1, Hex2HexNAc1deHex1Sul1, Hex2HexNAc2, Hex2HexNAc2deHex1, Hex2HexNAc2deHex1Sul1, Hex2HexNAc2deHex2, Hex2HexNAc2deHex2Sul1, Hex2HexNAc2Sul1, Hex2HexNAc3, Hex2HexNAc3deHex1, Hex2HexNAc3deHex1Sul1, Hex2HexNAc4, Hex2HexNAc4deHex1, Hex3HexNAc2deHex1, Hex3HexNAc2deHex1Sul1, Hex3HexNAc3deHex1, Hex3HexNAc3deHex1Sul1, Hex4HexNAc3deHex1Sul1, HexNAc1deHex1Sul1, HexNAc2, HexNAc2deHex1, HexNAc2Sul1, NeuAc1Hex1HexNAc1, NeuAc1Hex1HexNAc1deHex1, NeuAc1Hex1HexNAc2, NeuAc1Hex2HexNAc2, and NeuAc1HexNAc1; or
wherein the composition comprises at least 30 glycopeptide-bound oligosaccharide having a general formulae selected from Hex1HexNAc1, HexNAc2, Hex1HexNAc1Sul, Hex1NAc2Sul1, NeuAc1HexNAc1, NeuGc1HexNAc1, Hex1HexNAc1deHex1, Hex2HexNAc1, Hex1HexNAc2, HexNAc3, Hex1HexNAc2Sul1, NeuAc1Hex1HexNAc1, NeuGc1Hex1HexNAc1, Hex2HexNAc1deHex1, Hex1NAc3Sul1, NeuAc1HexNAc2, NeuGc1HexNAc2, Hex1HexNAc2deHex1, Hex2HexNAc2, Hex1HexNAc3, Hex1HexNAc2deHex1Sul1, NeuAc1Hex1HexNAc1deHex1, Hex2HexNAcSul1, NeuGc1Hex1HexNAc1deHex1, Hex1HexNAc3Sul1, NeuAc1Hex1HexNAc2, NeuGc1Hex1HexNAc2, Hex2HexNAc2deHex1, Hex3HexNAc2, Hex1HexNAc3deHex1, Hex2HexNAc3, NeuAc1Hex1HexNAc2Sul1, NeuAc2Hex1HexNAc1, NeuGc1Hex1HexNAc2Sul1, Hex2HexNAc2deHex1Sul1, NeuAc1NeuGc1Hex1HexNAc1, NeuGc2Hex1HexNAc1, Hex1HexNAc3deHex1Sul1, NeuAc1Hex1HexNAc2deHex1, Hex2HexNAc3Sul1, NeuGc1Hex1HexNAc2deHex1, NeuAc1Hex2HexNAc2, Hex2HexNAc2deHex2, NeuGc1Hex2HexNAc2, Hex2HexNAc3deHex1, NeuAc1Hex2HexNAc2Sul1, Hex2HexNAc2deHex2Sul1, NeuGc1Hex2HexNAc2Sul1, Hex1HexNAc4deHex1, NeuAc1Hex1HexNAc3Sul1, Hex1HexNAc3deHex2Sul1, NeuAc2Hex1HexNAc2, NeuGc1Hex1HexNAc3Sul1, Hex2HexNAc3deHex1Sul1, NeuAc1Hex2HexNAc2deHex1, NeuGc2Hex1HexNAc2, NeuGc1Hex2HexNAc2deHex1, NeuGc1Hex3HexNAc2, Hex1HexNAc4deHex1 Sul1, NeuAc1Hex1HexNAc3deHex1, Hex2HexNAc3deHex2 NeuGc1Hex2HexNAc3, NeuAc1Hex2HexNAc2deHex1Sul1, Hex2HexNAc3deHex2Sul1, NeuAc1Hex2HexNAc3deHex1Sul1, NeuGc1Hex2HexNAc3deHex1Sul1, Hex2HexNAc4deHex2Sul1, Hex2HexNAc5deHex2Sul1, NeuAc1Hex5HexNAc4deHex1, and NeuAc1Hex4HexNAc4deHex2Sul1.

12.-14. (canceled)

15. The method of claim 1, wherein the composition comprises Galβ1-3GalNAcol (T antigen) and NeuAcα2-3Galβ1-3GalNAcol (Sialyl T antigen), or wherein the composition comprises Galβ1-3(Fucα1-4)GlcNAcβ1-3Galβ1-3GalNAcol (Lewis A) and Galβ1-4(Fucα1-3)GlcNAcβ1-3Galβ1-3GalNAcol (Lewis X).

16.-17. (canceled)

18. The method of claim 11, wherein the composition has a salt content of less than about 2%, or wherein the composition has a pH of less than 7.5, or wherein the composition has a free glycan content of less than 0.1% by weight.

19.-21. (canceled)

22. The method of claim 1, wherein the composition comprises glycopeptide-bound oligosaccharides having each of the following general formulae: Hex2HexNAc3deHex1Sul2, Hex2HexNAc3deHex1Sul2, NeuGc1Hex2HexNAc3, NeuAc1Hex2HexNAc2deHex1Sul1, Hex2HexNAc4Sul2, Hex2HexNAc4Sul2, NeuAc1Hex2HexNAc3Sul1, Hex2HexNAc3deHex2Sul1, Hex2HexNAc3deHex2Sul1, Hex2HexNAc3deHex2Sul1, Hex2HexNAc3deHex2sul1, Hex2HexNAc3deHex1Sul3, NeuGc1Hex2HexNAc3Sul1, NeuGc1Hex2HexNAc3Sul1, Hex2HexNAc3deHex2Sul2, Hex2HexNAc3deHex2Sul2, Hex2HexNAc3deHex2Sul2, Hex2HexNAc3deHex2Sul2, NeuGc1Hex2HexNAc3deHex1, NeuGc1Hex2HexNAc3Sul2, Hex3HexNAc3deHex1Sul2, NeuGc1Hex2HexNAc2deHex2Sul1, Hex2HexNAc4deHex1Sul2, NeuAc1Hex2HexNAc3deHex1Sul1, Hex2HexNAc3deHex3 Sul1, NeuGc1Hex2HexNAc3deHex1Sul1, NeuGc1Hex2HexNAc3deHex1Sul1, Hex3HexNAc3deHex2Sul1, NeuGc1Hex2HexNAc2deHex2Sul2, NeuGc1Hex3HexNAc2deHex1Sul2, Hex2HexNAc4deHex2Sul1, Hex2HexNAc4deHex2Sul1, Hex2HexNAc4deHex2Sul1, NeuGc1Hex2HexNAc4Sul1, Hex3Hex4deHex1Sul1, Hex2HexNAc3deHex3 Sul2, Hex2HexNAc3deHex3 Sul2, NeuGc1Hex2HexNAc3deHex1Sul2, NeuGc1Hex2HexNAc3deHex1Sul2, NeuGc1Hex2HexNAc3deHex1Sul2, NeuGc1Hex3HexNAc2deHex2Sul1, NeuAc1Hex2HexNAc4deHex1, Hex2HexNAc4deHex2Sul2, Hex2HexNAc4deHex2Sul2, NeuGc1Hex2HexNAc4deHex1, Hex2HexNAc3deHex4Sul1, Hex3HexNAc4deHex1Sul2, NeuGc1Hex2HexNAc3deHex2Sul1, NeuGc1Hex2HexNAc3deHex2Sul1, NeuGc1Hex3HexNAc3deHex1Sul1, NeuGc1Hex2HexNAc2deHex3 Sul2, Hex2HexNAc4deHex3 Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc4deHex1Sul1, NeuGc1Hex2HexNAc3deHex2Sul2, Hex3HexNAc3deHex3 Sul2, NeuGc1Hex3HexNAc3deHex1Sul2, Hex2HexNAc5deHex2Sul1, Hex2HexNAc4deHex3Sul2, NeuGc1Hex2HexNAc4deHex2Sul1, NeuGc1Hex2HexNAc4deHex2Sul1, NeuGc1Hex3HexNAc4deHex1Sul1, Hex2HexNAc5deHex3 Sul1, NeuGc1Hex2HexNAc4deHex2Sul2, NeuGc1Hex2HexNAc4deHex2Sul2, NeuGc1Hex2HexNAc4deHex2Sul2, NeuGc1Hex3HexNAc4deHex1Sul2, Hex3HexNAc4deHex4Sul2, NeuAc1Hex5HexNAc4deHex1, NeuAc1Hex4HexNAc4deHex2Sul1, NeuGc1Hex3HexNAc4deHex4Sul1, NeuGc1Hex4HexNAc4deHex3 Sul1, and NeuGc1Hex3HexNAc4deHex4Sul2.

23. (canceled)

24. The method of claim 1, wherein the sialic acid content of the composition is greater than 25% and less than 50%.

25.-34. (canceled)

35. The method of claim 1, wherein the composition is administered daily for 28 days or more, or wherein the composition is administered in an amount effective to increase one or more of Akkermansia spp., Butyricicoccus spp., Clostridium spp., or Parabacteroides spp. in the gut of the subject.

36. (canceled)

37. The method of claim 1, wherein the subject has stage 3 or 4 cancer selected from melanoma, colorectal cancer (CRC), breast cancer or lung cancer.

38.-39. (canceled)

40. A method for treating a tumor in a subject who has been diagnosed with cancer comprising administering to the subject an effective amount of a composition comprising glycopeptides produced by a method comprising:
(a) dissolving gastrointestinal mucin from stomach of an animal in water comprising calcium hydroxide;
(b) adding diatomaceous earth to (a) and filtering the resulting solution;
(c) adding a cationic substance to (b); and
(d) filtering and concentrating the solution from (c), thereby producing the composition.

41. The method of claim 40, further comprising adjusting the pH of the solution in (b) using carbon dioxide.

42. The method of claim 40, wherein the dissolving was achieved at about 60° C., or wherein the cationic substance comprises an ion exchange hydrogen form resin.

43. (canceled)

44. A method for treating a tumor in a subject who has been diagnosed with cancer comprising administering to the subject an effective amount of a composition comprising glycopeptides produced by a method comprising:
(a) stabilizing gastrointestinal mucin at pH 5.0;
(b) desalinating the stabilized mucin using dialysis;
(c) concentrating the desalinated mucin;
(d) subjecting the concentrate from (d) to diafiltration; thereby producing the composition.

45. The method of claim 44, wherein the concentrating in (c) is achieved by evaporation with a rotary evaporator at least 80° C., or
wherein the effective amount of the composition is about 0.2-0.8 grams per kilogram of the subject.

46.-47. (canceled)

48. A composition comprising glycopeptides produced by a method comprising:
(a) stabilizing gastrointestinal mucin at pH 5.0;
(b) desalinating the stabilized mucin using dialysis;
(c) concentrating the desalinated mucin;
(d) subjecting the concentrate from (d) to diafiltration; thereby producing the composition.

49. The composition of claim 48, wherein the concentrating in (c) is achieved by evaporation with a rotary evaporator at least 80° C., or
wherein the effective amount of the composition is about 0.2-0.8 grams per kilogram of the subject.

50.-51. (canceled)

52. A composition comprising at least 10 glycopeptide-bound oligosaccharide having a general formulae selected from Hex1HexNAc1, HexNAc2, Hex1HexNAc1Sul, Hex1NAc2Sul1, NeuAc1HexNAc1, NeuGc1HexNAc1, Hex1HexNAc1deHex1, Hex2HexNAc1, Hex1HexNAc2, HexNAc3, Hex1HexNAc2Sul1, NeuAc1Hex1HexNAc1, NeuGc1Hex1HexNAc1, Hex2HexNAc1deHex1, Hex1NAc3Sul1, NeuAc1HexNAc2, NeuGc1HexNAc2, Hex1HexNAc2deHex1, Hex2HexNAc2, Hex1HexNAc3, Hex1HexNAc2deHex1Sul1, NeuAc1Hex1HexNAc1deHex1, Hex2HexNAcSul1, NeuGc1Hex1HexNAc1deHex1, Hex1HexNAc3Sul1, NeuAc1Hex1HexNAc2, NeuGc1Hex1HexNAc2, Hex2HexNAc2deHex1, Hex3HexNAc2, Hex1HexNAc3deHex1, Hex2HexNAc3, NeuAc1Hex1HexNAc2Sul1, NeuAc2Hex1HexNAc1, NeuGc1Hex1HexNAc2Sul1, Hex2HexNAc2deHex1Sul1, NeuAc1NeuGc1Hex1HexNAc1, NeuGc2Hex1HexNAc1, Hex1HexNAc3deHex1Sul1, NeuAc1Hex1HexNAc2deHex1, Hex2HexNAc3Sul1, NeuGc1Hex1HexNAc2deHex1, NeuAc1Hex2HexNAc2, Hex2HexNAc2deHex2, NeuGc1Hex2HexNAc2, Hex2HexNAc3deHex1, NeuAc1Hex2HexNAc2Sul1, Hex2HexNAc2deHex2Sul1, NeuGc1Hex2HexNAc2Sul1, Hex1HexNAc4deHex1, NeuAc1Hex1HexNAc3Sul1, Hex1HexNAc3deHex2Sul1, NeuAc2Hex1HexNAc2, NeuGc1Hex1HexNAc3Sul1, Hex2HexNAc3deHex1Sul1, NeuAc1Hex2HexNAc2deHex1, NeuGc2Hex1HexNAc2, NeuGc1Hex2HexNAc2deHex1, NeuGc1Hex3HexNAc2, Hex1HexNAc4deHex1 Sul1, NeuAc1Hex1HexNAc3deHex1, Hex2HexNAc3deHex2 NeuGc1Hex2HexNAc3, NeuAc1Hex2HexNAc2deHex1Sul1, Hex2HexNAc3deHex2Sul1, NeuAc1Hex2HexNAc3deHex1Sul1, NeuGc1Hex2HexNAc3deHex1Sul1, Hex2HexNAc4deHex2Sul1, Hex2HexNAc5deHex2Sul1, NeuAc1Hex5HexNAc4deHex1, and NeuAc1Hex4HexNAc4deHex2Sul1, and
a checkpoint inhibitor; or
a composition comprising at least 10 glycopeptide-bound oligosaccharides having a structure selected from Galβ1-3GalNAc, GalNAcα1-3GalNAc, GlcNAcβ1-6GalNAc, 3SGalβ1-3GalNAc, 6SGlcNAcβ1-3GalNAc, 6SGlcNAcβ1-6GalNAc, NeuAcα2-6GalNAc, NeuGcα2-6GalNAc, Fucα1-2(GalNAcα1-3)Gal, Fucα1-2Galβ1-3GalNAc, Fucα1-2Galβ1-4GlcNAc, Galβ1-4GlcNAcβ1-3Gal, Galβ1-3(GlcNAcβ1-6)GalNAc, Galβ1-4GlcNAcβ1-3GalNAc, GlcNAcβ1-3(GlcNAcβ1-6)GalNAc, Galβ1-3(6SGlcNAcβ1-6)GalNAc, Galβ1-3(NeuAcα2-6)GalNAc, NeuAcα2-3Galβ1-3GalNAc, Galβ1-3(NeuGcα2-6)GalNAc, NeuGcα2-3Galβ1-3GalNAc, Fucα1-2Galβ-4GlcNAcβ1-3Gal, 6SGlcNAcβ1-3(GlcNAcβ1-6)GalNAc, GlcNAcβ1-3(6S-GlcNAcβ1-6)GalNAc, GalNAcα1-3(NeuAcα2-6)GalNAc, GlcNAcβ1-3(NeuAcα2-6)GalNAc, GalNAcα1-3(NeuGcα2-6)GalNAc, GlcNAcβ1-3(NeuGcα2-6)GalNAc, Fucα1-2(GalNAcα1-3)Galβ1-3GalNAc, Fucα1-2Galβ1-3(GlcNAcβ1-6)GalNAc, Fucα1-2Galβ1-3GlcNAcβ1-3GalNAc, Galβ1-3(Galβ1-4GlcNAcβ1-6)GalNAc, Galβ1-3GlcNAcβ1-3Galβ1-3GalNAc, GlcNAcβ1-3(Galβ1-4GlcNAcβ1-6)GalNAc, Fucα1-2Galβ1-3(6S-GlcNAcβ1-6)GalNAc, Fucα1-2Galβ1-3(NeuAcα2-6)GalNAc, Galβ1-3[Galβ1-4(6S)GlcNAcβ1-6]GalNAc, Fucα1-2Galβ1-3(NeuGcα2-6)GalNAc, Galβ1-4GlcNAcβ1-3(6SGlcNAcβ1-6)GalNAc, GlcNAcα1-4Galβ1-3[(6S)GlcNAcβ1-6]GalNAc, GlcNAcβ1-3[Galβ1-4(6S)GlcNAcβ1-6]GalNAc, GlcNAca1-4Galβ1-3(NeuAcα2-6)GalNAc, NeuAcα2-3(GalNAcβ1-4)Galβ1-3GalNAc, GalNAcβ1-4(NeuGcα2-3)Galβ1-3GalNAc, NeuGcα2-3Galβ1-3(GlcNAcβ1-6)GalNAc, Fucα1-2Galβ1-3(Galβ1-4GlcNAcβ1-6)GalNAc, Galβ1-3(Fucα1-2Galβ1-4GlcNAcβ1-6)GalNAc, Galα1-3Galβ1-4GlcNAcβ1-3Galβ1-3GalNAc, Galβ1-3(Galα1-3Galβ1-4GlcNAcβ1-6)GalNAc, Fucα1-2(GalNAcα1-3)Galβ1-4GlcNAcβ1-3GalNAc, GlcNAcβ1-3(Fucα1-2Galβ1-4GlcNAcβ1-6)GalNAc, GlcNAcα1-4Galβ1-3(Galβ1-4GlcNAcβ1-6)GalNAc, NeuAcα2-3Galβ1-3[(6S)GlcNAcβ1-6]GalNAc, NeuAcα2-3Galβ1-3(NeuAcα2-6)GalNAc, NeuGcα2-6Galβ1-3(6SGlcNAcβ1-6)GalNAc, Fucα1-2Galβ1-3[Galβ1-4(6S)GlcNAcβ1-6]GalNAc, Galβ1-3[Fucα1-2Galβ1-4(6S)GlcNAcβ1-6]GalNAc, NeuAcα2-3Galβ1-3(NeuGcα2-6)GalNAc, NeuGcα2-3Galβ1-3(NeuGcα2-6)GalNAc, Fucα1-2Galβ1-4(6S)GlcNAcβ1-3(GlcNAcβ1-6)GalNAc, Fucα1-2Galβ1-4GlcNAcβ-3(6SGlcNAcβ1-6)GalNAc, GalNAcα1-3(Fucα1-2)Galβ1-3(6SGlcNAcβ1-6)GalNAc, GlcNAcβ1-3[Fucα1-2Galβ1-4(6S)GlcNAcβ1-6]GalNAc, Fucα1-2Galβ1-4GlcNAcβ1-3(NeuAcα2-6)GalNAc, Galβ1-4GlcNAcβ1-3[Galβ1-4(6S)GlcNAcβ1-6]GalNAc, Fucα1-2Galβ1-4GlcNAcβ1-3(NeuGcα2-6)GalNAc, NeuAcα2-3Galβ1-3(Galβ1-4GlcNAcβ1-6)GalNAc, Fucα1-2Galβ1-3(Fucα1-2Galβ1-4GlcNAcβ1-6)GalNAc, NeuGcα2-3Galβ1-3(Galβ1-4GlcNAcβ1-6)GalNAc, Galβ1-3[Fucα1-2(GalNAcα1-3)Galβ1-4GlcNAcβ1-6]GalNAc, NeuAcα2-3Galβ1-3[Galβ1-4(6S)GlcNAcβ1-6]GalNAc, Fucα1-2Galβ1-3[Fucα1-2Galβ1-4(6S)GlcNAcβ1-6]GalNAc, NeuGcα2-3Galβ1-3[Galβ1-4(6S)GlcNAcβ1-6]GalNAc, GlcNAcβ1-3[GalNAcα1-3(Fucα1-2)Galβ1-3GlcNAcβ1-6]GalNAc, NeuAcα2-3(GalNAcβ1-4)Galβ1-3(6SGlcNAcβ1-6)GalNAc, 6SGlcNAcβ1-3[Fucα1-2Galβ1-(Fucα1-)GlcNAcβ1-6]GalNAc, NeuAcα2-3(GalNAcβ1-4)Galβ1-3(NeuAcα2-6)GalNAc, GlcNAcβ1-3[NeuGcα2-3Galβ1-4(6S)GlcNAcβ1-6]GalNAc, Fucα1-2Galβ1-4GlcNAcβ1-3[Galβ1-4(6S)GlcNAcβ1-6]GalNAc, Galβ1-3[GalNAcα1-3(Fucα1-2)Galβ1-4(6S)GlcNAcβ1-6]GalNAc, Galβ1-3GlcNAcβ1-3[Fucα1-2Galβ1-4(6S)GlcNAcβ1-6]GalNAc, Galβ1-4GlcNAcβ1-3[Fucα1-2Galβ1-3(6S)GlcNAcβ1-6]GalNAc, NeuAcα2-3Galβ1-3(Fucα1-2Galβ1-4GlcNAcβ1-6)GalNAc, NeuGcα2-3(GalNAcβ1-4)Galβ1-3(NeuGcα2-6)GalNAc, NeuGcα2-3Galβ1-3 (Fucα1-2Galβ1-4GlcNAcβ1-6)GalNAc, Galα1-3Galβ1-3(NeuGcα2-3Galβ1-4GlcNAcβ1-6)GalNAc, GalNAcα1-3(Fucα1-2)Galβ1-3GlcNAcβ1-3(6SGlcNAcβ1-6)GalNAc, GlcNAcβ1-3[GalNAcα1-3(Fucα1-2)Galβ1-(6S)GlcNAcβ1-6]GalNAc, GlcNAcα1-4(Fucα1-2)Galβ1-4GlcNAcβ1-3(NeuAcα2-6)GalNAc, Fucα1-2Galβ1-4GlcNAcβ1-3(Fucα1-2Galβ1-4GlcNAcβ1-6)GalNAc, Galβ1-(Fuc)GlcNAcβ1-3(Fucα1-2Galβ1-4GlcNAcβ1-6)GalNAc, NeuGcα2-3Galβ1-3(GlcNAcα1-4Galβ1-4GlcNAcβ1-6)GalNAc, NeuAcα2-3Galβ1-3[Fucα1-2Galβ1-4(6S)GlcNAcβ1-6]GalNAc, Fucα1-2Galβ1-3[GalNAcα1-3(Fucα1-2)Galβ1-3(6S)GlcNAcβ1-6]GalNAc, Fucα1-2Galβ1-3[GalNAcα1-3(Fucα1-2)Galβ1-4(6S)GlcNAcβ1-6]GalNAc, Fucα1-2Galβ1-4GlcNAcβ1-3[Fucα1-2Galβ1-4(6S)GlcNAcβ1-6]GalNAc, NeuAcα2-3Galβ1-3[GalNAcα1-3(Fucα1-2)Gal-(6S)GlcNAcβ1-6]GalNAc, GlcNAcα1-4Galβ1-3[NeuGcα2-6Gal-(Fuc)(6S)GlcNAcβ1-6]GalNAc, GlcNAcα1-4(Fucα1-2)GlcNAcβ1-3[Fucα1-2)Galβ1-4(6S)GlcNAcβ1-6]GalNAc, GalNAcα1-3(Fucα1-2)Galβ1-4GlcNAcβ1-3[GalNAcα1-3(Fucα1-2)Galβ1-4(6S)GlcNAcβ1-6]GalNAc, and NeuAcα2-Galβ1-4GlcNAcβ1-2Manα1-3(Galβ1-4GlcNAcβ1-2Manα1-6)Manβ1-4GlcNAcβ1-4(Fucα1-6)GlcNAc, and
a checkpoint inhibitor.

53. The composition of claim 52, wherein the composition comprises glycopeptide-bound oligosaccharides having at least 20 of the general formulae or
wherein the composition comprises at least 10 of the general formulae, and each formula is present in the composition between 0.1% and 10% of all glycopeptide-bound oligosaccharides in the composition.

54.-60. (canceled)
